(19) United States
(12) Patent Application Publication
(10) Pub. No.: US 2025/0188068 A1
(43) Pub. Date: Jun. 12, 2025

(54) AMIDO CYCLOPROPYL DERIVATIVES AS LPA RECEPTOR INHIBITORS

(71) Applicant: CHIESI FARMACEUTICI S.P.A., Parma (IT)

(72) Inventors: Elisabetta ARMANI, Parma (IT); Gabriele AMARI, Parma (IT); Mafalda PAGANO, Parma (IT); Marta GIULIANI, Parma (IT)

(73) Assignee: CHIESI FARMACEUTICI S.P.A., Parma (IT)

(21) Appl. No.: 18/844,467

(22) PCT Filed: Mar. 7, 2023
(86) PCT No.: PCT/EP2023/055672

§ 371 Date: Sep. 6, 2024

(30) Foreign Application Priority Data
Mar. 8, 2022 (EP) 22160914.2

(51) Int. Cl. C07D 413/14; A61K 31/381; A61K 31/4192; A61K 31/42; A61K 31/421; A61K 31/422; A61K 31/4436; A61K 31/4439; A61K 31/497; A61K 31/506; C07D 207/34; C07D 249/06; C07D 261/08; C07D 261/14; C07D 263/48; C07D 333/36; C07D 401/04; C07D 409/04; C07D 413/04; C07D 413/12; C07D 417/04; C07D 417/14

(52) U.S. Cl. CPC C07D 413/14; A61K 31/381; A61K 31/4192; A61K 31/42; A61K 31/421; A61K 31/422; A61K 31/4436; A61K 31/4439; A61K 31/497; A61K 31/506; C07D 207/34; C07D 249/06; C07D 261/08; C07D 261/14; C07D 263/48; C07D 333/36; C07D 401/04; C07D 409/04; C07D 413/04; C07D 413/12; C07D 417/04; C07D 417/14

(57) ABSTRACT

The present invention relates to compounds of general formula (I) inhibiting lysophosphatidic acid receptor 1 (LPA1), particularly the invention relates to compounds that are amido cyclopropyl derivatives, methods of preparing such compounds, pharmaceutical compositions containing them and therapeutic use thereof. The compounds of the invention may be useful in the treatment of diseases or conditions associated with a dysregulation of LPA receptors, in particular fibrosis.

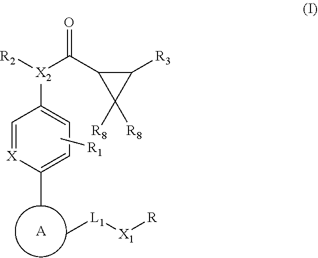

### FIELD OF THE INVENTION

The present invention generally relates to compounds inhibiting lysophosphatidic acid receptors (hereinafter LPA inhibitors); the invention relates to compounds that are amido cyclopropyl derivatives, methods of preparing such compounds, pharmaceutical compositions containing them and therapeutic use thereof.

The compounds of the invention may be useful for instance in the treatment of many disorders associated with LPA receptors mechanisms.

### BACKGROUND OF THE INVENTION

Lysophosphatidic acid (LPA) is a phospholipid mediator concentrated in serum that acts as a potent extracellular signalling molecule through at least six cognate G protein-coupled receptors (GPCRs) in numerous developmental and adult processes including cell survival, proliferation, migration, differentiation, vascular regulation, and cytokine release.

These LPA-mediated processes involve nervous system function, vascular development, immune system function, cancer, reproduction, fibrosis, and obesity (see e.g. Yung et al., J Lipid Res. 2014 July; 55(7):1192-214). The formation of an LPA species depends on its precursor phospholipid, which can vary typically by acyl chain length and degree of saturation. The term LPA generally refers to 18:1 oleoyl-LPA (1-acyl-2-hydroxy-sn-glycero3-phosphate), that is the most quantitatively abundant forms of LPA in human plasma with 16:0-, 18:2-, and 18:1-LPA (see e.g. Sano et al., J Biol Chem. 2002 Dec. 13; 277(50):21197-206). All LPA species are produced from membrane phospholipids via two major metabolic routes. Depending upon the site of synthesis, membrane phospholipids get converted to the corresponding lysophospholipids by the action of phospholipase A1 (PLA1), phospholipase A2 (PLA2), or PLA1 and lecithin-cholesterol acyltransferase (LCAT). Autotoxin (ATX) then acts on the lysophospholipids and converts them into LPA species. The second pathway first converts the phospholipids into phosphatidic acid by the action of phospholipase D. Then PLA1 or PLA2 metabolize phosphatidic acid to the lysophosphatidic acids (see e.g. Riaz et al., Int J Mol Sci. 2016 Feb. 17(2): 215).

ATX activity is the major source of plasma extracellular LPA but the source of tissue LPA that contributes to signalling pools likely involves not only ATX but other enzymes as well. The biological functions of LPA are mediated by at least six recognized cell-surface receptors.

All LPA receptors are rhodopsin-like 7-TM proteins that signal through at least two of the four Gα subunit families (Gα12/13, Gαq/11, Gαi/o and GαS). LPA receptors usually trigger response from multiple heterotrimeric G-proteins, resulting in diverse outcomes in a context and cell type dependent manner. Gα12/13-mediated LPA signalling regulates cell migration, invasion and cytoskeletal re-adjustments through activation of RHO pathway proteins. RAC activation downstream of Gαi/o-PI3K also regulates similar processes, but the most notable function of LPA-induced Gαi/o is mitogenic signalling through the RAF-MEK-MAPK cascade and survival signalling through the PI3K-AKT pathway. The LPA-coupled Gαq/11 protein primarily regulates Ca2+ homeostasis through PLC and the second messengers IP3 and DAG. Lastly, GαS can activate adenylyl cyclase and increase cAMP concentration upon LPA stimulation (see e.g. Riaz et al., Int J Mol Sci. 2016 February; 17(2): 215).

LPA, especially LPA1, LPA2 and LPA3, have been implicated in migration, invasion, metastasis, proliferation and survival and differ in their tissue distribution and downstream signalling pathways.

LPA1 is a 41-kD protein that is widely expressed, albeit at different levels, in all human adult tissues examined and the importance of LPA1 signalling during development and adult life has been demonstrated through numerous approaches (see e.g. Ye at al., 2002, Neuroreport. December 3; 13(17):2169-75). Wide expression of LPA1 is observed in adult mice, with clear presence in at least brain, uterus, testis, lung, small intestine, heart, stomach, kidney, spleen, thymus, placenta, and skeletal muscle. LPA1 is also widely expressed in humans where the expression is more spatially restricted during embryonic development. LPA1 couples with and activates three types of G proteins: Gαi/o, Gαq/11, and Gα12/13. LPA1 activation induces a range of cellular responses: cell proliferation and survival, cell migration, cytoskeletal changes, Ca2+ mobilization, adenylyl cyclase inhibition and activation of mitogen-activated protein kinase, phospholipase C, Akt, and Rho pathways (see e.g. Choi et al., Annu Rev Pharmacol Toxicol. 2010; 50:157-86).

LPA2 in humans is a 39-kD protein and shares ˜55% amino acid sequence homology with LPA1 (see e.g. Yung et al., J Lipid Res. 2014 July; 55(7):1192-214). In mouse, LPA2 is highly expressed in kidney, uterus, and testis and moderately expressed in lung; in human tissues, high expression of LPA2 is detected in testis and leukocytes, with moderate expression found in prostate, spleen, thymus, and pancreas.

In terms of signalling activity, LPA2 mostly activates the same pathways as triggered by LPA1 with some exceptions that regards its unique cross-talk behaviour. For example, LPA2 promotes cell migration through interactions with focal adhesion molecule TRIP6 (see e.g. Lai Y J, 2005, Mol.Cell.Biol. 25:5859-68), and several PDZ proteins and zinc finger proteins are also reported to interact directly with the carboxyl-terminal tail of LPA2 (see e.g. Lin F T, 2008, Biochim.Biophys.Acta 1781:558-62).

Human LPA3 is a 40-kD protein and shares sequence homology with LPA1 (˜54%) and LPA2 (˜49%). In adult humans LPA3 is highly expressed in heart, pancreas, prostate and testis. Moderate levels of expression are also found in brain, lungs and ovary. Like LPA1 and LPA2 the signalling activity of LPA3 results from its coupling to Gαi/o and Gαq/11 (see e.g. Ishii et al., Mol Pharmacol 58:895-902, 2000). Each LPA has multiple important regulatory functions throughout the body.

As LPA signalling has been strongly implicated in many disease states, great interest has been expressed in developing specific LPA inhibitors (see e.g. Stoddard et el., Biomol Ther (Seoul) 2015 Jan. 23(1):1-11). Different studies have demonstrated a positive role for LPA in the pathogenesis of pulmonary fibrosis (PF), a devastating disease characterized by alveolar epithelial cell injury, accumulation of myofibroblasts and deposition of extracellular matrix proteins leading to a loss of lung function and death (see e.g. Wilson M S, Wynn T A (2009), Mucosal Immunol 2: 103-121).

Evidences showed that lysophosphatidic acid levels dramatically increase in bronchoalveolar lavage fluid of PF patients where it mediates fibroblast migration in the injured lung acting through LPA1 (see e.g. Tager et al., Nat Med. 2008 Jan. 14(1):45-54). In addition, mice lacking LPA1 or LPA2 are markedly protected from fibrosis and mortality in a mouse model of the bleomycin induced pulmonary fibrosis (see e.g. Huang et al., Am J Respir Cell Mol Biol. 2013 December; 49(6): 912-922 and Tager et al., Nat Med. 2008 Jan. 14(1):45-54).

In vitro, LPA1 is known to induce the proliferation and differentiation of lung fibroblasts (see e.g. Shiomi et al., Wound Repair Regen. 2011 Mar.-Apr. 19(2): 229-240), and to augment the fibroblast-mediated contraction of released collagen gels (see e.g. Mio et al., Journal of Laboratory and Clinical Medicine, Volume 139, Issue 1, January 2002, Pages 20-27). In human lung fibroblasts, the knockdown of LPA2 attenuated the LPA-induced expression of TGF-β1 and the differentiation of lung fibroblasts to myofibroblasts, resulting in the decreased expression of different profibrotic markers such as FN, α-SMA, and collagen, as well as decreased activation of extracellular regulated kinase 1/2, Akt, Smad3, and p38 mitogen-activated protein kinase (see e.g. Huang et al., Am J Respir Cell Mol Biol. 2013 December; 49(6): 912-922). Moreover Xu et al., confirmed that the expression of LPA2 was also up-regulated in lungs from bleomycin-challenged mice where it is able to induce the activation of TGF-0 pathway, a key cytokine that play an essential role during the development of the disease, via a RhoA and Rho kinase pathway (see e.g. Xu et al., Am J Pathol. 2009 April; 174(4):1264-79). In in vivo preclinical model, the oral administration of an LPA1 antagonist significantly reduced bleomycin-induced pulmonary fibrosis in mice (Tager et al., Nat Med. 2008 Jan. 14(1):45-54; Swaney et al., Br J Pharmacol. 2010 August; 160(7): 1699-1713), and the intraperitoneal injection of an LPA1/3 antagonist ameliorated irradiation-induced lung fibrosis (see e.g. Gan et al., 2011, Biochem Biophys Res Commun 409: 7-13). In a renal fibrosis model, LPA1 administration of an LPA1 antagonist suppressed renal interstitial fibrosis (see e.g Pradere et al., J Am Soc Nephrol 2007; 18:3110-3118).

Various compounds have been described in the literature as LPA1 or LPA2 antagonist.

WO2019126086 and WO2019126087 (Bristol-Myers Squibb) disclose cyclohexyl acid isoxazole azines as LPA1 antagonists, useful for the treatment of disorder or condition associated with dysregulation of lysophosphatidic acid receptor 1.

WO2019126099 (Bristol-Myers Squibb) discloses isoxazole N-linked carbamoyl cyclohexyl acid as LPA1 antagonist for the treatment of disorder or condition associated with dysregulation of lysophosphatidic acid receptor 1.

WO2019126090 (Bristol-Myers Squibb) discloses triazole N-linked carbamoyl cyclohexyl acids as LPA1 antagonists. The compounds are selective LPA1 receptor inhibitors and are useful for the treatment of disorder or condition associated with dysregulation of lysophosphatidic acid receptor 1.

WO2017223016 (Bristol-Myers Squibb) discloses carbamoyloxymethyl triazole cyclohexyl acids as LPA1 antagonists for the treatment of fibrosis including idiopathic pulmonary fibrosis.

WO2012028243 (Merck) discloses pyrazolopyridinone derivatives according to formula (I) and a process of manufacturing thereof as LPA2 receptor antagonists for the treatment of various diseases.

WO2012100436 (Curegenix) discloses phenyl isoxazole carbamate derivatives as LPA1 antagonists for the treatment of LPA mediated disorder, such as fibrosis.

Amgen Inc. discloses in “Discovery of potent LPA2 (EDG4) antagonists as potential anticancer agents” Bioorg Med Chem Lett. 2008 Feb. 1; 18(3):1037-41, LPA2 antagonists. Key compounds were evaluated in vitro for inhibition of LPA2 mediated Erk activation and proliferation of HCT-116 cells. These compounds could be used as tool compounds to evaluate the anticancer effects of blocking LPA2 signalling.

Of note, antagonizing the LPA receptors may be useful for the treatment of fibrosis and disease, disorder and conditions that result from fibrosis, and antagonizing receptors LPA1 may be efficacious in the treatment of the above-mentioned disease, disorder and conditions.

Despite the above cited prior art, there remains a potential for developing novel inhibitors of receptors LPA1 with a proper permeability profile suitable for the inhalation route useful for the treatment of diseases or conditions associated with a dysregulation of LPA receptors, in particular fibrosis.

In this respect, the state of the art does not describe or suggest amido cyclopropyl derivatives of general formula (I) of the present invention having antagonist activity on receptors LPA1 and at the same time a suitable permeability profile for an inhaled candidate which represent a solution to the aforementioned need.

### SUMMARY OF THE INVENTION

In a first aspect the invention refers to a compound of formula (I)

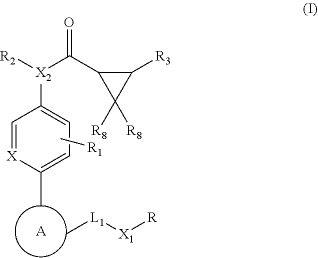

wherein X is —CH— or N,
X₁ is —NH— or —O—,
X₂ is —N— or —CH₂—N—,
L₁ is —(CH₂)ₙ
n is an integer between 0 and 3;
A is selected from the group consisting of

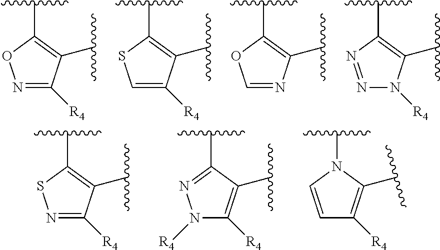

R₁H or —(C₁-C₄)alkyl;
R₂ is H or —(C₁-C₄)alkyl;
R₃ is selected from the group consisting of —C(O)OR₂ or heteroaryl wherein said heteroaryl is optionally substituted by oxo or thioxo,
R₄ is H or —(C₁-C₄)alkyl,
R is selected from the group consisting of —C(O)O(C₁-C₅)alkyl-R₅, —C(O)NR₆R₇, heteroaryl, wherein any of such heteroaryl is optionally substituted by one or more groups selected from —OR₂, —(C₁-C₄)alkyl, —(C₁-C₄)haloalkyl, —(C₃-C₆)cycloalkyl, aryl, heteroaryl;
R₅ is H or selected from the group consisting of —(C₁-C₄)alkyl, aryl, wherein any of such aryl is optionally substituted by one or more groups selected from —(C₁-C₄)alkyl or halo;
R₆ and R₇ are at each occurrence independently H or selected from the group consisting of —(C₁-C₄)alkyl, —(C₃-C₆)cycloalkyl, —(C₁-C₆)haloalkyl and halo,
R₈ is H or halo,
with the proviso that when A is

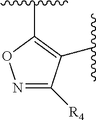

and X is CH, R₂ is —(C₁-C₄)alkyl or R₃ is heteroaryl, or n is 1 or greater than 1.

In a second aspect, the invention refers to pharmaceutical composition comprising a compound of formula (I) in a mixture with one or more pharmaceutically acceptable carrier or excipient.

In a third aspect, the invention refers to a compound of formula (I) for the use as a medicament.

In a further aspect, the invention refers to a compound of formula (I) for use in treating disease, disorder, or condition associated with dysregulation of lysophosphatidic acid receptor 1 (LPA1).

In a further aspect, the invention refers to a compound of formula (I) for use in the prevention and/or treatment of fibrosis and/or diseases, disorders, or conditions that involve fibrosis.

In a further aspect, the invention refers to a compound of formula (I) for use in the prevention and/or treatment idiopathic pulmonary fibrosis (IPF)

### DETAILED DESCRIPTION OF THE INVENTION

Unless otherwise provided, the term compound of formula (I) comprises in its meaning stereoisomer, tautomer or pharmaceutically acceptable salt or solvate.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, iodic acid, formic acid, benzoic acid, sulfuric acid, phosphoric acid, methane sulfonic acid, camphor sulfonic acid, nitric acid, acetic acid, oxalic acid, maleic acid, fumaric acid, succinic acid, p-toluenesulfonic acid, trifluoroacetic acid, 2-naphthoic acid, tartaric acid, 1-hydroxy-2-naphthoic acid, naphthalene-2,7-disulfonic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and Diastereomers are examples of stereoisomers.

The term “enantiomer” refers to one of a pair of molecular species that are mirror images of each other and are not superimposable.

The term “Diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “5-membered heterocyclyl” refers to a mono satured or unsatured group containing one or more heteroatoms selected from N and O.

The term “(Cₓ-C_{y}) alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y}) alkylene” wherein x and y are integers, refers to a Cₓ-C_{y}alkyl radical having in total two unsatisfied valences, such as a divalent methylene radical.

The expressions “(Cₓ-C_{y}) haloalkyl” wherein x and y are integers, refer to the above defined “(Cₓ-C_{y}) alkyl” groups wherein one or more hydrogen atoms are replaced by one or more halogen atoms, which can be the same or different.

Examples of said “(Cₓ-C_{y}) haloalkyl” groups may thus include halogenated, poly-halogenated and fully halogenated alkyl groups wherein all hydrogen atoms are replaced by halogen atoms, e.g. trifluoromethyl.

The term “(Cₓ-C_{y}) cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono cyclic carbon ring systems which have 6 ring atoms wherein the ring is aromatic. Examples of suitable aryl monocyclic ring systems include, for instance, phenyl.

The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond.

A bond pointing to a wavy or squiggly line, such as

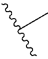

as used in structural formulas herein, depicts the bond that is the point of attachment of the moiety or substituent to the core or backbone structure.

A dash (“-”) that is not between two letters or symbols is meant to represent the point of attachment for a substituent.

The term “IC₅₀” refers to the half maximal inhibitory concentration as a measure of the potency of a substance in inhibiting a specific biological or biochemical function.

Whenever basic amino or quaternary ammonium groups are present in the compounds of formula I, physiologically acceptable anions may be present, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate, emipamoate, xinafoate and naphthalene disulfonate. Likewise, in the presence of acidic groups such as COOH groups, corresponding physiological cation salts may be present as well, for instance including alkaline or alkaline earth metal ions.

As above indicated, the present invention refers to a series of compounds represented by the general formula (I) as herein below described in details, which are endowed with an antagonist property versus receptor LPA1.

Differently from similar compounds of the prior art, the compounds of formula (I) of the present invention are able to act as antagonist LPA1 in a substantive and effective way, particularly appreciated by the skilled person when looking at a suitable and efficacious compounds useful for the treatment of fibrosis, in particular idiopatic pulmonary fibrosis.

As indicated in the experimental part, the compounds of formula (I) of the invention have an activity as shown in Table 4, wherein for each compound is reported the potency expressed as half maximal inhibitory concentration (IC₅₀) on receptors.

As it can be appreciated, all the compounds of the present invention according to Table 4, show a potency with respect to their inhibitory activity on receptor LPA1 below 200 nM, preferably below 100 nM and more preferably below 50 nM.

More advantageously, the compounds of formula (I) of the present invention are also endowed with a proper permeability profile that, in its turn, can ensure a suitable bioavailability for an administration by inhalation. The permeability was assessed in human Caco 2 cell line, an in vitro model that mimic human gastrointestinal barrier and so useful to predic oral absorption. A passive permeability value ≤5 nm/sec is considered suitable for an oral administration, as shown in Table 5.

Thus, in one aspect the present invention relates to a compound of general formula (I) as LPA1 antagonist

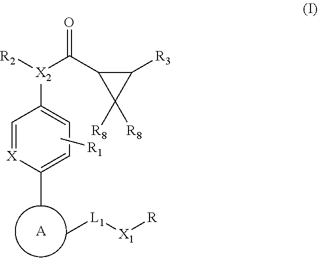

wherein X is —CH— or N,
X₁ is —NH— or —O—,
X₂ is —N— or —CH₂—N—,
L₁ is —(CH₂)ₙ
n is an integer between 0 and 3;
A is selected from the group consisting of

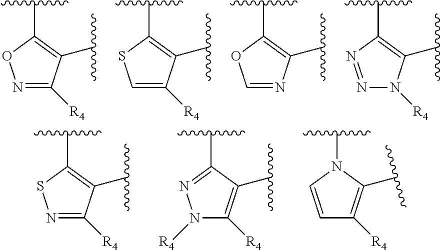

R₁H or —(C₁-C₄)alkyl;
R₂ is H or —(C₁-C₄)alkyl;
R₃ is selected from the group consisting of —C(O)OR₂ or heteroaryl, wherein said heteroaryl is optionally substituted by oxo or thioxo;
R₄ is H or (C₁-C₄)alkyl;
R is selected from the group consisting of —C(O)O(C₁-C₅)alkyl-R₅, —C(O)NR₆R₇, heteroaryl, wherein any of such heteroaryl is optionally substituted by one or more groups selected from —OR₂, —(C₁-C₄)alkyl, —(C₁-C₄)haloalkyl, —(C₃-C₆)cycloalkyl, aryl, heteroaryl;
R₅ is H or selected from the group consisting of —(C₁-C₄)alkyl, aryl, wherein any of such aryl is optionally substituted by one or more groups selected from —(C₁-C₄)alkyl or halo;
R₆ and R₇ are at each occurrence independently H or selected from the group consisting of —(C₁-C₄)alkyl, —(C₃-C₆)cycloalkyl, —(C₁-C₆)haloalkyl and halo;
R₈ is H or halo,
with the proviso that when A is

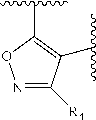

and X is CH, R₂ is —(C₁-C₄)alkyl or R₃ is heteroaryl, or n is 1 or greater than 1.

The invention further concerns the corresponding deuterated derivatives of compounds of formula (I).

In a preferred embodiment, the invention refers to at least one of the compounds listed in the Table 1 below and pharmaceutical acceptable salts thereof

TABLE 1
List of preferred compounds of Formula (I)

| Cmpd. No. | Structure | Name |
|---|---|---|
| Compound 1 | 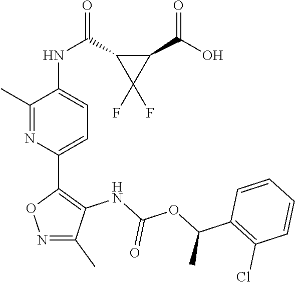 [SMILES: Cc1nc(-c2onc(C)c2NC(=O)O[C@H](C)c2ccccc2Cl)ccc1NC(=O)[C@@H]1[C@@H](C(=O)O)C1(F)F] | (1S,3S)-3-((6-(4-((((R)-1-(2- chlorophenyl)ethoxy)carbon- yl)amino)-3-methylisoxazol- 5-yl)-2-methylpyridin-3-yl)- carbamoyl)-2,2-difluorocyclo- propane-1-carboxylic acid |
| Compound 2 | 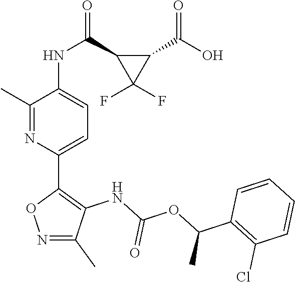 [SMILES: Cc1nc(-c2onc(C)c2NC(=O)O[C@H](C)c2ccccc2Cl)ccc1NC(=O)[C@H]1[C@H](C(=O)O)C1(F)F] | (1R,3R)-3-((6-(4-((((R)-1-(2- chlorophenyl)ethoxy)carbon- yl)amino)-3-methylisoxazol- 5-yl)-2-methylpyridin-3-yl)- carbamoyl)-2,2-difluoro- cyclo-propane-1-carboxylic acid |
| Compound 3 | 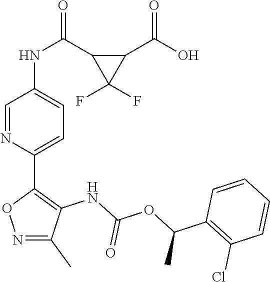 [SMILES: Cc1noc(-c2ccc(NC(=O)C3C(C(=O)O)C3(F)F)cn2)c1NC(=O)O[C@H](C)c1ccccc1Cl] | 3-((6-(4-((((R)-1-(2-chloro- phenyl)ethoxy)carbonyl)- amino)-3-methylisoxazol-5- yl)pyridin-3-yl)carbamoyl)- 2,2-difluorocyclopropane-1- carboxylic acid, mix of Trans diastereomers |
| Compound 4 | 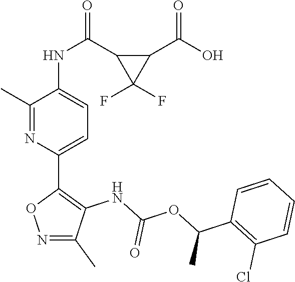 [SMILES: Cc1nc(-c2onc(C)c2NC(=O)O[C@H](C)c2ccccc2Cl)ccc1NC(=O)C1C(C(=O)O)C1(F)F] | 3-((6-(4-((((R)-1-(2-chloro- phenyl)ethoxy)carbonyl)- amino)-3-methylisoxazol-5- yl)-2-methylpyridin-3-yl)- carbamoyl)-2,2-difluoro- cyclopropane-1-carboxylic acid, mix of Trans diastereo- mers |
| Compound 5 | 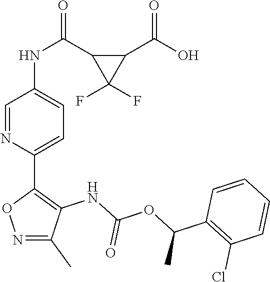 [SMILES: Cc1noc(-c2ccc(NC(=O)C3C(C(=O)O)C3(F)F)cn2)c1NC(=O)O[C@H](C)c1ccccc1Cl] | Single Diastereomer 1 of Trans-3-((6-(4-((((R)-1-(2- chlorophenyl)ethoxy)carbon- yl)amino)-3-methylisoxazol- 5-yl)pyridin-3-yl)carbamoyl)- 2,2-difluorocyclopropane-1- carboxylic acid |
| Compound 6 | 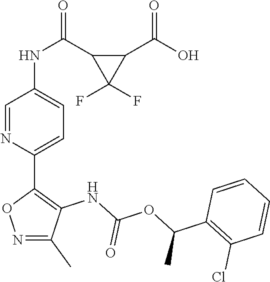 [SMILES: Cc1noc(-c2ccc(NC(=O)C3C(C(=O)O)C3(F)F)cn2)c1NC(=O)O[C@H](C)c1ccccc1Cl] | Single Diastereomer 2 of Trans-3-((6-(4-((((R)-1-(2- chlorophenyl)ethoxy)carbon- yl)amino)-3-methylisoxazol- 5-yl)pyridin-3-yl)carbamoyl)- 2,2-difluorocyclopropane-1- carboxylic acid |
| Compound 7 | 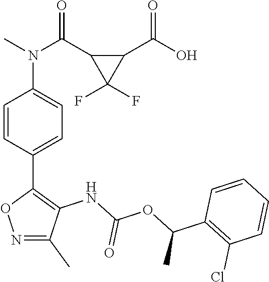 [SMILES: Cc1noc(-c2ccc(N(C)C(=O)C3C(C(=O)O)C3(F)F)cc2)c1NC(=O)O[C@H](C)c1ccccc1Cl] | 3-((4-(4-((((R)-1-(2-chloro- phenyl)ethoxy)carbonyl)- amino)-3-methylisoxazol-5- yl)phenyl)(methyl)carbamo- yl)-2,2-difluorocyclopropane- 1-carboxylic acid, mix of Trans diastereomers |
| Compound 8 | 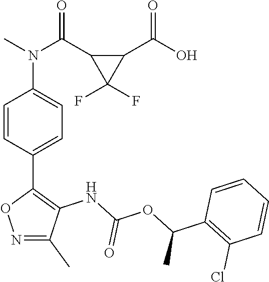 [SMILES: Cc1noc(-c2ccc(N(C)C(=O)C3C(C(=O)O)C3(F)F)cc2)c1NC(=O)O[C@H](C)c1ccccc1Cl] | Single Diastereomer 1 of Trans 3-((4-(4-((((R)-1-(2- chlorophenyl)ethoxy)carbon- yl)amino)-3-methylisoxazol- 5-yl)phenyl)(methyl)car- bamoyl)-2,2-difluorocyclo- propane-1-carboxylic acid |
| Compound 9 | 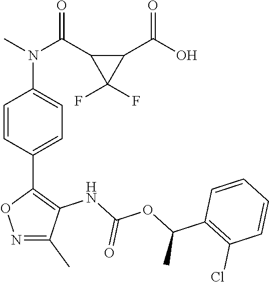 [SMILES: Cc1noc(-c2ccc(N(C)C(=O)C3C(C(=O)O)C3(F)F)cc2)c1NC(=O)O[C@H](C)c1ccccc1Cl] | Single Diastereomer 2 of Trans 3-((4-(4-((((R)-1-(2- chlorophenyl)ethoxy)carbon- yl)amino)-3-methylisoxazol- 5-yl)phenyl)(methyl)car- bamoyl)-2,2-difluorocyclo- propane-1-carboxylic acid |
| Compound 10 | 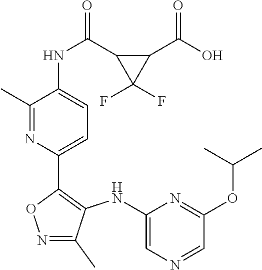 [SMILES: Cc1nc(-c2onc(C)c2Nc2cncc(OC(C)C)n2)ccc1NC(=O)C1C(C(=O)O)C1(F)F] | 2,2-difluoro-3-((6-(4-((6- isopropoxypyrazin-2-yl)- amino)-3-methylisoxazol-5- yl)-2-methylpyridin-3-yl)- carbamoyl)cyclopropane-1- carboxylic acid, mix of Trans diastereomers |
| Compound 11 | 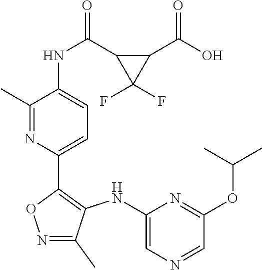 [SMILES: Cc1nc(-c2onc(C)c2Nc2cncc(OC(C)C)n2)ccc1NC(=O)C1C(C(=O)O)C1(F)F] | Single Enantiomer 2 of Trans-2,2-difluoro-3-((6-(4- ((6-isopropoxypyrazin-2-yl)- amino)-3-methylisoxazol-5- yl)-2-methylpyridin-3-yl)- carbamoyl)cyclopropane-1- carboxylic acid |
| Compound 12 | 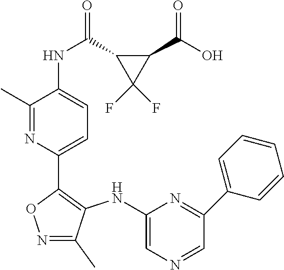 [SMILES: Cc1nc(-c2onc(C)c2Nc2cncc(-c3ccccc3)n2)ccc1NC(=O)[C@@H]1[C@@H](C(=O)O)C1(F)F] | (1S,3S)-2,2-difluoro-3-((2- methyl-6-(3-methyl-4-((6- phenylpyrazin-2-yl)amino)- isoxazol-5-yl)pyridin-3-yl)- carbamoyl)cyclopropane-1- carboxylic acid |
| Compound 13 | 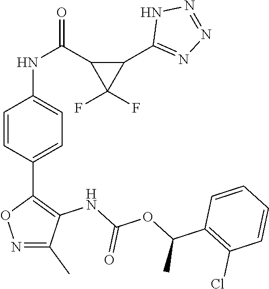 [SMILES: Cc1noc(-c2ccc(NC(=O)C3C(c4nnn[nH]4)C3(F)F)cc2)c1NC(=O)O[C@H](C)c1ccccc1Cl] | (R)-1-(2-chlorophenyl)ethyl (5-(4-(2,2-difluoro-3-(1H- tetrazol-5-yl)cyclopropane-1- carboxamido)phenyl)-3-meth- ylisoxazol-4-yl)carbamate, mix of Trans diastereomers |
| Compound 14 | 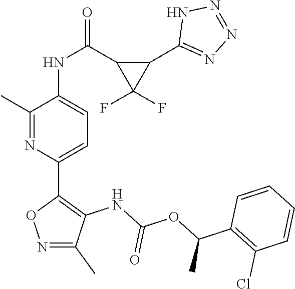 [SMILES: Cc1nc(-c2onc(C)c2NC(=O)O[C@H](C)c2ccccc2Cl)ccc1NC(=O)C1C(c2nnn[nH]2)C1(F)F] | (R)-1-(2-chlorophenyl)ethyl (5-(5-(2,2-difluoro-3-(1H- tetrazol-5-yl)cyclopropane-1- carboxamido)-6-methylpyr- idin-2-yl)-3-methylisoxazol- 4-yl)carbamate, mix of Trans diastereomers |
| Compound 15 | 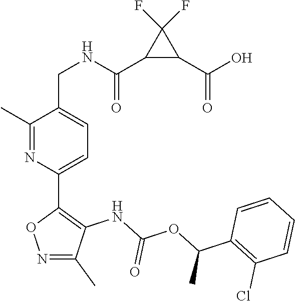 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1- carboxylic acid, mix of Cis and Trans diastereomers |
| Compound 16 | 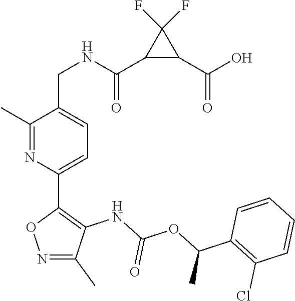 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1- carboxylic acid, mix of Cis diastereomers |
| Compound 17 | 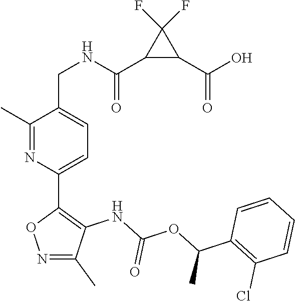 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1- carboxylic acid, mix of Trans diastereomers |
| Compound 18 | 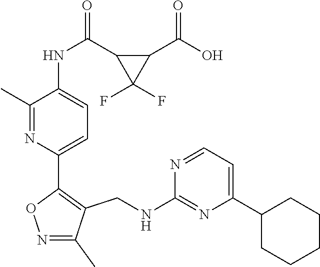 [SMILES: Cc1nc(-c2onc(C)c2CNc2nccc(C3CCCCC3)n2)ccc1NC(=O)C1C(C(=O)O)C1(F)F] | 3-((6-(4-(((4-cyclohexylpyr- imidin-2-yl)amino)methyl)- 3-methylisoxazol-5-yl)-2- methylpyridin-3-yl)carbamo- yl)-2,2-difluorocyclopropane- 1-carboxylic acid, mix of Trans diastereomers |
| Compound 19 | 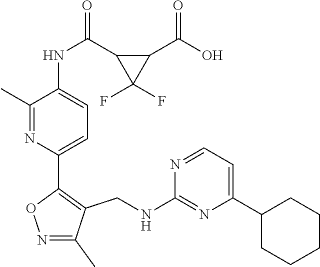 [SMILES: Cc1nc(-c2onc(C)c2CNc2nccc(C3CCCCC3)n2)ccc1NC(=O)C1C(C(=O)O)C1(F)F] | Single Enantiomer 1 of Trans-3-((6-(4-(((4-cyclo- hexylpyrimidin-2-yl)amino)- methyl)-3-methylisoxazol-5- yl)-2-methylpyridin-3-yl)- carbamoyl)-2,2-difluorocyclo- propane-1-carboxylic acid |
| Compound 20 | 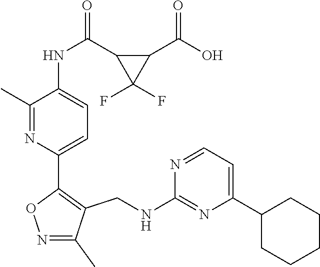 [SMILES: Cc1nc(-c2onc(C)c2CNc2nccc(C3CCCCC3)n2)ccc1NC(=O)C1C(C(=O)O)C1(F)F] | Single Enantiomer 2 of Trans- 3-((6-(4-(((4-cyclohexylpyr- imidin-2-yl)amino)methyl)-3- methylisoxazol-5-yl)-2-meth- ylpyridin-3-yl)carbamoyl)- 2,2-difluorocyclopropane-1- carboxylic acid |
| Compound 21 | 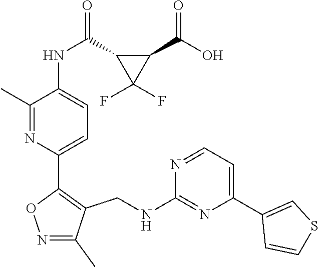 [SMILES: Cc1nc(-c2onc(C)c2CNc2nccc(-c3ccsc3)n2)ccc1NC(=O)[C@@H]1[C@@H](C(=O)O)C1(F)F] | (1S,3S)-2,2-difluoro-3-((2- methyl-6-(3-methyl-4-(((4- (thiophen-3-yl)pyrimidin-2- yl)amino)methyl)isoxazol-5- yl)pyridin-3-yl)carbamoyl)- cyclopropane-1-carboxylic acid |
| Compound 22 | 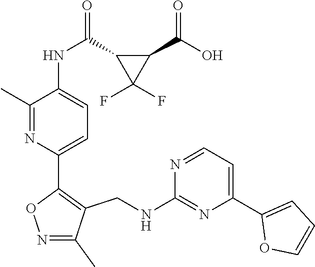 [SMILES: Cc1nc(-c2onc(C)c2CNc2nccc(-c3ccco3)n2)ccc1NC(=O)[C@@H]1[C@@H](C(=O)O)C1(F)F] | (1S,3S)-2,2-difluoro-3-((6-(4- (((4-(furan-2-yl)pyrimidin-2- yl)amino)methyl)-3-methyl- isoxazol-5-yl)-2-methylpyr- idin-3-yl)carbamoyl)cyclopro- pane-1-carboxylic acid |
| Compound 23 | 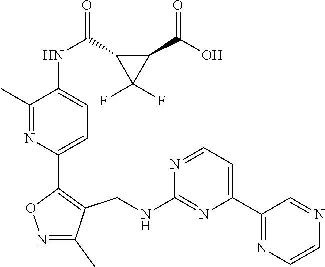 [SMILES: Cc1nc(-c2onc(C)c2CNc2nccc(-c3cnccn3)n2)ccc1NC(=O)[C@@H]1[C@@H](C(=O)O)C1(F)F] | (1S,3S)-2,2-difluoro-3-((2- methyl-6-(3-methyl-4-(((4- (pyrazin-2-yl)pyrimidin-2-yl)- amino)methyl)isoxazol-5-yl)- pyridin-3-yl)carbamoyl)cyclo- propane-1-carboxylic acid |
| Compound 24 | 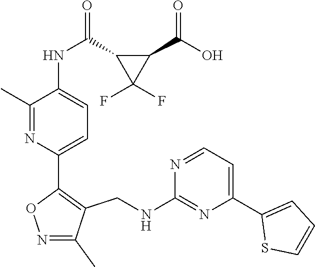 [SMILES: Cc1nc(-c2onc(C)c2CNc2nccc(-c3cccs3)n2)ccc1NC(=O)[C@@H]1[C@@H](C(=O)O)C1(F)F] | (1S,3S)-2,2-difluoro-3-((2- methyl-6-(3-methyl-4-(((4- (thiophen-2-yl)pyrimidin-2- yl)amino)methyl)isoxazol-5- yl)pyridin-3-l)carbamoyl)- cyclopropane-1-carboxylic acid |
| Compound 25 | 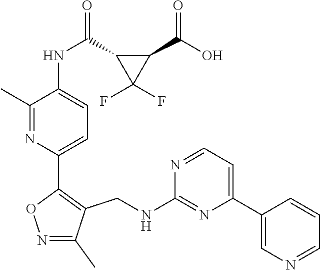 [SMILES: Cc1nc(-c2onc(C)c2CNc2nccc(-c3cccnc3)n2)ccc1NC(=O)[C@@H]1[C@@H](C(=O)O)C1(F)F] | (1S,3S)-2,2-difluoro-3-((2- methyl-6-(3-methyl-4-(((4- (pyridin-3-yl)pyrimidin-2-yl)- amino)methyl)isoxazol-5-yl)- pyridin-3-yl)carbamoyl)cyclo- propane-1-carboxylic acid |
| Compound 26 | 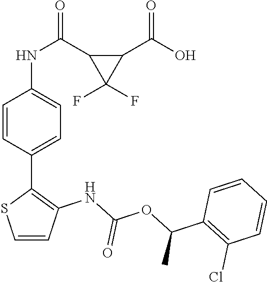 [SMILES: C[C@@H](OC(=O)Nc1ccsc1-c1ccc(NC(=O)C2C(C(=O)O)C2(F)F)cc1)c1ccccc1Cl] | 3-((4-(3-((((R)-1-(2-chloro- phenyl)ethoxy)carbonyl)- amino)thiophen-2-yl)phenyl)- carbamoyl)-2,2-difluorocyclo- propane-1-carboxylic acid, mix of Trans diastereomers |
| Compound 27 | 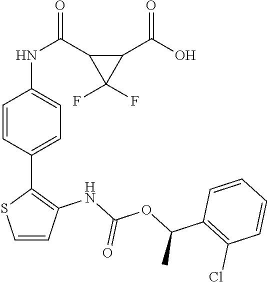 [SMILES: C[C@@H](OC(=O)Nc1ccsc1-c1ccc(NC(=O)C2C(C(=O)O)C2(F)F)cc1)c1ccccc1Cl] | Single Diastereomer 2 of Trans-3-((4-(3-((((R)-1-(2- chlorophenyl)ethoxy)carbon- yl)amino)thiophen-2-yl)phen- yl)carbamoyl)-2,2-difluoro- cyclopropane-1-carboxylic acid |
| Compound 28 | 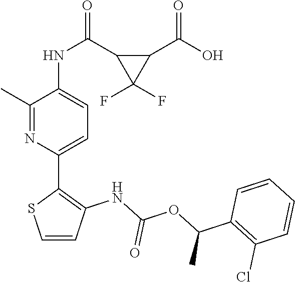 [SMILES: Cc1nc(-c2sccc2NC(=O)O[C@H](C)c2ccccc2Cl)ccc1NC(=O)C1C(C(=O)O)C1(F)F] | 3-((6-(3-((((R)-1-(2-chloro- phenyl)ethoxy)carbonyl)- amino)thiophen-2-yl)-2-meth- ylpyridin-3-yl)carbamoyl)- 2,2-difluorocyclopropane-1- carboxylic acid, mix of Trans diastereomers |
| Compound 29 | 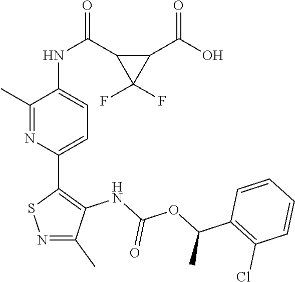 [SMILES: Cc1nc(-c2snc(C)c2NC(=O)O[C@H](C)c2ccccc2Cl)ccc1NC(=O)C1C(C(=O)O)C1(F)F] | 3-((6-(4-((((R)-1-(2-chloro- phenyl)ethoxy)carbonyl)- amino)-3-methylisothiazol-5- yl)-2-methylpyridin-3-yl)- carbamoyl)-2,2-difluorocyclo- propane-1-carboxylic acid, mix of Trans diastereomers |
| Compound 30 | 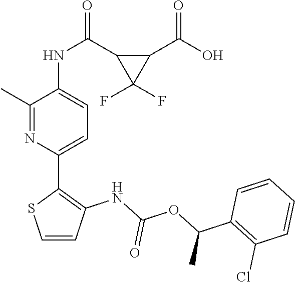 [SMILES: Cc1nc(-c2sccc2NC(=O)O[C@H](C)c2ccccc2Cl)ccc1NC(=O)C1C(C(=O)O)C1(F)F] | Single Diastereomer 2 of Trans-3-((6-(3-((((R)-1-(2- chlorophenyl)ethoxy)carbon- yl)amino)thiophen-2-yl)- 2-methylpyridin-3-yl)car- bamoyl)-2,2-difluorocyclo- propane-1-carboxylic acid |
| Compound 31 | 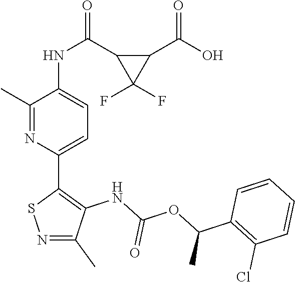 [SMILES: Cc1nc(-c2snc(C)c2NC(=O)O[C@H](C)c2ccccc2Cl)ccc1NC(=O)C1C(C(=O)O)C1(F)F] | Single Diastereomer 1 of Trans-3-((6-(4-((((R)-1-(2- chlorophenyl)ethoxy)carbon- yl)amino)-3-methylisothiazol- 5-yl)-2-methylpyridin-3-yl)- carbamoyl)-2,2-difluoro- cyclopropane-1-carboxylic acid |
| Compound 32 | 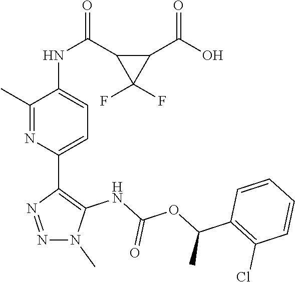 [SMILES: Cc1nc(-c2nnn(C)c2NC(=O)O[C@H](C)c2ccccc2Cl)ccc1NC(=O)C1C(C(=O)O)C1(F)F] | 3-((6-(5-((((R)-1-(2-chloro- phenyl)ethoxy)carbonyl)- amino)-1-methyl-1H-1,2,3- triazol-4-yl)-2-methylpyridin- 3-yl)carbamoyl)-2,2-difluoro- cyclopropane-1-carboxylic acid, mix of Trans diastereo- mers |
| Compound 33 | 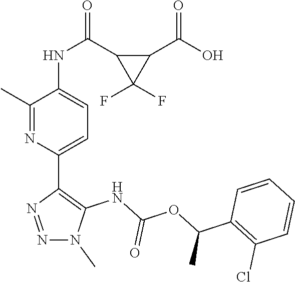 [SMILES: Cc1nc(-c2nnn(C)c2NC(=O)O[C@H](C)c2ccccc2Cl)ccc1NC(=O)C1C(C(=O)O)C1(F)F] | Single Diastereomer 1 of Trans-3-((6-(5-((((R)-1-(2- chlorophenyl)ethoxy)carbon- yl)amino)-1-methyl-1H-1,2,3- triazol-4-yl)-2-methylpyridin- 3-yl)carbamoyl)-2,2-difluoro- cyclopropane-1-carboxylic acid |
| Compound 34 | 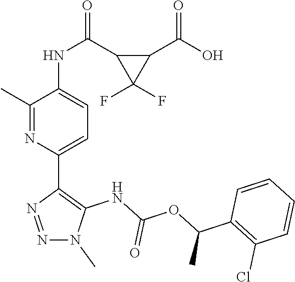 [SMILES: Cc1nc(-c2nnn(C)c2NC(=O)O[C@H](C)c2ccccc2Cl)ccc1NC(=O)C1C(C(=O)O)C1(F)F] | Single Diastereomer 2 of Trans-3-((6-(5-((((R)-1-(2- chlorophenyl)ethoxy)carbon- yl)amino)-1-methyl-1H-1,2,3- triazol-4-yl)-2-methylpyridin- 3-yl)carbamoyl)-2,2-difluoro- cyclopropane-1-carboxylic acid |
| Compound 35 | 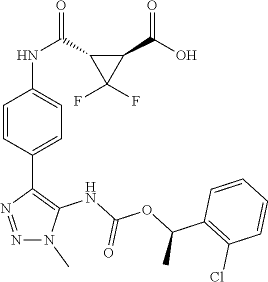 [SMILES: C[C@@H](OC(=O)Nc1c(-c2ccc(NC(=O)[C@@H]3[C@@H](C(=O)O)C3(F)F)cc2)nnn1C)c1ccccc1Cl] | (1S,3S)-3-((4-(5-((((R)-1-(2- chlorophenyl)ethoxy)carbon- yl)amino)-1-methyl-1H-1,2,3- triazol-4-yl)phenyl)carbamo- yl)-2,2-difluorocyclopropane- 1-carboxylic acid |
| Compound 36 | 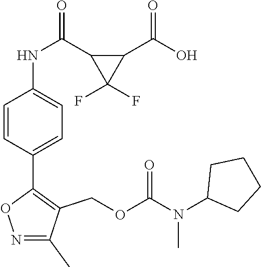 [SMILES: Cc1noc(-c2ccc(NC(=O)C3C(C(=O)O)C3(F)F)cc2)c1COC(=O)N(C)C1CCCC1] | 3-((4-(4-(((cyclopentyl(meth- yl)carbamoyl)oxy)methyl)-3- methylisoxazol-5-yl)phenyl)- carbamoyl)-2,2-difluorocyclo- propane-1-carboxylic acid, mix of Trans diastereomers |
| Compound 37 | 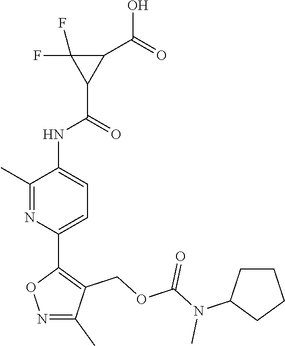 [SMILES: Cc1nc(-c2onc(C)c2COC(=O)N(C)C2CCCC2)ccc1NC(=O)C1C(C(=O)O)C1(F)F] | 3-((6-(4-(((cyclopentyl(meth- yl)carbamoyl)oxy)methyl)-3- methylisoxazol-5-yl)-2-meth- ylpyridin-3-yl)carbamoyl)- 2,2-difluorocyclopropane-1- carboxylic acid, mix of Trans diastereomers |
| Compound 38 | 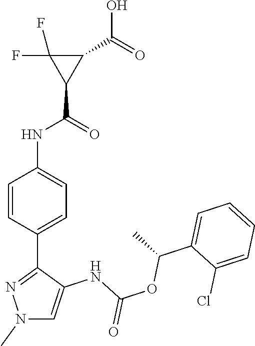 [SMILES: C[C@@H](OC(=O)Nc1cn(C)nc1-c1ccc(NC(=O)[C@@H]2[C@@H](C(=O)O)C2(F)F)cc1)c1ccccc1Cl] | (1S,3S)-3-((4-(4-((((R)-1-(2- chlorophenyl)ethoxy)carbon- yl)amino)-1-methyl-1H-pyr- azol-3-yl)phenyl)carbamoyl)- 2,2-difluorocyclopropane-1- carboxylic acid |
| Compound 39 | 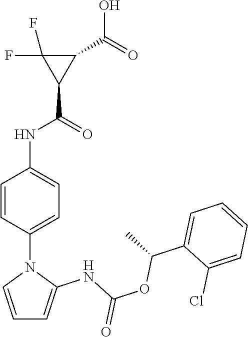 [SMILES: C[C@@H](OC(=O)Nc1cccn1-c1ccc(NC(=O)[C@@H]2[C@@H](C(=O)O)C2(F)F)cc1)c1ccccc1Cl] | (1S,3S)-3-((4-(2-((((R)-1-(2- chlorophenyl)ethoxy)carbon- yl)amino)-1H-pyrrol-1-yl)- phenyl)carbamoyl)-2,2- difluorocyclopropane-1- carboxylic acid |
| Compound 40 | 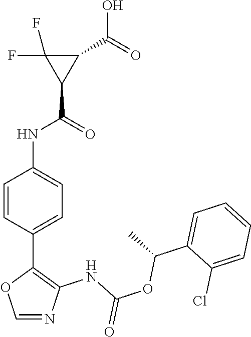 [SMILES: C[C@@H](OC(=O)Nc1ncoc1-c1ccc(NC(=O)[C@@H]2[C@@H](C(=O)O)C2(F)F)cc1)c1ccccc1Cl] | (1S,3S)-3-((4-(4-((((R)-1-(2- chlorophenyl)ethoxy)carbon- yl)amino)oxazol-5-yl)phenyl)- carbamoyl)-2,2-difluorocyclo- propane-1-carboxylic acid |
| Compound 41 | 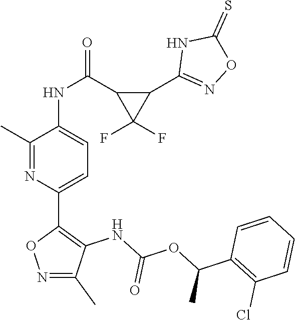 [SMILES: Cc1nc(-c2onc(C)c2NC(=O)O[C@H](C)c2ccccc2Cl)ccc1NC(=O)C1C(c2noc(=S)[nH]2)C1(F)F] | (R)-1-(2-chlorophenyl)ethyl (5-(5-(2,2-difluoro-3-(5- thioxo-4,5-dihydro-1,2,4- oxadiazol-3-yl)cyclopropane- 1-carboxamido)-6-methyl- pyridin-2-yl)-3-methylisox- azol-4-yl)carbamate, mix of Trans diastereomers |
| Compound 42 | 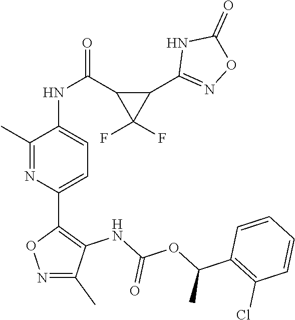 [SMILES: Cc1nc(-c2onc(C)c2NC(=O)O[C@H](C)c2ccccc2Cl)ccc1NC(=O)C1C(c2noc(=O)[nH]2)C1(F)F] | (R)-1-(2-chlorophenyl)ethyl (5-(5-(2,2-difluoro-3-(5-oxo- 4,5-dihydro-1,2,4-oxadiazol- 3-yl)cyclopropane-1-carbox- amido)-6-methylpyridin-2- yl)-3-methylisoxazol-4-yl)- carbamate, mix of Trans diastereomers |
| Compound 43 | 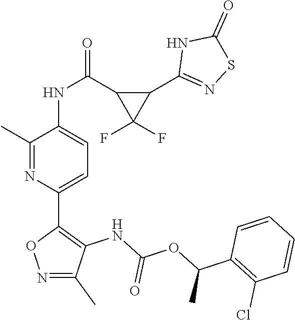 [SMILES: Cc1nc(-c2onc(C)c2NC(=O)O[C@H](C)c2ccccc2Cl)ccc1NC(=O)C1C(c2nsc(=O)[nH]2)C1(F)F] | (R)-1-(2-chlorophenyl)ethyl (5-(5-(2,2-difluoro-3-(5-oxo- 4,5-dihydro-1,2,4-thiadiazol- 3-yl)cyclopropane-1-carbox- amido)-6-methylpyridin-2- yl)-3-methylisoxazol-4-yl)- carbamate, mix of Trans diastereomers |
| Compound 44 | 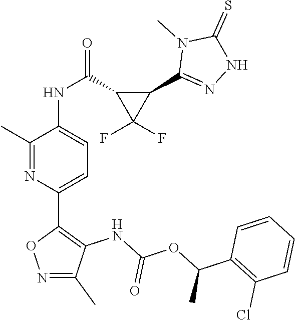 [SMILES: Cc1nc(-c2onc(C)c2NC(=O)O[C@H](C)c2ccccc2Cl)ccc1NC(=O)[C@@H]1[C@@H](c2n[nH]c(=S)n2C)C1(F)F] | (R)-1-(2-chlorophenyl)ethyl (5-(5-((1S,3S)-2,2-difluoro-3- (4-methyl-5-thioxo-4,5- dihydro-1H-1,2,4-triazol-3- yl)cyclopropane-1-carbox- amido)-6-methylpyridin-2- yl)-3-methylisoxazol-4-yl)- carbamate |

In a preferred embodiment, the invention refers to a compound of formula (I) as LPA1 antagonist, wherein X is —N, represented by the general formula (Ia)

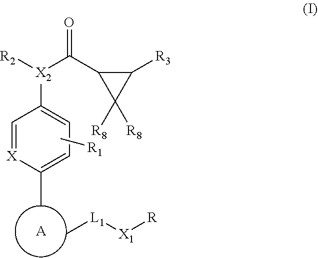

wherein
X₁ is —NH— or —O—;
X₂ is —N— or —CH₂—N—
L₁ is —(CH₂)ₙ
n is an integer between 0 and 3;
A is selected from the group consisting of

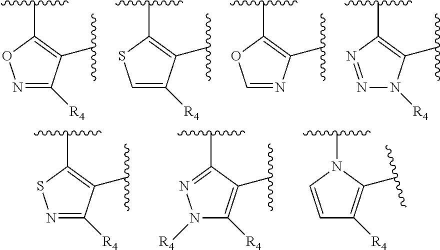

R₁H or —(C₁-C₄)alkyl;
R₂ is H or —(C₁-C₄)alkyl;
R₃ is selected from the group consisting of —C(O)OR₂ or heteroaryl, wherein said heteroaryl is optionally substituted by oxo or thioxo, R₄ is H or —(C₁-C₄)alkyl;
R is selected from the group consisting of —C(O)O(C₁-C₅)alkyl-R₅, —C(O)NR₆R₇, heteroaryl, wherein any of such heteroaryl is optionally substituted by one or more groups selected from —OR₂, (C₁-C₄)alkyl, (C₁-C₄)haloalkyl, (C₃-C₆)cycloalkyl, aryl, heteroaryl;
R₅ is H or selected from the group consisting of —(C₁-C₄)alkyl, aryl, wherein any of such aryl is optionally substituted by one or more groups selected from —(C₁-C₄)alkyl or halo;
R₆ and R₇ are at each occurrence independently H or selected from the group consisting of —(C₁-C₄)alkyl, —(C₃-C₆)cycloalkyl, —(C₁-C₆)haloalkyl and halo;
R₈ is H or halo.

It has been surprisingly found that the above indicated compounds are particularly effective as antagonists of LPA1 receptor, as e.g. indicated in Table 2 of the herein below experimental part.

In a preferred embodiment, the invention refers to at least one of the compounds listed in the Table 2 below and pharmaceutical acceptable salts thereof

TABLE 2
List of preferred compounds of Formula (I)

| Cmpd. No. | Structure | Name |
|---|---|---|
| Compound 1 | 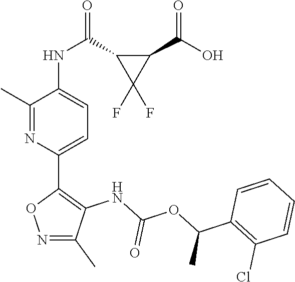 [SMILES: Cc1nc(-c2onc(C)c2NC(=O)O[C@H](C)c2ccccc2Cl)ccc1NC(=O)[C@@H]1[C@@H](C(=O)O)C1(F)F] | (1S,3S)-3-((6-(4-((((R)-1-(2- chlorophenyl)ethoxy)carbon- yl)amino)-3-methylisoxazol- 5-yl)-2-methylpyridin-3-yl)- carbamoyl)-2,2-difluorocyclo- propane-1-carboxylic acid |
| Compound 2 | 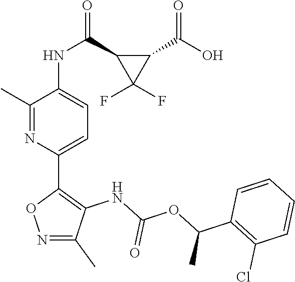 [SMILES: Cc1nc(-c2onc(C)c2NC(=O)O[C@H](C)c2ccccc2Cl)ccc1NC(=O)[C@H]1[C@H](C(=O)O)C1(F)F] | (1R,3R)-3-((6-(4-((((R)-1-(2- chlorophenyl)ethoxy)carbon- yl)amino)-3-methylisoxazol- 5-yl)-2-methylpyridin-3-yl)- carbamoyl)-2,2-difluorocyclo- propane-1-carboxylic acid |
| Compound 3 | 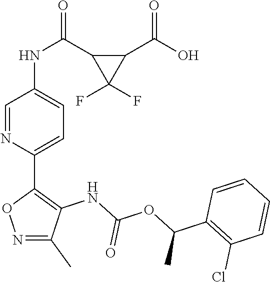 [SMILES: Cc1noc(-c2ccc(NC(=O)C3C(C(=O)O)C3(F)F)cn2)c1NC(=O)O[C@H](C)c1ccccc1Cl] | 3-((6-(4-((((R)-1-(2-chloro- phenyl)ethoxy)carbonyl)- amino)-3-methylisoxazol-5- yl)pyridin-3-yl)carbamoyl)- 2,2-difluorocyclopropane-1- carboxylic acid, mix of Trans diastereomers |
| Compound 4 | 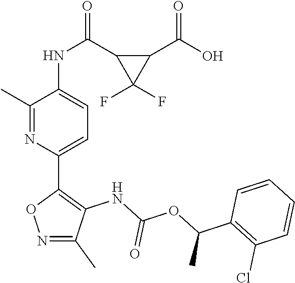 [SMILES: Cc1nc(-c2onc(C)c2NC(=O)O[C@H](C)c2ccccc2Cl)ccc1NC(=O)C1C(C(=O)O)C1(F)F] | 3-((6-(4-((((R)-1-(2-chloro- phenyl)ethoxy)carbonyl)- amino)-3-methylisoxazol-5- yl)-2-methylpyridin-3-yl)- carbamoyl)-2,2-difluorocyclo- propane-1-carboxylic acid, mix of Trans diastereomers |
| Compound 5 | 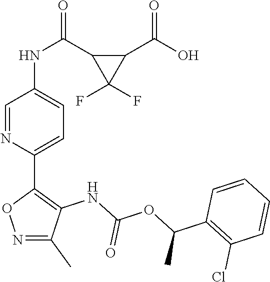 [SMILES: Cc1noc(-c2ccc(NC(=O)C3C(C(=O)O)C3(F)F)cn2)c1NC(=O)O[C@H](C)c1ccccc1Cl] | Single Diastereomer 1 of Trans-3-((6-(4-((((R)-1-(2- chlorophenyl)ethoxy)carbon- yl)amino)-3-methylisoxazol- 5-yl)pyridin-3-yl)carbamo- yl)-2,2-difluorocyclopropane- 1-carboxylic acid |
| Compound 6 | 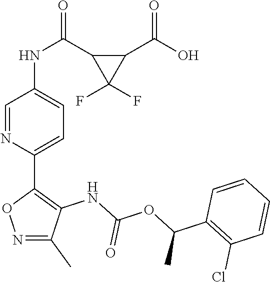 [SMILES: Cc1noc(-c2ccc(NC(=O)C3C(C(=O)O)C3(F)F)cn2)c1NC(=O)O[C@H](C)c1ccccc1Cl] | Single Diastereomer 2 of Trans-3-((6-(4-((((R)-1-(2- chlorophenyl)ethoxy)carbon- yl)amino)-3-methylisoxazol- 5-yl)pyridin-3-yl)carbamoyl)- 2,2-difluorocyclopropane-1- carboxylic acid |
| Compound 10 | 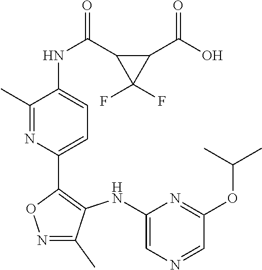 [SMILES: Cc1nc(-c2onc(C)c2Nc2cncc(OC(C)C)n2)ccc1NC(=O)C1C(C(=O)O)C1(F)F] | 2,2-difluoro-3-((6-(4-((6- isopropoxypyrazin-2-yl)- amino)-3-methylisoxazol-5- yl)-2-methylpyridin-3-yl)- carbamoyl)cyclopropane-1- carboxylic acid, mix of Trans diastereomers |
| Compound 11 | 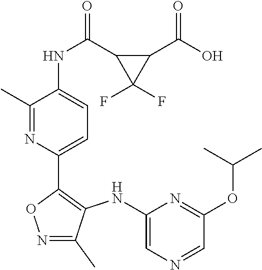 [SMILES: Cc1nc(-c2onc(C)c2Nc2cncc(OC(C)C)n2)ccc1NC(=O)C1C(C(=O)O)C1(F)F] | Single Enantiomer 2 of Trans-2,2-difluoro-3-((6-(4- ((6-isopropoxypyrazin-2-yl)- amino)-3-methylisoxazol-5- yl)-2-methylpyridin-3-yl)- carbamoyl)cyclopropane-1- carboxylic acid |
| Compound 12 | 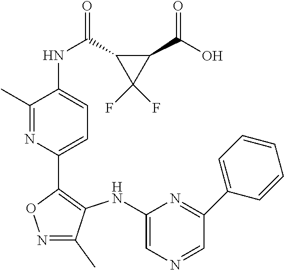 [SMILES: Cc1nc(-c2onc(C)c2Nc2cncc(-c3ccccc3)n2)ccc1NC(=O)[C@@H]1[C@@H](C(=O)O)C1(F)F] | (1S,3S)-2,2-difluoro-3-((2- methyl-6-(3-methyl-4-((6- phenylpyrazin-2-yl)amino)- isoxazol-5-yl)pyridin-3-yl)- carbamoyl)cyclopropane-1- carboxylic acid |
| Compound 14 | 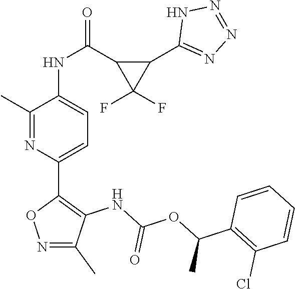 [SMILES: Cc1nc(-c2onc(C)c2NC(=O)O[C@H](C)c2ccccc2Cl)ccc1NC(=O)C1C(c2nnn[nH]2)C1(F)F] | (R)-1-(2-chlorophenyl)ethyl (5-(5-(2,2-difluoro-3-(1H- tetrazol-5-yl)cyclopropane-1- carboxamido)-6-methylpyr- idin-2-yl)-3-methylisoxazol- 4-yl)carbamate, mix of Trans diastereomers |
| Compound 15 | 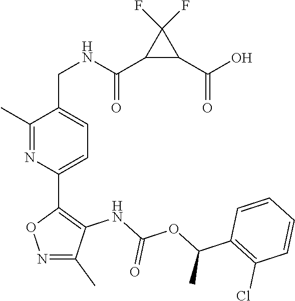 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2 chloro- phenyl)ethoxy)carbonyl)- amino)-3-methylisoxazol-5- yl)-2-methylpyridin-3-yl)- methyl)carbamoyl)-2,2- difluorocyclopropane-1-car- boxylic acid, mix of Cis and Trans diastereomers |
| Compound 16 | 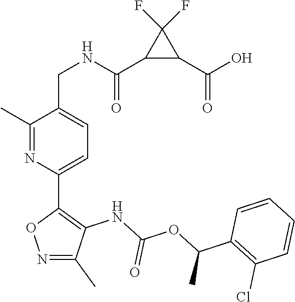 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1-car- boxylic acid, mix of Cis diastereomers |
| Compound 17 | 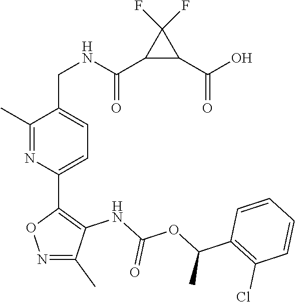 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1-car- boxylic acid, mix of Trans diastereomers |
| Compound 18 | 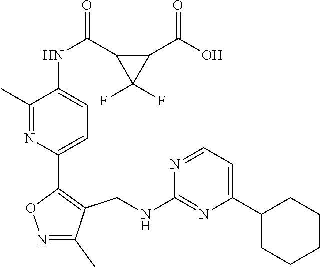 [SMILES: Cc1nc(-c2onc(C)c2CNc2nccc(C3CCCCC3)n2)ccc1NC(=O)C1C(C(=O)O)C1(F)F] | 3-((6-(4-(((4-cyclohexylpyr- imidin-2-yl)amino)methyl)- 3-methylisoxazol-5-yl)-2- methylpyridin-3-yl)car- bamoyl)-2,2-difluorocyclo- propane-1-carboxylic acid, mix of Trans diastereomers |
| Compound 19 | 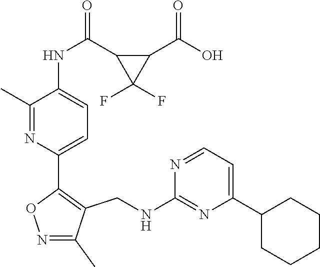 [SMILES: Cc1nc(-c2onc(C)c2CNc2nccc(C3CCCCC3)n2)ccc1NC(=O)C1C(C(=O)O)C1(F)F] | Single Enantiomer 1 of Trans- 3-((6-(4-(((4-cyclohexylpyr- imidin-2-yl)amino)methyl)-3- methylisoxazol-5-yl)-2-meth- ylpyridin-3-yl)carbamoyl)- 2,2-difluorocyclopropane-1- carboxylic acid |
| Compound 20 | 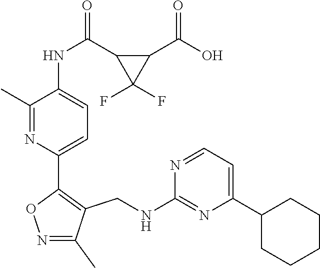 [SMILES: Cc1nc(-c2onc(C)c2CNc2nccc(C3CCCCC3)n2)ccc1NC(=O)C1C(C(=O)O)C1(F)F] | Single Enantiomer 2 of Trans- 3-((6-(4-(((4-cyclohexylpyr- imidin-2-yl)amino)methyl)-3- methylisoxazol-5-yl)-2-meth- ylpyridin-3-yl)carbamoyl)- 2,2-difluorocyclopropane-1- carboxylic acid |
| Compound 21 | 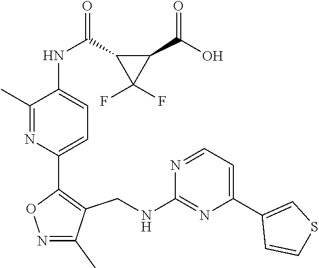 [SMILES: Cc1nc(-c2onc(C)c2CNc2nccc(-c3ccsc3)n2)ccc1NC(=O)[C@@H]1[C@@H](C(=O)O)C1(F)F] | (1S,3S)-2,2-difluoro-3-((2- methyl-6-(3-methyl-4-(((4- (thiophen-3-yl)pyrimidin-2- yl)amino)methyl)isoxazol-5- yl)pyridin-3-yl)carbamoyl)- cyclopropane-1-carboxylic acid |
| Compound 22 | 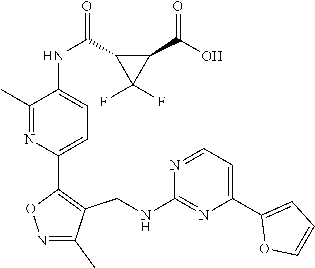 [SMILES: Cc1nc(-c2onc(C)c2CNc2nccc(-c3ccco3)n2)ccc1NC(=O)[C@@H]1[C@@H](C(=O)O)C1(F)F] | (1S,3S)-2,2-difluoro-3-((6-(4- (((4-(furan-2-yl)pyrimidin-2- yl)amino)methyl)-3-methyl- isoxazol-5-yl)-2-methylpyr- idin-3-yl)carbamoyl)cyclo- propane-1-carboxylic acid |
| Compound 23 | 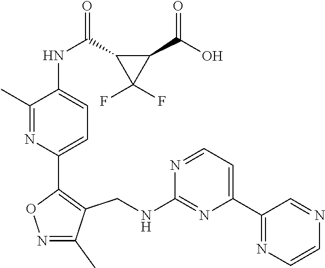 [SMILES: Cc1nc(-c2onc(C)c2CNc2nccc(-c3cnccn3)n2)ccc1NC(=O)[C@@H]1[C@@H](C(=O)O)C1(F)F] | (1S,3S)-2,2-difluoro-3-((2- methyl-6-(3-methyl-4-(((4- (pyrazin-2-yl)pyrimidin-2- yl)amino)methyl)isoxazol-5- yl)pyridin-3-yl)carbamoyl)- cyclopropane-1-carboxylic acid |
| Compound 24 | 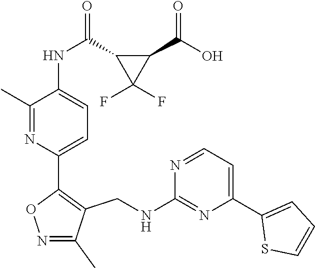 [SMILES: Cc1nc(-c2onc(C)c2CNc2nccc(-c3cccs3)n2)ccc1NC(=O)[C@@H]1[C@@H](C(=O)O)C1(F)F] | (1S,3S)-2,2-difluoro-3-((2- methyl-6-(3-methyl-4-(((4- (thiophen-2-yl)pyrimidin-2- yl)amino)methyl)isoxazol-5- yl)pyridin-3-yl)carbamoyl)- cyclopropane-1-carboxylic acid |
| Compound 25 | 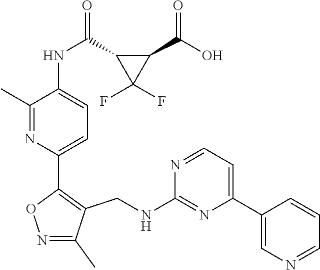 [SMILES: Cc1nc(-c2onc(C)c2CNc2nccc(-c3cccnc3)n2)ccc1NC(=O)[C@@H]1[C@@H](C(=O)O)C1(F)F] | (1S,3S)-2,2-difluoro-3-((2- methyl-6-(3-methyl-4-(((4- (pyridin-3-yl)pyrimidin-2- yl)amino)methyl)isoxazol-5- yl)pyridin-3-yl)carbamoyl)- cyclopropane-1-carboxylic acid |
| Compound 28 | 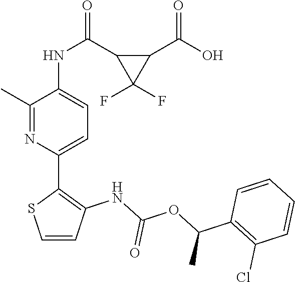 [SMILES: Cc1nc(-c2sccc2NC(=O)O[C@H](C)c2ccccc2Cl)ccc1NC(=O)C1C(C(=O)O)C1(F)F] | 3-((6-(3-((((R)-1-(2-chloro- phenyl)ethoxy)carbonyl)- amino)thiophen-2-yl)-2- methylpyridin-3-yl)car- bamoyl)-2,2-difluorocyclo- propane-1-carboxylic acid, mix of Trans diastereomers |
| Compound 29 | 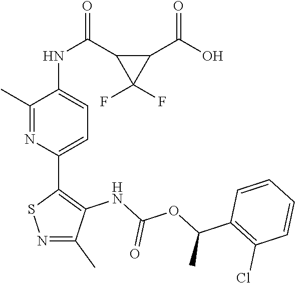 [SMILES: Cc1nc(-c2snc(C)c2NC(=O)O[C@H](C)c2ccccc2Cl)ccc1NC(=O)C1C(C(=O)O)C1(F)F] | 3-((6-(4-((((R)-1-(2-chloro- phenyl)ethoxy)carbonyl)- amino)-3-methylisothiazol- 5-yl)-2-methylpyridin-3-yl)- carbamoyl)-2,2-difluoro- cyclopropane-1-carboxylic acid, mix of Trans diastereo- mers |
| Compound 30 | 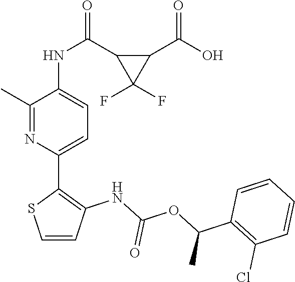 [SMILES: Cc1nc(-c2sccc2NC(=O)O[C@H](C)c2ccccc2Cl)ccc1NC(=O)C1C(C(=O)O)C1(F)F] | Single Diastereomer 2 of Trans-3-((6-(3-((((R)-1-(2- chlorophenyl)ethoxy)carbon- yl)amino)thiophen-2-yl)-2- methylpyridin-3-yl)car- bamoyl)-2,2-difluorocyclo- propane-1-carboxylic acid |
| Compound 31 | 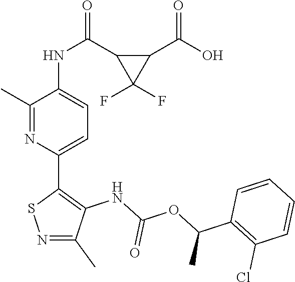 [SMILES: Cc1nc(-c2snc(C)c2NC(=O)O[C@H](C)c2ccccc2Cl)ccc1NC(=O)C1C(C(=O)O)C1(F)F] | Single Diastereomer 1 of Trans-3-((6-(4-((((R)-1-(2- chlorophenyl)ethoxy)carbon- yl)amino)-3-methylisothia- zol-5-yl)-2-methylpyridin-3- yl)carbamoyl)-2,2-difluoro- cyclopropane-1-carboxylic acid |
| Compound 32 | 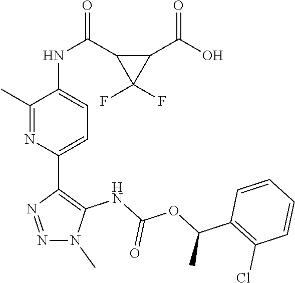 [SMILES: Cc1nc(-c2nnn(C)c2NC(=O)O[C@H](C)c2ccccc2Cl)ccc1NC(=O)C1C(C(=O)O)C1(F)F] | 3-((6-(5-((((R)-1-(2-chloro- phenyl)ethoxy)carbonyl)- amino)-1-methyl-1H-1,2,3- triazol-4-yl)-2-methylpyr- idin-3-yl)carbamoyl)-2,2- difluorocyclopropane-1- carboxylic acid, mix of Trans diastereomers |
| Compound 33 | 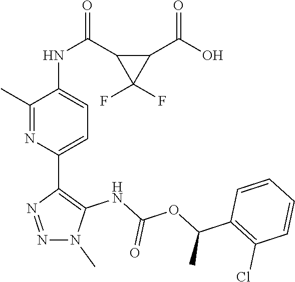 [SMILES: Cc1nc(-c2nnn(C)c2NC(=O)O[C@H](C)c2ccccc2Cl)ccc1NC(=O)C1C(C(=O)O)C1(F)F] | Single Diastereomer 1 of Trans-3-((6-(5-((((R)-1-(2- chlorophenyl)ethoxy)carbon- yl)amino)-1-methyl-1H-1,2,3- triazol-4-yl)-2-methylpyr- idin-3-yl)carbamoyl)-2,2- difluorocyclopropane-1- carboxylic acid |
| Compound 34 | 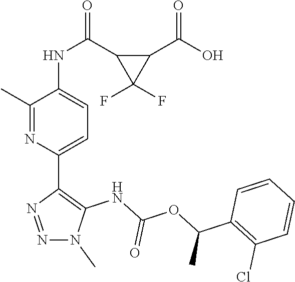 [SMILES: Cc1nc(-c2nnn(C)c2NC(=O)O[C@H](C)c2ccccc2Cl)ccc1NC(=O)C1C(C(=O)O)C1(F)F] | Single Diastereomer 2 of Trans-3-((6-(5-((((R)-1-(2- chlorophenyl)ethoxy)carbon- yl)amino)-1-methyl-1H-1,2,3- triazol-4-yl)-2-methylpyridin- 3-yl)carbamoyl)-2,2-difluoro- cyclopropane-1-carboxylic acid |
| Compound 37 | 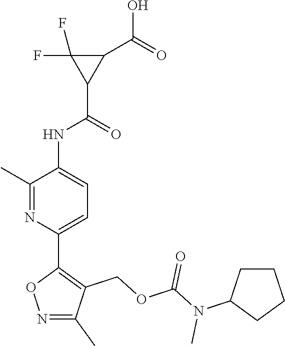 [SMILES: Cc1nc(-c2onc(C)c2COC(=O)N(C)C2CCCC2)ccc1NC(=O)C1C(C(=O)O)C1(F)F] | 3-((6-(4-(((cyclopentyl(meth- yl)carbamoyl)oxy)methyl)-3- methylisoxazol-5-yl)-2-meth- ylpyridin-3-yl)carbamoyl)- 2,2-difluorocyclopropane-1- carboxylic acid, mix of Trans diastereomers |
| Compound 41 | 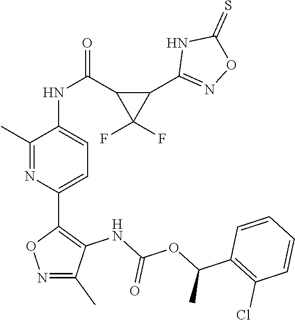 [SMILES: Cc1nc(-c2onc(C)c2NC(=O)O[C@H](C)c2ccccc2Cl)ccc1NC(=O)C1C(c2noc(=S)[nH]2)C1(F)F] | (R)-1-(2-chlorophenyl)ethyl (5-(5-(2,2-difluoro-3-(5- thioxo-4,5-dihydro-1,2,4- oxadiazol-3-yl)cyclopropane- 1-carboxamido)-6-methyl- pyridin-2-yl)-3-methylisox- azol-4-yl)carbamate, mix of Trans diastereomers |
| Compound 42 | 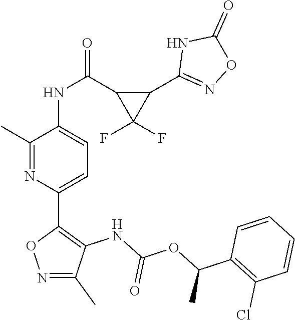 [SMILES: Cc1nc(-c2onc(C)c2NC(=O)O[C@H](C)c2ccccc2Cl)ccc1NC(=O)C1C(c2noc(=O)[nH]2)C1(F)F] | (R)-1-(2-chlorophenyl)ethyl (5-(5-(2,2-difluoro-3-(5-oxo- 4,5-dihydro-1,2,4-oxadiazol- 3-yl)cyclopropane-1-carbox- amido)-6-methylpyridin-2- yl)-3-methylisoxazol-4-yl)- carbamate, mix of Trans diastereomers |
| Compound 43 | 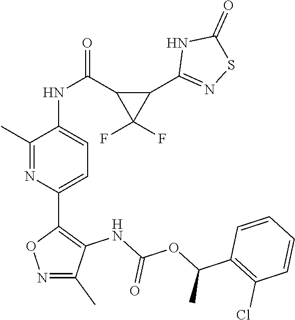 [SMILES: Cc1nc(-c2onc(C)c2NC(=O)O[C@H](C)c2ccccc2Cl)ccc1NC(=O)C1C(c2nsc(=O)[nH]2)C1(F)F] | (R)-1-(2-chlorophenyl)ethyl (5-(5-(2,2-difluoro-3-(5-oxo- 4,5-dihydro-1,2,4-thiadiazol- 3-yl)cyclopropane-1-carbox- amido)-6-methylpyridin-2- yl)-3-methylisoxazol-4-yl)- carbamate, mix of Trans diastereomers |
| Compound 44 | 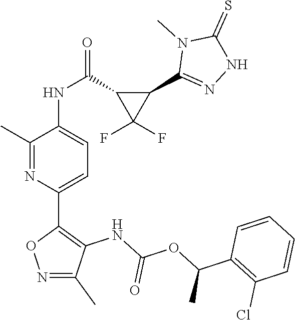 [SMILES: Cc1nc(-c2onc(C)c2NC(=O)O[C@H](C)c2ccccc2Cl)ccc1NC(=O)[C@@H]1[C@@H](c2n[nH]c(=S)n2C)C1(F)F] | (R)-1-(2-chlorophenyl)ethyl (5-(5-((1S,3S)-2,2-difluoro- 3-(4-methyl-5-thioxo-4,5- dihydro-1H-1,2,4-triazol-3- yl)cyclopropane-1-carbox- amido)-6-methylpyridin-2- yl)-3-methylisoxazol-4-yl)- carbamate |

In a preferred embodiment, the invention refers to a compound of formula (I) as LPA1

antagonist, wherein X is —N, A is

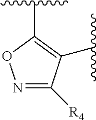

represented by the general formula (Ib)

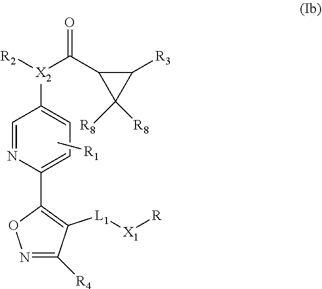

### Wherein

X₁ is —NH— or —O—
X₂ is —N— or —CH₂—N—,
L₁ is —(CH₂)ₙ
n is an integer between 0 and 3;
R₁H or —(C₁-C₄)alkyl;
R₂ is H or —(C₁-C₄)alkyl;
R₃ is selected from —C(O)OR₂ or heteroaryl wherein said heteroaryl is optionally substituted by oxo or thioxo;
R₄ is H or —(C₁-C₄)alkyl;
R is selected from the group consisting of —C(O)O(C₁-C₅)alkyl-R₅, —C(O)NR₆R₇, heteroaryl, wherein any of such heteroaryl may be optionally substituted by one or more groups selected from —OR₂, —(C₁-C₄)alkyl, —(C₁-C₄)haloalkyl, —(C₃-C₆)cycloalkyl, aryl, heteroaryl;
R₅ is H or selected from the group consisting of —(C₁-C₄)alkyl, aryl, wherein any of such aryl is optionally substituted by one or more groups selected from —(C₁-C₄)alkyl or halo;
R₆ and R₇ are at each occurrence independently H or selected from the group consisting of —(C₁-C₄)alkyl, —(C₃-C₆)cycloalkyl, —(C₁-C₆)haloalkyl and halo;
R₈ is H or halo.

In a preferred embodiment, the invention refers to at least one of the compounds listed in the Table 3 below and pharmaceutical acceptable salts thereof.

TABLE 3
List of preferred compounds of Formula (I)

| Cmpd. No. | Structure | Name |
|---|---|---|
| Compound 1 | 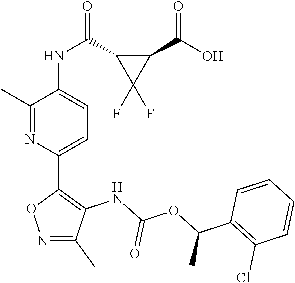 [SMILES: Cc1nc(-c2onc(C)c2NC(=O)O[C@H](C)c2ccccc2Cl)ccc1NC(=O)[C@@H]1[C@@H](C(=O)O)C1(F)F] | (1S,3S)-3-((6-(4-((((R)-1-(2- chlorophenyl)ethoxy)carbon- yl)amino)-3-methylisoxazol- 5-yl)-2-methylpyridin-3- yl)carbamoyl)-2,2-difluoro- cyclopropane-1-carboxylic acid |
| Compound 2 | 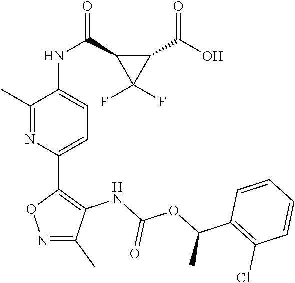 [SMILES: Cc1nc(-c2onc(C)c2NC(=O)O[C@H](C)c2ccccc2Cl)ccc1NC(=O)[C@H]1[C@H](C(=O)O)C1(F)F] | (1R,3R)-3-((6-(4-((((R)-1-(2- chlorophenyl)ethoxy)carbon- yl)amino)-3-methylisoxazol- 5-yl)-2-methylpyridin-3-yl)- carbamoyl)-2,2-difluorocyclo- propane-1-carboxylic acid |
| Compound 3 | 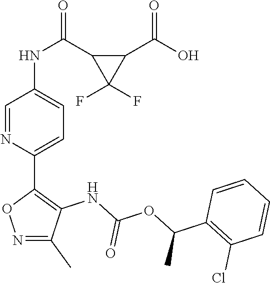 [SMILES: Cc1noc(-c2ccc(NC(=O)C3C(C(=O)O)C3(F)F)cn2)c1NC(=O)O[C@H](C)c1ccccc1Cl] | 3-((6-(4-((((R)-1-(2-chloro- phenyl)ethoxy)carbonyl)- amino)-3-methylisoxazol-5- yl)pyridin-3-yl)carbamoyl)- 2,2-difluorocyclopropane-1- carboxylic acid, mix of Trans diastereomers |
| Compound 4 | 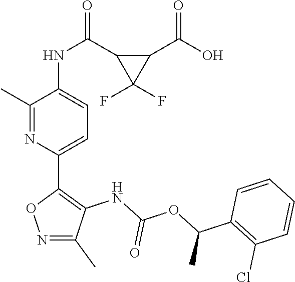 [SMILES: Cc1nc(-c2onc(C)c2NC(=O)O[C@H](C)c2ccccc2Cl)ccc1NC(=O)C1C(C(=O)O)C1(F)F] | 3-((6-(4-((((R)-1-(2-chloro- phenyl)ethoxy)carbonyl)- amino)-3-methylisoxazol-5- yl)-2-methylpyridin-3-yl)- carbamoyl)-2,2-difluoro- cyclopropane-1-carboxylic acid, mix of Trans diastereo- mers |
| Compound 5 | 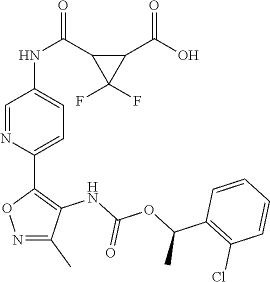 [SMILES: Cc1noc(-c2ccc(NC(=O)C3C(C(=O)O)C3(F)F)cn2)c1NC(=O)O[C@H](C)c1ccccc1Cl] | Single Diastereomer 1 of Trans-3-((6-(4-((((R)-1-(2- chlorophenyl)ethoxy)carbon- yl)amino)-3-methylisoxazol- 5-yl)pyridin-3-yl)carbamoyl)- 2,2-difluorocyclopropane-1- carboxylic acid |
| Compound 6 | 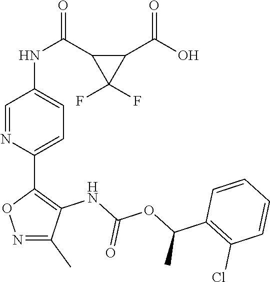 [SMILES: Cc1noc(-c2ccc(NC(=O)C3C(C(=O)O)C3(F)F)cn2)c1NC(=O)O[C@H](C)c1ccccc1Cl] | Single Diastereomer 2 of Trans-3-((6-(4-((((R)-1-(2- chlorophenyl)ethoxy)carbon- yl)amino)-3-methylisoxazol- 5-yl)pyridin-3-yl)carbamoyl)- 2,2-difluorocyclopropane-1- carboxylic acid |
| Compound 10 | 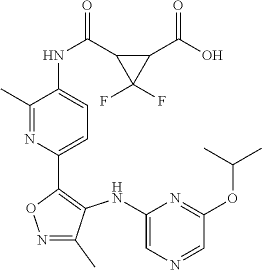 [SMILES: Cc1nc(-c2onc(C)c2Nc2cncc(OC(C)C)n2)ccc1NC(=O)C1C(C(=O)O)C1(F)F] | 2,2-difluoro-3-((6-(4-((6- isopropoxypyrazin-2-yl)- amino)-3-methylisoxazol-5- yl)-2-methylpyridin-3-yl)- carbamoyl)cyclopropane-1- carboxylic acid, mix of Trans diastereomers |
| Compound 11 | 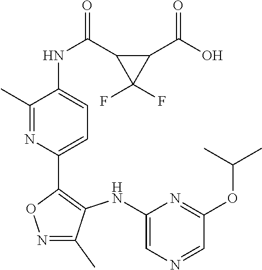 [SMILES: Cc1nc(-c2onc(C)c2Nc2cncc(OC(C)C)n2)ccc1NC(=O)C1C(C(=O)O)C1(F)F] | Single Enantiomer 2 of Trans-2,2-difluoro-3-((6-(4- ((6-isopropoxypyrazin-2-yl)- amino)-3-methylisoxazol-5- yl)-2-methylpyridin-3-yl)- carbamoyl)cyclopropane-1- carboxylic acid |
| Compound 12 | 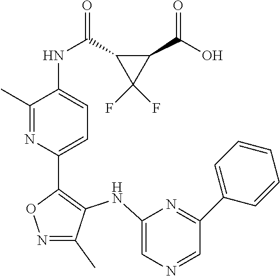 [SMILES: Cc1nc(-c2onc(C)c2Nc2cncc(-c3ccccc3)n2)ccc1NC(=O)[C@@H]1[C@@H](C(=O)O)C1(F)F] | (1S,3S)-2,2-difluoro-3-((2- methyl-6-(3-methyl-4-((6- phenylpyrazin-2-yl)amino)- isoxazol-5-yl)pyridin-3-yl)- carbamoyl)cyclopropane-1- carboxylic acid |
| Compound 14 | 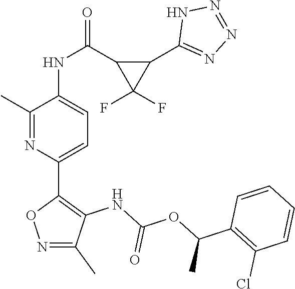 [SMILES: Cc1nc(-c2onc(C)c2NC(=O)O[C@H](C)c2ccccc2Cl)ccc1NC(=O)C1C(c2nnn[nH]2)C1(F)F] | (R)-1-(2-chlorophenyl)ethyl (5-(5-(2,2-difluoro-3-(1H- tetrazol-5-yl)cyclopropane-1- carboxamido)-6-methylpyr- idin-2-yl)-3-methylisoxazol- 4-yl)carbamate, mix of Trans diastereomers |
| Compound 15 | 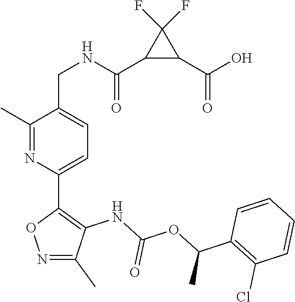 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1- carboxylic acid, mix of Cis and Trans diastereomers |
| Compound 16 | 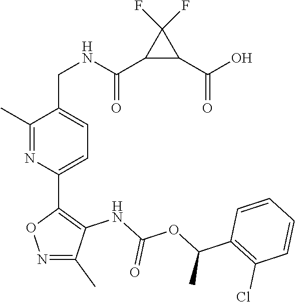 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1- carboxylic acid, mix of Cis diastereomers |
| Compound 17 | 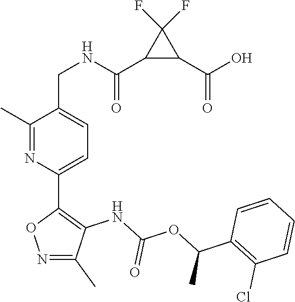 [SMILES: Cc1nc(-c2onc(C)c2NC(=O)O[C@H](C)c2ccccc2Cl)ccc1CNC(=O)C1C(C(=O)O)C1(F)F] | 3-(((6-(4-((((R)-1-(2-chloro- phenyl)ethoxy)carbonyl)- amino)-3-methylisoxazol-5- yl)-2-methylpyridin-3-yl)- methyl)carbamoyl)-2,2- difluorocyclopropane-1- carboxylic acid, mix of Trans diastereomers |
| Compound 18 | 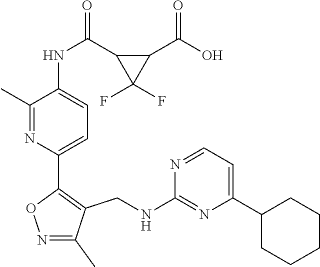 [SMILES: Cc1nc(-c2onc(C)c2CNc2nccc(C3CCCCC3)n2)ccc1NC(=O)C1C(C(=O)O)C1(F)F] | 3-((6-(4-(((4-cyclohexylpyr- imidin-2-yl)amino)methyl)- 3-methylisoxazol-5-yl)-2- methylpyridin-3-yl)carbam- oyl)-2,2-difluorocyclopro- pane-1-carboxylic acid, mix of Trans diastereomers |
| Compound 19 | 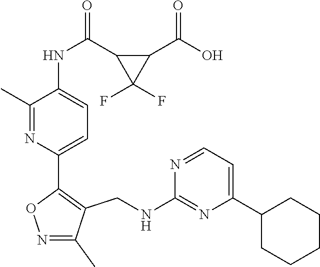 [SMILES: Cc1nc(-c2onc(C)c2CNc2nccc(C3CCCCC3)n2)ccc1NC(=O)C1C(C(=O)O)C1(F)F] | Single Enantiomer 1 of Trans-3-((6-(4-(((4-cyclohex- ylpyrimidin-2-yl)amino)meth- yl)-3-methylisoxazol-5-yl)-2- methylpyridin-3-yl)carbamo- yl)-2,2-difluorocyclopropane- 1-carboxylic acid |
| Compound 20 | 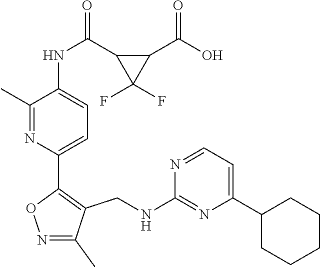 [SMILES: Cc1nc(-c2onc(C)c2CNc2nccc(C3CCCCC3)n2)ccc1NC(=O)C1C(C(=O)O)C1(F)F] | Single Enantiomer 2 of Trans- 3-((6-(4-(((4-cyclohexylpyr- imidin-2-yl)amino)methyl)-3- methylisoxazol-5-yl)-2-meth- ylpyridin-3-yl)carbamoyl)- 2,2-difluorocyclopropane-1- carboxylic acid |
| Compound 21 | 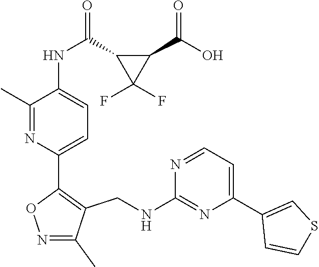 [SMILES: Cc1nc(-c2onc(C)c2CNc2nccc(-c3ccsc3)n2)ccc1NC(=O)[C@@H]1[C@@H](C(=O)O)C1(F)F] | (1S,3S)-2,2-difluoro-3-((2- methyl-6-(3-methyl-4-(((4- (thiophen-3-yl)pyrimidin-2- yl)amino)methyl)isoxazol-5- yl)pyridin-3-yl)carbamoyl)- cyclopropane-1-carboxylic acid |
| Compound 22 | 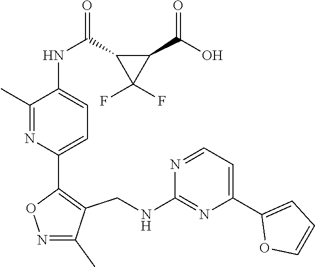 [SMILES: Cc1nc(-c2onc(C)c2CNc2nccc(-c3ccco3)n2)ccc1NC(=O)[C@@H]1[C@@H](C(=O)O)C1(F)F] | (1S,3S)-2,2-difluoro-3-((6-(4- (((4-(furan-2-yl)pyrimidin-2- yl)amino)methyl)-3-methyl- isoxazol-5-yl)-2-methylpyr- idin-3-yl)carbamoyl)cyclo- propane-1-carboxylic acid |
| Compound 23 | 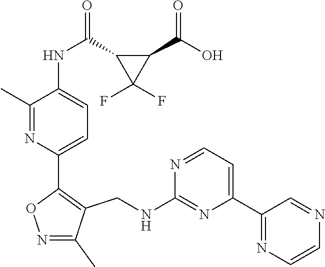 [SMILES: Cc1nc(-c2onc(C)c2CNc2nccc(-c3cnccn3)n2)ccc1NC(=O)[C@@H]1[C@@H](C(=O)O)C1(F)F] | (1S,3S)-2,2-difluoro-3-((2- methyl-6-(3-methyl-4-(((4- (pyrazin-2-yl)pyrimidin-2- yl)amino)methyl)isoxazol-5- yl)pyridin-3-yl)carbamoyl)- cyclopropane-1-carboxylic acid |
| Compound 24 | 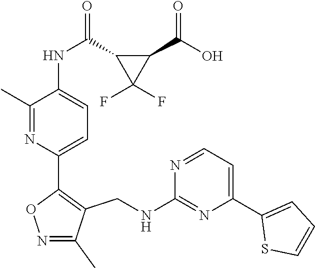 [SMILES: Cc1nc(-c2onc(C)c2CNc2nccc(-c3cccs3)n2)ccc1NC(=O)[C@@H]1[C@@H](C(=O)O)C1(F)F] | (1S,3S)-2,2-difluoro-3-((2- methyl-6-(3-methyl-4-(((4- (thiophen-2-yl)pyrimidin-2- yl)amino)methyl)isoxazol-5- yl)pyridin-3-yl)carbamoyl)- cyclopropane-1-carboxylic acid |
| Compound 25 | 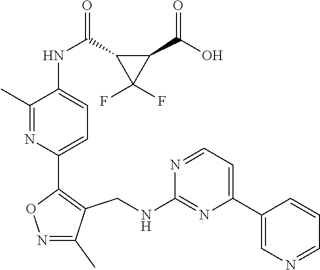 [SMILES: Cc1nc(-c2onc(C)c2CNc2nccc(-c3cccnc3)n2)ccc1NC(=O)[C@@H]1[C@@H](C(=O)O)C1(F)F] | (1S,3S)-2,2-difluoro-3-((2- methyl-6-(3-methyl-4-(((4- (pyridin-3-yl)pyrimidin-2- yl)amino)methyl)isoxazol-5- yl)pyridin-3-yl)carbamoyl)- cyclopropane-1-carboxylic acid |
| Compound 37 | 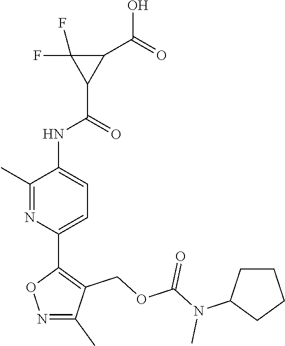 [SMILES: Cc1nc(-c2onc(C)c2COC(=O)N(C)C2CCCC2)ccc1NC(=O)C1C(C(=O)O)C1(F)F] | 3-((6-(4-(((cyclopentyl(meth- yl)carbamoyl)oxy)methyl)-3- methylisoxazol-5-yl)-2-meth- ylpyridin-3-yl)carbamoyl)- 2,2-difluorocyclopropane-1- carboxylic acid, mix of Trans diastereomers |
| Compound 41 | 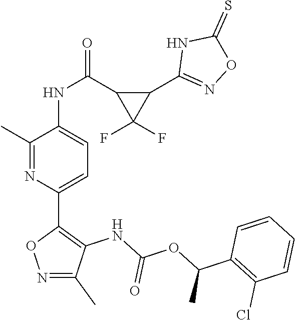 [SMILES: Cc1nc(-c2onc(C)c2NC(=O)O[C@H](C)c2ccccc2Cl)ccc1NC(=O)C1C(c2noc(=S)[nH]2)C1(F)F] | (R)-1-(2-chlorophenyl)ethyl (5-(5-(2,2-difluoro-3-(5- thioxo-4,5-dihydro-1,2,4- oxadiazol-3-yl)cyclopropane- 1-carboxamido)-6-methyl- pyridin-2-yl)-3-methyliso- xazol-4-yl)carbamate, mix of Trans diastereomers |
| Compound 42 | 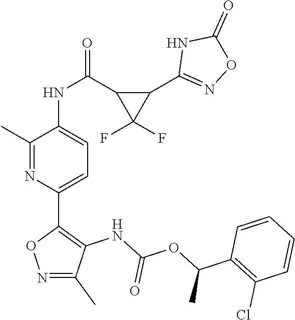 [SMILES: Cc1nc(-c2onc(C)c2NC(=O)O[C@H](C)c2ccccc2Cl)ccc1NC(=O)C1C(c2noc(=O)[nH]2)C1(F)F] | (R)-1-(2-chlorophenyl)ethyl (5-(5-(2,2-difluoro-3-(5-oxo- 4,5-dihydro-1,2,4-oxadiazol- 3-yl)cyclopropane-1-carbox- amido)-6-methylpyridin-2- yl)-3-methylisoxazol-4-yl)- carbamate, mix of Trans diastereomers |
| Compound 43 | 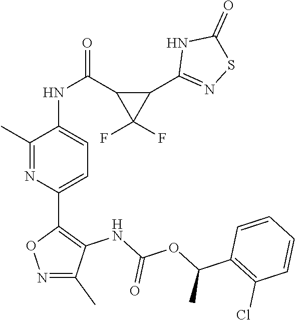 [SMILES: Cc1nc(-c2onc(C)c2NC(=O)O[C@H](C)c2ccccc2Cl)ccc1NC(=O)C1C(c2nsc(=O)[nH]2)C1(F)F] | (R)-1-(2-chlorophenyl)ethyl (5-(5-(2,2-difluoro-3-(5-oxo- 4,5-dihydro-1,2,4-thiadiazol- 3-yl)cyclopropane-1-carbox- amido)-6-methylpyridin-2- yl)-3-methylisoxazol-4-yl)- carbamate, mix of Trans diastereomers |
| Compound 44 | 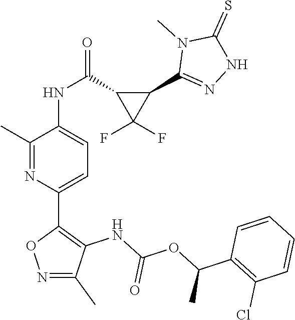 [SMILES: Cc1nc(-c2onc(C)c2NC(=O)O[C@H](C)c2ccccc2Cl)ccc1NC(=O)[C@@H]1[C@@H](c2n[nH]c(=S)n2C)C1(F)F] | (R)-1-(2-chlorophenyl)ethyl (5-(5-((1S,3S)-2,2-difluoro- 3-(4-methyl-5-thioxo-4,5- dihydro-1H-1,2,4-triazol-3- yl)cyclopropane-1-carbox- amido)-6-methylpyridin-2- yl)-3-methylisoxazol-4-yl)- carbamate |

In a preferred embodiment, the invention refers to a compound of formula (I) as LPA1 antagonist. In this respect, it has now been found that the compounds of formula (I) of the present invention have an antagonist drug potency expressed as half maximal inhibitory concentration (IC₅₀) on LPA1 lesser than 200 nM.

Preferably, the compounds of the present invention have an IC₅₀ on LPA1 lesser or equal than 100 nM.

More preferably, the compounds of the present invention have an IC₅₀ on LPA1 lesser or equal than 50 nM.

The compounds of the invention are also characterized by a passive permeability ≤5 nm/sec. In one aspect, the present invention refers to a compound of formula (I) for use as a medicament. Thus, the invention refers to a compound of formula (I) in the preparation of a medicament, preferably for use in the treatment of disorders associated with LPA receptors mechanism.

In a preferred embodiment, the invention refers to a compound of formula (I) for use in the treatment of disorders associated with LPA receptors mechanism.

In a further embodiment, the present invention refers to a compound of formula (I) for use in the treatment of a disease, disorder or condition associated with dysregulation of lysophosphatidic acid receptor 1 (LPA1).

In one embodiment, the present invention refers to a compound of formula (I) useful for the prevention and/or treatment of fibrosis and/or diseases, disorders, or conditions that involve fibrosis.

The terms “fibrosis” or “fibrosis disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Preferably, the compounds of formula (I) of the present invention are useful for the treatment and/or prevention of fibrosis such as pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

More preferably, the compounds of formula (I) of the present invention are useful for the treatment of idiopathic pulmonary fibrosis (IPF).

In one aspect, the invention also refers to a method for the prevention and/or treatment of disorders associated with LPA receptors mechanisms, said method comprises administering to a patient in need of such treatment a therapeutically effective amount of a compound of formula (I).

In a further aspect, the invention refers to a method for the prevention and/or treatment of disorder or condition associated with dysregulation of lysophosphatidic acid receptor 1 (LPA1) administering a patient in need of such treatment a therapeutically effective amount of a compound of formula (I).

In a further aspect, the invention refers to a method for the treatment and/or prevention of fibrosis such as pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

In a further aspect, the invention refers to the use of a compound of formula (I) according to the invention, for the treatment of disorders associated with LPA receptors mechanism.

In a further aspect, the invention refers to the use of the compound of formula (I) for the preparation of a medicament for the treatment of disorders associated with LPA receptors mechanism.

In a further aspect, the invention refers to the use of the compound of formula (I) for the preparation of a medicament for the treatment and/or prevention of fibrosis such as pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

In a further aspect, the present invention refers to the use of a compound of formula (I) for the treatment of a disease, disorder or condition associated with dysregulation of lysophosphatidic acid receptor 1 (LPA1).

As used herein, “safe and effective amount” in reference to a compound of formula (I) or a pharmaceutically acceptable salt thereof or other pharmaceutically active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) in admixture with at least one or more pharmaceutically acceptable carrier or excipient.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered by inhalation.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of Formula (I) according to the invention, in form of a single- or multi-dose dry powder inhaler or a metered dose inhaler.

All preferred groups or embodiments described above for compounds of formula I may be combined among each other and apply as well mutatis mutandis.

The compounds of the present invention can be prepared in a number of ways known to one skilled in the art of organic synthesis. It will be understood by those skilled in the art of organic synthesis that the functionality present on the molecule should be consistent with the transformation proposed. This will sometimes require a modification of the order of synthetic steps in order to obtain a desired compound of the invention. The compounds of Formula (I), including all the compounds here above listed, can be generally prepared according to the procedure outlined in Schemes shown below using generally known methods.

### PREPARATIONS OF INTERMEDIATES AND EXAMPLES

All reagents, for which the synthesis is not described in the experimental part, are either commercially available, or are known compounds or may be formed from known compounds by known methods by a person skilled in the art.

### Abbreviation—Meaning

Cs₂CO₃=Cesium carbonate
Cp*RuCl(PPh₃)₂=Pentamethylcyclopentadienylbis(triphenylphosphine)ruthenium(II) chloride
CyHex=Cyclohexane
CPME=Cyclopentyl methyl ether
CuI=Copper(I) iodide
DBU=1,8-Diazabicyclo[5.4.0]undec-7-ene
DCM=Dichloromethane
DMAP=4-Dimethylaminopyridine
DMF=Dimethylformamide
DMSO=Dimethylsulfoxide
DPPA=Diphenylphosphoryl azide
EDC═N-(3-Dimethylaminopropyl)-N′-ethylcarbodiimide hydrochloride
EtOAc=Ethyl acetate
Et₂O=diethyl ether
Fe=iron
h=hour
HATU=1-[Bis(dimethylamino)methylene]-1H-1,2,3-triazolo[4,5-b]pyridinium 3-oxid hexafluorophosphate
HCl=Hydrochloric acid
HCOOH=Formic acid
H₂O=Water
K₂CO₃=Potassium carbonate
KF=Potassium fluoride
KMnO₄=Potassium permanganate
K₃PO₄=Potassium phosphate tribasic
LC-MS=liquid chromatography/mass spectrometry
LiOH=Litium hydroxide
LiCl=Lithiun chloride
MeCN=Acetonitrile
MeOH=Methanol
N₂=Nitrogen
NaOH═Sodium hydroxide
Na₂CO₃=Sodium carbonate
NaHCO₃=Sodium bicarbonate
Na₂SO₄=Sodium sulfate
NBS═N-bromosuccinimmide
NH₃=ammonia
NH₄Cl=Ammonium chloride
PdCl₂(PPh₃)₂=Bis(triphenylphosphine)palladium(II) dichloride
Pd₂(dba)₃=Tris(dibenzylideneacetone)dipalladium(0)
Pd(dppf)Cl₂=[1,1′-Bis(diphenylphosphino)ferrocene]dichloropalladium(II)
Pd(PPh₃)₄=Palladium-tetrakis(triphenylphosphine)
PCy₃=Tricyclohexylphosphine
PPh₃=Triphenylphosphine
RT=room temperature
SnCl₂*2H₂O=Tin(II) chloride dehydrate
SnMe₃OH=trimethyltin hydroxide
TCFH=Chloro-N,N,N′,N′-tetramethylformamidinium hexafluorophosphate
TBAF=tetrabutylammonium fluoride
THF=Tetrahydrofuran
TMSN3=trymethylsilylazide
XantPhos=4,5-Bis(diphenylphosphino)-9,9-dimethylxanthene

### General Experimental Details and Methods

### Analytical Method

Instruments, materials and methods employed for analyses

¹H-NMR spectra were performed on a Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with transmitter offset frequency shift, or on AgilentVNMRS-500 or on a Bruker Avance 300/400 spectrometers or on a bruker Fourier 300. Chemical shifts are reported as 6 values in ppm relative to trimethylsilane (TMS) as an internal standard. Coupling constants (J values) are given in hertz (Hz) and multiplicities are reported using the following abbreviation (s=singlet, d=doublet, t=triplet, q=quartet, m=multiplet, br. s=broad singlet, nd=not determined).

### LC/UV/MS Analytical Methods

LC/MS retention times are estimated to be affected by an experimental error of ±0.5 min. LCMS may be recorded under the following conditions: diode array DAD chromatographic traces, mass chromatograms and mass spectra may be taken on UPLC/PDA/MS Acquity™ system coupled with Micromass ZQ™ or on a Waters Alliance e2695 HPLC with Photodiode Detector 2998 coupled with Column Oven and Mass Spectrometer ZQ in positive and/or negative electron spray ES ionization mode and/or Fractionlynx system used in analytical mode coupled with ZQ™ single quadrupole operated in positive and/or negative ES ionization mode or on a Shimadzu LCMS-2020 Single Quadrupole Liquid Chromatograph Mass Spectrometer and LCMS spectra were measured on Dionex UHPLC Ultimate 3000 with DAD detector/Thermo Scientific MSQ Plus.

Quality Control methods used operated under low pH conditions or under high pH conditions:

Method 1, low pH conditions column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 1 mL/min.

The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES-range was 100 to 1500 AMU.

Method 2, low pH conditions: column: Phenomenex Gemini-NX C18, 150×2.0 mm, 3 m with security guard Gemini-NX C18, 4×2.0 mm, 3 m, the column temperature was 25° C.; mobile phase solvent A was water+0.1% Formic acid filtered with 0.22 m nylon filter, mobile phase solvent B Acetonitrile+0.1% Formic acid filtered with 0.22 m nylon filter. The flow rate was 0.2 mL/min. The gradient table was t=0 min 80% A 20% B, t=10 min 10% A 90% B, t=35 min 10% A 90% B. The UV detection λ was 215 nm and ES+ range was 50 to 900 Da.

Method 3, high pH conditions: column Acquity UPLC 1.7 μm BEH-C18 (2.1×100 mm) 130A, the column temperature was 40° C.; mobile phase solvent A was 0.05% μQ water solution of ammonium hydroxide (28.0-30.0% NH₃ basis), mobile phase solvent B MeCN. The flow rate was 0.5 mL/min. The gradient table was t=0.00 min 80% A 20% B, t=0.10 min 80% A, 20% B, t=1.10 min 0% A, 100% B, t=2.00 min 0% A, 100% B, t=2.50 min 80% A, 20% B, t=3.00 min 80% A, 20% B, UV detection range was 200-400 nm and ES+/ES− range was 100 to 1000 AMU.

Method 4, low pH conditions: column Kinetex®2.6 μm XB—C18 (4.6×50 mm), 110A, the column temperature was 25° C.; mobile phase solvent A was μQ-water for LCMS+0.1% HCOOH, mobile phase solvent B MeCN+0.1% HCOOH. The flow rate was 1 mL/min. The gradient table was t=0.00 min 80% A 20% B, t=3.35 min 20% A, 80% B, t=3.75 min 20% A, 80% B, t=3.90 min 5% A, 95% B, t=4.75 min 5% A, 95% B, t=5.00 min 80% A, 20% B, t=6.00 min 80% A, 20% B, UV detection range was 1900-340 nm and ES+/ES− range was 100 to 1000 AMU.

Method 5, low pH conditions: column Kinetex®2.6 μm XB—C18 (4.6×50 mm), 110A, the column temperature was 25° C.; mobile phase solvent A was μQ-water for LCMS+0.1% HCOOH, mobile phase solvent B MeCN+0.1% HCOOH. The flow rate was 1 mL/min. The gradient table was t=0.00 min 90% A 10% B, t=3.35 min 30% A, 70% B, t=3.75 min 30% A, 70% B, t=3.90 min 5% A, 95% B, t=4.75 min 5% A, 95% B, t=5.00 min 90% A, 10% B, t=6.00 min 90% A, 10% B, UV detection range was 1900-340 nm and ES+/ES− range was 100 to 1000 AMU.

Method 6, low pH conditions: column Kinetex®2.6 μm XB—C18 (4.6×50 mm), 110A, the column temperature was 25° C.; mobile phase solvent A was μQ-water for LCMS+0.1% HCOOH, mobile phase solvent B MeCN+0.1% HCOOH. The flow rate was 1 mL/min. The gradient table was t=0.00 min 95% A 5% B, t=1.00 min 95% A, 5% B, t=4.75 min 20% A, 80% B, t=5.25 min 20% A, 80% B, t=6.00 min 95% A, 5% B, t=7.00 min 95% A, 5% B, UV detection range was 1900-340 nm and ES+/ES− range was 100 to 1000 AMU.

Method 7, low pH conditions: column Kinetex®2.6 μm XB—C18 (4.6×50 mm), 110A, the column temperature was 25° C.; mobile phase solvent A was μQ-water for LCMS+0.1% HCOOH, mobile phase solvent B MeCN+0.1% HCOOH. The flow rate was 1 mL/min. The gradient table was t=0.00 min 70% A, 30% B, t=3.35 min 20% A, 80% B, t=3.75 min 20% A, 80% B, t=3.90 min 5% A, 95% B, t=4.75 min 5% A, 95% B, t=5.00 min 70% A, 30% B, t=6.00 min 70% A, 30% B, UV detection range was 1900-340 nm and ES+/ES− range was 100 to 1000 AMU.

Method 8, low pH conditions: column Kinetex®2.6 μm XB—C18 (4.6×50 mm), 110A, the column temperature was 25° C.; mobile phase solvent A was μQ-water for LCMS +0.1% HCOOH, mobile phase solvent B MeCN+0.1% HCOOH. The flow rate was 1 mL/min. The gradient table was t=0.00 min 80% A, 20% B, t=3.35 min 20% A, 80% B, t=3.75 min 20% A, 80% B, t=3.90 min 5% A, 95% B, t=4.75 min 5% A, 95% B, t=5.00 min 80% A, 20% B, t=6.00 min 80% A, 20% B, UV detection range was 1900-340 nm and ES+/ES− range was 100 to 1000 AMU.

### Chiral Preparative HPLC for Chiral Compounds

Chiral resolutions were performed using a Semipreparative Waters 600 system or a Semipreparative Agilent 1100 system. The conditions are reported in the Examples.

Where the preparation of starting materials is not described, these are commercially available, known in the literature, or readily obtainable by those skilled in the art using standard procedures.

Flash chromatography is carried out using an Isolera MPLC system (manufactured by Biotage) using pre-packed silica gel or reverse-phase cartridges (supplied by Biotage or Sepachrom).

SPE-SCX cartridges are ion exchange solid phase extraction columns supplied by Agilent Technologies or Scharlab.

Semi-preparative SFC (supercritical fluid chromatography) Jasco system was employed for chiral separation Many of the compounds described in the following Examples have been prepared from stereochemically pure starting materials, for example 95% ee.

The stereochemistry of the compounds in the Examples, where indicated, has been assigned on the assumption that absolute configuration at resolved stereogenic centers of staring materials is maintained throughout any subsequent reaction conditions.

When reference is made to “peak 1” as will be appreciated by those skilled in the art refers to the first eluted enantiomer or diastereomer.

When reference is made to “peak 2” as will be appreciated by those skilled in the art refers to the second eluted enantiomer or diastereomer.

In the procedures that follow, after each starting material, reference to a compound number is sometimes provided. This is provided merely for assistance to the skilled chemist. The starting material may not necessarily have been prepared from the batch referred to.

When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions.

Chiral resolution of Trans-rac 2,2-difluoro-3-(methoxycarbonyl)cyclopropane-1-carboxylic acid

200 g of Trans-rac 2,2-difluoro-3-(methoxycarbonyl)cyclopropane-1-carboxylic acid were submitted to Chiral HPLC following the methods described below:

Conditions (analytical method): Column: Chiralpak OJ-H 5 um-250×4.6 mm Mobile phase n-heptane/propan-2-ol/TFA 80/20/0.1 Flow rate: 1 ml/min detection UV 210 nm temperature: 25 C

Conditions (Preparative method): Chiralpak ID 20 um-250×110 mm 5μ Mobile phase n-heptane/propan-2-ol/formic acid 80/20/0.1 v/v Flow rate 570 ml/min UV 220 nm detection temperature: 25 C

Peak 1: Single Enantiomer 1 of Trans-2,2-difluoro-3-(methoxycarbonyl)cyclopropane-1-carboxylic acid (Intermediate 1)

91.2 g of first eluted enantiomer (Enantiomer 1) were obtained as an oil.

¹H NMR (400 MHz, DMSO-d6) δ 13.53 (s, 1H), 3.72 (s, 3H), 3.06-3.21 (m, 2H).

LC-MS (ESI): m z (M+1): 181.0 (Method 1)

[α]_{D}=−90° c=1, EtOAc

Peak 2: Single Enantiomer 2 of Trans-2,2-difluoro-3-(methoxycarbonyl)cyclopropane-1-carboxylic acid (Intermediate 2)

95.5 g of second eluted enantiomer (Enantiomer 2) were obtained as an oil.

LC-MS (ESI): m z (M+1): 181.1 (Method 1)

¹H NMR (400 MHz, DMSO-d6) δ 13.54 (s, 1H), 3.72 (s, 3H), 3.04-3.22 (m, 2H).

[α]_{D}=+94° c=1, EtOAc

The isolated oil crystallized on standing. Single crystals were recovered and submitted to XRD to determine the absolute configuration of Intermediate 2.

XRD diffraction of single crystal of Intermediate 2 demonstrated that its absolute configuration was S,S. Consequently, the absolute configuration of Intermediate 1 was R,R.

### Example 1

### Process of Synthesis of Compound 1:

### (1S,3S)-3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylic acid

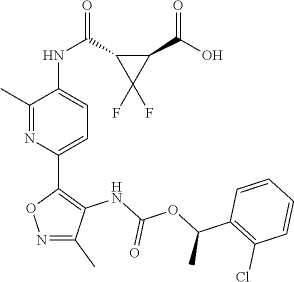 [SMILES: Cc1nc(-c2onc(C)c2NC(=O)O[C@H](C)c2ccccc2Cl)ccc1NC(=O)[C@@H]1[C@@H](C(=O)O)C1(F)F]

### Step 1: 5-bromo-6-methylpicolinoyl chloride (Intermediate A1)

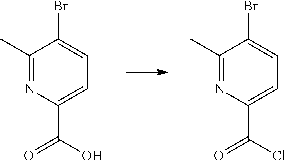

Oxalyl dichloride (39.7 mL, 462.9 mmol) was added at 10° C. to a suspension of 5-bromo-6-methylpicolinic acid (50.0 g, 231.45 mmol) in DCM (300 mL) and DMF (0.36 mL, 4.63 mmol).

The mixture was stirred at RT for 2 h, then was concentrated in vacuo to give 61.5 g of crude as a brown solid.

¹H NMR (400 MHz, DMSO-d6) δ ppm 8.19 (d, J 8.22 Hz, 1H), 7.78 (d, J 8.22 Hz, 1H), 2.64 (s, 3H)

### Step 2: methyl (E)-2-(5-bromo-6-methylpyridine-2-carbonyl)-3-(methylamino)but-2-enoate (Intermediate A2)

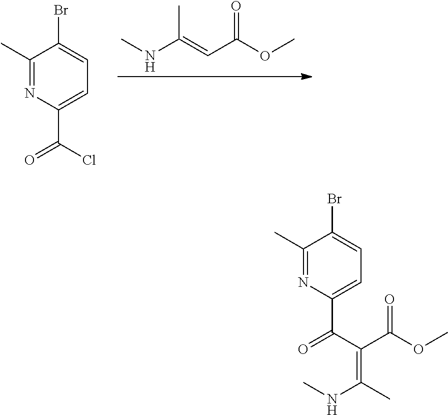

To a solution of methyl (E)-3-(methylamino)but-2-enoate (35.0 g, 270.98 mmol), prepared according to the procedure reported in J. Org. Chem., 1965, 30, 3033-3037, in THE (125 mL), pyridine (32.0 mL, 395.65 mmol) was added dropwise at RT. The mixture was cooled with an ice bath at 5-10° C. and a solution of 5-bromo-6-methylpicolinoyl chloride (Intermediate A1, 61.5 g, crude) in THE (250 mL) was added dropwise over 30 min, while keeping the process temperature at 10-15° C. The mixture was then allowed to warm up at RT and stirred overnight. THE was evaporated to ca. 150 mL of volume, then water (200 mL) was added and the mixture was extracted with EtOAc (3×150 mL). The mixed organic phases were washed with 1/1 brine/water (260 mL), dried over Na₂SO₄, filtered and concentrated in vacuo to give the title compound (85 g) as a brown oil, that was used in the following step without further purification.

LC-MS (ESI): m/z (M+1): 329.1 (Method 1)

### Step 3: methyl 5-(5-bromo-6-methylpyridin-2-yl)-3-methylisoxazole-4-carboxylate (Intermediate A3)

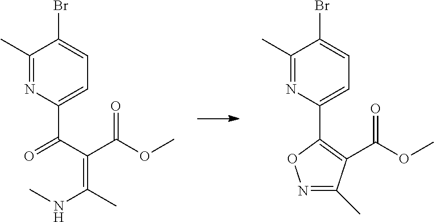

Methyl (E)-2-(5-bromo-6-methylpicolinoyl)-3-(methylamino)but-2-enoate (Intermediate A2, 85 g, crude) was dissolved in acetic acid (500 mL). Hydroxylamine hydrochloride (27.1 g, 390 mmol) was then added and the mixture was heated at 90° over 2 h. The solvent was evaporated under vacuum, then NaHCO₃ saturated aqueous solution (400 mL) was added and the mixture was extracted with EtOAc (3×500 mL). The combined organic layer was further washed with water and brine, dried over Na₂SO₄, and evaporated to dryness under reduced pressure. The crude was purified by flash chromatography eluting with a gradient of DCM/EtOAc from 100/0 to 90/10 and then the solid residue was triturated with EtOAc/CyHex to provide the title compound (44.6 g) as a white solid.

LC-MS (ESI): m/z (M+1): 313 (Method 1).

The Intermediates in the following table were prepared from reagents reported below by using methods analogous to Intermediate A3.

| Intermediate | Structure & Name | Reagents | Analytical data |
|---|---|---|---|
| A4 | 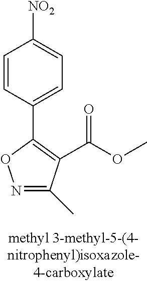 [SMILES: COC(=O)c1c(C)noc1-c1ccc([N+](=O)[O-])cc1] | 4- nitrobenzoyl chloride | LC-MS (ESI): m/z (M + 1): 263.1 (Method 1) 1H NMR (400 MHz, Chloroform-d) δ 2.56 (s, 3H), 3.90 (s, 3H), 8.07- 8.23 (m, 2H), 8.30-8.44 (m, 2H). |
| A5 | 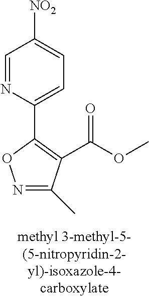 [SMILES: COC(=O)c1c(C)noc1-c1ccc([N+](=O)[O-])cn1] | 5- nitropicolinic acid | LC-MS (ESI): m/z (M + 1): 264.0 (Method 1) 1H NMR (400 MHz, Chloroform-d) δ 9.58 (dd, J = 2.6, 0.8 Hz, 1H), 8.66 (dd, J = 8.7, 2.6 Hz, 1H), 8.24 (dd, J = 8.7, 0.8 Hz, 1H), 3.91 (s, 3H), 2.56 (s, 3H). |
| A6 | 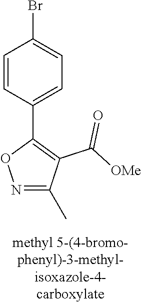 [SMILES: COC(=O)c1c(C)noc1-c1ccc(Br)cc1] | 4- bromobenzoic acid | LC-MS (ESI): m/z (M + 1): 298.0 (Method 1) |

### Step 4: 5-(5-bromo-6-methylpyridin-2-yl)-3-methylisoxazole-4-carboxylic acid (Intermediate A7)

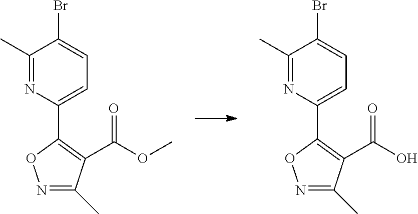

To a solution of methyl 5-(5-bromo-6-methylpyridin-2-yl)-3-methylisoxazole-4-carboxylate (Intermediate A3, 22.5 g, 72.32 mmol) in THE (100 mL) was added a solution of LiOH*H₂O (6.07 g, 144.63 mmol) in water (35 mL) and the biphasic reaction mixture was stirred at RT over 1 h. The mixture was cooled with an ice bath then a 2M aq. HCl solution (75 mL) was added to pH<2 while keeping the temperature below 15° C. The slurry was stirred over 10 min, the solid was filtered, washed with water (2×50 mL) and then with heptane (2×50 mL) and dried under vacuum to obtain the target product (22.2 g, crude) as a fine light yellow powder.

LC-MS (ESI): m/z (M+1): 299.1 (Method 1)

1H NMR (400 MHz, DMSO-d6) δ 14.87 (s, 1H), 8.36 (d, J=8.4 Hz, 1H), 7.90 (d, J=8.4 Hz, 1H), 2.67 (s, 3H), 2.46 (s, 3H)

The Intermediates in the following table were prepared from reagents reported below by using methods analogous to Intermediate A7.

| Intermediate | Structure & Name | Reagents | Analytical data |
|---|---|---|---|
| A8 | 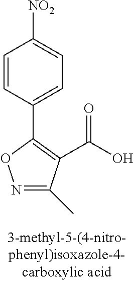 [SMILES: Cc1noc(-c2ccc([N+](=O)[O-])cc2)c1C(=O)O] | A4 | LC-MS (ESI): m/z (M + 1): 249.2 (Method 1) 1H NMR (400 MHz, DMSO-d6) δ ppm 13.67- 13.34 (m, 1 H), 8.39 (d, J = 9.02 Hz, 2 H), 8.17 (d, J = 9.02 Hz, 2 H), 2.46 (s, 3 H) |
| A5 | 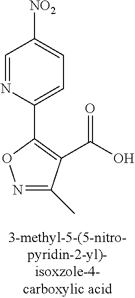 [SMILES: Cc1noc(-c2ccc([N+](=O)[O-])cn2)c1C(=O)O] | A5 | LC-MS (ESI): m/z (M + 1): 250.0 (Method 1) 1H NMR (400 MHz, DMSO-d6 δ 14.20-13.53 (m, 1 H), 9.53 (dd, J = 2.64, 0.66 Hz, 1 H), 8.83 (dd, J = 8.58, 2.64 Hz, 1 H), 8.35 (dd, J = 8.80, 0.66 Hz, 1 H) 2.47 (s, 3 H) |
| A10 | 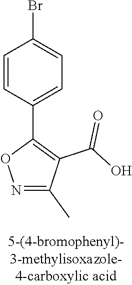 [SMILES: Cc1noc(-c2ccc(Br)cc2)c1C(=O)O] | A6 | LC-MS (ESI): m/z (M + 1): 282.0 (Method 1) |

### Step 5: (R)-1-(2-chlorophenyl)ethyl (5-(5-bromo-6-methylpyridin-2-yl)-3-methylisoxazol-4-yl)carbamate (Intermediate A11)

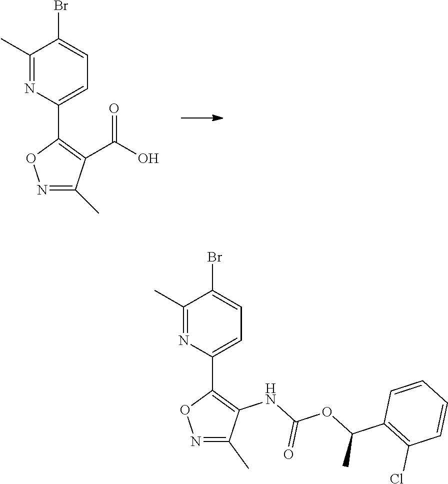 [SMILES: Cc1nc(-c2onc(C)c2C(=O)O)ccc1Br ; Cc1nc(-c2onc(C)c2NC(=O)O[C@H](C)c2ccccc2Cl)ccc1Br]

A 100 mL three-necked flask was charged with 5-(5-bromo-6-methylpyridin-2-yl)-3-methylisoxazole-4-carboxylic acid (Intermediate A7, 3.0 g, 10.1 mmol) and (R)-1-(2-chlorophenyl)ethan-1-ol (1.61 mL, 12.12 mmol). The system was closed and three cycles of vacuum/nitrogen back-filling were applied. Dry toluene (33 mL) was added, followed by TEA (2.81 mL, 20.2 mmol) and DPPA (3.26 mL, 15.15 mmol). The mixture was slowly heated at 90° C. and stirred at this temperature over 30 min. The mixture was cooled at RT, then sat. aq. KHCO₃ (30 mL) and EtOAc (30 mL) were added. The organic phase was separated and the aqueous phase was further extracted with EtOAc (30 mL). The collected organic fractions were washed with water (50 mL) and brine (50 mL), dried over Na₂SO₄, filtered and concentrated under reduced pressure. The supernatant oil was removed and the white crystals were washed with CyHex:EtOAc 9:1 (3×10 mL) to afford the target compound (4.6 g, crude) as a white solid.

LC-MS (ESI): m/z (M+1): 452.1 (Method 1)

The Intermediates in the following table were prepared from reagents reported below by using methods analogous to Intermediate A11.

| Intermediate | Structure & Name | Reagents | Analytical data |
|---|---|---|---|
| A12 | 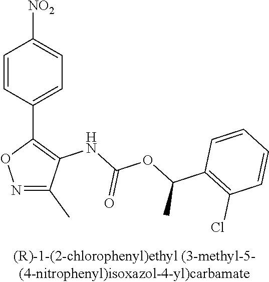 [SMILES: Cc1noc(-c2ccc([N+](=O)[O-])cc2)c1NC(=O)O[C@H](C)c1ccccc1Cl] | A8 | LC-MS (ESI): m/z (M + 1): 402.1 (Method 1) 1H NMR (400 MHz, DMSO-d6) d ppm 9.36- 9.75 (m, 1 H) 8.27-8.45 (m, 2 H) 8.01 (d, J = 8.80 Hz, 2 H) 7.22-7.77 (m, 4 H) 6.00 (q, J = 6.60 Hz, 1 H) 2.18 (s, 3 H) 1.55 (br. s., 3 H) |
| A13 | 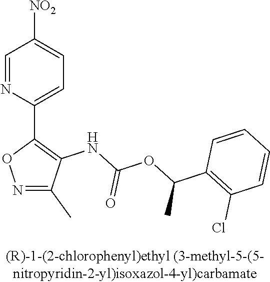 [SMILES: Cc1noc(-c2ccc([N+](=O)[O-])cn2)c1NC(=O)O[C@H](C)c1ccccc1Cl] | A9 | LC-MS (ESI): m/z (M + 1): 403.1 (Method 1) 1H NMR (400 MHz, Chloroform-d) δ 9.54 (d, J = 2.20 Hz, 1 H), , 8.70- 8.61 (m, 1 H), 8.52-8.34 (m, 1 H), 8.03 (d, J = 8.80 Hz, 1 H), 7.58-7.47 (m, 1 H), 7.43-7.24 (m, 3 H), 6.25 (m, 1 H), 2.49 (s, 3 H), 1.65 (d, J = 6.38 Hz, 3 H) |

### Step 6: (R)-1-(2-chlorophenyl)ethyl (5-(5-((tert-butoxycarbonyl)amino)-6-methylpyridin-2-yl)-3-methylisoxazol-4-yl)carbamate (Intermediate A14)

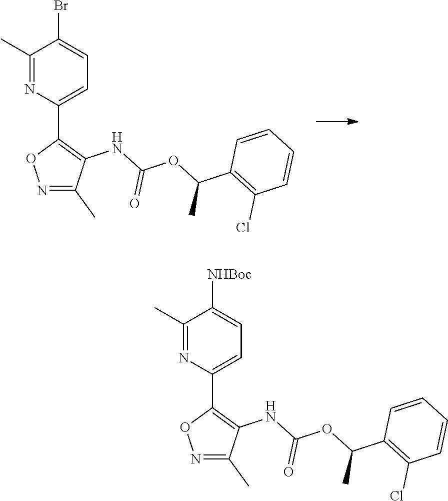 [SMILES: Cc1nc(-c2onc(C)c2NC(=O)O[C@H](C)c2ccccc2Cl)ccc1Br ; Cc1nc(-c2onc(C)c2NC(=O)O[C@H](C)c2ccccc2Cl)ccc1NC(=O)OC(C)(C)C]

A 100 mL three-necked flask equipped with stir bar, reflux condenser, thermometer and nitrogen/vacuum stopcock was charged with (R)-1-(2-chlorophenyl)ethyl (5-(5-bromo-6-methylpyridin-2-yl)-3-methylisoxazol-4-yl)carbamate (Intermediate A11, 4.6 g, crude), carbamic acid tert-butyl ester (1556 mg, 13.29 mmol), and K₃PO₄ (4700 mg, 22.14 mmol). The system was closed and three cycles of vacuum/nitrogen back-filling were applied. 1,4-Dioxane (45 mL) was added under nitrogen internal atmosphere, then three cycles of vacuum/nitrogen back-filling were applied to degas the suspension. XantPhos (576. mg, 1 mmol) and Pd₂(dba)₃ (355 mg, 0.390 mmol) were added. The mixture was heated at reflux for 1.5 h, then was cooled to RT, filtered over a plug of Celite and washed with EtOAc (3×10 mL). The filtrate was concentrated under reduced pressure to give a brown amorphous solid. n-Heptane (90 mL) and EtOAc (10 mL) were added and heated to reflux. The mixture was cooled to RT and stirred overnight. The crystalline solid was filtered, washed with fresh n-Heptane(3×15 mL) and dried under vacuum to afford the target product (5.6 g) as an off-white solid.

LC-MS (ESI): m/z (M+1): 487.3 (Method 1)

The Intermediate in the following table was prepared from reagents reported below by using methods analogous to Intermediate A14.

| Intermediate | Structure & Name | Reagents | Analytical data |
|---|---|---|---|
| A15 | 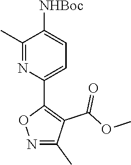 | A3 | LC-MS (ESI): m/z (M + 1): 348.3 (Method 1) 1H NMR (400 MHz, DMSO-d6) δ ppm 9.02 (s, 1 H) 8.05 (d, J = 8.36 Hz, 1 H) 7.80 (d, J = 8.58 Hz, 1 |
| | methyl 5-(5-((tert- | | H) 3.77 (s, |
| | butoxycarbonyl)amino)- | | 3 H) 2.41 (s, 3 |
| | 6-methylpyridin-2-y1)-3- | | H) 1.51 (s, |
| | methylisoxazole-4- | | 9 H) 1.40 (s, 3 |
| | carboxylate | | H) |

### Step 7: (R)-1-(2-chlorophenyl)ethyl (5-(5-amino-6-methylpyridin-2-yl)-3-methylisoxazol-4-yl)carbamate (Intermediate A16)

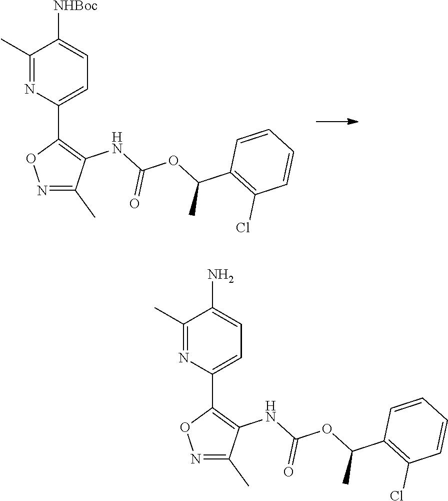 [SMILES: Cc1nc(-c2onc(C)c2NC(=O)O[C@H](C)c2ccccc2Cl)ccc1NC(=O)OC(C)(C)C ; Cc1nc(-c2onc(C)c2NC(=O)O[C@H](C)c2ccccc2Cl)ccc1N]

A 100 mL flask was charged with a solution of (R)-1-(2-chlorophenyl)ethyl (5-(5-((tert-butoxycarbonyl)amino)-6-methylpyridin-2-yl)-3-methylisoxazol-4-yl)carbamate (Intermediate A14, 5.6 g, 11.5 mmol) in 1,4-dioxane (20 mL). The mixture was kept at 20° C., then 4N HCl in 1,4-dioxane (20.0 mL, 80 mmol) was added dropwise. The mixture was stirred at RT for 16 h and concentrated under reduced pressure. The residue was suspended in diisopropyl ether, filtered, washed with diisopropyl ether and dried under vacuum to yield the target compound (4.3 g, 10.16 mmol, 88% yield) as an amorphous yellow solid. The product was used in the following step without purification.

LC-MS (ESI): m/z (M+1): 387.3 (Method 1)

### Step 8: methyl (1S,3S)-3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylate (Intermediate A17)

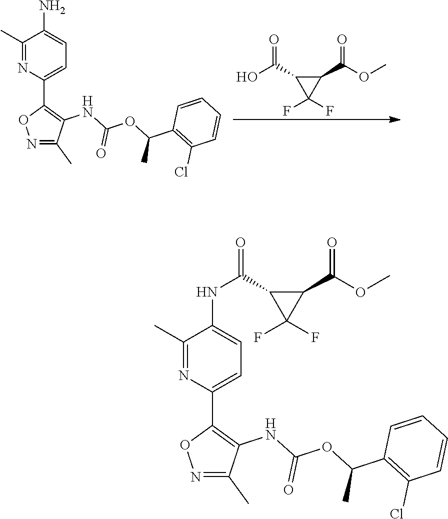 [SMILES: Cc1nc(-c2onc(C)c2NC(=O)O[C@H](C)c2ccccc2Cl)ccc1N ; COC(=O)[C@@H]1[C@@H](C(=O)O)C1(F)F ; COC(=O)[C@@H]1[C@@H](C(=O)Nc2ccc(-c3onc(C)c3NC(=O)O[C@H](C)c3ccccc3Cl)nc2C)C1(F)F]

To a solution (R)-1-(2-chlorophenyl)ethyl (5-(5-amino-6-methylpyridin-2-yl)-3-methylisoxazol-4-yl)carbamate (Intermediate A16, 100.0 mg, 0.260 mmol) and (1S,3S)-2,2-difluoro-3-(methoxycarbonyl)cyclopropane-1-carboxylic acid (Intermediate 2, 60.5 mg, 0.340 mmol) in MeCN (0.869 mL), 1-methylimidazole (0.07 mL, 0.900 mmol) was added, followed by TCFH (116. mg, 0.410 mmol). The mixture was stirred at 40° C. for 12 h. The solvent was evaporated under reduced pressure. The crude was loaded on a C18 Cartridge, washing first with 30% of MeCN in water and then with 80% of MeCN in water. This last fraction was evaporated to dryness under reduced pressure to afford the title compound (126.4 mg, 0.230 mmol, 89.% yield).

LC-MS (ESI): m/z (M+1): 549.1 (Method 1)

The Intermediates in the following table were prepared from reagents reported below following similar procedures as for Intermediate A17.

| Intermediate | Structure & Name | Reagents | Analytical data |
|---|---|---|---|
| A18 | 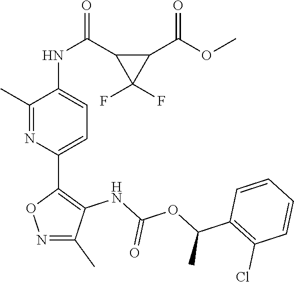 [SMILES: COC(=O)C1C(C(=O)Nc2ccc(-c3onc(C)c3NC(=O)O[C@H](C)c3ccccc3Cl)nc2C)C1(F)F] | A16 + Trans-rac 2,2- difluoro-3- (methoxy- carbonyl) cyclopropane- 1- carboxylic acid | LC-MS (ESI): m/z (M + 1): 549.1 (Method 1) 1H NMR (400 MHz, DMSO-d6) δ ppm 10.20 (br. s., 1 H) 9.05-9.42 (m, 1 H) 8.12 (d, J = 8.36 Hz, 1 H) 7.66 (d, J = 8.36 Hz, 2 H) 7.27-7.49 (m, 3 H) 6.00 (d, J = 5.72 Hz, 1 H) 3.76 (s, 3 H) 3.66-3.73 (m, 1 H) 3.18-3.28 (m, 1 H) 2.18 (s, 3 H) 1.42- 1.75 (m, 3 H) |
| | methyl 3-((6-(4-((((R)-1-(2- | | |
| | chlorophenyl)ethoxy)carbonyl) | | |
| | amino)-3-methylisoxazol-5- | | |
| | yl)-2-methylpyridin-3- | | |
| | yl)carbamoyl)-2,2- | | |
| | difluorocyclopropane-1- | | |
| | carboxylate mix of Trans | | |
| | diastereomers | | |
| A19 | 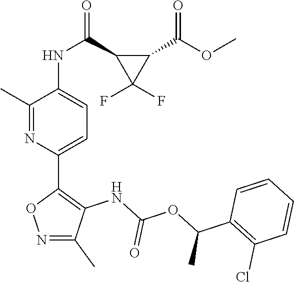 [SMILES: COC(=O)[C@H]1[C@H](C(=O)Nc2ccc(-c3onc(C)c3NC(=O)O[C@H](C)c3ccccc3Cl)nc2C)C1(F)F] | A16 + Intermediate 1 | LC-MS (ESI): m/z (M + 1): 549.1 (Method 1) |
| | methyl (1R,3R)-3-((6-(4- | | |
| | ((((R)-1-(2- | | |
| | chlorophenyl)ethoxy)carbonyl) | | |
| | amino)-3-methylisoxazol-5- | | |
| | yl)-2-methylpyridin-3- | | |
| | yl)carbamoyl)-2,2- | | |
| | difluorocyclopropane-1- | | |
| | carboxylate | | |

### Step 9: (1S,3S)-3-((6-(4-((((R)-1-(2-chlorophenyl)ethoxy)carbonyl)amino-3-methylisoxazol-5-yl)-2-methylpyridin-3-yl)carbamoyl)-2,2-difluorocyclopropane-1-carboxylic acid (Compound 1)

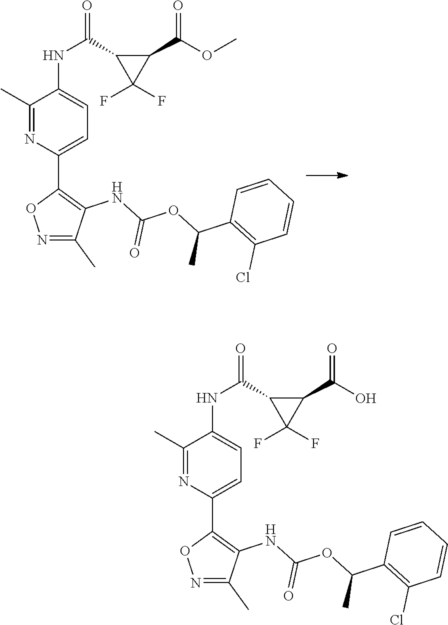 [SMILES: COC(=O)[C@@H]1[C@@H](C(=O)Nc2ccc(-c3onc(C)c3NC(=O)O[C@H](C)c3ccccc3Cl)nc2C)C1(F)F ; Cc1nc(-c2onc(C)c2NC(=O)O[C@H](C)c2ccccc2Cl)ccc1NC(=O)[C@@H]1[C@@H](C(=O)O)C1(F)F]

Methyl (1S,3S)-3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylate (Intermediate A17, 200.0 mg, 0.360 mmol) was dissolved in dry DCE (6 ml). SnMe₃OH (197.6 mg, 1.09 mmol) was added and the mixture was stirred at 60° C. overnight. The mixture was then acidified with a 1N HCl aqueous solution and evaporated. The crude was purified by reversed phase flash chromatography (eluent A: water+0.1% formic acid, eluent B: acetonitrile+0.100 formic acid, gradient A/B from 10/0 to 55/45) to give the target compound (62.7 mg, 0.117 mmol, 32. % yield) as a white solid.

LC-MS (ESI): m/z (M+1): 535.2 (Method 1)

¹H NMR (500 MH(z, DMSO-d6) δ ppm 12.83-14.17 (m, 1H), 10.18 (br s, 1H), 8.59-9.57 (m, 1H), 8.11 (d, J=8.4 Hz, 1H), 6.48-7.84 (m, 5H), 5.99 (br s, 1H), 3.61 (br dd, J=13.9, 7.8 Hz, 1H), 3.07 (br dd, J=14.1, 7.8 Hz, 1H), 2.47 (br s, 3H), 2.17 (br s, 3H), 1.53 (br s, 3H)

The Compound in the following table was prepared from reagents reported below following similar procedures as for Compound 1.

| Compound | Structure & Name | Reagents | Analytical data |
|---|---|---|---|
| 2 | 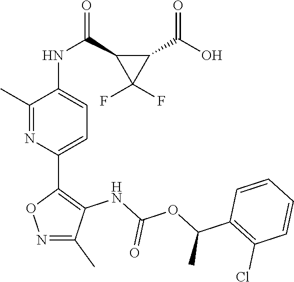 [SMILES: Cc1nc(-c2onc(C)c2NC(=O)O[C@H](C)c2ccccc2Cl)ccc1NC(=O)[C@H]1[C@H](C(=O)O)C1(F)F] | A19 | LC-MS (ESI): m/z (M + 1): 535.1 (Method 1) ¹H NMR (500 MHz, DMSO- d6) δ ppm 13.53 (br s, 1 H), 10.18 (br s, 1 H), 9.26 (br s, 1 H), 8.11 (d, J = 8.5 Hz, 1 H), 7.65 (d, J = 8.5 Hz, 1 H), 6.61- 7.75 (m, 4 H), 5.99 (br s, 1 H), 3.60 (br dd, J = 13.9, 7.8 Hz, 1 H), 3.07 (br dd, J = 14.3, 7.8 Hz, 1 H), 2.46 (s, 3 H), 2.17 (br s, 3 H), 1.53 (br s, 3 H) |
| | (1R,3R)-3-((6-(4-((((R)-1- | | |
| | (2- | | |
| | chlorophenyl)ethoxy)carbonyl) | | |
| | amino)-3- | | |
| | methylisoxazol-5-yl)-2- | | |
| | methylpyridin-3- | | |
| | yl)carbamoyl)-2,2- | | |
| | difluorocyclopropane-1- | | |
| | carboxylic acid | | |
| | methyl (1R,3R)-3-((6-(4- | | |
| | ((((R)-1-(2- | | |
| | chlorophenyl)ethoxy)carbonyl) | | |
| | amino)-3- | | |
| | methylisoxazol-5-yl)-2- | | |
| | methylpyridin-3- | | |
| | yl)carbamoyl)-2,2- | | |
| | difluorocyclopropane-1- | | |
| | carboxylic acid | | |

### Example 2

### Process synthesis of 3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid, mix of Trans diastereomers (Compound 3) and of its Single diastereomers Trans Compounds 5 and Compound 6

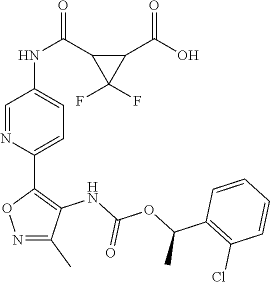 [SMILES: Cc1noc(-c2ccc(NC(=O)C3C(C(=O)O)C3(F)F)cn2)c1NC(=O)O[C@H](C)c1ccccc1Cl]

### Step 1: (R)-1-(2-chlorophenyl)ethyl (5-(5-aminopyridin-2-yl)-3-methylisoxazol-4-yl)carbamate (Intermediate B1)

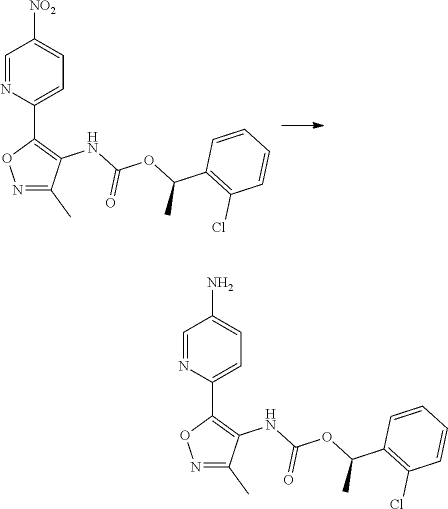 [SMILES: Cc1noc(-c2ccc([N+](=O)[O-])cn2)c1NC(=O)O[C@H](C)c1ccccc1Cl ; Cc1noc(-c2ccc(N)cn2)c1NC(=O)O[C@H](C)c1ccccc1Cl]

(R)-1-(2-chlorophenyl)ethyl (3-methyl-5-(5-nitropyridin-2-yl)isoxazol-4-yl)carbamate (Intermediate A13, 300.0 mg, 0.740 mmol) was dissolved in DCM (8.3 mL) and methanol (2.8 mL) at RT, then 37% HCl (0.06 mL, 0.740 mmol) was added dropwise, followed by portion-wise addition of Fe⁰ (291 mg, 5.21 mmol). The mixture was stirred at RT for 3 h, then concentrated under reduced pressure. The residue was basified with 2N NaOH solution and extracted with EtOAc, the organic phase was washed with brine, dried over Na₂SO₄, filtered and concentrated in vacuo. The crude compound was purified by flash chromatography (gradient EtOAc in CyHex 0% to 30%) to give the desired compound (70 mg, 0.188 mmol, 25.% yield) as a pale yellow solid.

LC-MS (ESI): m/z (M+1): 373.1 (Method 1)

1H NMR (400 MHz, DMSO-d6) δ ppm 9.02 (br. s., 1H), 8.02 (d, J=2.64 Hz, 1H), 7.53-7.75 (m, 1H), 7.50-7.28 (m, 4H), 6.96 (dd, J=8.58, 2.64 Hz, 1H), 6.01-5.85 (m, 3H), 2.09 (br. s., 3H), 1.35-1.66 (m, 3H)

The Intermediate in the following table was prepared from reagents reported below following similar procedures as for Intermediate B1.

| Intermediate | Structure & Name | Reagents | Analytical data |
|---|---|---|---|
| B2 | 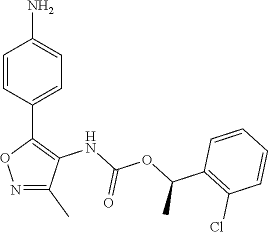 | A12 | LC-MS (ESI): m/z (M + 1):369.1 (Method 1) 1H NMR (400 MHz, CDCl3) δ 1.59 (br s, 3H), 2.24 (s, 3H), 3.96 (br s, 2H), 5.88 (br s, 1H), 6.24 (q, J = 6.5 Hz, 1H), 6.56- 6.80 (m, 2H), 6.93-7.45 (m, 3H), 7.59 (s, 3H) |
| | (R)-1-(2- | | |
| | chlorophenyl)ethyl (5-(4- | | |
| | aminophenyl)-3- | | |
| | methylisoxazol-4- | | |
| | yl)carbamate | | |

### Step 2: methyl 3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylate, mix of Trans diastereomers (Intermediate B3)

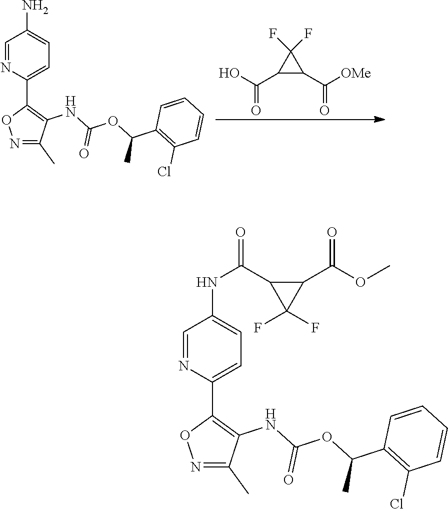 [SMILES: Cc1noc(-c2ccc(N)cn2)c1NC(=O)O[C@H](C)c1ccccc1Cl ; COC(=O)C1C(C(=O)O)C1(F)F ; COC(=O)C1C(C(=O)Nc2ccc(-c3onc(C)c3NC(=O)O[C@H](C)c3ccccc3Cl)nc2)C1(F)F]

Intermediate B3 (32 mg, 0.060 mmol, 33% yield) was obtained from (R)-1-(2-chlorophenyl)ethyl (5-(4-aminophenyl)-3-methylisoxazol-4-yl)carbamate (Intermediate B1, 70.0 mg, 0.180 mmol) and Trans-rac 2,2-difluoro-3-methoxycarbonylcyclopropane-1-carboxylic acid (42.64 mg, 0.240 mmol) using a similar method of Intermediate A17.

LC-MS (ESI): m z (M+1): 535.2 (Method 1)

### Step 3: 3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid, mix of Trans diastereomers (Compound 3)

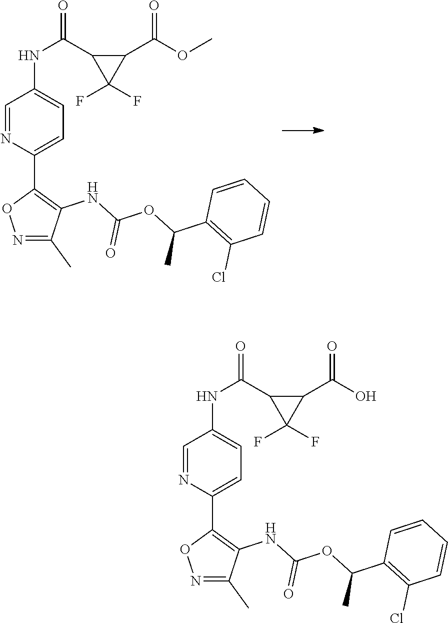 [SMILES: COC(=O)C1C(C(=O)Nc2ccc(-c3onc(C)c3NC(=O)O[C@H](C)c3ccccc3Cl)nc2)C1(F)F ; Cc1noc(-c2ccc(NC(=O)C3C(C(=O)O)C3(F)F)cn2)c1NC(=O)O[C@H](C)c1ccccc1Cl]

Methyl 3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylate, mix of Trans diastereomers (Intermediate B3, 32 mg, 0.060 mmol) was dissolved in MeOH (1.231 mL) and water (0.615 mL). LiOH (2.9 mg, 0.120 mmol) was added and the mixture was stirred at RT for 2h. 6M HCl was added to the mixture till pH<4 and the mixture was evaporated to obtain the crude Compound 3 (31 mg).

LC-MS (ESI): m z (M+1): 521.1 (Method 1)

The Compound in the following table was prepared from reagents reported below following similar procedures as for Compound 3.

| Compound | Structure & Name | Reagents | Analytical data |
|---|---|---|---|
| 4 | 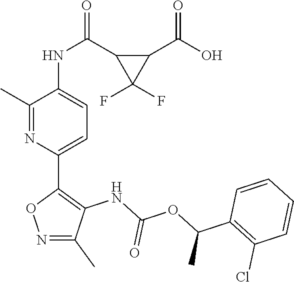 [SMILES: Cc1nc(-c2onc(C)c2NC(=O)O[C@H](C)c2ccccc2Cl)ccc1NC(=O)C1C(C(=O)O)C1(F)F] | A18 | LC-MS (ESI): m/z (M + 1): 535.2 (Method 1) 1H NMR (500 MHZ, DMSO-d6) δ ppm 12.83- 14.17 (m, 1 H), 10.18 (br s, 1 H), 8.59-9.57 (m, 1 H), 8.11 (d, J = 8.4 Hz, 1 H), 6.48-7.84 (m, 5 H), 5.99 (br s, 1 H), 3.61 (br dd, J = 13.9, 7.8 Hz, 1 H), 3.07 (br dd, J = 14.1, 7.8 Hz, 1 H), 2.47 (br s, 3 H), 2.17 (br s, 3 H), 1.53 (br s, 3 H) |
| | 3-((6-(4-((((R)-1-(2- | | |
| | chlorophenyl)ethoxy)carbonyl) | | |
| | amino)-3-methylisoxazol-5-y1)-2- | | |
| | methylpyridin-3-yl)carbamoyl)- | | |
| | 2,2-difluorocyclopropane-1- | | |
| | carboxylic acid, mix of Trans | | |
| | diastereomers | | |

Compound 3 was submitted to chiral preparative HPLC.

Conditions: Chiralpak IC (25×2.0 cm), 5μ Mobile phase n-Hexane/(Ethanol+0.1% formic acid) 70/30% v/v Flow rate (ml/min) 17 ml/min DAD detection 220 nm Loop 1000 μL

### Peak 1: Compound 5

Single Diastereomer 1 of Trans-3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid

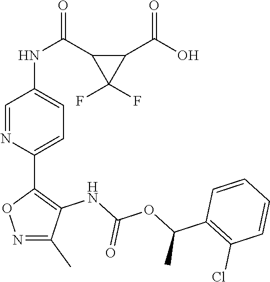 [SMILES: Cc1noc(-c2ccc(NC(=O)C3C(C(=O)O)C3(F)F)cn2)c1NC(=O)O[C@H](C)c1ccccc1Cl]

LC-MS (ESI): m/z (M+1): 521.0 (Method 1)

1H NMR (400 MHz, DMSO-d6) δ ppm 11.03 (s, 1H), 9.25 (br d, J=0.9 Hz, 1H), 8.86 (br s, 1H), 8.19 (dd, J=8.8, 2.4 Hz, 1H), 7.76 (d, J=8.8 Hz, 1H), 7.16-8.07 (m, 4H), 5.95 (br d, J=6.4 Hz, 1H), 3.11-3.23 (m, 1H), 2.70-2.80 (m, 1H), 2.15 (s, 3H), 1.42 (br s, 3H)

### Peak 2: Compound 6

Single Diastereomer 2 of Trans-3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid

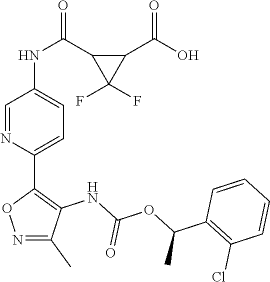 [SMILES: Cc1noc(-c2ccc(NC(=O)C3C(C(=O)O)C3(F)F)cn2)c1NC(=O)O[C@H](C)c1ccccc1Cl]

LC-MS (ESI): m/z (M+1): 521.0 (Method 1)

1H NMR (400 MHz, DMSO-d6) δ ppm 13.54 (br s, 1H), 11.03 (s, 1H), 9.03-9.53 (m, 1H), 8.83 (s, 1H), 8.15 (dd, J=8.8, 2.4 Hz, 1H), 7.78 (d, J=8.6 Hz, 1H), 7.06-8.03 (m, 4H), 5.95 (br d, J=6.6 Hz, 1H), 3.31-3.43 (m, 1H), 3.07 (br dd, J=14.5, 7.5 Hz, 1H), 2.15 (s, 3H), 1.53 (br s, 3H)

### Example 3

### Process synthesis of 3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid, mix of Trans diastereomers (Compound 7) and of its Single diastereomers Trans Compound 8 and Compound 9

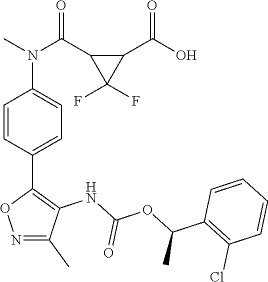 [SMILES: Cc1noc(-c2ccc(N(C)C(=O)C3C(C(=O)O)C3(F)F)cc2)c1NC(=O)O[C@H](C)c1ccccc1Cl]

### Step 1: (R)-1-(2-chlorophenyl)ethyl (3-methyl-5-(4-(methylamino)phenyl)isoxazol-4-yl)carbamate (Intermediate C1)

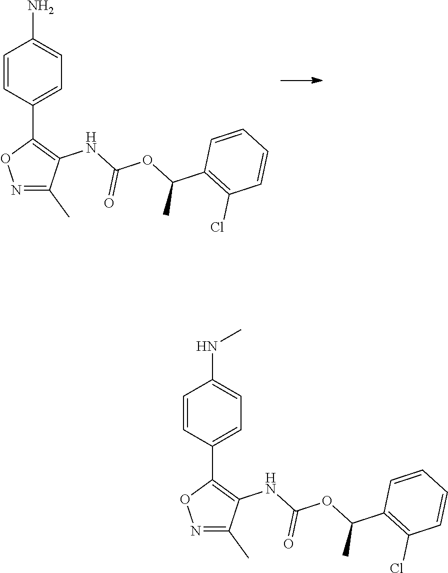 [SMILES: Cc1noc(-c2ccc(N)cc2)c1NC(=O)O[C@H](C)c1ccccc1Cl ; CNc1ccc(-c2onc(C)c2NC(=O)O[C@H](C)c2ccccc2Cl)cc1]

(R)-1-(2-chlorophenyl)ethyl (5-(4-aminophenyl)-3-methylisoxazol-4-yl)carbamate (Intermediate B2, 650.0 mg, 1.75 mmol) was dissolved in THE (4 mL), then formaldehyde (0.65 mL, 8.74 mmol), MeOH (8 mL) and sodium methoxide (472 mg, 8.74 mmol) were added. The reaction mixture was stirred at 65° C. overnight, then was cooled to RT and added with sodium borohydride (330.6 mg, 8.74 mmol). After stirring at 65° C. for further 3 h, the solvent was evaporated, then NaHCO₃ satd. sol. and EtOAc were added and the aqueous phase was extracted with EtOAc; collected organic phases were dried over Na₂SO₄, filtered and evaporated to give the crude material that was purified by flash chromatography (gradient CyHex/EtOAc from 9/1 to 1/1) to afford the title compound (215 mg, 0.557 mmol, 32% yield) as white solid.

LC-MS (ESI): m/z (M+1): 386.2 (Method 1)

### Step 2: methyl 3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylate, mix of Trans diastereomers (Intermediate C2)

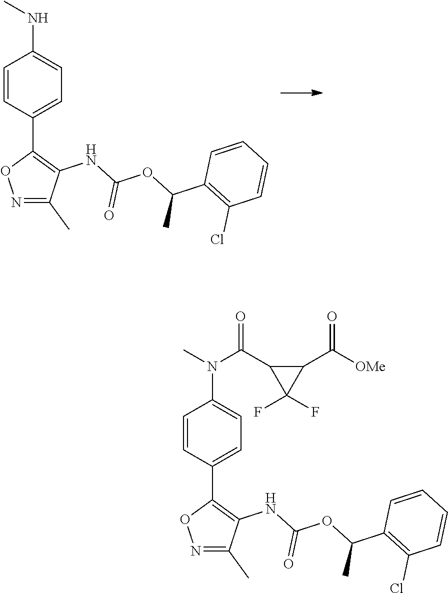 [SMILES: CNc1ccc(-c2onc(C)c2NC(=O)O[C@H](C)c2ccccc2Cl)cc1 ; COC(=O)C1C(C(=O)N(C)c2ccc(-c3onc(C)c3NC(=O)O[C@H](C)c3ccccc3Cl)cc2)C1(F)F]

Intermediate C2 (280 mg, 0.511 mmol, 92% yield) was obtained from (R)-1-(2-chlorophenyl)ethyl (3-methyl-5-(4-(methylamino)phenyl)isoxazol-4-yl)carbamate (Intermediate C1, 215.0 mg, 0.560 mmol) and Trans-rac 2,2-difluoro-3-(methoxycarbonyl)cyclopropane-1-carboxylic acid (0.13 g, 0.720 mmol) using a similar method as for Intermediate A17.

LC-MS (ESI): m/z (M+1): 548.3 (Method 1)

### Step 3: 3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid, mix of Trans diastereomers (Compound 7)

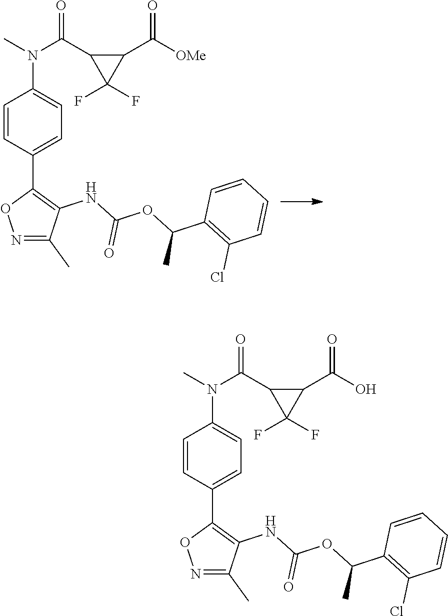 [SMILES: COC(=O)C1C(C(=O)N(C)c2ccc(-c3onc(C)c3NC(=O)O[C@H](C)c3ccccc3Cl)cc2)C1(F)F ; Cc1noc(-c2ccc(N(C)C(=O)C3C(C(=O)O)C3(F)F)cc2)c1NC(=O)O[C@H](C)c1ccccc1Cl]

Compound 7 (200 mg, 0.375 mmol, 73% yield) was obtained from methyl 3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylate, mix of Trans diastereomers (Intermediate C2, 280 mg, 0.511 mmol) using a similar method as for Compound 3, step 3..

LC-MS (ESI): m/z (M+1): 534.3 (Method 1) Compound 7 was submitted to chiral preparative HPLC.

Conditions: Chiralpak IC (25×3.0 cm), 5μ Mobile phase n-Hexane/(Ethanol+0.1% formic acid) 80/20% v/v Flow rate (ml/min) 35 ml/min DAD detection 220 nm Loop 200 μL

### Peak 1: Compound 8

Single Diastereomer 1 of Trans-3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid

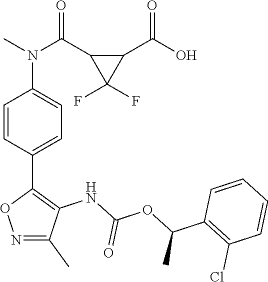 [SMILES: Cc1noc(-c2ccc(N(C)C(=O)C3C(C(=O)O)C3(F)F)cc2)c1NC(=O)O[C@H](C)c1ccccc1Cl]

LC-MS (ESI): m/z (M+1): 534.3 (Method 1) 1H NMR (400 MHz, DMSO-d6) δ ppm 13.18-13.57 (m, 1H) 9.38-9.54 (m, 1H) 7.89 (br. s., 2H) 7.43-7.67 (m, 5H) 7.31-7.40 (m, 1H) 6.01 (d, J=6.60 Hz, 1H) 2.91-3.06 (m, 1H) 2.68-2.83 (m, 1H) 2.5 (s, 3H) 2.14 (br. s., 3H) 1.56 (d, J=5.50 Hz, 3H).

Compound 8 was converted into the corresponding sodium salt following the procedure below.

Compound 8, 89.3 mg, 0.170 mmol), was dissolved in MeOH (3 mL), then 0.1 M NaOH (1.67 mL, 0.170 mmol) was added. The mixture was stirred at RT for 2h. Solvent was evaporated to afford the target compound (84.5 mg, 0.152 mmol, 91% yield) as off-white solid.

LC-MS (ESI): m/z (M+1-Na⁺): 534.3 (Method 1)

1H NMR (400 MHz, DMSO-d6) δ ppm 9.49 (br s, 1H), 7.84 (br d, J=7.5 Hz, 2H), 7.46 (br d, J=7.5 Hz, 2H), 6.92-8.06 (m, 4H), 6.00 (q, J=6.5 Hz, 1H), 3.25 (br s, 3H), 2.36-2.65 (m, 2H), 2.12 (s, 3H), 1.25-1.66 (m, 3H).

### Peak 2: Compound 9

Single Diastereomer 2 of Trans-3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid

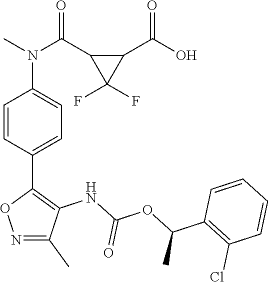 [SMILES: Cc1noc(-c2ccc(N(C)C(=O)C3C(C(=O)O)C3(F)F)cc2)c1NC(=O)O[C@H](C)c1ccccc1Cl]

LC-MS (ESI): m/z (M+1): 534.3 (Method 1)

1H NMR (400 MHz, DMSO-d6) δ ppm 12.95-13.46 (m, 1H) 9.37-9.55 (m, 1H) 7.80-8.01 (m, 2H) 7.42-7.70 (m, 5H) 7.31-7.41 (m, 1H) 5.92-6.10 (m, 1H) 2.92-3.01 (m, 1H) 2.66-2.80 (m, 1H) 2.5 (s, 3H) 2.06-2.22 (m, 3H) 1.44-1.63 (m, 3H)

The corresponding sodium salt of Compound 9 was obtained following similar procedure as for Compound 8.

LC-MS (ESI): m/z (M+1-Na+): 534.3 (Method 1)

1H NMR (400 MHz, DMSO-d6) δ ppm 9.49 (br s, 1H), 7.84 (br d, J=7.5 Hz, 2H), 7.46 (br d, J=7.5 Hz, 2H), 6.92-8.06 (m, 4H), 6.00 (q, J=6.5 Hz, 1H), 3.25 (br s, 3H), 2.36-2.65 (m, 2H), 2.12 (s, 3H), 1.25-1.66 (m, 3H).

### Example 4

### Process of synthesis of 2,2-difluoro-3-((6-(4-((6-isopropoxypyrazin-2-yl)amino)-3-methylisoxazol-5-yl)-2-methylpyridin-3-yl)carbamoyl)cyclopropane-1-carboxylic acid, mix of Trans diastereomers (Compound 10) and of its Single Enantiomer Trans Compound 11

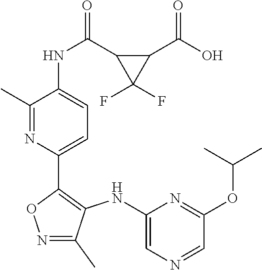 [SMILES: Cc1nc(-c2onc(C)c2Nc2cncc(OC(C)C)n2)ccc1NC(=O)C1C(C(=O)O)C1(F)F]

Step 1: 5-(5-((tert-butoxycarbonyl)amino)-6-methylpyridin-2-yl)-3-methylisoxazole-4-carboxylic acid (Intermediate D1)

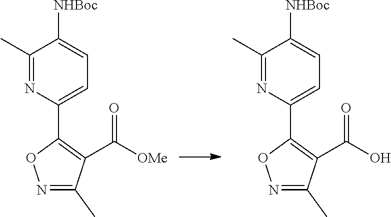 [SMILES: COC(=O)c1c(C)noc1-c1ccc(NC(=O)OC(C)(C)C)c(C)n1 ; Cc1nc(-c2onc(C)c2C(=O)O)ccc1NC(=O)OC(C)(C)C]

Intermediate D1 (1.71 g, 5.13 mmol, 75% yield) was prepared from methyl 5-(5-((tert-butoxycarbonyl)amino)-6-methylpyridin-2-yl)-3-methylisoxazole-4-carboxylate (Intermediate A15, 2.55 g, 6.82 mmol) using a similar method as for Intermediate A7.

LC-MS (ESI): m/z (M+1): 334.2 (Method 1)

### Step 2: benzyl (5-(5-((tert-butoxycarbonyl)amino)-6-methylpyridin-2-yl)-3-methylisoxazol-4-yl)carbamate (Intermediate D2)

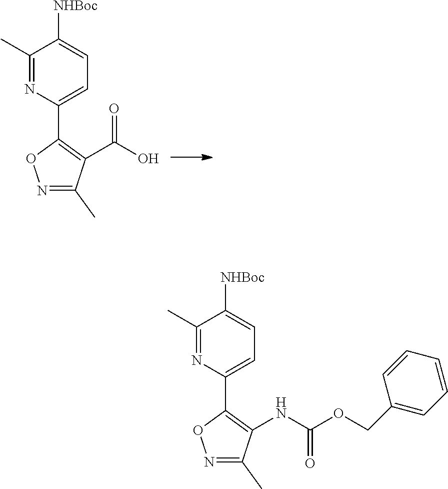 [SMILES: Cc1nc(-c2onc(C)c2C(=O)O)ccc1NC(=O)OC(C)(C)C ; Cc1nc(-c2onc(C)c2NC(=O)OCc2ccccc2)ccc1NC(=O)OC(C)(C)C]

Intermediate D2 (421 mg, 0.960 mmol, 27% yield) was prepared from 5-(5-((tert-butoxycarbonyl)amino)-6-methylpyridin-2-yl)-3-methylisoxazole-4-carboxylic acid (Intermediate D1, 1.17 g, 3.51 mmol) and phenylmethanol (0.73 mL, 7.02 mmol) using a similar method as for intermediate A11.

LC-MS (ESI): m/z (M+1): 439.3 (Method 1)

### Step 3: tert-butyl (6-(4-amino-3-methylisoxazol-5-yl)-2-methylpyridin-3-yl)carbamate (Intermediate D3)

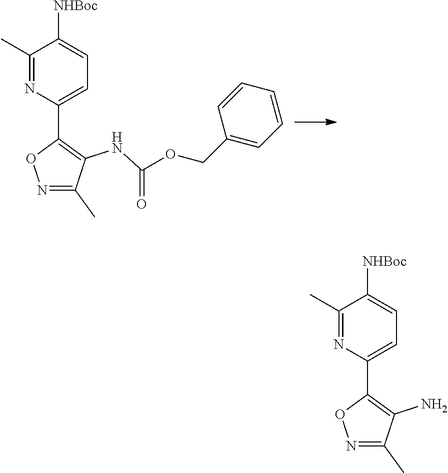 [SMILES: Cc1nc(-c2onc(C)c2NC(=O)OCc2ccccc2)ccc1NC(=O)OC(C)(C)C ; Cc1nc(-c2onc(C)c2N)ccc1NC(=O)OC(C)(C)C]

To a solution of benzyl (5-(5-((tert-butoxycarbonyl)amino)-6-methylpyridin-2-yl)-3-methylisoxazol-4-yl)carbamate (Intermediate D2, 421.0 mg, 0.960 mmol) in ethyl acetate (9.087 mL), palladium on charcoal (35.5 mg, 0.030 mmol) was added. The mixture was stirred at RT overnight under hydrogen atmosphere. The mixture was filtered over Celite, the pad was washed with MeOH (3×15 mL) and the filtrate was concentrated under reduced pressure. The title compound (307 mg, crude) was obtained as a white solid.

LC-MS (ESI): m/z (M+1): 305.2 (Method 1)

### Step 4: tert-butyl (6-(4-((6-isopropoxypyrazin-2-yl)amino)-3-methylisoxazol-5-yl)-2-methylpyridin-3-yl)carbamate (Intermediate D4)

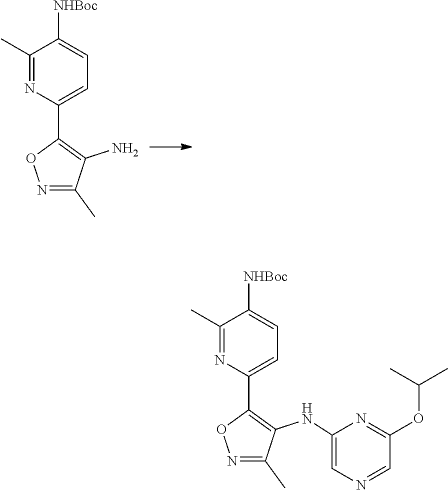 [SMILES: Cc1nc(-c2onc(C)c2N)ccc1NC(=O)OC(C)(C)C ; Cc1nc(-c2onc(C)c2Nc2cncc(OC(C)C)n2)ccc1NC(=O)OC(C)(C)C]

A 25 mL round bottom flask equipped with stirring bar was charged with tert-butyl (6-(4-amino-3-methylisoxazol-5-yl)-2-methylpyridin-3-yl)carbamate (Intermediate D3, 307 mg, crude) and sodium tert-butoxide (154 mg, 1.6 mmol). The flask was purged performing three cycles of vacuum-nitrogen back-filling, then toluene (10 mL) and 2-chloro-6-propan-2-yloxypyrazine (199 mg, 1.15 mmol) were added. The mixture was degassed (three cycles of vacuum-N₂ back-filling), then 2-(2-dicyclohexylphosphinophenyl)-N,N-dimethylaniline (59 mg, 0.150 mmol) and Pd₂(dba)₃ (69 mg, 0.080 mmol) were added and the mixture was further degassed (3 cycles vacuum-N₂ back-filling), then heated at 90° C. for 1 hour. The mixture was cooled at RT and diluted with EtOAc (30 mL) and sat. aq. NH₄C₁ (30 mL). The organic phase was separated and the aq. phase was extracted with EtOAc (20 mL). The collected organic layers were washed with water (30 mL), brine (30 mL), dried over Na₂SO₄, filtered and concentrated to dryness. The crude was purified by reversed phase flash chromatography, eluting with a gradient of EtOAc in CyHex from 0% to 50%, then with a gradient of Water/MeCN+0.1% HCOOH from 95:5 to 30:70 affording the title compound (245 mg) as a pale yellow solid.

LC-MS (ESI): m/z (M+1): 401.3 (Method 1)

The Intermediate in the following table was prepared from reagents reported below following similar procedures as for Intermediate D4.

| Intermediate | Structure & Name | Reagents | Analytical data |
|---|---|---|---|
| D5 | 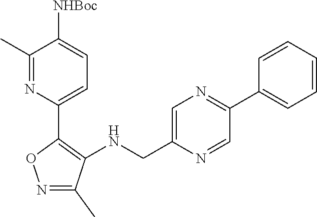 | D3 + 2- chloro-6- phenylpyrazine | LC-MS (ESI): m/z (M + 1): 459.3 (Method 1) 1H NMR (400 MHz, Acetone-d6) δ 8.57 (d, J = 0.58 Hz, 1H), 8.36 (dd, J = 1.85, 0.54 Hz, 1H), 8.29 (dd, J = 8.55, 2.94 Hz, 1H), 8.06-7.96 (m, 3H), 7.74 (d, J = 8.55 Hz, 1H), 7.53-7.41 (m, 4H), 2.56 (d, J = 0.80 Hz, 3H), 2.36 (d, J = 1.57 Hz, 3H), 1.50 (s, 9H) |
| | tert-butyl (2-methyl-6-(3- | | |
| | methyl-4-((6- | | |
| | phenylpyrazin-2- | | |
| | yl)amino)isoxazol-5- | | |
| | yl)pyridin-3- | | |
| | yl)carbamate | | |

### Step 5: 5-(5-amino-6-methylpyridin-2-yl)-N-(6-isopropoxypyrazin-2-yl)-3-methylisoxazo-4-amine (Intermediate D6)

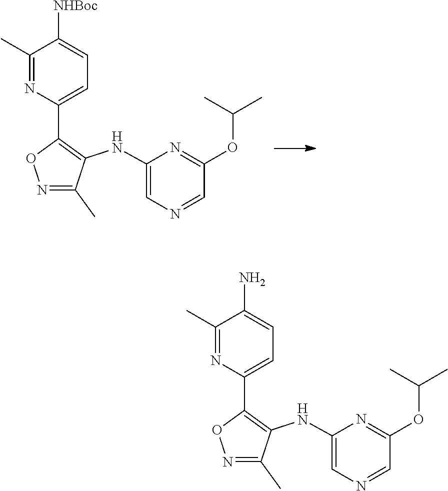 [SMILES: Cc1nc(-c2onc(C)c2Nc2cncc(OC(C)C)n2)ccc1NC(=O)OC(C)(C)C ; Cc1nc(-c2onc(C)c2Nc2cncc(OC(C)C)n2)ccc1N]

To a solution of tert-butyl (6-(4-((6-isopropoxypyrazin-2-yl)amino)-3-methylisoxazol-5-yl)-2-methylpyridin-3-yl)carbamate (Intermediate D4, 245 mg, 0.556 mmol) in DCM (10 mL), TFA (3 mL) was added and the mixture was stirred at RT for 2 hours. The mixture was concentrated under reduced pressure, then the residue was suspended in toluene and concentrated under reduced pressure (3 times). The crude was re-dissolved in DCM, a solution of Na₂CO₃ was slowly added to pH˜9 and the phases were separated. The aqueous layer was further extracted with DCM (2×20 mL), then the combined organic layer was evaporated to dryness under reduced pressure to afford the title compound (347 mg, crude) as a yellow solid.

LC-MS (ESI): m/z (M+1): 301.3 (Method 1)

The Intermediate in the following table was prepared from reagents reported below following similar procedures as for Intermediate D6.

| Intermediate | Structure & Name | Reagents | Analytical data |
|---|---|---|---|
| D7 | 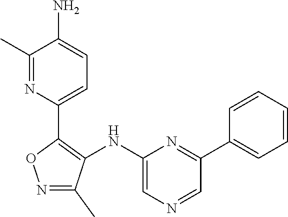 [SMILES: Cc1nc(-c2onc(C)c2Nc2cncc(-c3ccccc3)n2)ccc1N] | D5 | LC-MS (ESI): m/z (M + 1): 359.2 (Method 1) 1H NMR (400 MHz, DMSO-d6) δ 8.76 (s, 1H), 8.47 (s, 1H), 8.13 (s, 1H), 7.90-7.82 (m, 2H), 7.46- 7.40 (m, 4H), 6.95 (d, J = 8.38 Hz, 1H), 5.55 (s, 2H), 2.50 (s, 3H), 2.20 (d, J = 1.64 Hz, 6H) |
| | 5-(5-amino-6- | | |
| | methylpyridin-2-y1)-3- | | |
| | methyl-N-(6- | | |
| | phenylpyrazin-2- | | |
| | yl)isoxazol-4-amine | | |

### Step 6: methyl 2-((6-(4-((6-isopropoxypyrazin-2-yl)amino)-3-methylisoxazol-5-yl)-2-methylpyridin-3-yl)carbamoyl)cyclopropane-1-carboxylate, mix of Trans diastereomers (Intermediate d 8)

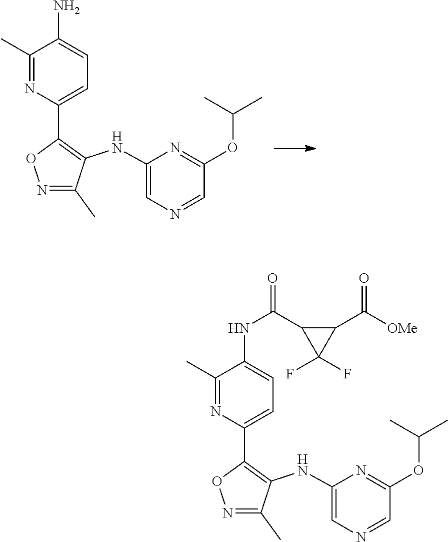 [SMILES: Cc1nc(-c2onc(C)c2Nc2cncc(OC(C)C)n2)ccc1N ; COC(=O)C1C(C(=O)Nc2ccc(-c3onc(C)c3Nc3cncc(OC(C)C)n3)nc2C)C1(F)F]

Intermediate D8 (387 mg) was prepared from 5-(5-amino-6-methylpyridin-2-yl)-N-(6-sopropoxypyrazin-2-yl)-3-methylisoxazol-4-amine (Intermediate D6, 347.0 mg, crude) and Trans-rae 2,2-difluoro-3-(methoxycarbonyl)cyclopropane-1-carboxylic acid (238.7 mg, 1.33 mmol) using a similar method as for Intermediate A17.

LC-MS (ESI): m/z (M+1): 503.3 (Method 1)

The Intermediate in the following table was prepared from reagents reported below following similar procedures as for Intermediate A17.

| Intermediate | Structure & Name | Reagents | Analytical data |
|---|---|---|---|
| D9 | 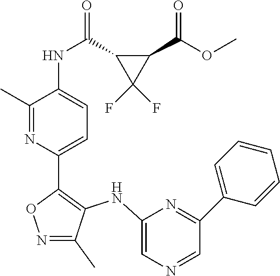 [SMILES: COC(=O)[C@@H]1[C@@H](C(=O)Nc2ccc(-c3onc(C)c3Nc3cncc(-c4ccccc4)n3)nc2C)C1(F)F] | D7 + intermediate 2 | LC-MS (ESI): m/z (M + 1): 521.2 (Method 1) 1H NMR (400 MHz, DMSO-d6) δ 10.14 (s, 1H), 8.98 (s, 1H), 8.49 (s, 1H), 8.20 (s, 1H), 8.09 (d, J = 8.47 Hz, 1H), 7.79- 7.69 (m, 3H), 7.51-7.35 (m, 3H), 3.74 (s, 3H), 3.65 (dd, J = 14.10, 7.69 Hz, 1H), 3.24-3.13 (m, 1H), 2.38 (s, 3H), 2.26 (s, 3H). |
| | methyl (1S,3S)-2,2- | | |
| | difluoro-3-((2-methyl-6- | | |
| | (3-methyl-4-((6- | | |
| | phenylpyrazin-2- | | |
| | yl)amino)isoxazol-5- | | |
| | yl)pyridin-3- | | |
| | yl)carbamoyl)cyclopropane- | | |
| | 1-carboxylate | | |

### Step 7: 2-((6-(4-((6-isopropoxypyrazin-2-yl)amino)-3-methylisoxazol-5-yl)-2-methylpyridin-3-yl)carbamoyl)cyclopropane-1-carboxylic acid, mix of Trans diastereomers (Compound 10)

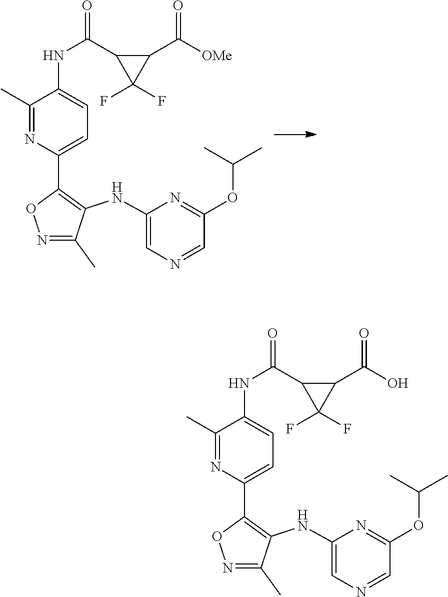 [SMILES: COC(=O)C1C(C(=O)Nc2ccc(-c3onc(C)c3Nc3cncc(OC(C)C)n3)nc2C)C1(F)F ; Cc1nc(-c2onc(C)c2Nc2cncc(OC(C)C)n2)ccc1NC(=O)C1C(C(=O)O)C1(F)F]

Compound 10 (267 mg, 0.547 mmol, 41% yield) was prepared from methyl 2-((6-(4-((6-isopropoxypyrazin-2-yl)amino)-3-methylisoxazol-5-yl)-2-methylpyridin-3-yl)carbamoyl)cyclopropane-1-carboxylate, mix of Trans diastereomers (Intermediate D8, 238.7 mg, 1.33 mmol) using a similar method as for Compound 3, step 3.

LC-MS (ESI): m/z (M+1): 489.2 (Method 1)

Compound 10 was submitted to chiral preparative SFC.

Conditions: Column Whelk 01 (R,R) (25×3.0 cm), 10 Modifier (Methanol+0.1% formic acid) 28% Flow rate (ml/min) 100 Pressure (bar) 120 Temperature (° C.) 38 UV detection 220 nm Loop 500 μL

### Peak 1: Compound 11

Single Diastereomer 2 of Trans-2-((6-(4-((6-isopropoxypyrazin-2-yl)amino)-3-methylisoxazol-5-yl)-2-methylpyridin-3-yl)carbamoyl)cyclopropane-1-carboxylic acid

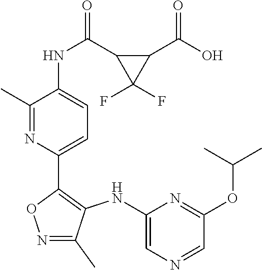 [SMILES: Cc1nc(-c2onc(C)c2Nc2cncc(OC(C)C)n2)ccc1NC(=O)C1C(C(=O)O)C1(F)F]

LC-MS (ESI): m/z (M+1): 489.2 (Method 1)

The corresponding sodium salt of Compound 11 was prepared using a similar method as for Compound 8. Compound 11 was characterized as sodium salt.

LC-MS (ESI): m/z (M+1-Na⁺): 489.2 (Method 1)

1H NMR (500 MHz, DMSO-d6) δ ppm 10.14 (s, 1H), 8.72 (s, 1H), 8.06 (d, J=8.5 Hz, 1H), 7.68 (s, 1H), 7.66 (d, J=8.5 Hz, 1H), 7.40 (s, 1H), 4.70 (spt, J=6.2 Hz, 1H), 3.54 (br dd, J=13.9, 7.8 Hz, 1H), 3.01 (br dd, J=14.0, 7.7 Hz, 1H), 2.36 (s, 3H), 2.23 (s, 3H), 1.06 (d, J=6.2 Hz, 6H)

The Compound in the following table was prepared from reagents reported below following similar procedures as for Compound 3, step 3.

| Compound | Structure & Name | Reagents | Analytical data |
|---|---|---|---|
| 12 | 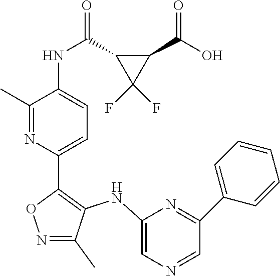 [SMILES: Cc1nc(-c2onc(C)c2Nc2cncc(-c3ccccc3)n2)ccc1NC(=O)[C@@H]1[C@@H](C(=O)O)C1(F)F] | D9 | LC-MS (ESI): m/z (M + 1): 507.2 (Method 1) 1H NMR (400 MHz, DMSO-d6) δ ppm 13.53 (br s, 1 H), 10.10 (s, 1 H), 8.99 (s, 1 H), 8.49 (s, 1 H), 8.19 (s, 1 H), 8.09 (d, J = 8.6 Hz, 1 H), 7.73-7.79 (m, 2 H), 7.71 (d, J = 8.4 Hz, 1 H), 7.31-7.48 (m, 3 H), 3.52 (br dd, J = 14.0, 7.9 Hz, 1 H), 2.99 (br dd, J = 14.5, 7.7 Hz, 1 H), 2.37 (s, 3 H), 2.25 (s, 3 H) |
| | (1S,3S)-2,2-difluoro-3- | | |
| | ((2-methyl-6-(3-methyl- | | |
| | 4-((6-phenylpyrazin-2- | | |
| | yl)amino)isoxazol-5- | | |
| | y1)pyridin-3- | | |
| | y1)carbamoyl)cyclopropane- | | |
| | 1-carboxylic acid | | |

### Example 5

### Process synthesis of (R)-1-(2-chlorophenyl)ethyl (5-(4-(2,2-difluoro-3-(1H-tetrazol-5-yl)cyclopropane-1-carboxamido)phenyl)-3-methylisoxazol-4-yl)carbamate, mix of Trans diastereomers (Compound 13)

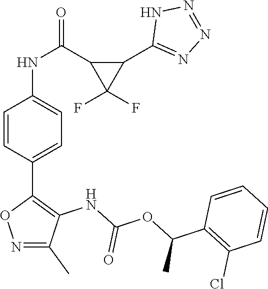 [SMILES: Cc1noc(-c2ccc(NC(=O)C3C(c4nnn[nH]4)C3(F)F)cc2)c1NC(=O)O[C@H](C)c1ccccc1Cl]

### Step 1: methyl 3-((4-(4-((((R)-1-(2-chlorophenyl)ethoxy)carbonyl)amino)-3-methylisoxazol-5-yl)phenyl)carbamoyl)-2,2-difluorocyclopropane-1-carboxylate, mix of Trans diastereomers

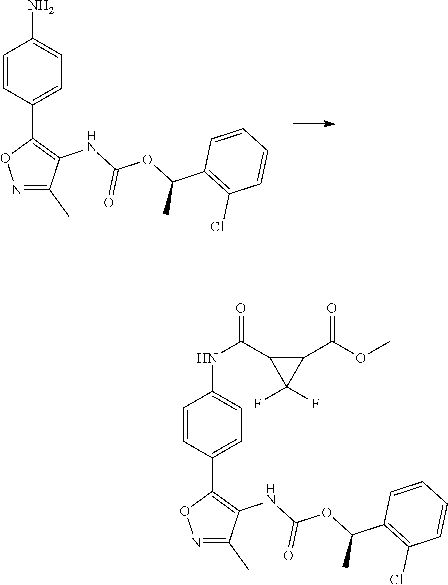 [SMILES: Cc1noc(-c2ccc(N)cc2)c1NC(=O)O[C@H](C)c1ccccc1Cl ; COC(=O)C1C(C(=O)Nc2ccc(-c3onc(C)c3NC(=O)O[C@H](C)c3ccccc3Cl)cc2)C1(F)F]

methyl 3-((4-(4-((((R)-1-(2-chlorophenyl)ethoxy)carbonyl)amino)-3-methylisoxazol-5-yl)phenyl)carbamoyl)-2,2-difluorocyclopropane-1-carboxylate, mix of Trans diastereomers (149 mg, 0.279 mmol, 73% yield) was obtained from (R)-1-(2-chlorophenyl)ethyl (5-(4-aminophenyl)-3-methylisoxazol-4-yl)carbamate (Intermediate B2, 150.0 mg, 0.380 mmol)) and Trans-rac 2,2-difluoro-3-methoxycarbonylcyclopropane-1-carboxylic acid (89.7 mg, 0.500 mmol) using a similar method of Intermediate A17.

LC-MS (ESI): m z (M+1): 534.2 (Method 1)

### Step 2: 3-((4-(4-((((R)-1-(2-chlorophenyl)ethoxy)carbonyl)amino)-3-methylisoxazol-5-yl)phenyl)carbamoyl)-2,2-difluorocyclopropane-1-carboxylic acid, mix of Trans diastereomers

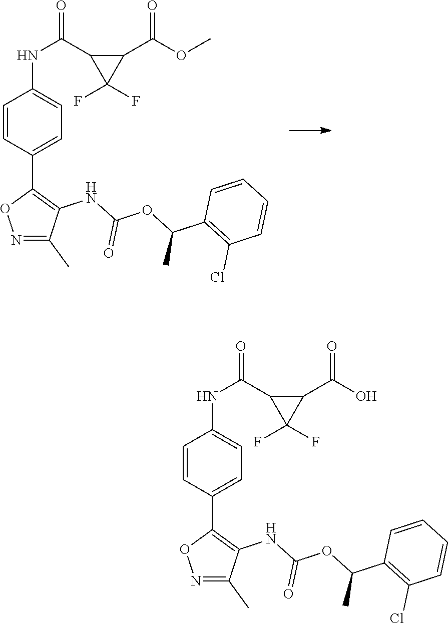 [SMILES: COC(=O)C1C(C(=O)Nc2ccc(-c3onc(C)c3NC(=O)O[C@H](C)c3ccccc3Cl)cc2)C1(F)F ; Cc1noc(-c2ccc(NC(=O)C3C(C(=O)O)C3(F)F)cc2)c1NC(=O)O[C@H](C)c1ccccc1Cl]

3-((4-(4-((((R)-1-(2-chlorophenyl)ethoxy)carbonyl)amino)-3-methylisoxazol-5-yl)phenyl)carbamoyl)-2,2-difluorocyclopropane-1-carboxylic acid, mix of Trans diastereomers (145 mg, crude) was obtained from methyl 3-[[4-[4-[[(1R)-1-(2-chlorophenyl)ethoxy]carbonylamino]-3-methyl-1,2-oxazol-5-yl]phenyl]carbamoyl]-2,2-difluorocyclopropane-1-carboxylate mix of Trans diastereomers (149.0 mg, 0.280 mmol) using a similar method as for Compound 3, step 3.

LC-MS (ESI): m z (M+1): 520.2 (Method 1)

### Step 3: (R)-1-(2-chlorophenyl)ethyl (5-(4-(3-carbamoyl-2,2-difluorocyclopropane-1-carboxamido)phenyl)-3-methylisoxazol-4-yl)carbamate, mix of Trans diastereomers (Intermediate E1)

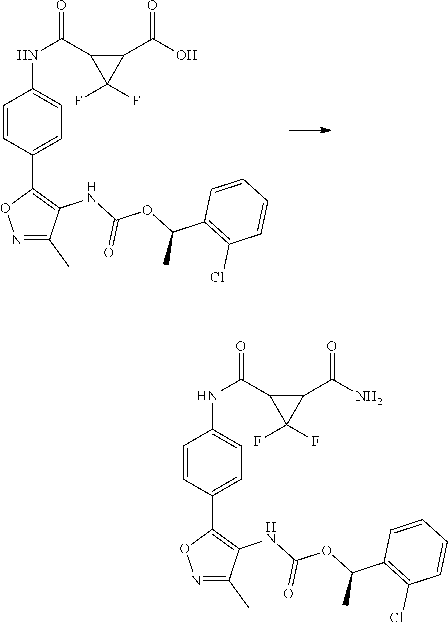 [SMILES: Cc1noc(-c2ccc(NC(=O)C3C(C(=O)O)C3(F)F)cc2)c1NC(=O)O[C@H](C)c1ccccc1Cl ; Cc1noc(-c2ccc(NC(=O)C3C(C(N)=O)C3(F)F)cc2)c1NC(=O)O[C@H](C)c1ccccc1Cl]

To a stirred solution of 3-((4-(4-((((R)-1-(2-chlorophenyl)ethoxy)carbonyl)amino)-3-methylisoxazol-5-yl)phenyl)carbamoyl)-2,2-difluorocyclopropane-1-carboxylic acid, mix of Trans diastereomers (221.0 mg, 0.430 mmol), in DMF (2 mL) cooled at 0° C. was added DIPEA (0.22 mL, 1.28 mmol) and HATU (177.8 mg, 0.470 mmol) and the reaction mixture was stirred at RT for 20 min. Aqueous 30% NH₃ (0.71 mL, 11.6 mmol) was added and the mixture was stirred at RT for 14 h. A second amount of NH₃ (0.71 mL, 11.6 mmol) was added and the mixture was stirred at RT for additional 14 h. The mixture was poured into ice and extracted with EtOAc. The collected organic fractions were washed with brine, dried over Na₂SO₄, filtered and concentrated to dryness under reduced pressure. The product (314 mg, crude) was used without any additional purification.

LC-MS (ESI): m/z (M+1): 519.2 (Method 1)

The Intermediate in the following table was prepared from reagents reported below following similar procedures as for Intermediate E1.

| Intermediate | Structure & Name | Reagents | Analytical data |
|---|---|---|---|
| E2 | 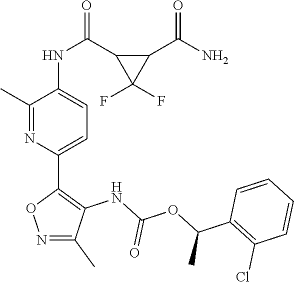 [SMILES: Cc1nc(-c2onc(C)c2NC(=O)O[C@H](C)c2ccccc2Cl)ccc1NC(=O)C1C(C(N)=O)C1(F)F] | Compound 4 | LC-MS (ESI): m/z (M + 1): 516.3 (Method 1) |
| | (R)-1-(2-chlorophenyl)ethyl | | |
| | (5-(5-(3-carbamoy1-2,2- | | |
| | difluorocyclopropane-1- | | |
| | carboxamido)-6- | | |
| | methylpyridin-2-y1)-3- | | |
| | methylisoxazol-4- | | |
| | yl)carbamate, mix of Trans | | |
| | diastereomers | | |

### Step 4: (R)-1-(2-chlorophenyl)ethyl (5-(4-(3-cyano-2,2-difluorocyclopropane-1-carboxamido)phenyl)-3-methylisoxazol-4-yl)carbamate, mix of Trans diastereomers (Intermediate E3)

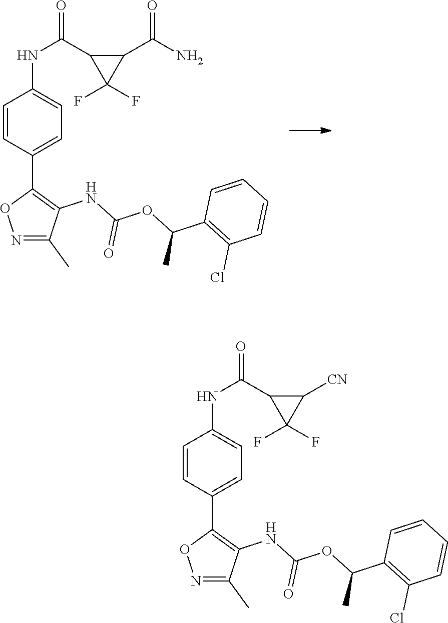 [SMILES: Cc1noc(-c2ccc(NC(=O)C3C(C(N)=O)C3(F)F)cc2)c1NC(=O)O[C@H](C)c1ccccc1Cl ; Cc1noc(-c2ccc(NC(=O)C3C(C#N)C3(F)F)cc2)c1NC(=O)O[C@H](C)c1ccccc1Cl]

Phosphorus(V) oxychloride (0.1 mL, 1.05 mmol) was added dropwise at 0° C. over a period of 10 min. to a solution of (R)-1-(2-chlorophenyl)ethyl (5-(4-(3-carbamoyl-2,2-difluorocyclopropane-1-carboxamido)phenyl)-3-methylisoxazol-4-yl)carbamate, mix of Trans diastereomers (Intermediate E1, 314 mg, crude), and imidazole (39.3 mg, 0.580 mmol) in dry pyridine (5 mL) under N₂ atmosphere. Water was added dropwise, followed by EtOAc, and the mixture was stirred overnight at RT. The organic phase was separated and evaporated to dryness.

The crude was purified by flash chromatography (gradient CyHex/EtOAc from 8/2 to 0/10) to afford the desired product (97.3 mg) as a pale yellow solid.

LC-MS (ESI): m/z (M+1): 501.1 (Method 1)

The Intermediate in the following table was prepared from reagents reported below following similar procedures as for Intermediate E3.

| Intermediate | Structure & Name | Reagents | Analytical data |
|---|---|---|---|
| E4 | 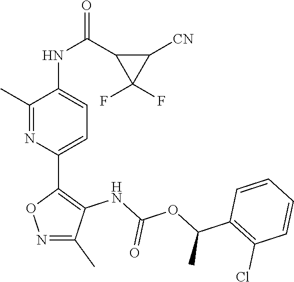 [SMILES: Cc1nc(-c2onc(C)c2NC(=O)O[C@H](C)c2ccccc2Cl)ccc1NC(=O)C1C(C#N)C1(F)F] | E2 | LC-MS (ESI): m/z (M + 1): 516.3 (Method 1) |
| | (R)-1-(2-chlorophenyl)ethyl | | |
| | (5-(5-(3-cyano-2,2- | | |
| | difluorocyclopropane-1- | | |
| | carboxamido)-6- | | |
| | methylpyridin-2-yl)-3- | | |
| | methylisoxazol-4- | | |
| | y1)carbamate, mix of Trans | | |
| | diastereomers | | |

Step 5: (R)-1-(2-chlorophenyl)ethyl (5-(4-(2,2-difluoro-3-(1H-tetrazol-5-yl)cyclopropane-1-carboxamido)phenyl)-3-methylisoxazol-4-yl)carbamate, mix of Trans diastereomers (Compound 13)

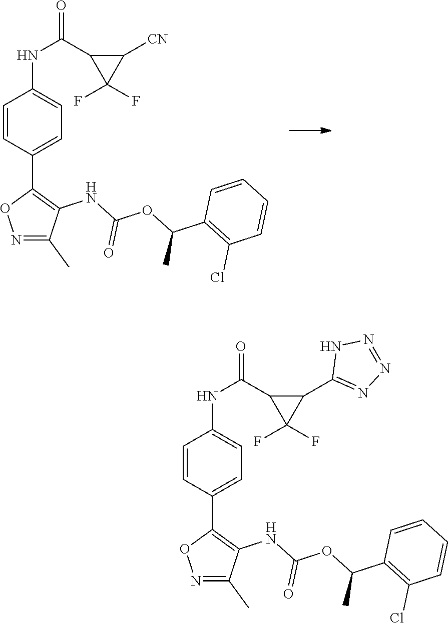 [SMILES: Cc1noc(-c2ccc(NC(=O)C3C(C#N)C3(F)F)cc2)c1NC(=O)O[C@H](C)c1ccccc1Cl ; Cc1noc(-c2ccc(NC(=O)C3C(c4nnn[nH]4)C3(F)F)cc2)c1NC(=O)O[C@H](C)c1ccccc1Cl]

[((R)-1-(2-chlorophenyl)ethyl (5-(4-(3-cyano-2,2-difluorocyclopropane-1-carboxamido) phenyl)-3-methylisoxazol-4-yl)carbamate, mix of Trans diastereomers (Intermediate E3, 83.0 mg, 0.170 mmol), and dibutyl(oxo)tin (20.6 mg, 0.080 mmol) were suspended in dry 1,2-dimethoxyethane (1.25 mL) under nitrogen, then azido(trimethyl)silane (0.13 mL, 0.990 mmol) was added and the mixture was heated under MW irradiation at 90° C. for 3 cycles of 30 minutes. The crude mixture was purified by reversed phase flash chromatography (gradient MeCN/water+0.1% HCOOH from 0/100 to 80/20) to give the title compound (48 mg, 0.088 mmol, 53% yield) as a white solid.

LC-MS (ESI): m/z (M+1): 544.3 (Method 1)

The corresponding sodium salt of Compound 13 was obtained using a similar method as for Compound 8. Compound 13 was characterized as sodium salt.

LC-MS (ESI): m/z (M+1-Na⁺): 544.3 (Method 1)

1H NMR (500 MHz, DMSO-d6) δ ppm 10.82 (s, 1H), 9.33 (s, 1H), 7.67-7.83 (m, 4H), 7.60 (br d, J=7.3 Hz, 1H), 7.42-7.51 (m, 2H), 7.33-7.41 (m, 1H), 5.99 (q, J=6.2 Hz, 1H), 3.39-3.58 (m, 2H), 2.10 (s, 3H), 1.55 (br d, J=6.4 Hz, 3H)

The Compound in the following table was prepared from reagents reported below following similar procedures as for Compound 13

| Compound | Structure & Name | Reagents | Analytical data |
|---|---|---|---|
| 14 | 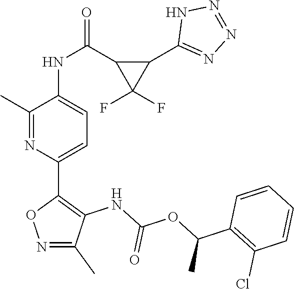 [SMILES: Cc1nc(-c2onc(C)c2NC(=O)O[C@H](C)c2ccccc2Cl)ccc1NC(=O)C1C(c2nnn[nH]2)C1(F)F] | E4 | LC-MS (ESI): m/z (M + 1): 559.3 (Method 1) 1H NMR (400 MHz, DMSO-d6) δ ppm 10.29 (s, 1 H), 9.26 (br s, 1 H), 8.10-8.20 (m, 1 H), 7.67 (d, J = 8.4 Hz, 1 H), 7.22- 7.76 (m, 4 H), 5.99 (br q, J = 4.3 Hz, 1 H), 3.72- 4.00 (m, 2 H), 2.46-2.53 (m, 3 H), 2.17 (s, 3 H), 1.28-1.69 (m, 3 H) |
| | (R)-1-(2-chlorophenyl)ethyl | | |
| | (5-(5-(2,2-difluoro-3-(1H- | | |
| | tetrazol-5-yl)cyclopropane- | | |
| | 1-carboxamido)-6- | | |
| | methylpyridin-2-y1)-3- | | |
| | methylisoxazol-4- | | |
| | yl)carbamate, mix of Trans | | |
| | diastereomers | | |

The corresponding sodium salt of Compound 14 was obtained using a similar method as for Compound 8.

LC-MS (ESI): m/z (M+1-Na+): 544.3 (Method 1)

1H NMR (500 MHz, DMSO-d6) δ ppm 10.82 (s, 1H), 9.33 (s, 1H), 7.67-7.83 (m, 4H), 7.60 (br d, J=7.3 Hz, 1H), 7.42-7.51 (m, 2H), 7.33-7.41 (m, 1H), 5.99 (q, J=6.2 Hz, 1H), 3.39-3.58 (m, 2H), 2.10 (s, 3H), 1.55 (br d, J=6.4 Hz, 3H)

### Example 6

### Process synthesis of 3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic, mix of Cis and Trans diastereomers (Compound 16) and Compound 17 mix of Trans diastereomers

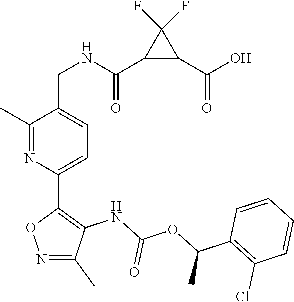 [SMILES: Cc1nc(-c2onc(C)c2NC(=O)O[C@H](C)c2ccccc2Cl)ccc1CNC(=O)C1C(C(=O)O)C1(F)F]

### Step 1: (R)-1-(2-chlorophenyl)ethyl (5-(5-(hydroxymethyl)-6-methylpyridin-2-yl)-3-methylisoxazol-4-yl)carbamate (Intermediate F1)

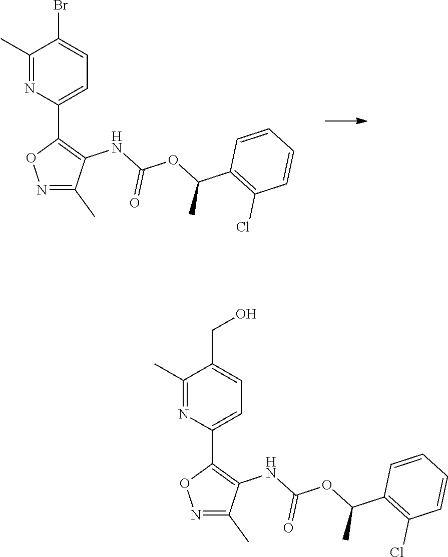 [SMILES: Cc1nc(-c2onc(C)c2NC(=O)O[C@H](C)c2ccccc2Cl)ccc1Br ; Cc1nc(-c2onc(C)c2NC(=O)O[C@H](C)c2ccccc2Cl)ccc1CO]

A solution of (R)-1-(2-chlorophenyl)ethyl (5-(5-bromo-6-methylpyridin-2-yl)-3-methylisoxazol-4-yl)carbamate (Intermediate all, 0.400 g, 0.887 mmol) and (tributylstannyl)methanol (0.427 g, 1.331 mmol) in 1,4-dioxane (6 mL) was degassed for 15 min under a nitrogen stream. Pd(PPh₃)₄ (0.154 g, 0.133 mmol) was added and the mixture was heated at 90° C. for 9 h, then stirred at RT overnight. The reaction was diluted with EtOAc, aqueous KF was added and the mixture was stirred for 15 minutes. The phases were separated and the precipitate in the organic phase was filtered off and washed with EtOAc. The filtrate was washed with brine, dried over Na₂SO₄, filtered and concentrated to dryness. The crude was purified by flash chromatography (DCM: EtOAc from 90:10 to 80:20) to obtain the target compound (0.164 g, 0.408 mmol, 46% yield) as a pale brown solid.

LC-MS (ESI): m/z (M+1): 402.2 (Method 2)

### Step 2: (R)-1-(2-chlorophenyl)ethyl (5-(5-(azidomethyl)-6-methylpyridin-2-yl)-3-methylisoxazol-4-yl)carbamate (Intermediate F2)

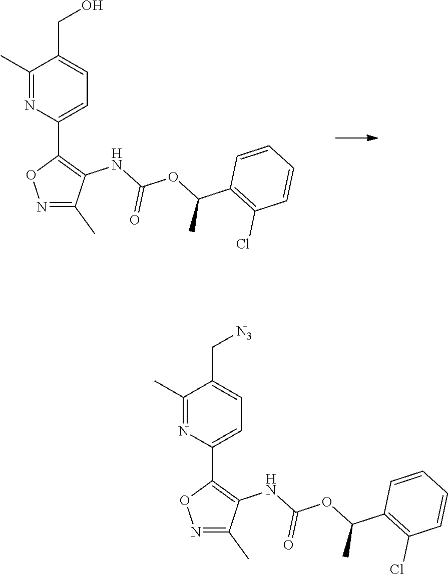 [SMILES: Cc1nc(-c2onc(C)c2NC(=O)O[C@H](C)c2ccccc2Cl)ccc1CO ; Cc1nc(-c2onc(C)c2NC(=O)O[C@H](C)c2ccccc2Cl)ccc1CN=[N+]=[N-]]

To a solution of (R)-1-(2-chlorophenyl)ethyl (5-(5-(hydroxymethyl)-6-methylpyridin-2-yl)-3-methylisoxazol-4-yl)carbamate (Intermediate F1, 0.730 g, 1.817 mmol) in THE (24 mL) cooled to 0° C., diphenylphosphoryl azide (0.783 mL, 3.63 mmol) was added followed by DBU (0.548 mL, 3.63 mmol) and the resulting mixture was allowed to reach RT and stirred overnight. The mixture was partitioned between EtOAc and water and the organic phase was washed with brine, dried over Na₂SO₄, filtered and concentrated to dryness. The crude was purified by flash chromatography (hexane:EtOAc from 90:10 to 83:17) to obtain the title compound (0.641 g, 1.502 mmol, 83% yield) as a white solid.

LC-MS (ESI): m/z (M+1): 427.3 (Method 2)

### Step 3: (R)-1-(2-chlorophenyl)ethyl (5-(5-(aminomethyl)-6-methylpyridin-2-yl)-3-methylisoxazol-4-yl)carbamate (Intermediate F3)

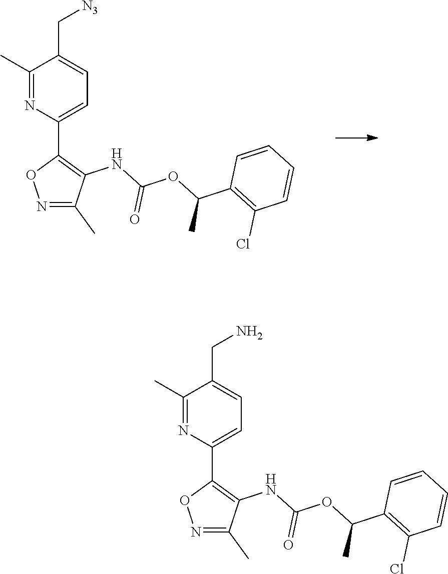 [SMILES: Cc1nc(-c2onc(C)c2NC(=O)O[C@H](C)c2ccccc2Cl)ccc1CN=[N+]=[N-] ; Cc1nc(-c2onc(C)c2NC(=O)O[C@H](C)c2ccccc2Cl)ccc1CN]

To a solution of (R)-1-(2-chlorophenyl)ethyl (5-(5-(azidomethyl)-6-methylpyridin-2-yl)-3-methylisoxazol-4-yl)carbamate (Intermediate F2, 0.641 g, 1.502 mmol) in THE (16 mL), PPh₃ (0.591 g, 2.252 mmol) was added and the mixture was stirred at RT for 6 h. Water (4.5 mL) was added and the resulting mixture was stirred at RT for 4 days. The solvent was removed under reduced pressure and the residue was dissolved in MeOH and charged on SCX cartridge washing with MeOH/DCM. The product was eluted with 2M NH₃ in MeOH and the volatiles were removed under reduced pressure to afford the desired compound (0.593 g, 1.479 mmol, 99% yield) as a yellowish solid that was used without any additional purification.

LC-MS (ESI): m/z (M+1): 401.2 (Method 2)

### Step 4: methyl 3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylate, mix of Cis and Trans diastereomers (Intermediate F4)

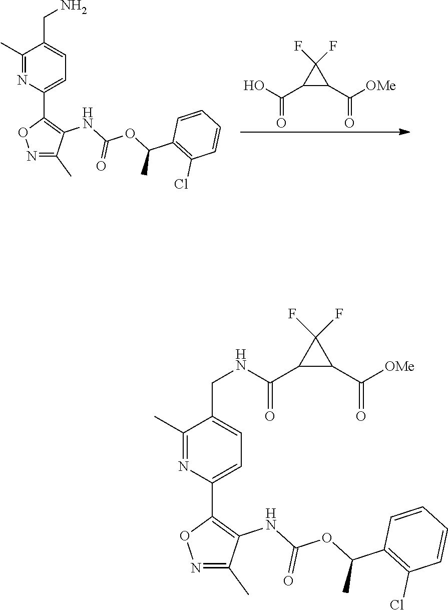 [SMILES: Cc1nc(-c2onc(C)c2NC(=O)O[C@H](C)c2ccccc2Cl)ccc1CN ; COC(=O)C1C(C(=O)O)C1(F)F ; COC(=O)C1C(C(=O)NCc2ccc(-c3onc(C)c3NC(=O)O[C@H](C)c3ccccc3Cl)nc2C)C1(F)F]

Intermediate F4 (110 mg, 0.195 mmol, 51% yield) was prepared from (R)-1-(2-chlorophenyl)ethyl (5-(5-(aminomethyl)-6-methylpyridin-2-yl)-3-methylisoxazol-4-yl)carbamate (Intermediate F3, 152.0 mg, 0.380 mmol) and 2,2-difluoro-3-(methoxycarbonyl)cyclopropane-1-carboxylic acid, (88.8 mg, 0.490 mmol), using a similar method as for Intermediate A17.

LC-MS (ESI): m/z (M+1): 563.2 (Method 1)

### Step 5: 3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic acid, mix of Cis and Trans diastereomers (Compound 15)

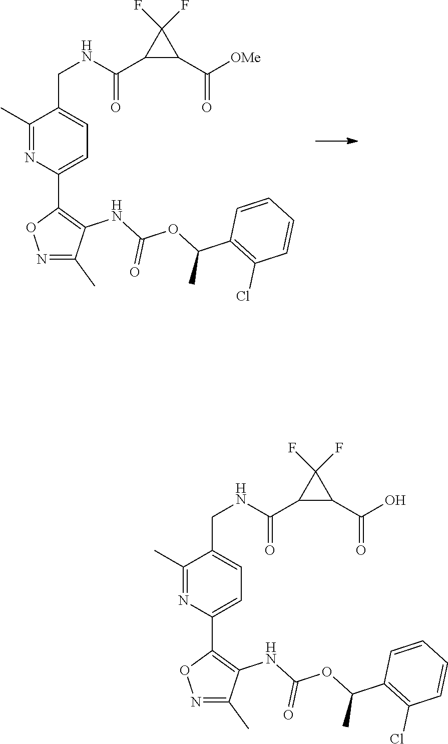 [SMILES: COC(=O)C1C(C(=O)NCc2ccc(-c3onc(C)c3NC(=O)O[C@H](C)c3ccccc3Cl)nc2C)C1(F)F ; Cc1nc(-c2onc(C)c2NC(=O)O[C@H](C)c2ccccc2Cl)ccc1CNC(=O)C1C(C(=O)O)C1(F)F]

Compound 15 (135 mg, crude) was prepared from methyl 3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylate, mix of Cis and Trans diastereomers (Intermediate F4, 110.0 mg, 0.180 mmol), using a similar method as for Compound 3, step 3.

LC-MS (ESI): m/z (M+1): 563.2 (Method 1)

Compound 15 was submitted to preparative HPLC.

Conditions: CSH C18 (2.1×50 mm, 1.7 μm). Conditions: [A1: Waters+0.1% HCOOH]; [B1: MeCN+0.1% HCOOH]. Gradient: from 3% B1 to 99.9% B1 in 1.5 min (flow: 1.00 mL/min).

Detection: UV/Vis detection range 210 nm to 350 nm

### Peak 1: Compound 16

3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic acid, mix of Cis diastereomers

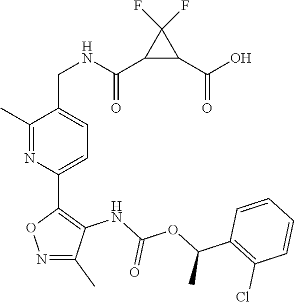 [SMILES: Cc1nc(-c2onc(C)c2NC(=O)O[C@H](C)c2ccccc2Cl)ccc1CNC(=O)C1C(C(=O)O)C1(F)F]

LC-MS (ESI): m/z (M+1): 563.2 (Method 1)

1H NMR (500 MHz, DMSO-d6) δ ppm 12.84 (br s, 1H), 9.27 (br s, 1H), 8.95 (br s, 1H), 7.72 (d, J=8.0 Hz, 1H), 7.59 (d, J=8.0 Hz, 1H), 6.73-7.84 (m, 4H), 5.98 (br s, 1H), 4.33 (br s, 2H), 2.93-3.12 (m, 2H), 2.48 (br s, 3H), 2.17 (s, 3H), 1.53 (br s, 3H)

### Peak 2: Compound 17

3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic acid, mix of Trans diastereomers

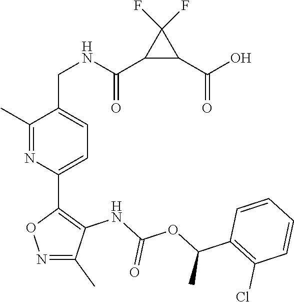 [SMILES: Cc1nc(-c2onc(C)c2NC(=O)O[C@H](C)c2ccccc2Cl)ccc1CNC(=O)C1C(C(=O)O)C1(F)F]

LC-MS (ESI): m/z (M+1): 563.2 (Method 1) 1H NMR (500 MHz, DMSO-d6) δ ppm 13.41 (br s, 1H), 9.00 (t, J=5.6 Hz, 1H), 9.26 (br s, 1H), 7.65-7.72 (m, 1H), 7.57-7.64 (m, 1H), 6.92-7.75 (m, 4H), 5.98 (br s, 1H), 4.27-4.47 (m, 2H), 3.14-3.24 (m, 1H), 2.98 (br dd, J=13.9, 7.8 Hz, 1H), 2.48 (s, 3H), 2.17 (s, 3H), 1.53 (br s, 3H)

### Example 7

### Process synthesis of 3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid, mix of Trans diastereomers (Compound 18) and of its Single Enantiomers Trans Compound 19 and Compound 20

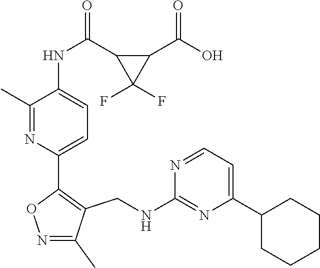 [SMILES: Cc1nc(-c2onc(C)c2CNc2nccc(C3CCCCC3)n2)ccc1NC(=O)C1C(C(=O)O)C1(F)F]

### Step 1: (5-(5-bromo-6-methylpyridin-2-yl)-3-methylisoxazol-4-yl)methanol (Intermediate

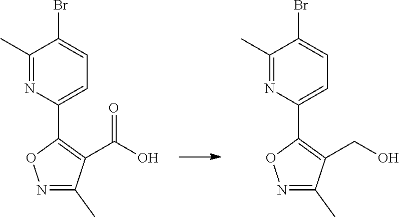 [SMILES: Cc1nc(-c2onc(C)c2C(=O)O)ccc1Br ; Cc1nc(-c2onc(C)c2CO)ccc1Br]

A solution of ethyl chloroformate (0.97 mL, 10.1 mmol) in dry THE (10 mL) was added to a solution of 5-(5-bromo-6-methylpyridin-2-yl)-3-methyl-1,2-oxazole-4-carboxylic acid (Intermediate A7, 2.5 g, 8.41 mmol) and TEA (1.52 mL, 10.94 mmol) in dry THE (20 mL) at 0° C. The mixture was stirred at 0° C. for 1h then a solution of sodium borohydride (795.8 mg, 21.04 mmol) in water (9.802 mL) was added dropwise over 10 min and the mixture was stirred at 0° C. for 2 h. 2N NaOH solution was added and the mixture was stirred at RT for 5 min, then extracted with EtOAc (3 times). The organic layer was washed with brine, dried over Na₂SO₄, filtered and concentrated in vacuo. The crude was purified by flash chromatography (gradient CyHex/EtOAc from 90/10 to 40/60) to give the title compound (1.58 g, 5.581 mmol, 66% yield) as a white solid.

LC-MS (ESI): m/z (M+1): 285.1 (Method 1)

The Intermediate in the following table was prepared from reagents reported below following similar procedures as for intermediate H1.

| Intermediate | Structure & Name | Reagents | Analytical data |
|---|---|---|---|
| H2 | 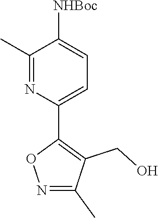 [SMILES: Cc1nc(-c2onc(C)c2CO)ccc1NC(=O)OC(C)(C)C] | D1 | LC-MS (ESI): m/z 320.7 (M + 1) (Method 3) 1H NMR (300 MHz, DMSO-d6) δ 9.01 (s, 1H), 8.02 (d, J = 8.5 Hz, 1H), 7.72 (d, J = 8.4 Hz, 1H), 5.23 (t, J = 5.7 Hz, 1H), |
| | tert-butyl (6-(4- | | 4.73 (d, |
| | (hydroxymethyl)-3- | | J = 5.7 Hz, 2H), |
| | methylisoxazol-5-y1)-2- | | 2.52 (s, 3H), |
| | methylpyridin-3- | | 2.31 (s, 3H), |
| | yl)carbamate | | 1.50 (s, 9H). |

### Step 2: 5-(5-bromo-6-methylpyridin-2-yl)-4-(chloromethyl)-3-methylisoxazole (Intermediate H3)

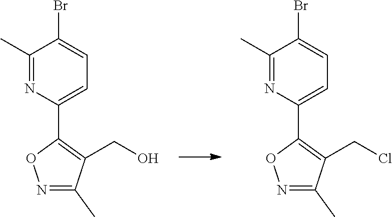 [SMILES: Cc1nc(-c2onc(C)c2CO)ccc1Br ; Cc1nc(-c2onc(C)c2CCl)ccc1Br]

To an ice-cooled solution of (5-(5-bromo-6-methylpyridin-2-yl)-3-methylisoxazol-4-yl)methanol (Intermediate H1, 1.58 g, 5.581 mmol) in DCM (39.5 mL), TEA (2.33 mL, 16.74 mmol) was added followed by methanesulfonyl chloride (1.08 mL, 13.95 mmol). The mixture was allowed to reach RT and stirred overnight. Volatiles were removed at reduced pressure to provide the desired compound (2.63 g, crude). The material was used in the next step without further purification.

LC-MS (ESI): m/z (M+1): 303.0 (Method 1)

The Intermediate in the following table was prepared from reagents reported below following similar procedures as for Intermediate H3.

| Intermediate | Structure & Name | Reagents | Analytical data |
|---|---|---|---|
| H4 | 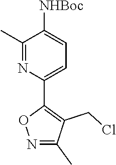 [SMILES: Cc1nc(-c2onc(C)c2CCl)ccc1NC(=O)OC(C)(C)C] | H2 | LC-MS (ESI): m/z 337.1 (M + 1) (Method 3) 1H NMR (300 MHz, DMSO-d6) δ 9.04 (s, 1H), 8.06 (d, J = 8.5 Hz, 1H), 7.75 (d, J = 8.5 Hz, 1H), 5.22 (s, 2H), |
| | tert-butyl (6-(4- | | 2.53 (s, 3H), |
| | (chloromethyl)-3- | | 2.35 (s, 3H), |
| | methylisoxazol-5-yl)-2- | | 1.50 (s, 9H). |
| | methylpyridin-3- | | |
| | yl)carbamate | | |

### Step 3: (5-(5-bromo-6-methylpyridin-2-yl)-3-methylisoxazol-4-yl)methanamine (Intermediate H5)

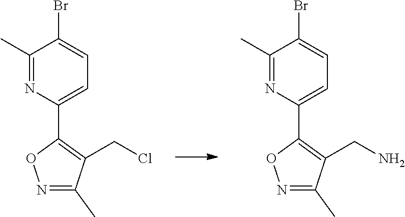 [SMILES: Cc1nc(-c2onc(C)c2CCl)ccc1Br ; Cc1nc(-c2onc(C)c2CN)ccc1Br]

5-(5-bromo-6-methylpyridin-2-yl)-4-(chloromethyl)-3-methylisoxazole (Intermediate H3, 2.63 g, crude) was dissolved in 1,4-dioxane (50 mL), then ammonium hydroxide 28-30% in water (50.95 mL, 366.28 mmol) was added and the mixture was stirred at RT overnight. The mixture was concentrated under reduced pressure and partitioned between EtOAc and brine. The aqueous phase was basified and extracted with DCM/MeOH 9/1. The combined organic layers were dried using a hydrophobic phase separator and concentrated under reduced pressure to afford the title compound (623 mg) as a creamy solid.

LC-MS (ESI): m/z (M+1): 284.0 (Method 2)

The Intermediate in the following table was prepared from reagents reported below following similar procedures as for Intermediate H5.

| Intermediate | Structure & Name | Reagents | Analytical data |
|---|---|---|---|
| H6 | 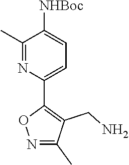 [SMILES: Cc1nc(-c2onc(C)c2CN)ccc1NC(=O)OC(C)(C)C] | H4 | LC-MS (ESI): m/z 319.3 (M + 1) (Method 3) 1H NMR (300 MHz, DMSO-d6) δ 8.99 (s, 1H), 8.00 (d, J = 8.5 Hz, 1H), |
| | tert-butyl (6-(4- | | 7.71 (d, J = |
| | (aminomethyl)-3- | | 8.4 Hz, 1H), |
| | methylisoxazol-5-yl)-2- | | 3.79 (s, 2H), |
| | methylpyridin-3- | | 2.52 (s, 3H), |
| | yl)carbamate | | 2.30 (s, 3H), |
| | | | 1.97 (bs, 2H), |
| | | | 1.49 (s, 9H). |

Step 4: 2-chloro-4-cyclohexylpyrimidine (intermediate H7)

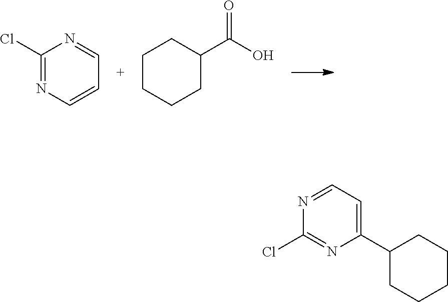

To a mixture of 2-chloropyrimidine (1.12 g, 9.75 mmol), cyclohexanecarboxylic acid (1.0 g, 7.8 mmol) and silver nitrate (265.07 mg, 1.56 mmol) in DCM (58 mL) and water (58 mL), dipotassium sulfonatooxy sulfate (2.12 g, 7.8 mmol) was added and the mixture was stirred at RT overnight. The organic phase was concentrated under reduced pressure, then the aqueous mixture was extracted with EtOAc and discarded. The organic layer was washed with H₂O, dried over Na₂SO₄, filtered and concentrated under reduced pressure. The crude was purified by flash chromatography eluting with CyHex:EtOAc from 0% to 40% to afford the desired compound (700 mg, 3.559 mmol, 46% yield) as a colorless oil.

LC-MS (ESI): m/z (M+1): 197.1 (Method 1)

### Step 5: N-((5-(5-bromo-6-methylpyridin-2-yl)-3-methylisoxazol-4-yl)methyl)-4-cyclohexylpyrimidin-2-amine (Intermediate H8)

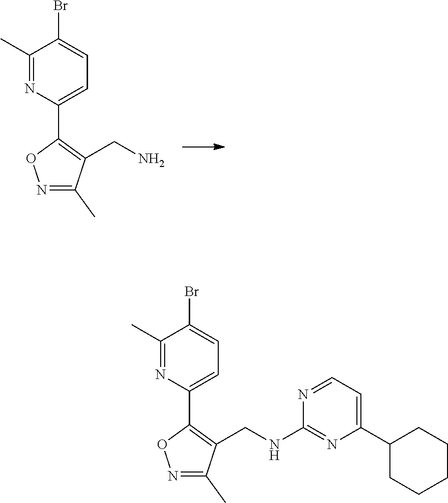 [SMILES: Cc1nc(-c2onc(C)c2CN)ccc1Br ; Cc1nc(-c2onc(C)c2CNc2nccc(C3CCCCC3)n2)ccc1Br]

To a mixture of (5-(5-bromo-6-methylpyridin-2-yl)-3-methylisoxazol-4-yl)methanamine (Intermediate H5, 300.0 mg, 1.01 mmol) and 2-chloro-4-cyclohexylpyrimidine (Intermediate H7, 198.7 mg, 1.01 mmol) in DMF (5 mL), K₂CO₃ (209.4 mg, 1.52 mmol) was added and the mixture was stirred at 80° C. for 2 days. The mixture was allowed to cool to RT, poured into saturated NaHCO₃ aqueous solution and extracted with EtOAc. The organic phase was separated, filtered through a hydrophobic phase separator and concentrated under reduced pressure. The crude was purified by flash chromatography (isocratic elution CyHex/EtOAc 9/1). Evaporation of proper fractions provided the target compound (129 mg, 0.292 mmol, 2900 yield) as a pale yellow solid.

LC-MS (ESI): m/z (M+1): 444.1 (Method 1)

The Intermediates in the following table were prepared from reagents reported below following similar procedures as for Intermediate H8.

| Intermediate | Structure & Name | Reagents | Analytical data |
|---|---|---|---|
| H9 | 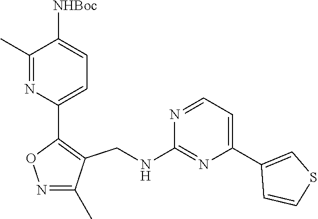 [SMILES: Cc1nc(-c2onc(C)c2CNc2nccc(-c3ccsc3)n2)ccc1NC(=O)OC(C)(C)C] | H6 + 2-chloro- 4-(thiophen-3- yl)pyrimidine | LC-MS (ESI): m/z 479.4 (M + 1) (Method 3) 1H NMR (300 MHz, DMSO-d6) δ 9.03 (s, 1H), 8.29 (d, J = 5.1 Hz, 1H), 8.03 (d, J = 8.5 Hz, 1H), 7.91 − 7.82 (m, 1H), 7.75 (d, J = 8.5 Hz, 1H), 7.71 (d, J = 4.9 Hz, 1H), 7.53 (t, J = 5.5 Hz, 1H), 7.17 (q, J = 5.0, 3.7 Hz, 1H), 7.10 (d, J = 5.2 Hz, 1H), 4.93 − 4.54 (m, 2H), 2.34 (s, 3H), 1.50 (s, 9H). |
| | tert-butyl (2-methyl-6-(3- | | |
| | methyl-4-(((4-(thiophen-3- | | |
| | yl)pyrimidin-2- | | |
| | yl)amino)methyl)isoxazol-5- | | |
| | yl)pyridin-3-yl)carbamate | | |
| H10 | 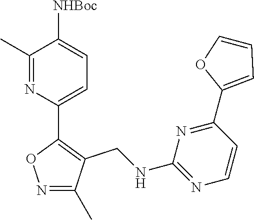 [SMILES: Cc1nc(-c2onc(C)c2CNc2nccc(-c3ccco3)n2)ccc1NC(=O)OC(C)(C)C] | H6 + 2-chloro- 4-(furan-2- yl)pyrimidine | LC-MS (ESI): m/z 463.3 (M + 1) (Method 3) 1H NMR (300 MHz, DMSO-d6) δ 9.02 (s, 1H), 8.32 (d, J = 5.1 Hz, 1H), 8.03 (d, J = 8.5 Hz, 1H), 7.88 (dd, J = 1.7, 0.8 Hz, 1H), 7.74 (d, J = 8.5 Hz, 1H), 7.46 (t, J = 5.8 Hz, 1H), 7.01 (s, 1H), 6.90 (d, J = 5.1 Hz, 1H), 6.64 (dd, J = 3.5, 1.8 Hz, 1H), 4.75 (d, J = 5.8 Hz, 2H), 2.34 (s, 3H), 1.49 (s, 9H) |
| | tert-butyl (6-(4-(((4-(furan-2- | | |
| | yl)pyrimidin-2- | | |
| | yl)amino)methyl)-3- | | |
| | methylisoxazol-5-yl)-2- | | |
| | methylpyridin-3-yl)carbamate | | |
| H11 | 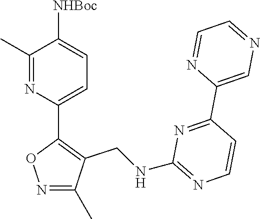 [SMILES: Cc1nc(-c2onc(C)c2CNc2nccc(-c3cnccn3)n2)ccc1NC(=O)OC(C)(C)C] | H6 + 2-chloro- 4-(pyrazin-2- yl)pyrimidine | LC-MS (ESI): m/z 475.3 (M + 1) (Method 3) H NMR (400 MHz, DMSO-d6) δ 9.41 (d, J = 1.2 Hz, 1H), 8.99 (s, 1H), 8.79 − 8.72 (m, 2H), 8.56 − 8.47 (m, 1H), 8.04 (d, J = 8.4 Hz, 1H), 7.75 (d, J = 8.5 Hz, 1H), 7.71 (t, J = 5.7 Hz, 1H), 7.47 (d, J = 5.0 Hz, 1H), 4.88 (s, 2H), 2.53 (s, 3H), 2.37 − 2.31 (m, 3H), 1.50 (s, 9H). |
| | tert-butyl (2-methyl-6-(3- | | |
| | methyl-4-(((4-(pyrazin-2- | | |
| | yl)pyrimidin-2- | | |
| | yl)amino)methyl)isoxazol-5- | | |
| | yl)pyridin-3-yl)carbamate | | |
| H12 | 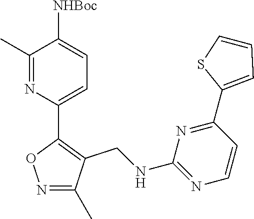 [SMILES: Cc1nc(-c2onc(C)c2CNc2nccc(-c3cccs3)n2)ccc1NC(=O)OC(C)(C)C] | H6 + 2-chloro- 4-(thiophen-2- yl)pyrimidine | LC-MS (ESI): m/z 479.3 (M + 1) (Method 3) 1H NMR (400 MHz, DMSO-d6) δ 9.02 (s, 1H), 8.29 (d, J = 5.2 Hz, 1H), 8.02 (d, J = 8.5 Hz, 1H), 7.85 (s, 1H), 7.74 (d, J = 8.5 Hz, 1H), 7.71 − 7.68 (m, 1H), 7.51 (s, 1H), 7.17 (dd, J = 5.0, 3.7 Hz, 1H), 7.09 (d, J = 5.2 Hz, 1H), 4.76 (s, 2H), 2.34 (s, 3H), 1.49 (s, 9H). |
| | tert-butyl (2-methyl-6-(3- | | |
| | methyl-4-(((4-(thiophen-2- | | |
| | yl)pyrimidin-2- | | |
| | yl)amino)methyl)isoxazol-5- | | |
| | yl)pyridin-3-yl)carbamate | | |
| H13 | 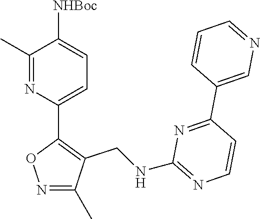 | H6 + 2-chloro- 4-(pyridin-3- yl)pyrimidine | LC-MS (ESI): m/z 546.2 (M + 1) (Method 3) 1H NMR (400 MHz, DMSO-d6) δ 10.26 (s, 1H), 9.21 (d, J = 2.3 Hz, 1H), 8.65 (dd, J = 4.6, 1.6 Hz, 1H), 8.41 (d, J = 5.0 Hz, 1H), 8.29 (s, 1H), 8.18 (d, J = 8.5 Hz, 1H), 7.78 (d, J = 8.5 Hz, 1H), 7.62 (t, J = 5.7 Hz, 1H), 7.51 − 7.39 (m, 1H), 7.27 (d, J = 5.1 Hz, 1H), 4.87 (s, 2H), 3.75 (s, 3H), 3.74 − 3.67 (m, 1H), 3.24 (dd, J = 13.2, 7.9 Hz, 1H), 2.54 (s, 3H), 2.33 (s, 3H). |
| | tert-butyl (2-methyl-6-(3- | | |
| | methyl-4-(((4-(pyridin-3- | | |
| | yl)pyrimidin-2- | | |
| | yl)amino)methyl)isoxazol-5- | | |
| | yl)pyridin-3-yl)carbamate | | |

### Step 6: tert-butyl (6-(4-(((4-cyclohexylpyrimidin-2-yl)amino)methyl)-3-methylisoxazol-5-yl)-2-methylpyridin-3-yl)carbamate (Intermediate H14)

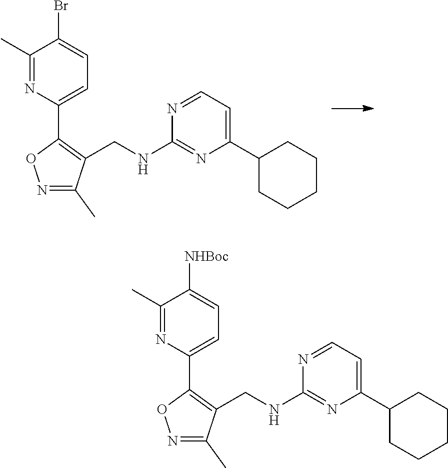 [SMILES: Cc1nc(-c2onc(C)c2CNc2nccc(C3CCCCC3)n2)ccc1Br ; Cc1nc(-c2onc(C)c2CNc2nccc(C3CCCCC3)n2)ccc1NC(=O)OC(C)(C)C]

A vial was charged with Cs₂CO₃ (190 mg, 0.580 mmol), N-((5-(5-bromo-6-methylpyridin-2-yl)-3-methylisoxazol-4-yl)methyl)-4-cyclohexylpyrimidin-2-amine (Intermediate H8, 129.0 mg, 0.292 mmol) and carbamic acid tert-butyl ester (41.0 mg, 0.350 mmol). The system was closed and three cycles of vacuum/nitrogen back-filling were applied. CPMIE (1.5 mL) was added and three cycles of vacuum/nitrogen back-filling were applied. XantPhos (15.9 mg, 0.030 mmol) and Pd₂(dba)₃ (9.8 mg, 0.010 mmol) were then added and the mixture was heated at 100° C. for 2 hours. The reaction was cooled to RT, filtered through a pad of celite, washing with EtOAc, and evaporated. The crude was purified by flash chromatography (gradient CyHex/EtOAc from 95/5 to 60/40) to give the desired compound (114 mg, 0.240 mmol, 8200 yield) as a pale yellow oil.

LC-MS (ESI): m/z (M+1): 479.4 (Method 1).

Step 7: N-((5-(5-amino-6-methylpyridin-2-yl)-3-methylisoxazol-4-yl)methyl)-4-cyclohexylpyrimidin-2-amine (Intermediate H15)

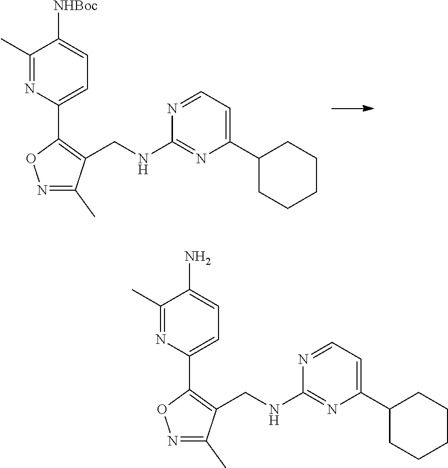 [SMILES: Cc1nc(-c2onc(C)c2CNc2nccc(C3CCCCC3)n2)ccc1NC(=O)OC(C)(C)C ; Cc1nc(-c2onc(C)c2CNc2nccc(C3CCCCC3)n2)ccc1N]

To a solution of tert-butyl (6-(4-(((4-cyclohexylpyrimidin-2-yl)amino)methyl)-3-methylisoxazol-5-yl)-2-methylpyridin-3-yl)carbamate (Intermediate H14, 114.0 mg, 0.240 mmol) in DCM (4.7 mL), TFA (1.6 mL) was added and the mixture was stirred at RT for 2 hours. The mixture was concentrated under reduced pressure, then the residue was suspended in toluene and the solvent was evaporated under reduced pressure (3 times). The crude was re-dissolved in DCM, a solution of NaHCO₃ was slowly added to pH 9 and the phases were separated. The aqueous layer was further extracted with DCM (2×20 mL), then the combined organic layer was dried over Na₂SO₄ and evaporated to afford the title compound (90 mg, 0.238 mmol, 99.8% yield) as a yellow oil.

LC-MS (ESI): m/z (M+1): 379.3 (Method 1)

The Intermediates in the following table were prepared from reagents reported below following similar procedures as for Intermediate H15.

| Intermediate | Structure & Name | Reagents | Analytical data |
|---|---|---|---|
| H16 | 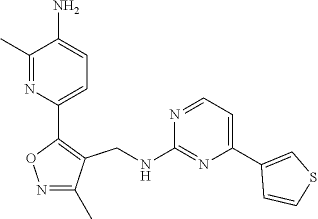 [SMILES: Cc1nc(-c2onc(C)c2CNc2nccc(-c3ccsc3)n2)ccc1N] | H9 | LC-MS (ESI): m/z 379.4 (M + 1) (Method 3) 1H NMR (400 MHz, DMSO-d6) δ 8.31 (s, 1H), 8.20 (s, 1H), 7.71 − 7.58 (m, 2H), 7.50 (d, J = 8.4 Hz, 2H), 7.12 − 7.01 (m, 2H), 5.71 (s, 2H), 4.73 (s, 2H), 2.38 (s, 3H), 2.31 (s, 3H). |
| | N-((5-(5-amino-6- | | |
| | methylpyridin-2-yl)-3- | | |
| | methylisoxazol-4-yl)methyl)- | | |
| | 4-(thiophen-3-yl)pyrimidin-2- | | |
| | amine | | |
| H17 | 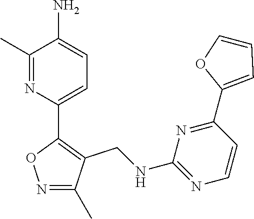 [SMILES: Cc1nc(-c2onc(C)c2CNc2nccc(-c3ccco3)n2)ccc1N] | H10 | LC-MS (ESI): m/z 363.2 (M + 1) (Method 3) 1H NMR (300 MHz, DMSO-d6) δ 8.32 (s, 1H), 7.89 (dd, J = 1.8, 0.8 Hz, 1H), 7.50 (d, J = 8.3 Hz, 2H), 7.13 (d, J = 3.4 Hz, 1H), 7.03 (d, J = 8.4 Hz, 1H), 6.90 (d, J = 5.1 Hz, 1H), 6.66 (dd, J = 3.4, 1.8 Hz, 1H), 5.70 (s, 2H), 4.66 (d, J = 5.9 Hz, 2H), 2.37 (s, 3H), 2.32 (s, 3H). |
| | N-((5-(5-amino-6- | | |
| | methylpyridin-2-yl)-3- | | |
| | methylisoxazol-4-yl)methyl)- | | |
| | 4-(furan-2-yl)pyrimidin-2- | | |
| | amine | | |
| H18 | 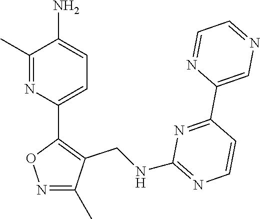 [SMILES: Cc1nc(-c2onc(C)c2CNc2nccc(-c3cnccn3)n2)ccc1N] | H11 | LC-MS (ESI): m/z 375.8 (M + 1) (Method 3) 1H NMR (300 MHz, DMSO-d6) δ 9.45 (s, 1H), 8.81 − 8.71 (m, 2H), 8.52 (s, 1H), 7.79 (s, 1H), 7.51 (d, J = 8.3 Hz, 1H), 7.47 (d, J = 5.0 Hz, 1H), 7.04 (d, J = 8.4 Hz, 1H), 5.70 (s, 2H), 4.80 (s, 2H), 2.37 (s, 3H), 2.31 (s, 3H). |
| | N-((5-(5-amino-6- | | |
| | methylpyridin-2-yl)-3- | | |
| | methylisoxazol-4-yl)methyl)- | | |
| | 4-(pyrazin-2-yl)pyrimidin-2- | | |
| | amine | | |
| H19 | 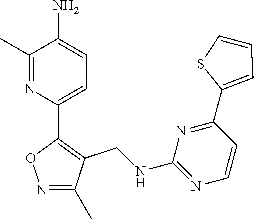 [SMILES: Cc1nc(-c2onc(C)c2CNc2nccc(-c3cccs3)n2)ccc1N] | H12 | LC-MS (ESI): m/z 379.1 (M + 1) (Method 4) 1H NMR (300 MHz, DMSO-d6) δ 8.30 (s, 1H), 7.91 − 7.84 (m, 1H), 7.75 − 7.69 (m, 1H), 7.59 (s, 1H), 7.50 (d, J = 8.4 Hz, 1H), 7.18 (dd, J = 5.0, 3.7 Hz, 1H), 7.10 (d, J = 5.2 Hz, 1H), 7.04 (d, J = 8.3 Hz, 1H), 5.70 (s, 2H), 4.67 (s, 2H), 2.38 (s, 3H), 2.31 (s, 3H). |
| | N-((5-(5-amino-6- | | |
| | methylpyridin-2-yl)-3- | | |
| | methylisoxazol-4-yl)methyl)- | | |
| | 4-(thiophen-2-yl)pyrimidin-2- | | |
| | amine | | |
| H20 | 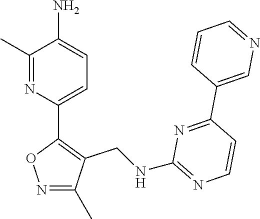 [SMILES: Cc1nc(-c2onc(C)c2CNc2nccc(-c3cccnc3)n2)ccc1N] | H13 | LC-MS (ESI): m/z 372.3 (M + 1) (Method 3) 1H NMR (300 MHz, DMSO-d6) δ 9.25 (s, 1H), 8.67 (dd, J = 4.8, 1.7 Hz, 1H), 8.41 (s, 1H), 8.34 (d, J = 8.1 Hz, 1H), 7.67 (s, 1H), 7.50 (d, J = 8.3 Hz, 2H), 7.27 (d, J = 5.1 Hz, 1H), 7.04 (d, J = 8.4 Hz, 1H), 5.70 (s, 2H), 4.76 (s, 2H), 2.36 (s, 3H), 2.29 (s, 3H). |
| | N-((5-(5-amino-6- | | |
| | methylpyridin-2-yl)-3- | | |
| | methylisoxazol-4-yl)methyl)- | | |
| | 4-(pyridin-3-yl)pyrimidin-2- | | |
| | amine | | |

### Step 8: methyl 3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylate, mix of Trans diastereomers (Intermediate H21)

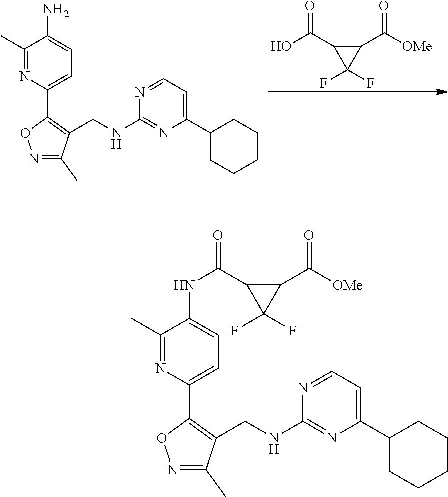 [SMILES: Cc1nc(-c2onc(C)c2CNc2nccc(C3CCCCC3)n2)ccc1N ; COC(=O)C1C(C(=O)O)C1(F)F ; COC(=O)C1C(C(=O)Nc2ccc(-c3onc(C)c3CNc3nccc(C4CCCCC4)n3)nc2C)C1(F)F]

Intermediate H21 (69 mg, 0.128 mmol, 53.7% yield) was obtained from N-((5-(5-amino-6-methylpyridin-2-yl)-3-methylisoxazol-4-yl)methyl)-4-cyclohexylpyrimidin-2-amine (Intermediate H15, 90.0 mg, 0.240 mmol) and Trans-rac 2,2-difluoro-3-methoxycarbonyl-cyclopropanecarboxylic acid (55.68 mg, 0.310 mmol) using a similar method as for Intermediate A17.

LC-MS (ESI): m/z (M+1): 541.2 (Method 1)

The Intermediates in the following table were prepared from reagents reported below following similar procedures as for Intermediate A17.

| Intermediate | Structure & Name | Reagents | Analytical data |
|---|---|---|---|
| H22 | 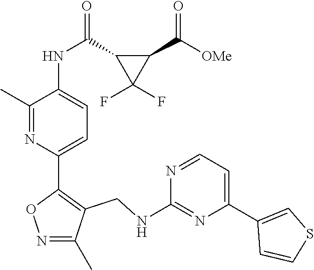 [SMILES: COC(=O)[C@@H]1[C@@H](C(=O)Nc2ccc(-c3onc(C)c3CNc3nccc(-c4ccsc4)n3)nc2C)C1(F)F] | H16 + Intermediate 2 | LC-MS (ESI): m/z 542.1 (M + 1) (Method 4) 1H NMR (400 MHz, DMSO-d6) δ 10.27 (s, 1H), 8.49 (s, 1H), 8.40 (s, 1H), 8.19 (d, J = 8.4 Hz, 1H), 7.89 − 7.62 (m, 3H), 7.13 − 6.93 (m, 2H), 4.86 (s, 2H), 3.76 (s, 3H), 3.74 − 3.67 (m, 1H), 3.24 (dd, J = 13.4, 7.8 Hz, 1H), 2.54 (s, 3H), 2.33 (s, 3H) |
| | methyl (1S,3S)-2,2-difluoro-3- | | |
| | ((2-methyl-6-(3-methyl-4-(((4- | | |
| | (thiophen-3-yl)pyrimidin-2- | | |
| | yl)amino)methyl)isoxazol-5- | | |
| | yl)pyridin-3- | | |
| | yl)carbamoyl)cyclopropane-1- | | |
| | carboxylate | | |
| H23 | 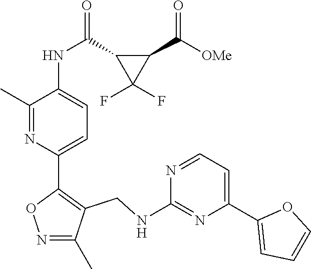 | H17 + Intermediate 2 | LC-MS (ESI): m/z 551.4 (M + 1) (Method 4) 1H NMR (300 MHz, DMSO-d6) δ 10.27 (s, 1H), 8.32 (d, J = 5.2 Hz, 1H), 8.17 (d, J = 8.5 Hz, 1H), 7.87 (d, J = 1.8 Hz, 1H), 7.79 (d, J = 8.4 Hz, 1H), 7.47 (t, J = 6.0 Hz, 1H), 7.00 (s, 1H), 6.90 (d, J = 5.1 Hz, 1H), 6.64 (dd, J = 3.5, 1.7 Hz, 1H), 4.78 (d, J = 5.8 Hz, 2H), 3.75 (s, 3H), 3.70 (dd, J = 11.3, 3.9 Hz, 1H), 3.24 (dd, J = 13.5, 7.8 Hz, 1H), 2.55 (s, 3H), 2.35 (s, 3H) |
| | methyl (1S,3S)-2,2-difluoro-3- | | |
| | ((6-(4-(((4-(furan-2- | | |
| | yl)pyrimidin-2- | | |
| | yl)amino)methyl)-3- | | |
| | methylisoxazol-5-yl)-2- | | |
| | methylpyridin-3- | | |
| | yl)carbamoyl)cyclopropane-1- | | |
| | carboxylate | | |
| H24 | 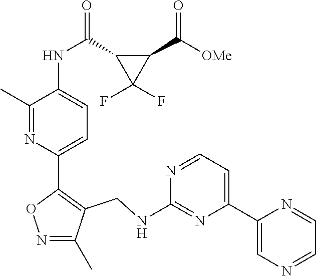 [SMILES: COC(=O)[C@@H]1[C@@H](C(=O)Nc2ccc(-c3onc(C)c3CNc3nccc(-c4cnccn4)n3)nc2C)C1(F)F] | H18 + Intermediate 2 | LC-MS (ESI): m/z 537.2 (M + 1) (Method 4) 1H NMR (300 MHz, DMSO-d6) δ 10.25 (s, 1H), 9.37 (s, 1H), 8.77 (s, 2H), 8.52 (d, J = 5.0 Hz, 1H), 8.19 (d, J = 8.4 Hz, 1H), 7.79 (d, J = 8.4 Hz, 1H), 7.72 (t, J = 5.6 Hz, 1H), 7.47 (d, J = 5.0 Hz, 1H), 4.90 (s, 2H), 3.76 (s, 3H), 3.75 − 3.65 (m, 1H), 3.30 − 3.17 (m, 1H), 2.55 (s, 3H), 2.35 (s, 3H) |
| | methyl (1S,3S)-2,2-difluoro-3- | | |
| | ((2-methyl-6-(3-methyl-4-(((4- | | |
| | (pyrazin-2-yl)pyrimidin-2- | | |
| | yl)amino)methyl)isoxazol-5- | | |
| | yl)pyridin-3- | | |
| | yl)carbamoyl)cyclopropane-1- | | |
| | carboxylate | | |
| H25 | 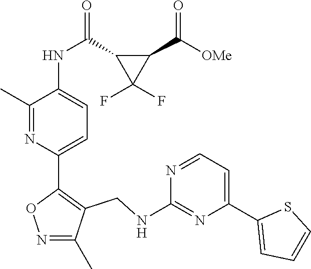 [SMILES: COC(=O)[C@@H]1[C@@H](C(=O)Nc2ccc(-c3onc(C)c3CNc3nccc(-c4cccs4)n3)nc2C)C1(F)F] | H19 + Intermediate 2 | LC-MS (ESI): m/z 541.3 (M + 1) (Method 4) |
| | methyl (1S,3S)-2,2-difluoro-3- | | |
| | ((2-methyl-6-(3-methyl-4-(((4- | | |
| | (thiophen-2-yl)pyrimidin-2- | | |
| | yl)amino)methyl)isoxazol-5- | | |
| | yl)pyridin-3- | | |
| | yl)carbamoyl)cyclopropane-1- | | |
| | carboxylate | | |
| H26 | 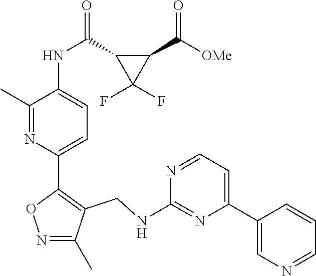 | H20 + Intermediate 2 | LC-MS (ESI): m/z 546.2 (M + 1) (Method 4) 1H NMR (300 MHz, DMSO-d6) δ 10.26 (s, 1H), 9.21 (d, J = 2.3 Hz, 1H), 8.65 (dd, J = 4.6, 1.6 Hz, 1H), 8.41 (d, J = 5.0 Hz, 1H), 8.29 (s, 1H), 8.18 (d, J = 8.5 Hz, 1H), 7.78 (d, J = 8.5 Hz, 1H), 7.62 (t, J = 5.7 Hz, 1H), 7.51 − 7.39 (m, 1H), 7.27 (d, J = 5.1 Hz, 1H), 4.87 (s, 2H), 3.75 (s, 3H), 3.74 − 3.67 (m, 1H), 3.24 (dd, J = 13.2, 7.9 Hz, 1H), 2.54 (s, 3H), 2.33 (s, 3H) |
| | methyl (1S,3S)-2,2-difluoro-3- | | |
| | ((2-methyl-6-(3-methyl-4-(((4- | | |
| | (pyridin-3-yl)pyrimidin-2- | | |
| | yl)amino)methyl)isoxazol-5- | | |
| | yl)pyridin-3- | | |
| | yl)carbamoyl)cyclopropane-1- | | |
| | carboxylate | | |

### Step 9: 3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid, mix of Trans diastereomers (Compound 18)

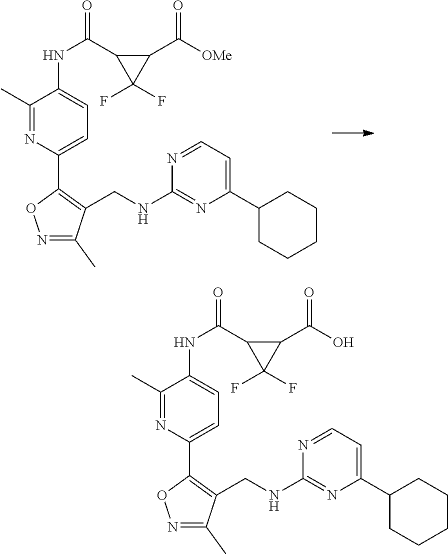

Compound 18 (36 mg, 0.068 mmol, 56% yield) was obtained as a white solid from methyl 3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylate, mix of Trans diastereomers (Intermediate H21, 69.0 mg, 0.120 mmol), using a similar method as for Compound 3, step 3.

LC-MS (ESI): m/z (M+1): 527.3 (Method 1)

1H NMR (600 MHz, DMSO-d6) δ ppm 12.54-14.40 (m, 1H), 10.19 (s, 1H), 8.18 (d, J=8.6 Hz, 1H), 8.09-8.15 (m, 1H), 7.77 (d, J=8.6 Hz, 1H), 7.25 (t, J=5.9 Hz, 1H), 6.46 (br d, J=4.9 Hz, 1H), 4.72 (br d, J=3.6 Hz, 2H), 3.54-3.63 (m, 1H), 3.02 (br dd, J=14.3, 8.1 Hz, 1H), 2.55 (s, 3H), 2.22-2.36 (m, 4H), 1.58-1.78 (m, 5H), 1.01-1.38 (m, 5H)

Compound 18 was submitted to chiral preparative HPLC.

Conditions: Column Chiralpak IC (25×3.0 cm), 5μ Mobile phase n-Hexane/(Ethanol+0.1% formic acid) 88/12% v/v Flow rate (ml/min) 35 ml/min DAD detection 220 nm Loop 600 μL.

### Peak 1: Compound 19

Single Enantiomer 1 of Trans-3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid

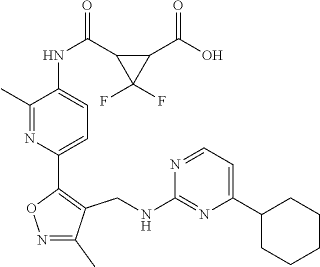 [SMILES: Cc1nc(-c2onc(C)c2CNc2nccc(C3CCCCC3)n2)ccc1NC(=O)C1C(C(=O)O)C1(F)F]

LC-MS (ESI): m/z (M+1): 527.2 (Method 1)

The corresponding sodium salt of Compound 19 was prepared using a similar method as for Compound 8. Compound 19 was characterized as sodium salt.

LC-MS (ESI): m/z (M+1-Na+): 527.2 (Method 1)

1H NMR (500 MHz, DMSO-d6) δ ppm 10.08 (s, 1H), 8.05-8.24 (m, 2H), 7.73 (d, J=8.5 Hz, 1H), 7.26 (br t, J=5.8 Hz, 1H), 6.46 (br d, J=4.9 Hz, 1H), 4.71 (br d, J=4.5 Hz, 2H), 3.21-3.29 (m, 1H), 2.56-2.66 (m, 1H), 2.54 (s, 3H), 2.24-2.35 (m, 4H), 1.59-1.82 (m, 5H), 1.01-1.43 (m, 5H)

### Peak 2: Compound 20

Single Enantiomer 2 of Trans-3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid

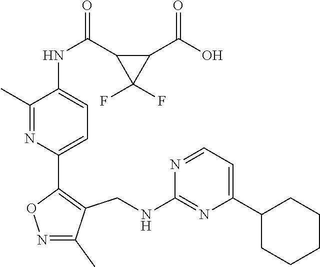 [SMILES: Cc1nc(-c2onc(C)c2CNc2nccc(C3CCCCC3)n2)ccc1NC(=O)C1C(C(=O)O)C1(F)F]

LC-MS (ESI): m/z (M+1): 527.2 (Method 1)

The corresponding sodium salt of Compound 20 was prepared using a similar method as for Compound 8. Compound 20 was characterized as sodium salt.

LC-MS (ESI): m/z (M+1-Na+): 527.2 (Method 1)

1H NMR (500 MHz, DMSO-d6) δ ppm 10.08 (s, 1H), 8.08-8.26 (m, 2H), 7.73 (d, J=8.4 Hz, 1H), 7.26 (br t, J=5.8 Hz, 1H), 6.46 (br d, J=4.9 Hz, 1H), 4.55-4.85 (m, 2H), 3.18-3.30 (m, 1H), 2.56-2.65 (m, 1H), 2.54 (s, 3H), 2.25-2.35 (m, 4H), 1.54-1.85 (m, 5H), 1.03-1.43 (m, 5H)

The Compounds in the following table were prepared from reagents reported below following similar procedures as for Compound 1, step 9.

| Compound | Structure & Name | Reagents | Analytical data |
|---|---|---|---|
| 21 | 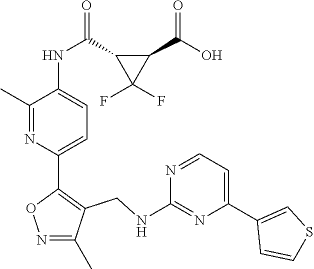 [SMILES: Cc1nc(-c2onc(C)c2CNc2nccc(-c3ccsc3)n2)ccc1NC(=O)[C@@H]1[C@@H](C(=O)O)C1(F)F] | H22 | LC-MS (ESI): m/z 527.1 (M + 1) (Method 4) 1H NMR (300 MHz, DMSO-d6) δ 10.24 (s, 1H), 8.31 (d, J = 5.2 Hz, 1H), 8.24 − 8.12 (m, 2H), 7.79 (d, J = 8.4 Hz, 1H), 7.59 (s, 2H), 7.46 (t, J = 5.9 Hz, 1H), 7.06 (d, J = 5.1 Hz, 1H), 4.83 (s, 2H), 3.67 − 3.49 (m, 1H), 3.09 − 2.94 (m, 1H), 2.56 (s, 3H), 2.34 (s, 3H). |
| | (1S,3S)-2,2-difluoro-3-((2- | | |
| | methyl-6-(3-methyl-4-(((4- | | |
| | (thiophen-3-yl)pyrimidin-2- | | |
| | yl)amino)methyl)isoxazol-5- | | |
| | yl)pyridin-3- | | |
| | yl)carbamoyl)cyclopropane- | | |
| | 1-carboxylic acid | | |
| 22 | 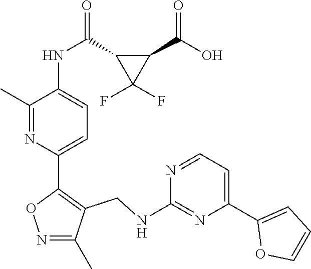 [SMILES: Cc1nc(-c2onc(C)c2CNc2nccc(-c3ccco3)n2)ccc1NC(=O)[C@@H]1[C@@H](C(=O)O)C1(F)F] | H23 | LC-MS (ESI): m/z 511.1 (M + 1) (Method 5) 1H NMR (400 MHz, DMSO- d6) δ 10.18 (s, 1H), 8.32 (d, J = 3.8 Hz, 1H), 8.16 (d, J = 8.4 Hz, 1H), 7.88 − 7.86 (m, 1H), 7.77 (d, J = 8.4 Hz, 1H), 7.46 (t, J = 5.4 Hz, 1H), 7.02 (bs, 1H), 6.90 (d, J = 5.0 Hz, 1H), 6.67 − 6.61 (m, 1H), 4.77 (d, J = 5.8 Hz, 2H), 3.51 − 3.40 (m, 1H), 2.88 (m, J = 13.4, 7.1 Hz, 1H), 2.55 (s, 3H), 2.35 (s, 3H). |
| | (1S,3S)-2,2-difluoro-3-((6- | | |
| | (4-(((4-(furan-2- | | |
| | yl)pyrimidin-2- | | |
| | yl)amino)methyl)-3- | | |
| | methylisoxazol-5-yl)-2- | | |
| | methylpyridin-3- | | |
| | yl)carbamoyl)cyclopropane- | | |
| | 1-carboxylic acid | | |
| 23 | 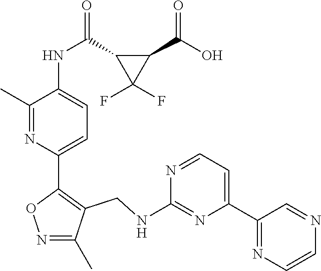 [SMILES: Cc1nc(-c2onc(C)c2CNc2nccc(-c3cnccn3)n2)ccc1NC(=O)[C@@H]1[C@@H](C(=O)O)C1(F)F] | H24 | LC-MS (ESI): m/z 523.2 (M + 1) (Method 6) 1H NMR (300 MHz, DMSO- d6) δ 10.22 (s, 1H), 9.38 (s, 1H), 8.77 (s, 2H), 8.51 (d, J = 5.0 Hz, 1H), 8.23 − 8.13 (m, 1H), 7.79 (d, J = 8.4 Hz, 1H), 7.72 (t, J = 5.7 Hz, 1H), 7.47 (d, J = 5.0 Hz, 1H), 4.90 (s, 2H), 3.59 (dd, J = 13.7, 7.8 Hz, 1H), 3.03 (dd, J = 14.6, 7.7 Hz, 1H), 2.55 (s, 3H), 2.35 (s, 3H). |
| | (1S,3S)-2,2-difluoro-3-((2- | | |
| | methyl-6-(3-methyl-4-(((4- | | |
| | (pyrazin-2-yl)pyrimidin-2- | | |
| | yl)amino)methyl)isoxazol- | | |
| | 5-yl)pyridin-3- | | |
| | yl)carbamoyl)cyclopropane- | | |
| | 1-carboxylic acid | | |
| 24 | 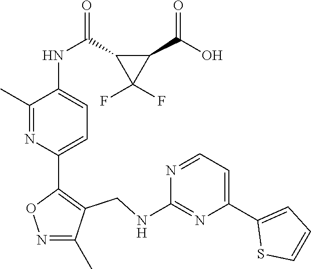 [SMILES: Cc1nc(-c2onc(C)c2CNc2nccc(-c3cccs3)n2)ccc1NC(=O)[C@@H]1[C@@H](C(=O)O)C1(F)F] | H25 | LC-MS (ESI): m/z 527.4 (M + 1) (Method 7) 1H NMR (300 MHz, DMSO- d6) δ 10.27 (s, 1H), 8.29 (d, J = 5.2 Hz, 1H), 8.14 (d, J = 8.5 Hz, 1H), 7.85 (s, 1H), 7.76 (d, J = 8.4 Hz, 1H), 7.68 (d, J = 5.0 Hz, 1H), 7.52 (t, J = 6.0 Hz, 1H), 7.16 (t, J = 4.3 Hz, 1H), 7.09 (d, J = 5.2 Hz, 1H), 4.87 − 4.72 (m, 2H), 2.87 − 2.75 (m, 1H), 2.55 (s, 3H), 2.34 (s, 3H). |
| | (1S,3S)-2,2-difluoro-3-((2- | | |
| | methyl-6-(3-methyl-4-(((4- | | |
| | (thiophen-2-yl)pyrimidin-2- | | |
| | yl)amino)methyl)isoxazol- | | |
| | 5-yl)pyridin-3- | | |
| | yl)carbamoyl)cyclopropane- | | |
| | 1-carboxylic acid | | |
| 25 | 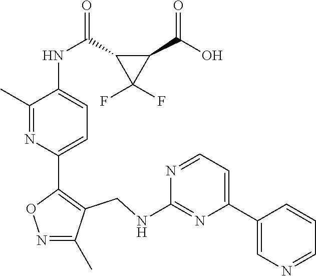 [SMILES: Cc1nc(-c2onc(C)c2CNc2nccc(-c3cccnc3)n2)ccc1NC(=O)[C@@H]1[C@@H](C(=O)O)C1(F)F] | H26 | LC-MS (ESI): m/z 522.2 (M + 1) (Method 8) 1H NMR (400 MHz, DMSO- d6) δ 13.46 (bs, 1H), 10.20 (s, 1H), 9.22 (d, J = 2.2 Hz, 1H), 8.66 (dd, J = 4.8, 1.7 Hz, 1H), 8.42 (d, J = 5.0 Hz, 1H), 8.31 (s, 1H), 8.18 (d, J = 8.4 Hz, 1H), 7.78 (d, J = 8.4 Hz, 1H), 7.61 (t, J = 5.8 Hz, 1H), 7.45 (s, 1H), 7.27 (d, J = 5.1 Hz, 1H), 4.88 (s, 2H), 3.59 − 3.47 (m, 1H), 3.02 − 2.93 (m, 1H), 2.54 (d, J = 4.6 Hz, 3H), 2.34 (d, J = 1.8 Hz, 3H). |
| | (1S,3S)-2,2-difluoro-3-((2- | | |
| | methyl-6-(3-methyl-4-(((4- | | |
| | (pyridin-3-yl)pyrimidin-2- | | |
| | yl)amino)methyl)isoxazol- | | |
| | 5-yl)pyridin-3- | | |
| | yl)carbamoyl)cyclopropane-1- | | |
| | carboxylic acid | | |

### Example 8

### Process synthesis of 3-((4-(3-((((R)-1-(2-chlorophenyl)ethoxy)carbonyl)amino) thiophen-2-yl)phenyl)carbamoyl)-2,2-difluorocyclopropane-1-carboxylic acid, mix of Trans diastereomers (Compound 26) and of it Single diastereomers Trans Compound 27

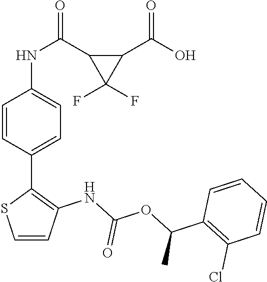 [SMILES: C[C@@H](OC(=O)Nc1ccsc1-c1ccc(NC(=O)C2C(C(=O)O)C2(F)F)cc1)c1ccccc1Cl]

### Step 1: (R)-1-(2-chlorophenyl)ethyl thiophen-3-ylcarbamate (Intermediate I1)

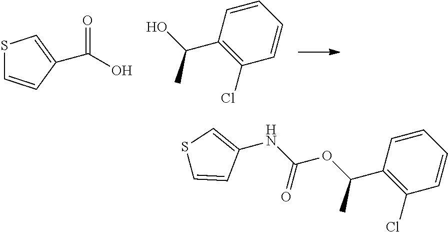

A 100 mL three-necked flask equipped with reflux condenser, stir bar, thermometer and nitrogen inlet was charged with thiophene-3-carboxylic acid (2.0 g, 15.61 mmol) and (R)-1-(2-chlorophenyl)ethan-1-ol (2.44 g, 15.61 mmol). Three cycles of vacuum-nitrogen back-filling were performed. Dry toluene (30 mL) was added, followed by TEA (4.35 mL, 31.21 mmol) and DPPA (4.04 mL, 18.73 mmol). The mixture was heated at 125° C. over 1 h. The mixture was cooled at RT and concentrated under reduced pressure. Sat. aq. NaHCO₃ (30 mL) was added and the mixture was extracted with EtOAc (3×30 mL). The collected organic phases were washed with water (50 mL) and brine (50 mL), dried over Na₂SO₄, filtered and concentrated to dryness in vacuo. The crude material was purified by flash chromatography (gradient CyHex/EtOAc from 95/5 to 60/40) to give the title compound (3.68 g, 13.06 mmol, 84% yield) as an off-white solid.

LC-MS (ESI): m/z (M+1): 282.4 (Method 1)

The Intermediate in the following table was prepared from reagents reported below following similar procedures as for Intermediate I1.

| Intermediate | Structure & Name | Reagents | Analytical data |
|---|---|---|---|
| I2 | 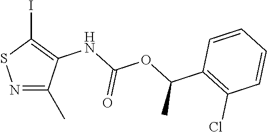 [SMILES: Cc1nsc(I)c1NC(=O)O[C@H](C)c1ccccc1Cl] | 5-iodoisothiazole- 4-carboxylic acid | LC-MS (ESI): m/z (M + 1) 423.1 (Method 1) 1H NMR (400 MHz, CHLOROFORM-d) δ ppm 7.64 − 7.51 (m, 1 H), 7. 41 − 7.24 (m, 4 H) , 6.29 − 6.21 (m, 1 H), 2.47 − 2.41 (m, 3 H), 1.62 − 1.59 (m, 3 H) |
| | 5-iodoisothiazole-4- | | |
| | carboxylic acid | | |

### Step 2: (R)-1-(2-chlorophenyl)ethyl (2-bromothiophen-3-yl)carbamate (Intermediate 13)

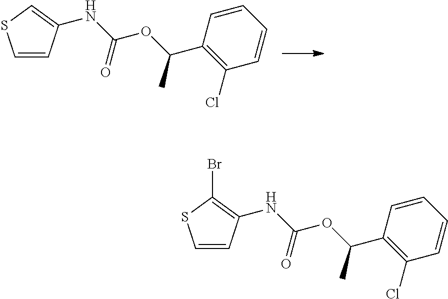 [SMILES: C[C@@H](OC(=O)Nc1ccsc1)c1ccccc1Cl ; C[C@@H](OC(=O)Nc1ccsc1Br)c1ccccc1Cl]

A 25 mL round bottom as equipped with stir bar an re flux condenser was charged with (R)-1-(2-chlorophenyl)ethyl thiophen-3-ylcarbamate (intermediate I1, 2.0 g, 6.39 mmol) and DCM (20 mL). NBS (1137.01 mg, 6.39 mmol) was added and the mixture was heated at reflux for 1 h. The reaction mixture was cooled to RT, diluted with DCM (10 mL), washed with sat. aq. K₂CO₃ (2×15 mL) and brine (20 mL), dried over Na₂SO₄, filtered, and evaporated to dryness. The crude product (2.86 g, crude) was obtained as an off-white solid.

LC-MS (ESI): m/z (M+1): 362.0 (Method 1)

### Step 3: (R)-1-(2-chlorophenyl)ethyl (2-(4-nitrophenyl)thiophen-3-yl)carbamate (Intermediate 14) NO₂

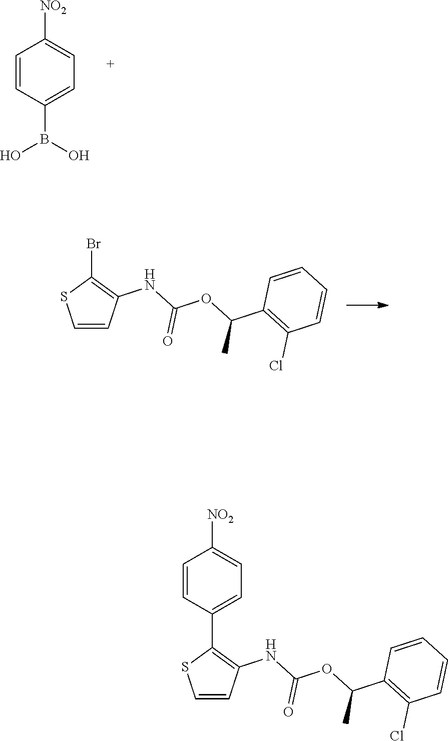 [SMILES: O=[N+]([O-])c1ccc(B(O)O)cc1 ; C[C@@H](OC(=O)Nc1ccsc1Br)c1ccccc1Cl ; C[C@@H](OC(=O)Nc1ccsc1-c1ccc([N+](=O)[O-])cc1)c1ccccc1Cl]

A mixture of (R)-1-(2-chlorophenyl)ethyl (2-bromothiophen-3-yl)carbamate (Intermediate 13, 500.0 mg, 1.39 mmol), (4-nitrophenyl)boronic acid (231.43 mg, 1.39 mmol), Na₂CO₃ (440.83 mg, 4.16 mmol) and Pd(dppf)Cl₂ (50.72 mg, 0.070 mmol) in 1,2-dimethoxyethane (9 mL) and water (3 mL) was degassed (vacuum/N₂ cycles) and stirred at 80° C. for 1h. The mixture was allowed to cool to RT and filtered through a pad of Celite washing with EtOAc. The filtrate was washed with saturated NaHCO₃ aqueous solution then the organic phase was separated, filtered through a hydrophobic phase separator and concentrated to dryness under reduced pressure. The crude was purified by flash chromatography (gradient CyHex/EtOAc from 100:0 to 90:10). Evaporation of proper fractions provided the target compound (514 mg) as a yellow solid.

LC-MS (ESI): m/z (M+1): 403.3 (Method 1)

### Step 4: (R)-1-(2-chlorophenyl)ethyl (2-(4-aminophenyl)thiophen-3-yl)carbamate (Intermediate I5)

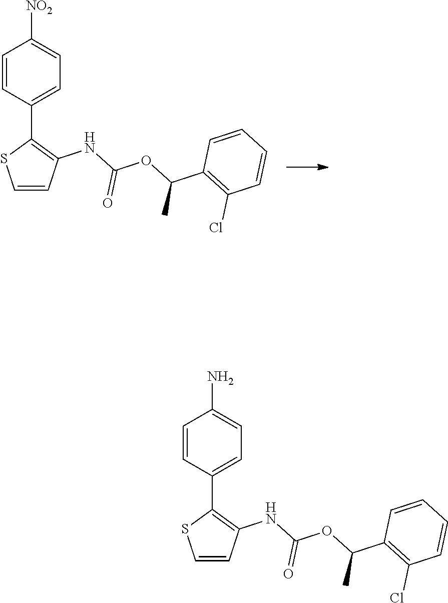 [SMILES: C[C@@H](OC(=O)Nc1ccsc1-c1ccc([N+](=O)[O-])cc1)c1ccccc1Cl ; C[C@@H](OC(=O)Nc1ccsc1-c1ccc(N)cc1)c1ccccc1Cl]

(R)-1-(2-chlorophenyl)ethyl (2-(4-nitrophenyl)thiophen-3-yl)carbamate (Intermediate 14, 514.0 mg, 1.28 mmol) was dissolved in DCM (5 mL) and MeOH (2 mL) then concentrated HCl (0.43 mL, 5.1 mmol) was added followed by the addition of Fe⁰ (499 mg, 8.93 mmol). The mixture was stirred at RT for 3 h, then was concentrated under reduced pressure and pH was adjusted to ˜8-9 with 2N NaOH. The mixture was extracted with DCM and the organic phase was evaporated to dryness to provide the title compound (472 mg, 1.266 mmol, 99% yield) as a pale yellow solid.

LC-MS (ESI): m/z (M+1): 373.1 (Method 1)

### Step 5: methyl 3-((4-(3-((((R)-1-(2-chlorophenyl)ethoxy)carbonyl)amino)thiophen-2-yl)phenyl)carbamoyl)-2,2-difluorocyclopropane-1-carboxylate, mix of Trans diastereomers (Intermediate 16)

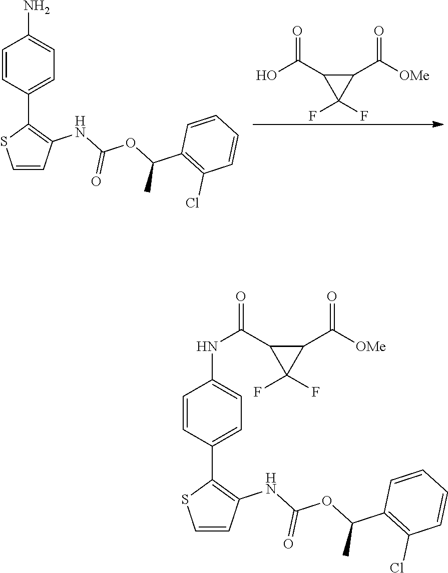 [SMILES: C[C@@H](OC(=O)Nc1ccsc1-c1ccc(N)cc1)c1ccccc1Cl ; COC(=O)C1C(C(=O)O)C1(F)F ; COC(=O)C1C(C(=O)Nc2ccc(-c3sccc3NC(=O)O[C@H](C)c3ccccc3Cl)cc2)C1(F)F]

Intermediate 16 (141 mg, 0.264 mmol, 65% yield) was obtained from (R)-1-(2-chlorophenyl)ethyl (2-(4-aminophenyl)thiophen-3-yl)carbamate (Intermediate I5, 194 mg, 0.520 mmol) and Trans-rac-2,2-difluoro-3-(methoxycarbonyl)cyclopropane-1-carboxylic acid (122 mg, 0.68 mmol) using a similar method as for Intermediate A17.

LC-MS (ESI): m/z (M+1): 535.2 (Method 1)

### Step 6: 3-((4-(3-((((R)-1-(2-chlorophenyl)ethoxy)carbonyl)amino)thiophen-2-yl)phenyl)carbamoyl)-2,2-difluorocyclopropane-1-carboxylic acid, mix of Trans diastereomers (Compound 26)

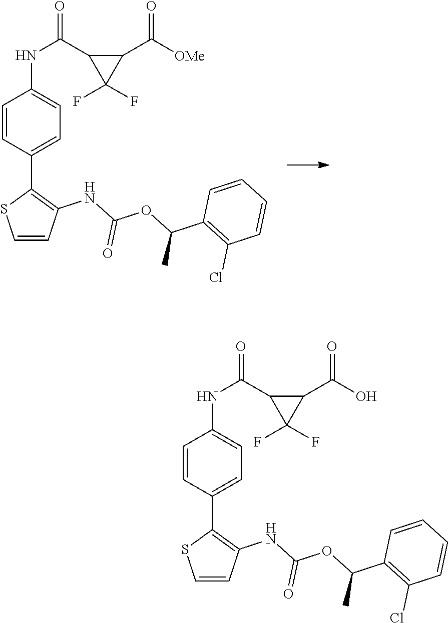 [SMILES: COC(=O)C1C(C(=O)Nc2ccc(-c3sccc3NC(=O)O[C@H](C)c3ccccc3Cl)cc2)C1(F)F ; C[C@@H](OC(=O)Nc1ccsc1-c1ccc(NC(=O)C2C(C(=O)O)C2(F)F)cc1)c1ccccc1Cl]

Compound 26 (112 mg, 0.215 mmol, 82% yield) was obtained from methyl 3-((4-(3-((((R)-1-(2-chlorophenyl)ethoxy)carbonyl)amino)thiophen-2-yl)phenyl)carbamoyl)-2,2-difluorocyclopropane-1-carboxylate, mix of Trans diastereomers (Intermediate 16, 141 mg, 0.264 mmol), using a similar method as for Compound 3, step 3.

LC-MS (ESI): m/z (M+1): 535.2 (Method 1)

Compound 26 was submitted to chiral preparative HPLC.

Conditions: Column Chiralcel OJ-H (25×2.0 cm), 5μ Mobile phase n-Hexane/(Ethanol+0.1% formic acid) 80/20% v/v Flow rate (ml/min) 17 ml/min DAD detection 220 nm Loop 750 μL

### Peak 2: Compound 27

Single Diastereomer 2 of Trans-3-((4-(3-((((R)-1-(2-chlorophenyl) ethoxy)carbonyl)amino)thiophen-2-yl)phenyl)carbamoyl)-2,2-difluorocyclopropane-1-carboxylic acid

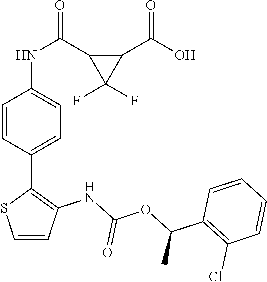 [SMILES: C[C@@H](OC(=O)Nc1ccsc1-c1ccc(NC(=O)C2C(C(=O)O)C2(F)F)cc1)c1ccccc1Cl]

LC-MS (ESI): m/z (M+1): 535.2 (Method 1)

1H NMR (400 MHz, DMSO-d6) δ 13.49 (s, 1H), 10.67 (s, 1H), 9.20 (s, 1H), 7.73-7.21 (m, 9H), 7.06 (d, J=5.36 Hz, 1H), 5.96 (d, J=6.67 Hz, 1H), 3.42-3.35 (m, 1H), 3.07 (dd, J=14.27, 7.71 Hz, 1H), 1.47 (s, 3H).

The corresponding sodium salt of Compound 27 was obtained using a similar method as for Compound 8.

LC-MS (ESI): m/z (M+1-Na+): 535.2 (Method 1)

1H NMR (500 MHz, DMSO-d6) δ ppm 10.58 (s, 1H), 9.23 (br s, 1H), 7.64 (d, J=8.6 Hz, 2H), 7.40-7.47 (m, 3H), 7.19-7.59 (m, 4H), 7.06 (br d, J=5.2 Hz, 1H), 5.84-6.07 (m, 1H), 3.07 (dd, J=13.2, 7.8 Hz, 1H), 2.56-2.69 (m, 1H), 1.48 (br s, 3H)

### Example 9

### Process synthesis of 3-((6-(3-((((R)-1-(2-chlorophenyl)ethoxy)carbonyl)amino) thiophen-2-yl)-2-methylpyridin-3-yl)carbamoyl)-2,2-difluorocyclopropane-1-carboxylic acid, mix of Trans diastereomers (Compound 28) and of it Single diastereomer Trans Compound 30

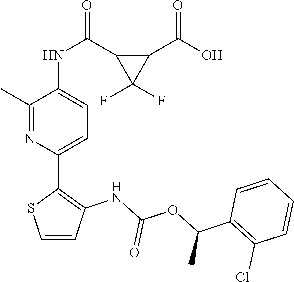 [SMILES: Cc1nc(-c2sccc2NC(=O)O[C@H](C)c2ccccc2Cl)ccc1NC(=O)C1C(C(=O)O)C1(F)F]

### Step 1: 2-methyl-6-(tetrabutyl-15-stannyl)pyridin-3-amine (intermediate L1)

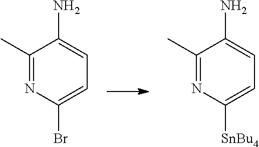

A 40 mL vial was charged with 6-bromo-2-methylpyridin-3-amine (500.0 mg, 2.67 mmol) and LiCl (680 mg, 16.04 mmol).The vial was sealed and three cycles of vacuum/nitrogen back-filling were applied. CPME (12.78 mL) and Bis(tributyltin) (1.62 mL, 3.21 mmol) were added.

Pd₂(dba)₃ (122.4 mg, 0.130 mmol) and PCy₃ (75 mg, 0.270 mmol) were added to the mixture, then three cycles of vacuum/nitrogen back-filling were repeated and the mixture was heated at 95° C. for 24 h. The mixture was cooled to RT, diluted with EtOAc and filtered over celite washing with EtOAc. The filtrate was evaporated affording the product (2.7 g, crude) as a light brown oil, that was used in the following step without further purification.

LC-MS (ESI): m/z (M+1): 399.3 (Method 1)

### Step 2: (R)-1-(2-chlorophenyl)ethyl (2-(5-amino-6-methylpyridin-2-yl)thiophen-3-yl)carbamate (Intermediate L2)

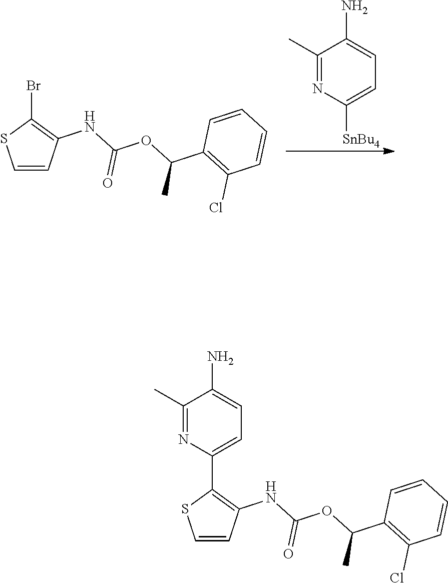

2-methyl-6-(tetrabutyl-15-stannyl)pyridin-3-amine (intermediate L1, 853.5 mg, 1.92 mmol) and (R)-1-(2-chlorophenyl)ethyl (2-bromothiophen-3-yl)carbamate (Intermediate 13, 600.0 mg, 1.56 mmol) was dissolved in DMF (12 mL). PPh₃ (178.8 mg, 0.18 mmol) and CuI (59.2 mg, 0.30 mmol) were added, then three cycles of vacuum/nitrogen back-filling were applied and the mixture was heated at 120° C. overnight. The mixture was cooled to RT, added with EtOAc (10 mL) and with a solution of 10% KF (8 mL), then was stirred for 30 min. The mixture was filtered and the filtrate was washed with brine (40 mL), the aqueous phase was further extracted with EtOAc (2 times) and the combined organic phase was concentrated under reduced pressure. The residue was purified by column chromatography (gradient EtOAc in CyHex from 0% to 40%) to afford the target compound (201.7 mg, 0.52 mmol, 33% yield) as a pale yellow solid.

LC-MS (ESI): m/z (M+1): 388.5 (Method 1)

The Intermediate in the following table was prepared from reagents reported below following similar procedures as for Intermediate L2.

| Intermediate | Structure & Name | Reagents | Analytical data |
|---|---|---|---|
| L3 | 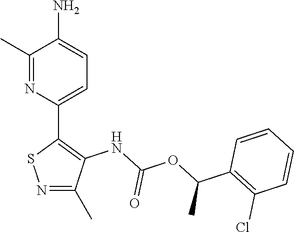 [SMILES: Cc1nc(-c2snc(C)c2NC(=O)O[C@H](C)c2ccccc2Cl)ccc1N] | I2 + L1 | LC-MS (ESI): m/z (M + 1): 403.2 (Method 1) |
| | (R)-1-(2- | | |
| | chlorophenyl)ethyl (5-(5- | | |
| | amino-6-methylpyridin-2- | | |
| | yl)-3-methylisothiazol-4- | | |
| | yl)carbamate | | |

### Step 3: methyl 3-((6-(3-((((R)-1-(2-chlorophenyl)ethoxy)carbonyl)amino)thiophen-2-yl)-2-methylpyridin-3-yl)carbamoyl)-2,2-difluorocyclopropane-1-carboxylate, mix of Trans diastereomers (Intermediate L4)

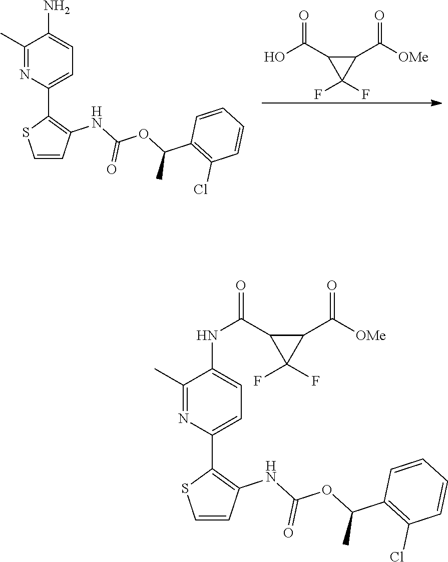 [SMILES: Cc1nc(-c2sccc2NC(=O)O[C@H](C)c2ccccc2Cl)ccc1N ; COC(=O)C1C(C(=O)O)C1(F)F ; COC(=O)C1C(C(=O)Nc2ccc(-c3sccc3NC(=O)O[C@H](C)c3ccccc3Cl)nc2C)C1(F)F]

Intermediate L4 (169 mg, 0.308 mmol, 59% yield)) was obtained from (R)-1-(2-chlorophenyl)ethyl (2-(5-amino-6-methylpyridin-2-yl)thiophen-3-yl)carbamate (Intermediate L2, 201.7 mg, 0.52 mmol) and Trans-rac 2,2-difluoro-3-(methoxycarbonyl)cyclopropane-1-carboxylic acid (121.7 mg, 0.680 mmol) using a similar method as for Intermediate A17.

LC-MS (ESI): m/z (M+1): 535.2 (Method 1)

The Intermediate in the following table was prepared from reagents reported below following similar procedures as for Intermediate L4.

| Intermediate | Structure & Name | Reagents | Analytical data |
|---|---|---|---|
| L5 | 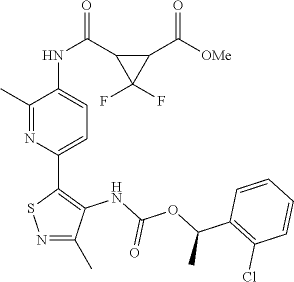 [SMILES: COC(=O)C1C(C(=O)Nc2ccc(-c3snc(C)c3NC(=O)O[C@H](C)c3ccccc3Cl)nc2C)C1(F)F] | L3 | LC-MS (ESI): m/z (M + 1): 565.2 (Method 1) 1H NMR (400 MHz, CDCl3) δ 8.67 (s, 1H), 8.24 (d, J = 8.4 Hz, 1H), 7.49-7.32 (m, 4H), 7.26- 7.19 (m, 2H), 6.21 (q, J = 6.5 Hz, 1H), 3.84 (s, 3H), 3.38-3.29 (m, 1H), 3.24-3.15 (m, 1H), 2.59 (s, 3H), 2.48 (s, 3H), 1.60 (d, J = 6.7 Hz, 3H). |
| | methyl 3-((6-(4-((((R)-1-(2- | | |
| | chlorophenyl)ethoxy)carbonyl) | | |
| | amino)-3-methylisothiazol-5-yl)- | | |
| | 2-methylpyridin-3- | | |
| | yl)carbamoyl)-2,2- | | |
| | difluorocyclopropane-1- | | |
| | carboxylate, mix of Trans | | |
| | diastereomers | | |

### Step 4: 3-((6-(3-((((R)-1-(2-chlorophenyl)ethoxy)carbonyl)amino)thiophen-2-yl)-2-methylpyridin-3-yl)carbamoyl)-2,2-difluorocyclopropane-1-carboxylic acid, mix of Trans diastereomers (Compound 28)

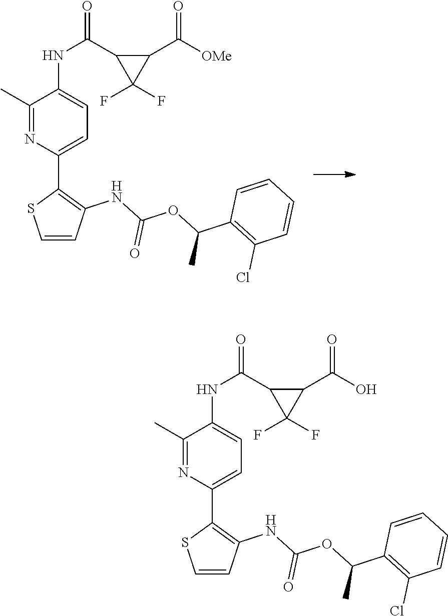 [SMILES: COC(=O)C1C(C(=O)Nc2ccc(-c3sccc3NC(=O)O[C@H](C)c3ccccc3Cl)nc2C)C1(F)F ; Cc1nc(-c2sccc2NC(=O)O[C@H](C)c2ccccc2Cl)ccc1NC(=O)C1C(C(=O)O)C1(F)F]

Compound 28 (278 mg, crude) was obtained from methyl 3-((6-(3-((((R)-1-(2-chlorophenyl)ethoxy)carbonyl)amino)thiophen-2-yl)-2-methylpyridin-3-yl)carbamoyl)-2,2-difluorocyclopropane-1-carboxylate, mix of Trans diastereomers (Intermediate L4, 169 mg, 0.308 mmol), using a similar method as for Compound 3, step 3.

LC-MS (ESI): m/z (M+1): 535.2 (Method 1)

The Compound in the following table was prepared from reagents reported below following similar procedures as for Compound 3, step 3.

| Compound | Structure & Name | Reagents | Analytical data |
|---|---|---|---|
| 29 | 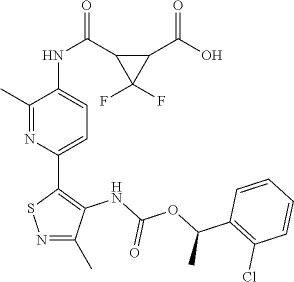 [SMILES: Cc1nc(-c2snc(C)c2NC(=O)O[C@H](C)c2ccccc2Cl)ccc1NC(=O)C1C(C(=O)O)C1(F)F] | L5 | LC-MS (ESI): m/z (M + 1): 551.3 (Method 1) |
| | 3-((6-(4-((((R)-1-(2- | | |
| | chlorophenyl)ethoxy)carbonyl) | | |
| | amino)-3-methylisothiazol-5-yl)-2- | | |
| | methylpyridin-3-yl)carbamoyl)-2,2- | | |
| | difluorocyclopropane-1-carboxylic | | |
| | acid, mix of Trans diastereomers | | |

Compound 28 was submitted to chiral preparative SFC.

Conditions: Column Whelk O1 (R,R) (25×3.0 cm), 10μ Modifier (Methanol+0.1% isopropylamine) 45% Flow rate (ml/min) 100 Pressure (bar) 120 Temperature (° C.) 40 UV detection 220 nm Loop 1200 M1

### Peak 2: Compound 30

Single Diastereomer 2 of Trans-3-((6-(3-((((R)-1-(2-chlorophenyl)ethoxy)carbonyl)amino) thiophen-2-yl)-2-methylpyridin-3-yl)carbamoyl)-2,2-difluorocyclopropane-1-carboxylic acid

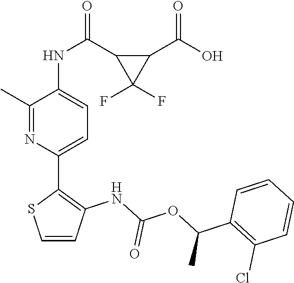 [SMILES: Cc1nc(-c2sccc2NC(=O)O[C@H](C)c2ccccc2Cl)ccc1NC(=O)C1C(C(=O)O)C1(F)F]

LC-MS (ESI): m/z (M+1): 536.2 (Method 1)

1H NMR (400 MHz, DMSO-d6) δ ppm 13.11-13.84 (m, 1H), 11.64 (br s, 1H), 10.21 (s, 1H), 7.98 (d, J=8.6 Hz, 1H), 7.52-7.68 (m, 3H), 7.31-7.50 (m, 4H), 6.02-6.14 (m, 1H), 3.56 (br dd, J=13.7, 8.0 Hz, 1H), 3.07 (br dd, J=14.1, 7.7 Hz, 1H), 2.56 (s, 3H), 1.55 (d, J=6.6 Hz, 3H)

The corresponding sodium salt of Compound 30 was obtained using a similar method as for Compound 8.

LC-MS (ESI): m/z (M+1-Na+): 536.1 (Method 1)

1H NMR (400 MHz, DMSO-d6) δ ppm 11.81 (br s, 1H), 10.03 (br s, 1H), 7.79-8.10 (m, 1H), 7.25-7.72 (m, 7H), 6.07 (q, J=6.5 Hz, 1H), 3.05-3.16 (m, 1H), 2.52-2.65 (m, 1H), 2.50 (br s, 3H), 1.53 (br d, J=6.4 Hz, 3H)

### Example 10

### Process synthesis of Single Diastereomer 1 of Trans-3-((6-(4-((((R)-1-(2-chlorophenyl)ethoxy)carbonyl)amino)-3-methylisothiazol-5-yl)-2-methylpyridin-3-yl)carbamoyl)-2,2-difluorocyclopropane-1-carboxylic acid (Compound 31)

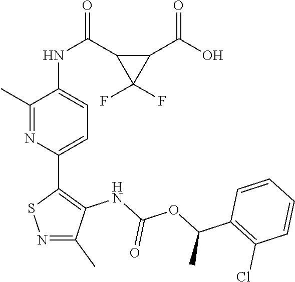 [SMILES: Cc1nc(-c2snc(C)c2NC(=O)O[C@H](C)c2ccccc2Cl)ccc1NC(=O)C1C(C(=O)O)C1(F)F]

Step 1: Single Diastereomer 1 of Trans-3-((6-(4-((((R)-1-(2-chlorophenyl) ethoxy)carbonyl)amino)-3-methylisothiazol-5-yl)-2-methylpyridin-3-yl)carbamoyl)-2,2-difluorocyclopropane-1-carboxylic acid (Compound 31)

Compound 29 was submitted to chiral preparative HPLC.

Conditions: Column Chiralpak AD-H (25×3.0 cm), 5 Mobile phase n-Hexane/(Ethanol+0.1% formic acid) 82/18% v/v Flow rate (ml/min) 17 ml/min DAD detection 220 nm Loop 1000 Id.

### Peak 1: Compound 31

Single Diastereomer 1 of Trans-3-((6-(4-((((R)-1-(2-chlorophenyl)ethoxy) carbonyl)amino)-3-methylisothiazol-5-yl)-2-methylpyridin-3-yl)carbamoyl)-2,2-difluorocyclopropane-1-carboxylic acid

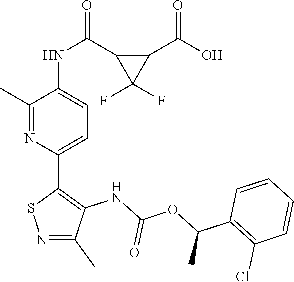 [SMILES: Cc1nc(-c2snc(C)c2NC(=O)O[C@H](C)c2ccccc2Cl)ccc1NC(=O)C1C(C(=O)O)C1(F)F]

LC-MS (ESI): m/z (M+1): 551.3 (Method 1)

1H NMR (500 MHz, DMSO-d6) δ ppm 13.51 (br s, 1H), 10.18 (s, 1H), 9.54 (br s, 1H), 8.06 (d, J=8.5 Hz, 1H), 7.63 (br d, J=6.7 Hz, 1H), 7.56 (br d, J=8.4 Hz, 1H), 7.41-7.50 (m, 2H), 7.30-7.39 (m, 1H), 5.81-6.14 (m, 1H), 3.58 (br dd, J=13.7, 7.8 Hz, 1H), 3.06 (br dd, J=14.0, 7.7 Hz, 1H), 2.47 (s, 3H), 2.24 (br s, 3H), 1.56 (br d, J=5.5 Hz, 3H)

The corresponding sodium salt of Compound 31 was obtained using a similar method as for Compound 8.

LC-MS (ESI): m/z (M+1-Na+): 551.3 (Method 1)

1H NMR (500 MHz, DMSO-d6) δ ppm 10.00 (br s, 1H), 9.53 (br s, 1H), 8.02 (br s, 1H), 7.63 (br d, J=5.2 Hz, 1H), 7.56 (br s, 1H), 7.45 (br s, 2H), 7.35 (br s, 1H), 5.83-6.13 (m, 1H), 3.18-3.26 (m, 1H), 2.58 (br dd, J=18.7, 7.5 Hz, 1H), 2.45 (s, 3H), 2.23 (br s, 3H), 1.55 (br s, 3H)

### Example 11

### Process synthesis of 3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid, mix of Trans diastereomers (Compound 32) and of its Single diastereomers Trans Compound 33 and Compound 34

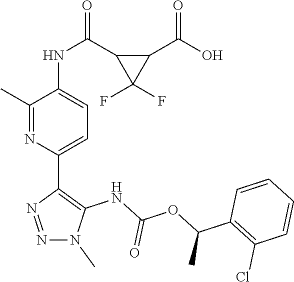 [SMILES: Cc1nc(-c2nnn(C)c2NC(=O)O[C@H](C)c2ccccc2Cl)ccc1NC(=O)C1C(C(=O)O)C1(F)F]

Step 1: 3-(5-bromo-6-methylpyridin-2-yl)prop-2-yn-1-ol (Intermediate M1)

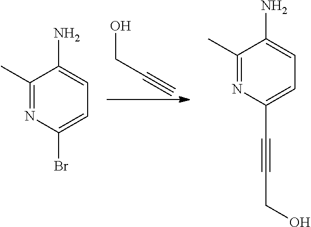

To an ice-cooled mixture of 3,6-dibromo-2-methylpyridine (7.0 g, 27.9 mmol) and 2-propyn-1-ol (2.44 mL, 41.85 mmol) in MeCN (45 mL), TEA (9.33 mL, 66.95 mmol) was added followed by the addition of CuI (212.5 mg, 1.12 mmol) and PdCl₂(PPh₃)₂ (785.5 mg, 1.12 mmol).

The mixture was allowed to reach RT and stirred for 2.5h. The reaction mixture was filtered through a pad of Celite, washing with EtOAc, and the filtrate was concentrated under reduced pressure. The crude was purified by flash chromatography (gradient of CyHex/EtOAc from 100:0 to 20:80) and proper fractions were evaporated to provide the title compound (5.524 g, 24.43 mmol, 88% yield) as an orange solid.

LC-MS (ESI): m/z (M+1): 227.9 (Method 1).

1H NMR (400 MHz, DMSO-d6) δ 8.02 (d, J=8.17 Hz, 1H), 7.27 (dd, J=8.24, 0.68 Hz, 1H), 5.42 (t, J=5.98 Hz, 1H), 4.32 (d, J=5.93 Hz, 2H), 2.56 (s, 3H).

The Intermediate in the following table was prepared from reagents reported below following similar procedures as for Intermediate M1.

| Intermediate | Structure & Name | Reagents | Analytical data |
|---|---|---|---|
| M2 | 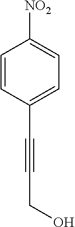 [SMILES: O=[N+]([O-])c1ccc(C#CCO)cc1] | 1-nitro-4- nitrobenzene | LC-MS (ESI): m/z (M + 1): 178.1 (Method 1) 1H NMR (400 MHz, Chloroform-d) δ 8.26-8.12 (m, 2H), 7.66-7.55 (m, 2H), 4.56 (d, J = 6.22 Hz, 2H), 1.76 (t, J = 6.22 Hz, 1H). |
| | 3-(4-nitrophenyl)prop-2-yn-1-ol | | |

### Step 2: 3-(5-bromo-6-methylpyridin-2-yl)prop-2-yn-1-ol (intermediate M3)

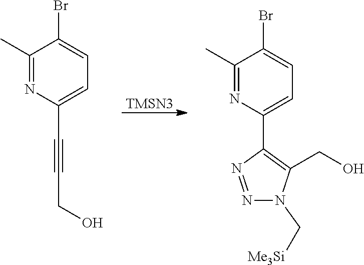

To a solution of 3-(5-bromo-6-methylpyridin-2-yl)prop-2-yn-1-ol (intermediate M1, 5.524 g, 24.43 mmol) in 1,2-dimethoxyethane (60 mL), TMSN3 (34.51 mL, 34.51 mmol) was added followed by the addition of Cp*RuCl(PPh₃)₂ (1107 mg, 1.38 mmol) and the mixture was stirred overnight at 50° C. The reaction mixture was concentrated under reduced pressure to provide the crude product (9.2 g, crude) that was used in the next step without further purification.

LC-MS (ESI): m z (M+1): 357.0 (Method 1)

The Intermediate in the following table was prepared from reagents reported below following similar procedures as for Intermediate M3.

| Intermediate | Structure & Name | Reagents | Analytical data |
|---|---|---|---|
| M4 | 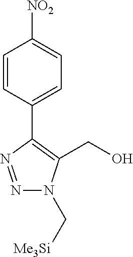 [SMILES: C[Si](C)(C)Cn1nnc(-c2ccc([N+](=O)[O-])cc2)c1CO] | M2 | LC-MS (ESI): m/z (M + 1): 307.2 (Method 1) |
| | (4-(4-nitrophenyl)-1- | | |
| | ((trimethylsilyl)methyl)-1H- | | |
| | 1,2,3-triazol-5-yl)methanol | | |

Step 3: (4-(5-bromo-6-methylpyridin-2-yl)-1-methyl-1H-1,2,3-triazol-5-yl)methanol (Intermediate M5)

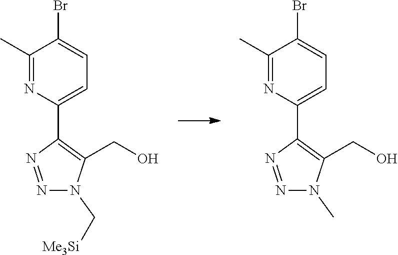 [SMILES: Cc1nc(-c2nnn(C[Si](C)(C)C)c2CO)ccc1Br ; Cc1nc(-c2nnn(C)c2CO)ccc1Br]

To an ice-cooled solution of 3-(5-bromo-6-methylpyridin-2-yl)prop-2-yn-1-ol (intermediate M3, 9.2 g, crude) in THE (30 mL), TBAF 1M in THE (23.0 mL, 23 mmol) was added then the reaction mixture was allowed to reach RT and stirred for 30 minutes. Solid NaHCO₃ was added and the mixture was stirred vigorously for 15 minutes. Solids were removed filtering through a pad of Celite and washing with EtOAc. The filtrate was concentrated under reduced pressure and the crude was purified by flash chromatography (gradient CyHex/EtOAc from 95:25 to 25:75). Pure fractions were evaporated to provide the title compound (2093 mg) as a pale yellow solid.

LC-MS (ESI): m/z (M+1): 284.9 (Method 1)

1H NMR (400 MHz, Chloroform-d) δ 8.06 (dd, J=8.38, 0.68 Hz, 1H), 7.98 (d, J=8.42 Hz, 1H), 6.86 (t, J=6.73 Hz, 1H), 4.86 (d, J=6.71 Hz, 2H), 4.11 (s, 3H), 2.74 (s, 3H).

The Intermediate in the following table was prepared from reagents reported below following similar procedures as for Intermediate M5.

| Intermediate | Structure & Name | Reagents | Analytical data |
|---|---|---|---|
| M6 | 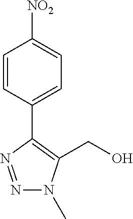 [SMILES: Cn1nnc(-c2ccc([N+](=O)[O-])cc2)c1CO] | M4 | LC-MS (ESI): m/z (M + 1): 235.1 (Method 1) 1H NMR (400 MHz, DMSO-d6) d ppm 8.32-8.39 (m, 2 H) 8.01-8.12 (m, 2 H) 5.65-5.74 (m, 1 H) 4.69-4.78 (m, 2 H) 4.12 (s, 3 H) |
| | (1-methyl-4-(4- | | |
| | nitrophenyl)-1H-1,2,3- | | |
| | triazol-5-yl)methanol | | |

Step 4: 4-(5-bromo-6-methylpyridin-2-yl)-1-methyl-1H-1,2,3-triazole-5-carboxylic acid (intermediate M7)

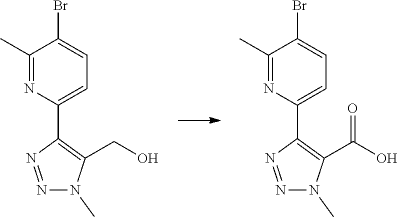 [SMILES: Cc1nc(-c2nnn(C)c2CO)ccc1Br ; Cc1nc(-c2nnn(C)c2C(=O)O)ccc1Br]

To a suspension of [(4-(5-bromo-6-methylpyridin-2-yl)-1-methyl-1H-1,2,3-triazol-5-yl)methanol (Intermediate M5, 2.093 g, 7.392 mmol) in water (25 mL), NaOH (591.4 mg, 14.79 mmol) was added followed by the addition of KMnO₄ (2.34 g, 14.79 mmol). The mixture was stirred at 100° C. for 1.5h, then was allowed to cool to RT and added with 3N HCl to pH-2-3. The mixture was extracted with DCM (3 times). Combined organic layers were dried over Na₂SO₄, filtered and concentrated under reduced pressure to provide the desired compound (1156 mg, 3.891 mmol, 5300 yield) as a white solid.

LC-MS (ESI): m/z (M+1): 298.9 (Method 1)

The Intermediate in the following table was prepared from reagents reported below following similar procedures as for Intermediate M7.

| Intermediate | Structure & Name | Reagents | Analytical data |
|---|---|---|---|
| M8 | 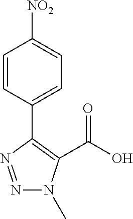 [SMILES: Cn1nnc(-c2ccc([N+](=O)[O-])cc2)c1C(=O)O] | M6 | LC-MS (ESI): m/z (M + 1): 249.1 (Method 1) |
| | 1-methyl-4-(4-nitrophenyl)- | | |
| | 1H-1,2,3-triazole-5- | | |
| | carboxylic acid | | |

### Step 5: methyl 4-(5-bromo-6-methylpyridin-2-yl)-1-methyl-1H-1,2,3-triazole-5-carboxylate (Intermediate M9)

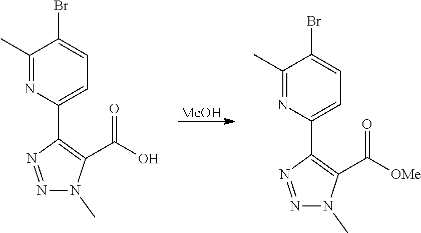 [SMILES: "MeOH" ; Cc1nc(-c2nnn(C)c2C(=O)O)ccc1Br ; COC(=O)c1c(-c2ccc(Br)c(C)n2)nnn1C]

To a suspension of 4-(5-bromo-6-methylpyridin-2-yl)-1-methyl-1H-1,2,3-triazole-5-carboxylic acid (intermediate M7, 500.0 mg, 1.68 mmol) in DCM (10 mL), MeOH (0.14 mL, 3.37 mmol) was added followed by the addition of DMAP (20.6 mg, 0.170 mmol) and EDC (645 mg, 3.37 mmol). The mixture was stirred at RT for 24h. Volatiles were removed under reduced pressure and the crude was purified by flash chromatography (gradient DCM/Methanol from 100:0 to 95:5). Proper fractions were evaporated to provide the desired compound (280 mg, 0.900 mmol, 53% yield) as a white solid.

LC-MS (ESI): m/z (M+1): 312.9 (Method 1)

### Step 5: methyl 4-(5-((tert-butoxycarbonyl)amino)-6-methylpyridin-2-yl)-1-methyl-1H-1,2,3-triazole-5-carboxylate (Intermediate M10)

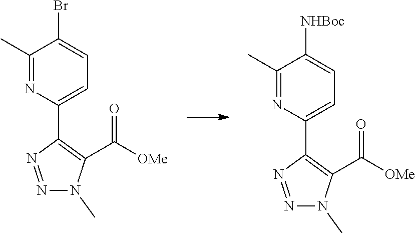 [SMILES: COC(=O)c1c(-c2ccc(Br)c(C)n2)nnn1C ; COC(=O)c1c(-c2ccc(NC(=O)OC(C)(C)C)c(C)n2)nnn1C]

A mixture of methyl 4-(5-bromo-6-methylpyridin-2-yl)-1-methyl-1H-1,2,3-triazole-5-carboxylate (Intermediate M9, 280.0 mg, 0.900 mmol), carbamic acid tert-butyl ester (147.6 mg, 1.26 mmol), XantPhos (62.5 mg, 0.110 mmol), Pd₂(dba)₃ (49.4 mg, 0.050 mmol) and Cs₂CO₃ (469.2 mg, 1.44 mmol) in CPME (6 mL) was degassed (3 vacuum/N₂ cycles) and shaken at 100° C. for 3.5h. A second amount of carbamic acid tert-butyl ester (147.6 mg, 1.26 mmol), XantPhos (62.5 mg, 0.110 mmol Pd₂(dba)₃ (49.5 mg, 0.050 mmol) and Cs₂CO₃ (469 mg, 1.44 mmol) was added. After 1h the reaction mixture was allowed to cool to RT and filtered through a pad of Celite, washing with EtOAc. The organic phase was evaporated under reduced pressure and the crude was purified by two sequential flash chromatographies (first chromatography: gradient DCM/Methanol from 100:0 to 96:4; second reversed phase chromatography: gradient water/MeCN with 0.1% of formic acid from 95:5 to 60:40). Evaporation of proper fractions provided the desired compound (210 mg, 0.600 mmol, 67% yield) as a colorless oil.

LC-MS (ESI): m/z (M+1): 348.3 (Method 1)

### Step 6: 4-(5-((tert-butoxycarbonyl)amino)-6-methylpyridin-2-yl)-1-methyl-1H-1,2,3-triazole-5-carboxylic acid (Intermediate M11)

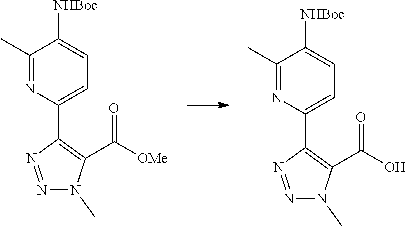 [SMILES: COC(=O)c1c(-c2ccc(NC(=O)OC(C)(C)C)c(C)n2)nnn1C ; Cc1nc(-c2nnn(C)c2C(=O)O)ccc1NC(=O)OC(C)(C)C]

A solution of LiOH (14.48 mg, 0.600 mmol) in water (1 mL) was added to a solution of methyl 4-(5-((tert-butoxycarbonyl)amino)-6-methylpyridin-2-yl)-1-methyl-1H-1,2,3-triazole-5-carboxylate (Intermediate M10, 210.0 mg, 0.600 mmol) in THE (3 mL) and the mixture was shaken at RT overnight. Volatiles were removed under reduced pressure to provide the title compound (288 mg, crude) as a white solid.

LC-MS (ESI): m/z (M+1): 334.2 (Method 1)

### Step 7: (R)-1-(2-chlorophenyl)ethyl (4-(5-((tert-butoxycarbonyl)amino)-6-methylpyridin-2-yl)-1-methyl-1H-1,2,3-triazol-5-yl)carbamate (Intermediate M12)

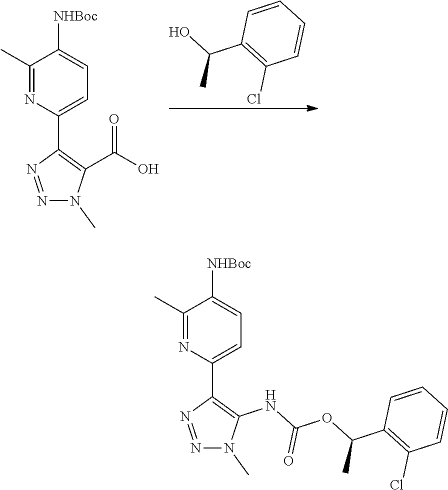

To a mixture of 4-(5-((tert-butoxycarbonyl)amino)-6-methylpyridin-2-yl)-1-methyl-1H-1,2,3-triazole-5-carboxylic acid (Intermediate M11, 205. mg, 0.610 mmol), TEA (0.17 mL, 1.21 mmol) and (R)-1-(2-chlorophenyl)ethan-1-ol (0.1 mL, 0.730 mmol) in Toluene (5 mL), DPPA (0.2 mL, 0.910 mmol) was added and the mixture was stirred at 125° C. for 7 h. The reaction mixture was allowed to cool to RT then partitioned between EtOAc and saturated NaHCO₃ aqueous solution. The organic phase was separated, filtered through a hydrophobic phase separator and concentrated under reduced pressure. The crude was purified by flash chromatography (gradient CyHex/EtOAc from 100:0 to 60:40 as eluent) and proper fractions were evaporated to provide the target compound (177 mg, 0.363 mmol, 60% yield) as an off-white solid.

LC-MS (ESI): m/z (M+1): 487.2 (Method 1)

1H NMR (400 MHz, Chloroform-d) δ 8.23 (d, J=8.63 Hz, 1H), 7.98 (d, J=8.55 Hz, 1H), 7.46 (d, J=7.33 Hz, 1H), 7.38-7.34 (m, 1H), 7.27-7.20 (m, 3H), 6.29 (s, 1H), 6.22 (q, J=6.52 Hz, 1H), 4.06 (s, 3H), 2.54 (s, 3H), 1.62 (d, J=6.53 Hz, 3H), 1.54 (s, 9H).

The Intermediate in the following table was prepared from reagents reported below following similar procedures as for Intermediate M12.

| Intermediate | Structure & Name | Reagents | Analytical data |
|---|---|---|---|
| M13 | 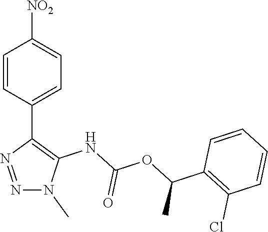 [SMILES: C[C@@H](OC(=O)Nc1c(-c2ccc([N+](=O)[O-])cc2)nnn1C)c1ccccc1Cl] | M8 | LC-MS (ESI): m/z (M + 1): 402.3 (Method 1) 1H NMR (400 MHz, DMSO-d6 ) d ppm 10.00- 10.42 (m, 1 H) 8.17-8.41 (m, 2 H) 7.87-8.09 (m, 2 H) 7.17-7.76 (m, 4 H) 6.02 (d, J =6.60 Hz, 1 H) 3.90 (s, 3 H) 1.34-1.73 (m, 3 H) |
| | (R)-1-(2-chlorophenyl)ethyl | | |
| | (1-methyl-4-(4- | | |
| | nitrophenyl)-1H-1,2,3- | | |
| | triazol-5-yl)carbamate | | |

### Step 8: (R)-1-(2-chlorophenyl)ethyl (4-(5-amino-6-methylpyridin-2-yl)-1-methyl-1H-1,2,3-triazol-5-yl)carbamate (Intermediate M14)

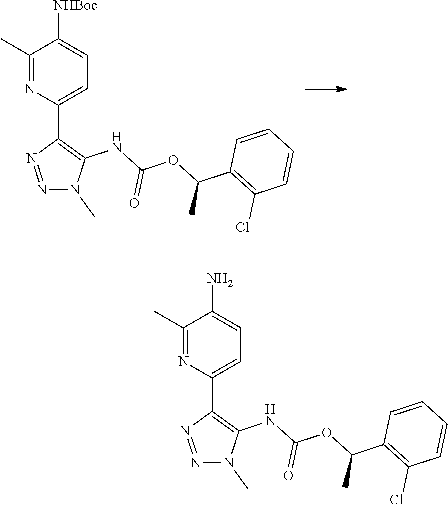 [SMILES: Cc1nc(-c2nnn(C)c2NC(=O)O[C@H](C)c2ccccc2Cl)ccc1NC(=O)OC(C)(C)C ; Cc1nc(-c2nnn(C)c2NC(=O)O[C@H](C)c2ccccc2Cl)ccc1N]

To a solution of (R)-1-(2-chlorophenyl)ethyl (4-(5-((tert-butoxycarbonyl)amino)-6-methylpyridin-2-yl)-1-methyl-1H-1,2,3-triazol-5-yl)carbamate (Intermediate M12, 177.0 mg, 0.360 mmol) in DCM (3 mL), 3M HCl in CPME (1.21 mL, 3.63 mmol) was added and the mixture was stirred at RT overnight. Volatiles were removed under reduced pressure and the residue was partitioned between DCM and saturated NaHCO₃ aqueous solution. The organic phase was separated, filtered through a hydrophobic phase separator and concentrated under reduced pressure to provide the title compound (121 mg, 0.313 mmol, 86% yield) as a yellow oil. The material was used in the next step without further purification.

LC-MS (ESI): m/z (M+1): 387.2 (Method 1)

### Step 9: methyl 3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylate, mix of Trans diastereomers (Intermediate M15)

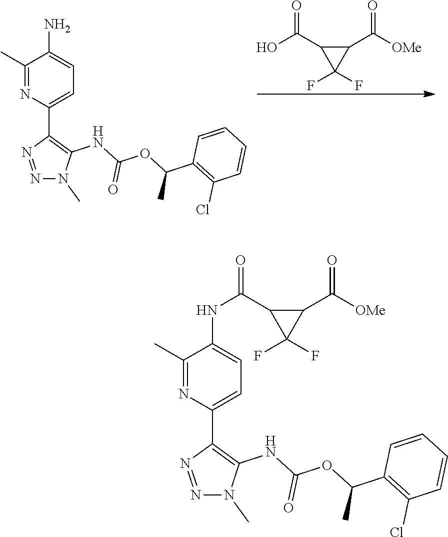 [SMILES: Cc1nc(-c2nnn(C)c2NC(=O)O[C@H](C)c2ccccc2Cl)ccc1N ; COC(=O)C1C(C(=O)O)C1(F)F ; COC(=O)C1C(C(=O)Nc2ccc(-c3nnn(C)c3NC(=O)O[C@H](C)c3ccccc3Cl)nc2C)C1(F)F]

Intermediate M15 (55 mg, 0.100 mmol, 40% yield) was prepared from (R)-1-(2-chlorophenyl)ethyl (4-(5-amino-6-methylpyridin-2-yl)-1-methyl-1H-1,2,3-triazol-5-yl)carbamate (Intermediate M14, 98 mg, 0.250 mmol) and trans-rac 2,2-difluoro-3-(methoxycarbonyl)cyclopropane-1-carboxylic acid (59 mg, 0.330 mmol) using a similar method as for Intermediate A17.

LC-MS (ESI): m/z (M+1): 549.3 (Method 1)

### Step 10: 3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid, mix of Trans diastereomers (Compound 32)

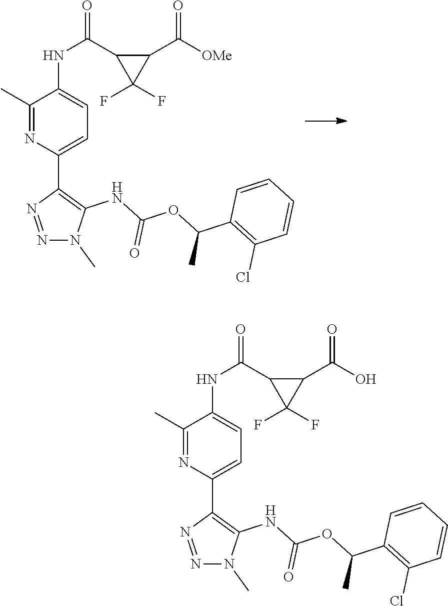 [SMILES: COC(=O)C1C(C(=O)Nc2ccc(-c3nnn(C)c3NC(=O)O[C@H](C)c3ccccc3Cl)nc2C)C1(F)F ; Cc1nc(-c2nnn(C)c2NC(=O)O[C@H](C)c2ccccc2Cl)ccc1NC(=O)C1C(C(=O)O)C1(F)F]

To an ice-cooled solution of methyl 3-((6-(5-((((R)-1-(2-chlorophenyl) ethoxy)carbonyl)amino)-1-methyl-1H-1,2,3-triazol-4-yl)-2-methylpyridin-3-yl)carbamoyl)-2,2-difluorocyclopropane-1-carboxylate (Intermediate M15, 55.0 mg, 0.100 mmol) in THE (1 mL) and Water (0.700 mL), LiOH (4.8 mg, 0.200 mmol) was added and the mixture was stirred at RT for 2h. HCl 1N was added until pH<4. Volatiles were removed at reduced pressure to provide the crude as a mixture of Cis—and Trans diastereomers that was submitted to semipreparative HPLC.

Conditions: Column: CSH C18 (2.1×50 mm, 1.7 μm). Conditions: [A1: Waters+0.1% HCOOH]; [B1: MeCN+0.1% HCOOH]. Gradient: from 3% B1 to 99.9% B1 in 1.5 min (flow: 1.00 mL/min).Detection: UV/Vis detection range 210 nm to 350 nm

### Peak 2: Compound 32

3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid, mix of trans diastereomers

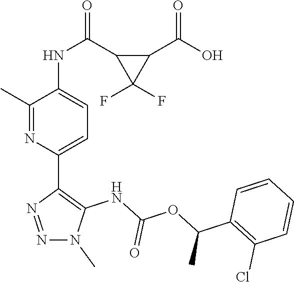 [SMILES: Cc1nc(-c2nnn(C)c2NC(=O)O[C@H](C)c2ccccc2Cl)ccc1NC(=O)C1C(C(=O)O)C1(F)F]

LC-MS (ESI): m/z (M+1): 535.2 (Method 1)

1H NMR (400 MHz, DMSO-d6) δ 12.91 (s, 1H), 9.71 (s, 1H), 7.90 (dd, J=8.42, 1.52 Hz, 1H), 7.78 (d, J=8.34 Hz, 1H), 7.39 (m, J=43.92 Hz, 4H), 6.01 (s, 1H), 3.89 (s, 3H), 3.22 (t, J=12.36 Hz, 1H), 3.08 (t, J=12.38 Hz, 1H), 2.37 (s, 3H), 1.52 (s, 3H).

Compound 32 was submitted to chiral preparative HPLC.

Conditions: Column Whelk 01 (R,R) (25×3.0 cm), 10 Mobile phase n-Hexane/(Ethanol +0.1% formic acid) 55/45% v/v Flow rate (ml/min) 40 ml/min DAD detection 220 nm Loop 1500 μL

### Peak 1: Compound 33

Single Diastereomer 1 of Trans-3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid

LC-MS (ESI): m/z (M+1): 535.2 (Method 1)

1H NMR (400 MHz, DMSO-d6) δ 10.08 (s, 1H), 9.73 (s, 1H), 7.95 (d, J=8.39 Hz, 1H), 7.79 (d, J=8.36 Hz, 1H), 7.38 (d, J=43.97 Hz, 4H), 6.01 (s, 1H), 3.89 (s, 3H), 3.50 (dd, J=14.01, 7.89 Hz, 1H), 3.01 (dd, J=14.61, 7.65 Hz, 1H), 2.38 (s, 3H), 1.44 (d, J=64.20 Hz, 3H).

The corresponding sodium salt of Compound 33 was obtained using a similar method as for Compound 8.

LC-MS (ESI): m/z (M+1-Na+): 535.2 (Method 1)

1H NMR (500 MHz, DMSO-d6) δ ppm 9.92 (br s, 1H), 9.67 (br s, 1H), 7.91 (br d, J=8.2 Hz, 1H), 7.76 (d, J=8.4 Hz, 1H), 7.04-7.70 (m, 4H), 6.01 (br s, 1H), 3.87 (br s, 3H), 3.17 (br dd, J=12.8, 7.9 Hz, 1H), 2.58 (br dd, J=18.4, 7.7 Hz, 1H), 2.36 (s, 3H), 1.54 (br s, 3H)

### Peak 2: Compound 34

Single Diastereomer 2 of Trans-3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid

LC-MS (ESI): m/z (M+1): 535.2 (Method 1)

1H NMR (400 MHz, DMSO-d6) δ 10.08 (s, 1H), 9.73 (s, 1H), 7.95 (d, J=8.39 Hz, 1H), 7.79 (d, J=8.36 Hz, 1H), 7.38 (d, J=43.97 Hz, 4H), 6.01 (s, 1H), 3.89 (s, 3H), 3.50 (dd, J=14.01, 7.89 Hz, 1H), 3.01 (dd, J=14.61, 7.65 Hz, 1H), 2.38 (s, 3H), 1.44 (d, J=64.20 Hz, 3H).

The corresponding sodium salt of Compound 34 was obtained using a similar method as for Compound 8.

LC-MS (ESI): m/z (M+1-Na+): 535.2 (Method 1)

1H NMR (500 MHz, DMSO-d6) δ ppm 9.90 (br s, 1H), 9.71 (br s, 1H), 7.85 (br s, 1H), 7.73 (br d, J=8.1 Hz, 1H), 7.03-7.65 (m, 4H), 5.98 (br s, 1H), 3.82 (br s, 3H), 3.16 (br dd, J=12.9, 7.8 Hz, 1H), 2.58 (br dd, J=18.3, 7.7 Hz, 1H), 2.36 (s, 3H), 1.48 (br s, 3H)

### Example 12

### Process synthesis of (1S,3S)-3-((4-(5-((((R)-1-(2-chlorophenyl)ethoxy)carbonyl)amino)-1-methyl-1H-1,2,3-triazol-4-yl)phenyl)carbamoyl)-2,2-difluorocyclopropane-1-carboxylic acid (Compound 35)

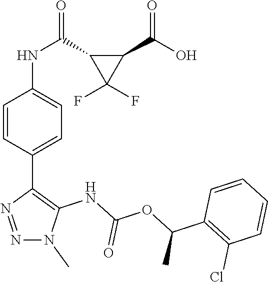 [SMILES: C[C@@H](OC(=O)Nc1c(-c2ccc(NC(=O)[C@@H]3[C@@H](C(=O)O)C3(F)F)cc2)nnn1C)c1ccccc1Cl]

### Step 1: (R)-1-(2-chlorophenyl)ethyl (4-(4-aminophenyl)-1-methyl-1H-1,2,3-triazol-5-yl)carbamate (Intermediate M16)

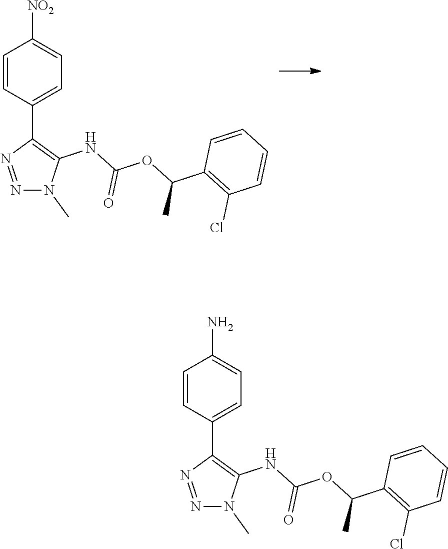 [SMILES: C[C@@H](OC(=O)Nc1c(-c2ccc([N+](=O)[O-])cc2)nnn1C)c1ccccc1Cl ; C[C@@H](OC(=O)Nc1c(-c2ccc(N)cc2)nnn1C)c1ccccc1Cl]

To an ice-cooled solution of (R)-1-(2-chlorophenyl)ethyl (1-methyl-4-(4-nitrophenyl)-1H-1,2,3-triazol-5-yl)carbamate, Intermediate M13, 458.0 mg, 1.14 mmol) in MeOH (10 mL), 37% w/w HCl (1.57 mL, 15.96 mmol) was added followed by the addition of SnCl₂*2H₂O (1029 mg, 4.56 mmol). The mixture was allowed to reach RT and stirred for 7 h. The mixture was cooled with an ice bath, diluted with water and quenched with 10% NaOH to pH-8. The mixture was extracted with EtOAc (2 times) then collected organics were dried over Na₂SO₄, filtered and concentrated at reduced pressure to provide the crude product (411 mg, 1.105 mmol, 97% yield) as a yellow solid, that was used without purification

LC-MS (ESI): m/z (M+1): 372.2 (Method 1)

### Step 2: methyl (1S,3S)-3-((4-(5-((((R)-1-(2-chlorophenyl)ethoxy)carbonyl)amino)-1-methyl-1H-1,2,3-triazol-4-yl)phenyl)carbamoyl)-2,2-difluorocyclopropane-1-carboxylate (Intermediate M17)

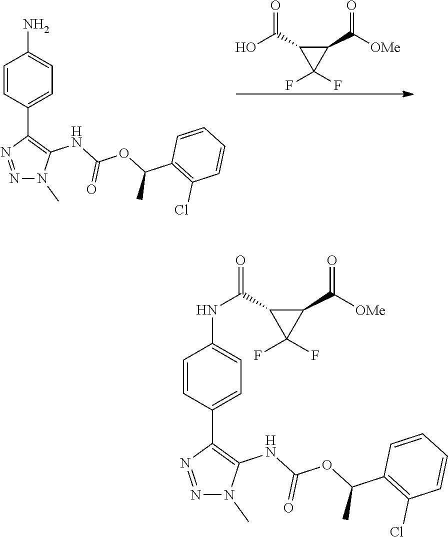 [SMILES: C[C@@H](OC(=O)Nc1c(-c2ccc(N)cc2)nnn1C)c1ccccc1Cl ; COC(=O)[C@@H]1[C@@H](C(=O)O)C1(F)F ; COC(=O)[C@@H]1[C@@H](C(=O)Nc2ccc(-c3nnn(C)c3NC(=O)O[C@H](C)c3ccccc3Cl)cc2)C1(F)F]

Intermediate M17 (49.5 mg, 0.093 mmol, 43.1% yield) was prepare rom (R)-1-(2-chlorophenyl)ethyl (4-(4-aminophenyl)-1-methyl-1H-1,2,3-triazol-5-yl)carbamate (Intermediate M16, 80.0 mg, 0.220 mmol) and (1S,3S)-2,2-difluoro-3-methoxycarbonyl-cyclopropanecarboxylic acid (Intermediate 2, 50.4 mg, 0.280 mmol) using a similar method as for Intermediate A17.

LC-MS (ESI): m/z (M+1): 534.3 (Method 1)

### Step 3: (1S,3S)-3-((4-(5-((((R)-1-(2-chlorophenyl)ethoxy)carbonyl)amino)-1-methyl-1H-1,2,3-triazol-4-yl)phenyl)carbamoyl)-2,2-difluorocyclopropane-1-carboxylic acid (Compound

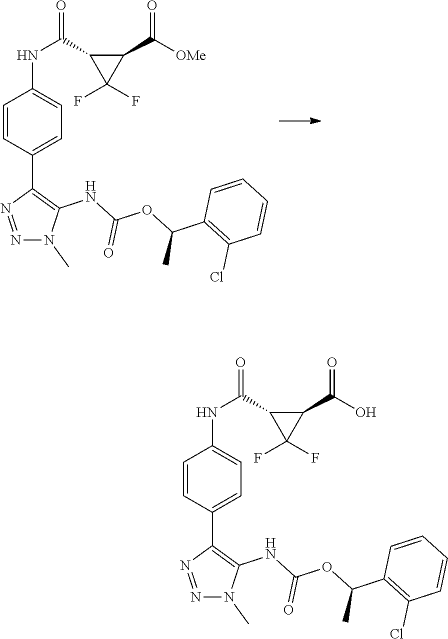 [SMILES: COC(=O)[C@@H]1[C@@H](C(=O)Nc2ccc(-c3nnn(C)c3NC(=O)O[C@H](C)c3ccccc3Cl)cc2)C1(F)F ; C[C@@H](OC(=O)Nc1c(-c2ccc(NC(=O)[C@@H]3[C@@H](C(=O)O)C3(F)F)cc2)nnn1C)c1ccccc1Cl]

Compound 35 (41 mg, 0.079 mmol, 85% yield) was obtained as a white solid (1S,3S)-3-((4-(5-((((R)-1-(2-chlorophenyl)ethoxy)carbonyl)amino)-1-methyl-1H-1,2,3-triazol-4-yl)phenyl)carbamoyl)-2,2-difluorocyclopropane-1-carboxylate (Intermediate M17, 49.5 mg, 0.090 mmol) and SnMe₃OH (50.6 mg, 0.280 mmol) using a similar method as for Compound 1, step 9.

LC-MS (ESI): m/z (M+1): 520.2 (Method 1)

The corresponding sodium salt of Compound 35 was obtained using a similar method as for Compound 8. Compound 35 was characterized as sodium salt.

LC-MS (ESI): m/z (M+1): 520.2 (Method 1)

1H NMR (400 MHz, DMSO-d6) δ ppm 10.52 (s, 1H), 8.85-10.22 (m, 1H), 6.84-7.91 (m, 8H), 5.87-6.16 (m, 1H), 3.82 (s, 3H), 3.06 (dd, J=13.2, 7.7 Hz, 1H), 2.55-2.66 (m, 1H), 1.25-1.79 (m, 3H)

### Example 13

### Process synthesis of 3-((4-(4-(((cyclopentyl(methyl)carbamoyl)oxy)methyl)-3-methylisoxazol-5-yl)phenyl)carbamoyl)-2,2-difluorocyclopropane-1-carboxylic acid, mix of Trans diastereomers (Compound 36)

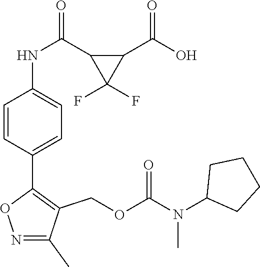 [SMILES: Cc1noc(-c2ccc(NC(=O)C3C(C(=O)O)C3(F)F)cc2)c1COC(=O)N(C)C1CCCC1]

### Step 1: (5-(4-bromophenyl)-3-methylisoxazol-4-yl)methanol (Intermediate N1)

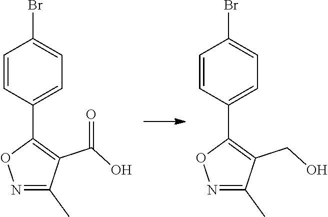 [SMILES: Cc1noc(-c2ccc(Br)cc2)c1C(=O)O ; Cc1noc(-c2ccc(Br)cc2)c1CO]

To a solution of 5-(4-bromophenyl)-3-methylisoxazole-4-carboxylic acid (Intermediate A10, 0.8 g, 2.84 mmol) in THE (20 mL) cooled to 0° C., borane tetrahydrofuran complex 1M in THE (11.3 mL, 11.30 mmol) was added dropwise. The reaction was allowed to reach RT and stirred overnight. Additional borane tetrahydrofuran complex 1M in THE (3 mL, 3.00 mmol) was added at RT and the reaction was heated at 50° C. for 10 h, then stirred at RT overnight. Additional borane tetrahydrofuran complex 1M in THE (3 mL, 3.00 mmol) was added and the reaction was heated at 50° C. for further 4 h, then cooled to r.t. 1.2 M HCl (30 mL) was added and the mixture was stirred at RT for 30 min and extracted with EtOAc (2 times). The combined organic layers were washed with sat. NaHCO₃ (2 times) and brine, dried over Na₂SO₄, filtered and concentrated. The crude was purified by flash chromatography (hexane:EtOAc from 85:15 to 60:40) to afford the title compound (0.390 g, 1.455 mmol, 51% yield) as a light yellow solid.

LC-MS (ESI): m/z (M+1): 270.0 (Method 2)

### Step 2: (5-(4-bromophenyl)-3-methylisoxazol-4-yl)methyl (4-nitrophenyl) carbonate (Intermediate N2)

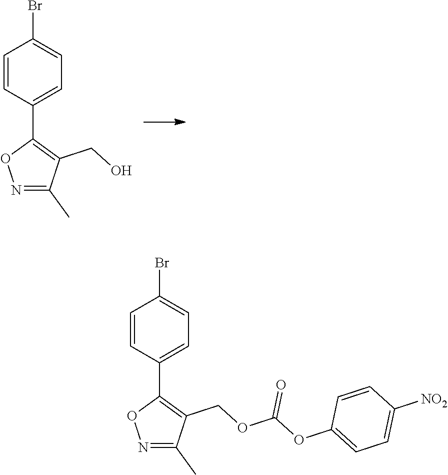 [SMILES: Cc1noc(-c2ccc(Br)cc2)c1CO ; Cc1noc(-c2ccc(Br)cc2)c1COC(=O)Oc1ccc([N+](=O)[O-])cc1]

To a solution of (5-(4-bromophenyl)-3-methylisoxazol-4-yl)methanol (Intermediate N1, 0.250 g, 0.932 mmol) in DCM (8 mL), pyridine (0.377 mL, 4.66 mmol) was added followed by portion-wise addition of 4-nitrophenyl chloroformate (0.301 g, 1.492 mmol) and the resulting mixture was stirred at RT for 3 h. The solvent was removed under reduced pressure and the crude was purified by flash chromatography (hexane:EtOAc from 90:10 to 75:25) to afford the title compound (0.310 g, 0.716 mmol, 77% yield) as a pale yellow solid.

LC-MS (ESI): m z (M+1): 435.1 (Method 2)

### Step 3: (5-(4-bromophenyl)-3-methylisoxazol-4-yl)methyl cyclopentyl(methyl)carbamate (Intermediate N3)

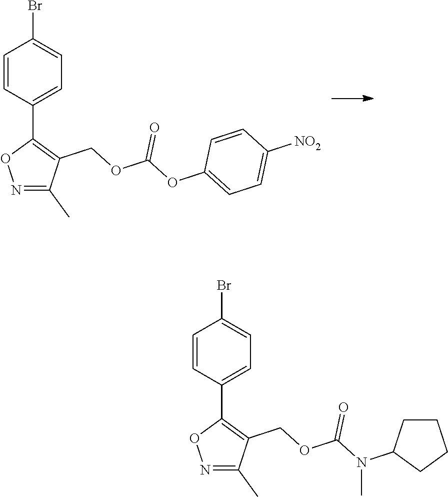 [SMILES: Cc1noc(-c2ccc(Br)cc2)c1COC(=O)Oc1ccc([N+](=O)[O-])cc1 ; Cc1noc(-c2ccc(Br)cc2)c1COC(=O)N(C)C1CCCC1]

To a solution of (5-(4-bromophenyl)-3-methylisoxazol-4-yl)methyl (4-nitrophenyl) carbonate (Intermediate N2, 0.310 g, 0.716 mmol) in THE (6 mL), DIPEA (0.375 mL, 2.147 mmol) was added followed by N-methylcyclopentanamine (0.142 g, 1.431 mmol) and the mixture was stirred at RT overnight. The mixture was diluted with EtOAc and washed with 1M HCl, sat. NaHCO₃ (2 times) and brine. The organic phase was dried over Na₂SO₄, filtered and concentrated. The crude was purified by flash chromatography (hexane:EtOAc from 95:5 to 83:17) to afford the title compound (0.169 g, 0.430 mmol, 60% yield) as a whitish solid.

LC-MS (ESI): m z (M+1): 395.2 (Method 2)

The Intermediate in the following table was prepared from reagents reported below by using methods analogous to Intermediate N3.

| Intermediate | Structure & Name | Reagents | Analytical data |
|---|---|---|---|
| N4 | 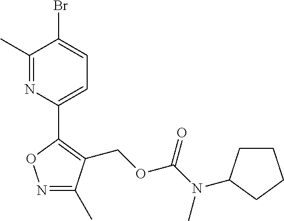 [SMILES: Cc1nc(-c2onc(C)c2COC(=O)N(C)C2CCCC2)ccc1Br] | H1 | LC-MS (ESI): m/z (M + 1): 410.2 (Method 1) |
| | (5-(5-bromo-6-methylpyridin-2- | | |
| | yl)-3-methylisoxazol-4- | | |
| | yl)methyl cyclopentyl(methyl) | | |
| | carbamate | | |

### Step 4: (5-(4-((diphenylmethylene)amino)phenyl)-3-methylisoxazol-4-yl)methyl cyclopentyl(methyl)carbamate (Intermediate N5)

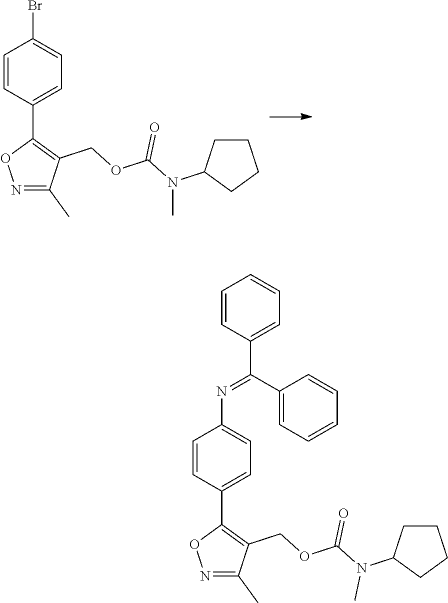 [SMILES: Cc1noc(-c2ccc(Br)cc2)c1COC(=O)N(C)C1CCCC1 ; Cc1noc(-c2ccc(N=C(c3ccccc3)c3ccccc3)cc2)c1COC(=O)N(C)C1CCCC1]

A mixture of (5-(4-bromophenyl)-3-methylisoxazol-4-yl)methyl cyclopentyl (methyl) carbamate (Intermediate N3, 0.211 g, 0.537 mmol), benzophenone imine (0.135 mL, 0.805 mmol) and Cs₂CO₃ (0.524 g, 1.610 mmol) in 1,4-dioxane (4 mL) was degassed applying alternatively N₂ and vacuum. Xantphos (0.047 g, 0.080 mmol) was added followed by Pd₂(dba)₃ (0.025 g, 0.027 mmol) and the resulting mixture was heated at 90° C. for 5 h. The mixture was cooled to RT and partitioned between EtOAc and water. The organic phase was washed with brine, dried over Na₂SO₄, filtered and concentrated to dryness. The crude was purified by flash chromatography (hexane:EtOAc from 90:10 to 75:25) to obtain the title compound (0.220 g, 0.446 mmol, 83% yield) as an orange amorphous.

LC-MS (ESI): m z (M+1): 494.5 (Method 2)

### Step 5: (5-(4-aminophenyl)-3-methylisoxazol-4-yl)methyl cyclopentyl(methyl)carbamate (Intermediate N6)

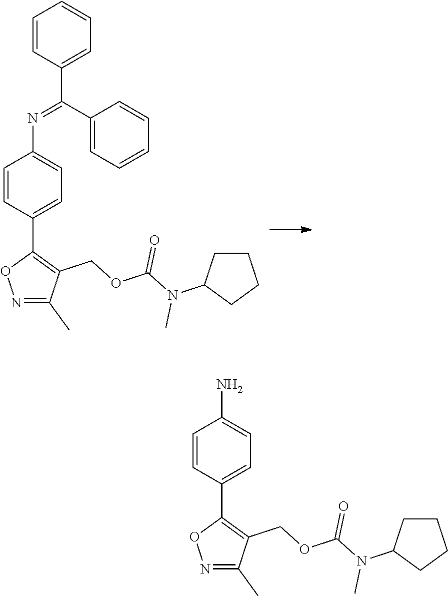 [SMILES: Cc1noc(-c2ccc(N=C(c3ccccc3)c3ccccc3)cc2)c1COC(=O)N(C)C1CCCC1 ; Cc1noc(-c2ccc(N)cc2)c1COC(=O)N(C)C1CCCC1]

To a solution of (5-(4-((diphenylmethylene)amino)phenyl)-3-methylisoxazol-4-yl)methyl cyclopentyl(methyl)carbamate (Intermediate N5, 0.220 g, 0.446 mmol) in THE (2.5 mL) cooled to 0° C., 2M HCl (0.669 mL, 1.337 mmol) was added and the mixture was allowed to reach RT and stirred for 1.5 h. The mixture was partitioned between EtOAc and sat. NaHCO₃. The organic phase was washed with brine, dried over Na₂SO₄, filtered and concentrated to dryness. The crude was purified by flash chromatography (hexane:EtOAc from 80:20 to 60:40) to afford the title compound (0.141 g, 0.428 mmol, 96% yield) as a colorless solid.

LC-MS (ESI): m z (M+1): 330.3 (Method 2)

The Intermediate in the following table was prepared from reagents reported below by using methods analogous to Intermediate N6.

| Intermediate | Structure & Name | Reagents | Analytical data |
|---|---|---|---|
| N7 | 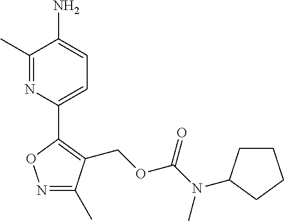 [SMILES: Cc1nc(-c2onc(C)c2COC(=O)N(C)C2CCCC2)ccc1N] | N4 | LC-MS (ESI): m/z (M + 1): 345.3 (Method 2) |
| | (5-(5-amino-6-methylpyridin- | | |
| | 2-yl)-3-methylisoxazol-4- | | |
| | yl)methyl | | |
| | cyclopentyl(methyl)carbamate | | |

### Step 3: methyl 3-((4-(4-(((cyclopentyl(methyl)carbamoyl)oxy)methyl)-3-methylisoxazol-5-yl)phenyl)carbamoyl)-2,2-difluorocyclopropane-1-carboxylate, mix of Trans diastereomers (Intermediate N8)

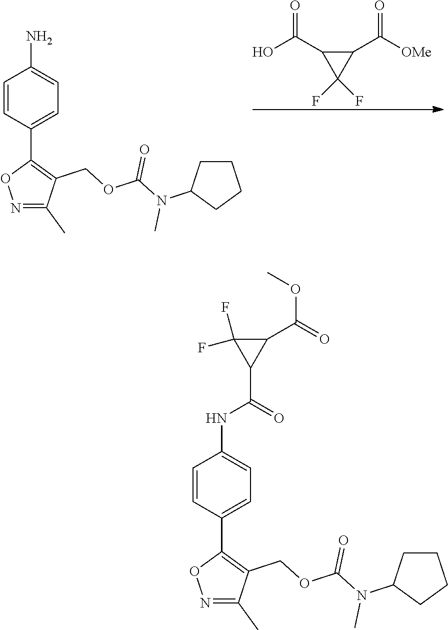 [SMILES: Cc1noc(-c2ccc(N)cc2)c1COC(=O)N(C)C1CCCC1 ; COC(=O)C1C(C(=O)O)C1(F)F ; COC(=O)C1C(C(=O)Nc2ccc(-c3onc(C)c3COC(=O)N(C)C3CCCC3)cc2)C1(F)F]

Intermediate N8 (0.152 g, 0.309 mmol, 72% yield) was obtained from (5-(4-aminophenyl)-3-methylisoxazol-4-yl)methyl cyclopentyl(methyl)carbamate N₆ (0.141 g, 0.428 mmol) and Trans-rac-2,2-difluoro-3-(methoxycarbonyl)cyclopropane-1-carboxylic acid (0.085 g, 0.471 mmol) using a similar method as for Intermediate A17.

LC-MS (ESI): m z (M+1): 492.4 (Method 2)

The Intermediate in the following table was prepared from reagents reported below by using methods analogous to Intermediate A17.

| Intermediate | Structure & Name | Reagents | Analytical data |
|---|---|---|---|
| N9 | 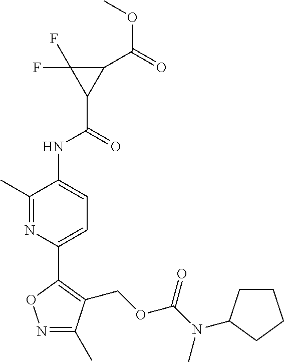 [SMILES: COC(=O)C1C(C(=O)Nc2ccc(-c3onc(C)c3COC(=O)N(C)C3CCCC3)nc2C)C1(F)F] | N7 | LC-MS (ESI): m/z (M + 1): 507.4 (Method 2) |
| | methyl 3-((6-(4- | | |
| | (((cyclopentyl(methyl)carbamoyl) | | |
| | oxy)methyl)-3-methylisoxazol-5-yl)- | | |
| | 2-methylpyridin-3-yl)carbamoyl)-2,2- | | |
| | difluorocyclopropane-1-carboxylate, | | |
| | mix of Trans diastereomers | | |

### Step 4: 3-((4-(4-(((cyclopentyl(methyl)carbamoyl)oxy)methyl)-3-methylisoxazol-5-yl)phenyl)carbamoyl)-2,2-difluorocyclopropane-1-carboxylic acid, mix of Trans diastereomers (Compound 36)

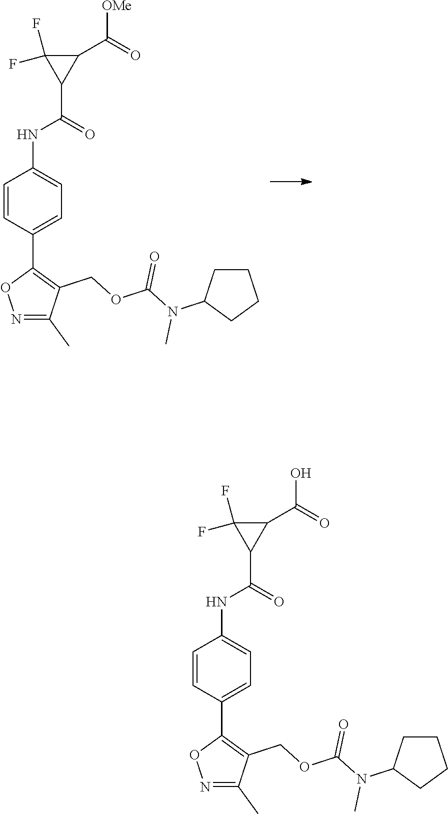 [SMILES: COC(=O)C1C(C(=O)Nc2ccc(-c3onc(C)c3COC(=O)N(C)C3CCCC3)cc2)C1(F)F ; Cc1noc(-c2ccc(NC(=O)C3C(C(=O)O)C3(F)F)cc2)c1COC(=O)N(C)C1CCCC1]

Compound 36 (0.133 g, 0.279 mmol, 90% yield) was obtained from methyl 3-((4-(4-(((cyclopentyl(methyl)carbamoyl)oxy)methyl)-3-methylisoxazol-5-yl)phenyl)carbamoyl)-2,2-difluorocyclopropane-1-carboxylate, mix of Trans diastereomers (Intermediate N8, 0.152 g, 0.309 mmol), using a similar method as for Compound 3, step 3.

LC-MS (ESI): m/z (M+1): 478.4 (Method 2)

1H NMR (300 MHz, DMSO-d6) δ ppm 13.33 (br s, 1H), 10.86 (s, 1H), 7.70-7.86 (m, 4H), 5.08 (s, 2H), 4.10-4.51 (m, 1H), 3.30-3.44 (m, 1H), 3.02-3.14 (m, 1H), 2.67 (s, 3H), 2.32 (s, 3H), 1.53-1.77 (m, 4H), 1.33-1.52 (m, 4H).

The Compound in the following table was prepared from reagents reported below following similar procedures as for Compound 3, step 3.

| Compound | Structure & Name | Reagents | Analytical data |
|---|---|---|---|
| 37 | 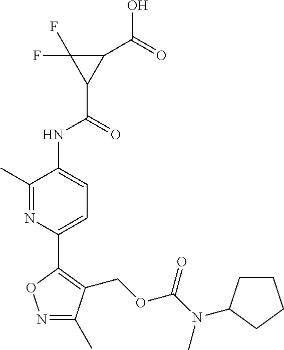 [SMILES: Cc1nc(-c2onc(C)c2COC(=O)N(C)C2CCCC2)ccc1NC(=O)C1C(C(=O)O)C1(F)F] | N9 | LC-MS (ESI): m/z (M + 1): 493.3 (Method 2) 1H NMR (300 MHz, DMSO-d₆) δ ppm 13.47 (br s, 1H), 10.23 (s, 1H), 8.12-8.23 (m, 1H), 7.78 (d, J =8.5 Hz, 1H), 5.39 (s, 2H), 4.12-4.53 (m, 1H), 3.56-3.72 (m, 1H), 3.02-3.16 (m, 1H), 2.66 (s, 3H), 2.53 (s, 3H), 2.33 (s, 3H), 1.53-1.78 (m, 4H), 1.32-1.53 (m, 4H). |
| | 3-((6-(4- | | |
| | (((cyclopentyl(methyl) | | |
| | carbamoyl)oxy)methyl)-3- | | |
| | methylisoxazol-5-yl)-2- | | |
| | methylpyridin-3- | | |
| | yl)carbamoyl)-2,2- | | |
| | difluorocyclopropane-1- | | |
| | carboxylic acid, mix of Trans | | |
| | diastereomers | | |

### Example 14

### Process Synthesis of (1S,3S)-3-((4-(4-((((R)-1-(2-chlorophenyl)ethoxy)carbonyl)amino)-1-methyl-1H-pyrazol-3-yl)phenyl)carbamoyl)-2,2-difluorocyclopropane-1-carboxylic acid (Compound 38)

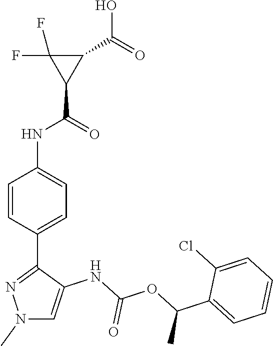 [SMILES: C[C@@H](OC(=O)Nc1cn(C)nc1-c1ccc(NC(=O)[C@@H]2[C@@H](C(=O)O)C2(F)F)cc1)c1ccccc1Cl]

### Step 1: 3-(4-((tert-butoxycarbonyl)amino)phenyl)-1-methyl-1H-pyrazole-4-carboxylic acid (Intermediate O1)

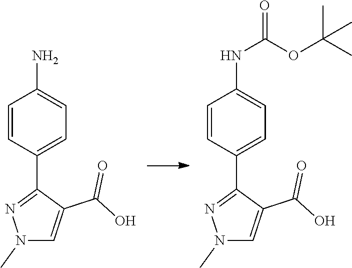 [SMILES: Cn1cc(C(=O)O)c(-c2ccc(N)cc2)n1 ; Cn1cc(C(=O)O)c(-c2ccc(NC(=O)OC(C)(C)C)cc2)n1]

3-(4-Aminophenyl)-1-methyl-1H-pyrazole-4-carboxylic acid (0.875 g, 4.023 mmol) was dissolved in DCM (20.00 ml). A solution of NaHCO₃ (1.02 g, 12.04 mmol) in water (20.00 mL) was added dropwise at RT and the reaction was heated to 50° C. Boc₂O (13.2 g, 6.04 mmol) was added and the mixture was stirred overnight at RT. The layers were separated and water phase was washed with DCM (2×30 mL). Combined organic layers were washed with brine (30 mL) and dried over Na₂SO₄. The solvent was evaporated to dryness and the residue was purified by flash chromatography (EtOAc in hexane from 0% to 50%) to give the title compound (0.34 g, 1.0 mmol, 27% yield) as white solid

LC-MS (ESI): m/z (M+1): 318.7 (Method 3)

### Step 2: tert-butyl (R)-(4-(4-(((1-(2-chlorophenyl)ethoxy)carbonyl)amino)-1-methyl-1H-pyrazol-3-yl)phenyl)carbamate (Intermediate O2)

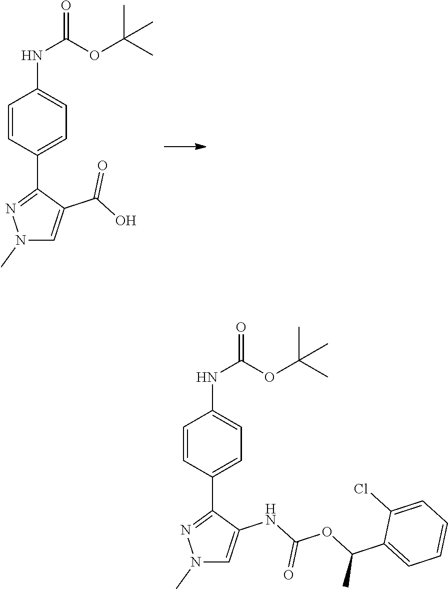 [SMILES: Cn1cc(C(=O)O)c(-c2ccc(NC(=O)OC(C)(C)C)cc2)n1 ; C[C@@H](OC(=O)Nc1cn(C)nc1-c1ccc(NC(=O)OC(C)(C)C)cc1)c1ccccc1Cl]

3-(4-((tert-butoxycarbonyl)amino)phenyl)-1-methyl-1H-pyrazole-4-carboxylic acid (Intermediate O1, 0.34 g, 1.071 mmol) and (R)-1-(3-chlorophenyl)ethanol (0.201 g, 1.286 mmol) were dissolved in anhydrous toluene (21.43 ml, L) in a sealed tube under argon atmosphere followed by addition of TEA (0.298 ml, 2.143 mmol) and DPPA (0.442 g, 1.607 mmol). The tube was transferred to a pre-heated bath at 95° C. and the reaction was stirred overnight. The solvent was evaporated and the crude product was purified by flash chromatography (0-40% EtOAc in hexane) to give the target compound (0.24 g, 0.51 mmol 48% yield).

LC-MS (ESI): m/z (M+1): 472.0 (Method 3)

### Step 3: (R)-1-(2-chlorophenyl)ethyl (3-(4-aminophenyl)-1-methyl-1H-pyrazol-4-yl)carbamate (Intermediate O3)

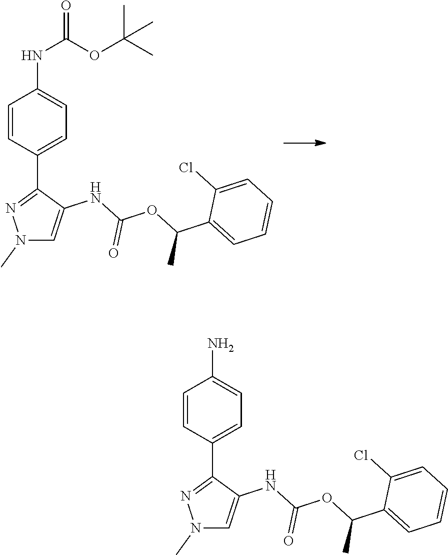 [SMILES: C[C@@H](OC(=O)Nc1cn(C)nc1-c1ccc(NC(=O)OC(C)(C)C)cc1)c1ccccc1Cl ; C[C@@H](OC(=O)Nc1cn(C)nc1-c1ccc(N)cc1)c1ccccc1Cl]

(tert-butyl (R)-(4-(4-(((1-(2-chlorophenyl)ethoxy)carbonyl)amino)-1-methyl-1H-pyrazol-3-yl)phenyl)carbamate (Intermediate O2, 0.24 g, 0.51 mmol, 1.0 eq) was dissolved in 1,4-dioxane (2.55 ml, 0.2 M), then 4M HCl in 1,4-dioxane (0.63 ml, 2.54 mmol) was added and the mixture was stirred at RT overnight. A further amount of 4M HCl (1.27 ml, 5.10 mmol) in 1,4-dioxane was added. After additional 24h the solvent was evaporated, the residue was diluted with DCM/water (15/15 ml) and basified to pH-10 with 25% NH₄₀H. The layers were separated and water phase was washed with DCM (2×15 ml). Combined organic layers were washed with brine and dried over Na₂SO₄ and concentrated to dryness. The crude product was purified by flash chromatography (0-10% MeOH in DCM) to give the title compound (85 mg, 0.23 mmol, 45% yield) as a white solid.

LC-MS (ESI): m/z (M+1): 371.1 (Method 4)

### Step 4: methyl (1S,3S)-3-((4-(4-((((R)-1-(2-chlorophenyl)ethoxy)carbonyl)amino)-1-methyl-1H-pyrazol-3-yl)phenyl)carbamoyl)-2,2-difluorocyclopropane-1-carboxylate (Intermediate O4)

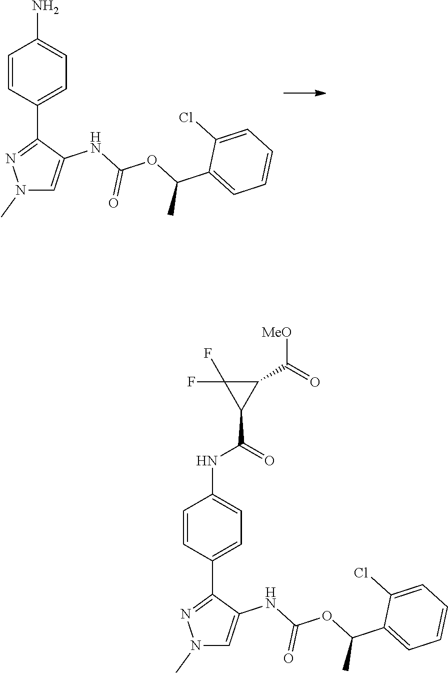 [SMILES: C[C@@H](OC(=O)Nc1cn(C)nc1-c1ccc(N)cc1)c1ccccc1Cl ; COC(=O)[C@@H]1[C@@H](C(=O)Nc2ccc(-c3nn(C)cc3NC(=O)O[C@H](C)c3ccccc3Cl)cc2)C1(F)F]

Intermediate O4 (108 mg, 0.203 mmol, 93% yield) was obtained from (R)-1-(2-chlorophenyl)ethyl (3-(4-aminophenyl)-1-methyl-1H-pyrazol-4-yl)carbamate (Intermediate O3, 81.0 mg, 0.220 mmol) and (1S,3S)-2,2-difluoro-3-methoxycarbonyl-cyclopropanecarboxylic acid (Intermediate 2, 51.14 mg, 0.280 mmol) using a similar method as for Intermediate A17.

LC-MS (ESI): m/z (M+1): 533.4 (Method 1)

### Step 5: methyl (1S,3S)-3-((4-(4-((((R)-1-(2-chlorophenyl)ethoxy)carbonyl)amino)-1-methyl-1H-pyrazol-3-yl)phenyl)carbamoyl)-2,2-difluorocyclopropane-1-carboxylic acid (Compound 38)

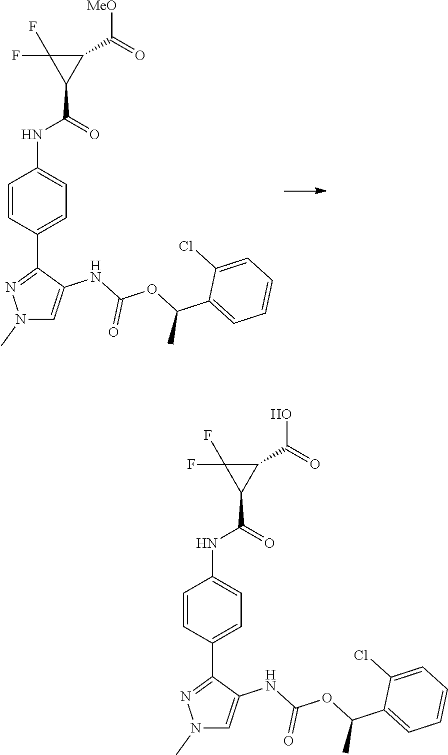 [SMILES: COC(=O)[C@@H]1[C@@H](C(=O)Nc2ccc(-c3nn(C)cc3NC(=O)O[C@H](C)c3ccccc3Cl)cc2)C1(F)F ; C[C@@H](OC(=O)Nc1cn(C)nc1-c1ccc(NC(=O)[C@@H]2[C@@H](C(=O)O)C2(F)F)cc1)c1ccccc1Cl]

Compound 38 (108 mg, 0.203 mmol, 98% yield) was obtained from methyl (1S,3S)-3-((4-(4-((((R)-1-(2-chlorophenyl)ethoxy)carbonyl)amino)-1-methyl-1H-pyrazol-3-yl)phenyl)carbamoyl)-2,2-difluorocyclopropane-1-carboxylate (Intermediate O4, 108.0 mg, 0.200 mmol) using a similar method as for Compound 1.

LC-MS (ESI): m/z (M+1): 519.4 (Method 1)

The corresponding sodium salt of Compound 38 was obtained using a similar method as for Compound 8. The Compound 38 was characterized as sodium salt.

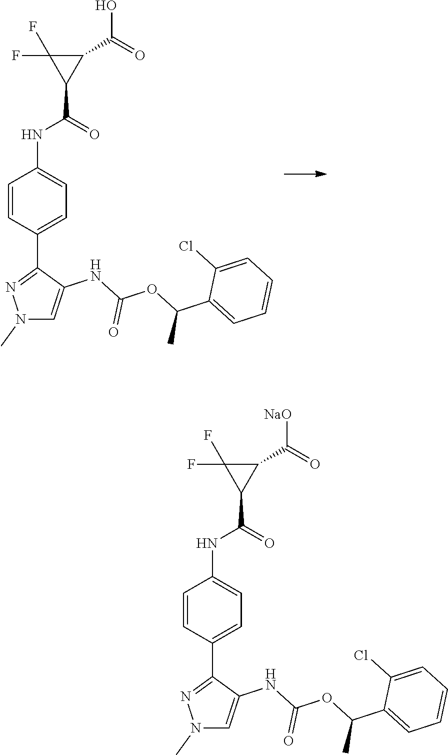

LC-MS (ESI): m/z (M+1-Na+): 519.4 (Method 1)

1H NMR (400 MHz, DMSO-d6) δ ppm 10.39 (br s, 1H), 8.89 (br s, 1H), 7.73 (s, 1H), 6.72-7.69 (m, 8H), 5.69-6.20 (m, 1H), 3.78 (br s, 3H), 3.04 (dd, J=13.2, 7.9 Hz, 1H), 2.55-2.65 (m, 1H), 1.14-1.56 (m, 3H)

### Example 15

### Process Synthesis of (1S,3S)-3-((4-(2-((((R)-1-(2-chlorophenyl)ethoxy)carbonyl)amino)-1H-pyrrol-1-yl)phenyl)carbamoyl)-2,2-difluorocyclopropane-1-carboxylic acid (Compound 39)

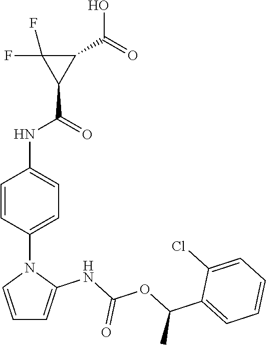 [SMILES: C[C@@H](OC(=O)Nc1cccn1-c1ccc(NC(=O)[C@@H]2[C@@H](C(=O)O)C2(F)F)cc1)c1ccccc1Cl]

### Step 1: (R)-1-(2-chlorophenyl)ethyl (1-(4-nitrophenyl)-1H-pyrrol-2-yl)carbamate (intermediate P1)

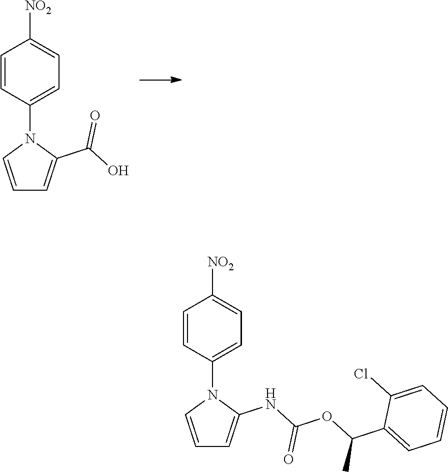 [SMILES: O=C(O)c1cccn1-c1ccc([N+](=O)[O-])cc1 ; C[C@@H](OC(=O)Nc1cccn1-c1ccc([N+](=O)[O-])cc1)c1ccccc1Cl]

1-(4-Nitrophenyl)-1H-pyrrole-2-carboxylic acid (0.9 g, 3.876 mmol) and (R)-1-(2-chlorophenyl)ethanol (0.728 g, 4.651 mmol) were dissolved in anhydrous toluene (8.61 ml) in a sealed tube under argon atmosphere followed by addition of TEA (1.079 ml, 7.752 mmol) and DPPA (1.6 g, 5.814 mmol). The tube was transferred to a pre-heated bath at 95° C. and the reaction mixture was stirred overnight. The solvent was evaporated and the crude material was purified by reversed phase flash chromatography (0-30% EtOAc in hexane) to give the title compound (1.19 g, 3.09 mmol, 79% yield) as a yellow oil.

LC-MS (ESI): m/z (M+1): 386.8 (Method 3)

1H NMR (300 MHz, DMSO-d6) δ 9.30 (s, 1H), 8.28 (d, J=8.6 Hz, 2H), 7.60 (d, J=8.6 Hz, 2H), 7.43-7.22 (m, 4H), 7.07 (s, 1H), 6.23 (s, 1H), 6.13 (s, 1H), 5.83 (q, J=7.2 Hz, 1H), 1.42 (d, J=6.6 Hz, 3H).

The Intermediate in the following table was prepared from reagents reported below following similar procedures as for Intermediate P1.

| Intermediate | Structure & Name | Reagents | Analytical data |
|---|---|---|---|
| P2 | 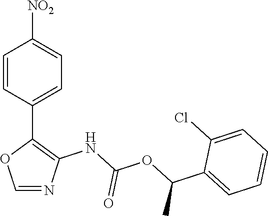 [SMILES: C[C@@H](OC(=O)Nc1ncoc1-c1ccc([N+](=O)[O-])cc1)c1ccccc1Cl] | 5-(4- nitrophenyl) oxazole-4- carboxylic acid | LC-MS (ESI): m/z (M + 1): 388.6 (Method 3) 1H NMR (300 MHz, DMSO- d6) δ 9.99 (s, 1H), 8.55 (s, 1H), 8.27 (d, J = 8.9 Hz, 2H), 7.80 (d, J = 8.9 Hz, 2H), 7.53 (bs, 1H), 7.50-7.31 (m, 3H), 5.99 (q, J = 6.5 Hz, 1H), 1.50 (d, J = 6.5 Hz, 3H). |
| | (R)-1-(2-chlorophenyl)ethyl | | |
| | (5-(4-nitrophenyl)oxazol-4- | | |
| | yl)carbamate | | |

### Step 2: (R)-1-(2-chlorophenyl)ethyl (1-(4-aminophenyl)-1H-pyrrol-2-yl)carbamate

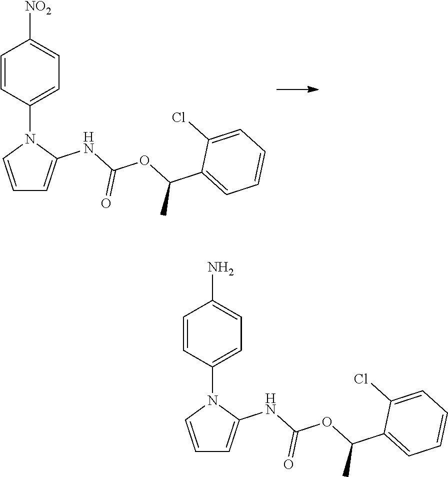 [SMILES: C[C@@H](OC(=O)Nc1cccn1-c1ccc([N+](=O)[O-])cc1)c1ccccc1Cl ; C[C@@H](OC(=O)Nc1cccn1-c1ccc(N)cc1)c1ccccc1Cl]

Ammonium formate (0.816 g, 15.257 mmol) was added to a solution of (R)-1-(2-chlorophenyl)ethyl (1-(4-nitrophenyl)-1H-pyrrol-2-yl)carbamate (intermediate P1, 1.09 g, 2.543 mmol) in ethanol (21.20 ml) and water (6.40 ml). The reaction mixture was stirred for 30 min. Then Zinc powder (0.997 g, 15.257 mmol) was added. The mixture was stirred overnight at RT, then was filtered through a celite pad, washing with ethanol (20 mL). The solvent was evaporated to dryness, water (50 mL) was added and the mixture was extracted with EtOAc (3×30 mL). Organic layers were dried over Na₂SO₄ and concentrated under reduced pressure. The crude product was purified by flash chromatography (0-60% ACN in water) to give the title compound (95 mg, 0.25 mmol, 10% yield) as a pink solid.

LC-MS (ESI): m/z (M+1): 388.6 (Method 4)

The Intermediate in the following table was prepared from reagents reported below following similar procedures as for Intermediate P3.

| Intermediate | Structure & Name | Reagents | Analytical data |
|---|---|---|---|
| P4 | 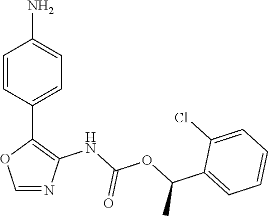 [SMILES: C[C@@H](OC(=O)Nc1ncoc1-c1ccc(N)cc1)c1ccccc1Cl] | P2 | LC-MS (ESI): m/z (M + 1): 358.2 (Method 4) 1H NMR (400 MHz, DMSO-d6, at 80° C.) δ 8.97 (s, 1H), 8.09 (s, 1H), 7.49 (d, J = 7.6 Hz, 1H), 7.46-7.41 (m, 1H), 7.41-7.34 (m, 2H), 7.32 (d, J = 8.0 Hz, 2H), 6.62 (d, J = 8.5 Hz, 2H), 6.02 (q, J = 6.6 Hz, 1H), 5.20 (s, 2H), 1.49 (d, J = 6.5 Hz, 3H). |
| | (R)-1-(2-chlorophenyl)ethyl | | |
| | (5-(4-aminophenyl)oxazol- | | |
| | 4-yl)carbamate | | |

### Step 3: methyl (1S,3S)-2,2-difluoro-3-((4-(2-((((R)-1-(o-tolyl)ethoxy)carbonyl)amino)-1H-pyrrol-1-yl)phenyl)carbamoyl)cyclopropane-1-carboxylate (Intermediate P5)

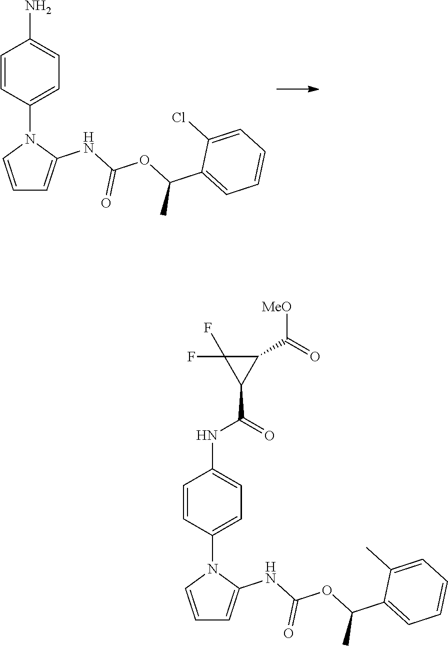 [SMILES: C[C@@H](OC(=O)Nc1cccn1-c1ccc(N)cc1)c1ccccc1Cl ; COC(=O)[C@@H]1[C@@H](C(=O)Nc2ccc(-n3cccc3NC(=O)O[C@H](C)c3ccccc3C)cc2)C1(F)F]

Intermediate P5 (130 mg, 0.251 mmol, 96. % yield) was obtained from (R)-1-(2-chlorophenyl)ethyl (1-(4-aminophenyl)-1H-pyrrol-2-yl)carbamate (Intermediate P3, 93.0 mg, 0.260 mmol) and (1S,3S)-2,2-difluoro-3-methoxycarbonyl-cyclopropanecarboxylic acid (Intermediate 2, 61 mg, 0.340 mmol) using a similar method as for Intermediate A17.

LC-MS (ESI): m/z (M+1): 518.3 (Method 1)

The Intermediate in the following table was prepared from reagents reported below following similar procedures as for Intermediate P5.

| Intermediate | Structure & Name | Reagents | Analytical data |
|---|---|---|---|
| P6 | 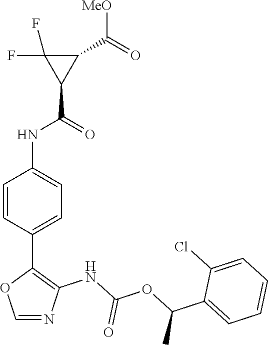 [SMILES: COC(=O)[C@@H]1[C@@H](C(=O)Nc2ccc(-c3ocnc3NC(=O)O[C@H](C)c3ccccc3Cl)cc2)C1(F)F] | P4 | LC-MS (ESI): m/z (M + 1): 520.3 (Method 1) |
| | methyl (1S,3S)-3-((4-(4-((((R)- | | |
| | 1-(2-chlorophenyl)ethoxy) | | |
| | carbonyl)amino)oxazol-5- | | |
| | yl)phenyl)carbamoyl)-2,2- | | |
| | difluorocyclopropane-1-carboxylate | | |

### Step 4: methyl (1S,3S)-2,2-difluoro-3-((4-(2-((((R)-1-(o-tolyl)ethoxy)carbonyl)amino)-1H-pyrrol-1-yl)phenyl)carbamoyl)cyclopropane-1-carbocylic acid (Compound 39)

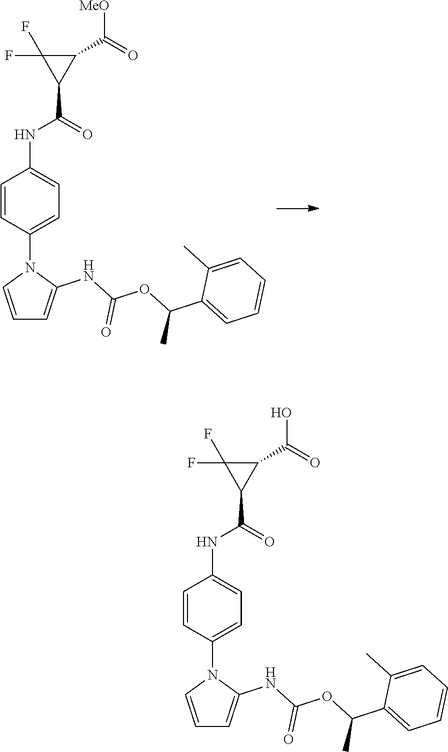 [SMILES: COC(=O)[C@@H]1[C@@H](C(=O)Nc2ccc(-n3cccc3NC(=O)O[C@H](C)c3ccccc3C)cc2)C1(F)F ; Cc1ccccc1[C@@H](C)OC(=O)Nc1cccn1-c1ccc(NC(=O)[C@@H]2[C@@H](C(=O)O)C2(F)F)cc1]

Compound 39 (94 mg, 0.187 mmol, 74% yield) was obtained from Intermediate P5 (136 mg, 0.750 mmol) using a similar method as for Compound 1, step 9.

LC-MS (ESI): m/z (M+1): 504.3 (Method 1)

The corresponding sodium salt of Compound 39 was obtained using a similar method as for Compound 8. Compound 39 was characterized as sodium salt.

LC-MS (ESI): m/z (M+1-Na+): 504.3 (Method 1)

1H NMR (500 MHz, DMSO-d6) δ ppm 10.58 (s, 1H), 8.59-9.24 (m, 1H), 7.63 (br d, J=7.8 Hz, 2H), 7.20 (br d, J=7.8 Hz, 2H), 6.79-6.97 (m, 1H), 6.72-7.47 (m, 4H), 6.09 (br s, 1H), 5.99 (br s, 1H), 5.60-5.91 (m, 1H), 3.08 (br dd, J=13.0, 7.8 Hz, 1H), 2.57-2.70 (m, 1H), 0.92-1.64 (m, 3H)

The Compound in the following table was prepared from reagents reported below following similar procedures as for Compound 39.

| Compound | Structure & Name | Reagents | Analytical data |
|---|---|---|---|
| 40 | 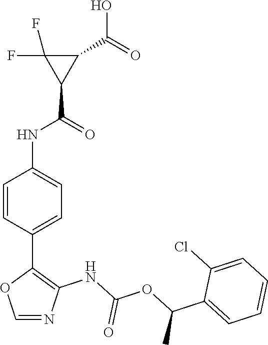 [SMILES: C[C@@H](OC(=O)Nc1ncoc1-c1ccc(NC(=O)[C@@H]2[C@@H](C(=O)O)C2(F)F)cc1)c1ccccc1Cl] | P6 | LC-MS (ESI): m/z (M + 1): 508.3 (Method 1) |
| | methyl (1S,3S)-3-((4-(4- | | |
| | ((((R)-1-(2-chlorophenyl) | | |
| | ethoxy)carbonyl)amino)oxazol- | | |
| | 5-yl)phenyl)carbamoyl)- | | |
| | 2,2-difluorocyclopropane- | | |
| | 1-carboxlic acid | | |

The corresponding sodium salt of Compound 40 was obtained using a similar method as for Compound 8. Compound 40 was characterized as sodium salt.

LC-MS (ESI): m/z (M+1-Na+): 508.3 (Method 1).

1H NMR (500 MHz, DMSO-d6) δ ppm 10.56 (br s, 1H), 9.54 (br s, 1H), 8.25 (br s, 1H), 7.13-7.74 (m, 8H), 5.99 (q, J=6.4 Hz, 1H), 3.05 (dd, J=13.2, 7.8 Hz, 1H), 2.55-2.72 (m, 1H), 1.47 (br s, 3H)

### Example 16

### Process synthesis of R)-1-(2-chlorophenyl)ethyl (5-(5-(2,2-difluoro-3-(5-thioxo-4,5-dihydro-1,2,4-oxadiazol-3-yl)cyclopropane-1-carboxamido)-6-methylpyridin-2-yl)-3-methylisoxazol-4-yl)carbamate, mix of Trans diastereomers (Compound 41)

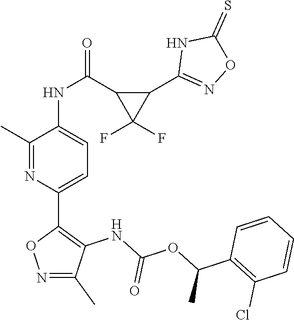 [SMILES: Cc1nc(-c2onc(C)c2NC(=O)O[C@H](C)c2ccccc2Cl)ccc1NC(=O)C1C(c2noc(=S)[nH]2)C1(F)F]

Step 1: (R)-1-(2-chlorophenyl)ethyl (5-(5-(2,2-difluoro-3-((Z)—N′-hydroxycarbamimidoyl) cyclopropane-1-carboxamido)-6-methylpyridin-2-yl)-3-methylisoxazol-4-yl)carbamate, mix of Trans diastereomers (Intermediate Q1)

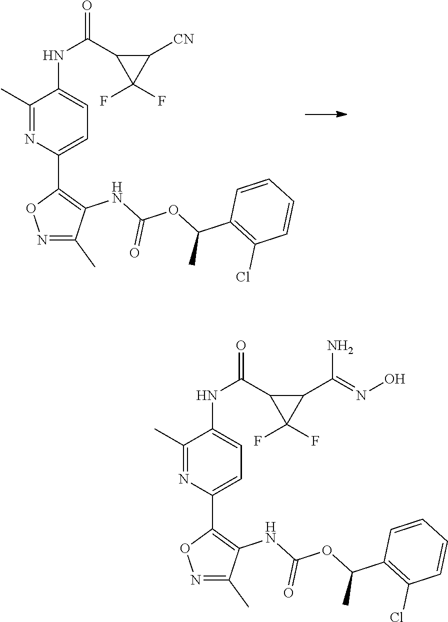 [SMILES: Cc1nc(-c2onc(C)c2NC(=O)O[C@H](C)c2ccccc2Cl)ccc1NC(=O)C1C(C#N)C1(F)F ; Cc1nc(-c2onc(C)c2NC(=O)O[C@H](C)c2ccccc2Cl)ccc1NC(=O)C1C(/C(N)=N/O)C1(F)F]

To a solution of hydroxylamine hydrochloride (134.7 mg, 1.94 mmol) in DMSO (4 mL), TEA (0.28 mL, 1.98 mmol) was added. The solid formed was filtered off and washed with THF. THE was evaporated under vacuum, then the filtrate was added with (R)-1-(2-chlorophenyl)ethyl (5-(5-(3-cyano-2,2-difluorocyclopropane-1-carboxamido)-6-methylpyridin-2-yl)-3-methylisoxazol-4-yl)carbamate, mix of Trans diastereomers (Intermediate E4, 200.0 mg, 0.390 mmol). The mixture was stirred at 75° C. for 5 h, then at RT overnight. Methyl tert-butyl ether and water were added, the phases were separated and the organic layer was collected, dried over Na₂SO₄ and evaporated to dryness to give the title compound (209 mg, 0.381 mmol, 98. % yield) as white solid that was used in the next step without further purification.

LC-MS (ESI): m/z (M+1): 549.2 (Method 1)

### Step 2: (R)-1-(2-chlorophenyl)ethyl (5-(5-(2,2-difluoro-3-(5-thioxo-4,5-dihydro-1,2,4-oxadiazol-3-yl)cyclopropane-1-carboxamido)-6-methylpyridin-2-yl)-3-methylisoxazol-4-yl)carbamate, mix of Trans diastereomers (Compound 41)

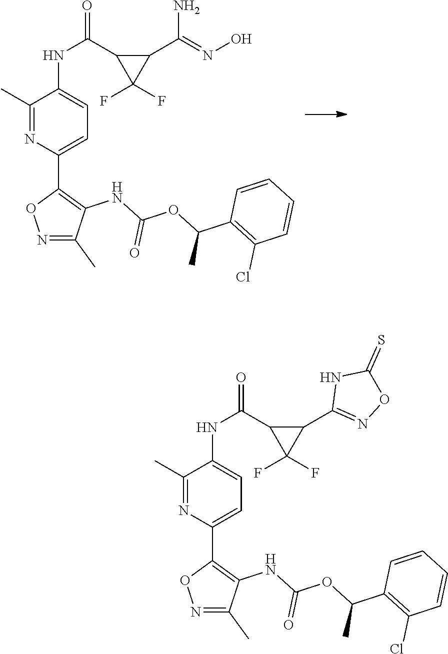 [SMILES: Cc1nc(-c2onc(C)c2NC(=O)O[C@H](C)c2ccccc2Cl)ccc1NC(=O)C1C(/C(N)=N/O)C1(F)F ; Cc1nc(-c2onc(C)c2NC(=O)O[C@H](C)c2ccccc2Cl)ccc1NC(=O)C1C(c2noc(=S)[nH]2)C1(F)F]

To a solution of (R)-1-(2-chlorophenyl)ethyl (5-(5-(2,2-difluoro-3-((Z)—N′-hydroxycarbamimidoyl)cyclopropane-1-carboxamido)-6-methylpyridin-2-yl)-3-methylisoxazol-4-yl)carbamate, mix of Trans diastereomers (Intermediate Q1, 209.0 mg, 0.380 mmol), in MeCN (4 mL), 2,3,4,6,7,8,9,10-octahydropyrimido[1,2-a]azepine (0.23 mL, 1.52 mmol) and bis(1-imidazolyl)methanethione (101.78 mg, 0.570 mmol) were added and the mixture was stirred at RT for 3h. MeCN was evaporated, then 0.6 M HCl and EtOAc were added and the aqueous layer was extracted again with EtOAc; collected organic phases were dried over Na₂SO₄, filtered and evaporated to dryness. The crude material was purified by reversed phase flash chromatography using a gradient of MeCN (+0.1% HCOOH) in water (+0.1% HCOOH) from 0% to 60% to give the target compound (17.5 mg, 0.030 mmol, 7.7% yield) as a brownish solid.

LC-MS (ESI): m/z (M+1): 591.0 (Method 1)

1H NMR (500 MHz, DMSO-d6) δ ppm 10.27 (br s, 1H), 9.27 (br s, 1H), 8.12 (d, J=8.4 Hz, 1H), 7.67 (d, J=8.4 Hz, 1H), 6.67-7.74 (m, 4H), 5.99 (br s, 1H), 3.76 (br dd, J=14.3, 7.9 Hz, 1H), 3.56 (br dd, J=11.2, 8.2 Hz, 1H), 2.48 (s, 3H), 2.17 (br s, 3H), 1.53 (br s, 3H)

### Example 17

### Process synthesis of (R)-1-(2-chlorophenyl)ethyl (5-(5-(2,2-difluoro-3-(5-oxo-4,5-dihydro-1,2,4-oxadiazol-3-yl)cyclopropane-1-carboxamido)-6-methylpyridin-2-yl)-3-methylisoxazol-4-yl)carbamate, mix of Trans diastereomers (Compound 42)

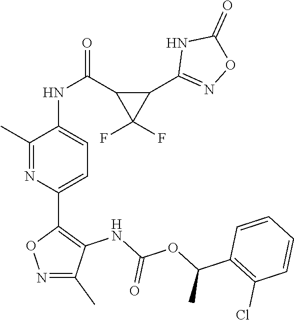 [SMILES: Cc1nc(-c2onc(C)c2NC(=O)O[C@H](C)c2ccccc2Cl)ccc1NC(=O)C1C(c2noc(=O)[nH]2)C1(F)F]

### Step 1: (R)-1-(2-chlorophenyl)ethyl (5-(5-(2,2-difluoro-3-(5-oxo-4,5-dihydro-1,2,4-oxadiazol-3-yl)cyclopropane-1-carboxamido)-6-methylpyridin-2-yl)-3-methylisoxazol-4-yl)carbamate, mix of Trans diastereomers (Compound 42)

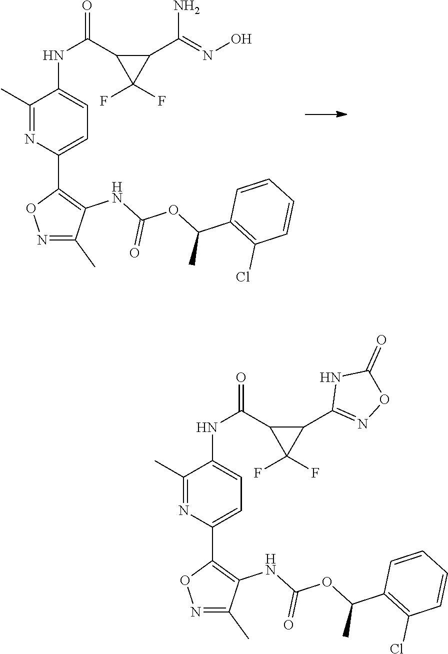 [SMILES: Cc1nc(-c2onc(C)c2NC(=O)O[C@H](C)c2ccccc2Cl)ccc1NC(=O)C1C(/C(N)=N/O)C1(F)F ; Cc1nc(-c2onc(C)c2NC(=O)O[C@H](C)c2ccccc2Cl)ccc1NC(=O)C1C(c2noc(=O)[nH]2)C1(F)F]

To a solution of (R)-1-(2-chlorophenyl)ethyl (5-(5-(2,2-difluoro-3-((Z)—N′-hydroxycarbamimidoyl)cyclopropane-1-carboxamido)-6-methylpyridin-2-yl)-3-methylisoxazol-4-yl)carbamate, mix of Trans diastereomers (Intermediate Q1, 190.0 mg, 0.350 mmol), in DMF (4 mL), pyridine (0.03 mL, 0.380 mmol) and 2-ethylhexyl carbonochloridate (0.01 mL, 0.030 mmol) were added at 0° C. and the mixture was stirred at 0° C. for 1h. The mixture was diluted with water and EtOAc and the phases were separated. Aqueous phase was extracted with EtOAc and collected organic phases were dried over Na₂SO₄, filtered and evaporated to give a residue that was dissolved in 5 ml of xylene and heated at reflux overnight. The solvent was evaporated to give the crude material that was purified by reversed phase flash chromatography using a gradient of MeCN (+0.1% HCOOH) in water (+0.1% HCOOH) from 0% to 50% to give the title compound (28.7 mg, 0.050 mmol, 14% yield) as white solid.

LC-MS (ESI): m/z (M+1): 573.1 (Method 1)

1H NMR (500 MHz, DMSO-d6) δ ppm 12.86 (br s, 1H), 10.29 (br s, 1H), 9.49 (s, 1H), 8.12 (d, J=8.5 Hz, 1H), 7.67 (d, J=8.4 Hz, 1H), 6.78-7.64 (m, 4H), 5.79-6.20 (m, 1H), 3.68-3.80 (m, 1H), 3.54 (br dd, J=10.9, 8.3 Hz, 1H), 2.47 (s, 3H), 2.11-2.24 (m, 3H), 1.40 (br s, 3H)

### Example 18

### Process synthesis of (R)-1-(2-chlorophenyl)ethyl (5-(5-(2,2-difluoro-3-(5-oxo-4,5-dihydro-1,2,4-thiadiazol-3-yl)cyclopropane-1-carboxamido)-6-methylpyridin-2-yl)-3-methylisoxazol-4-yl)carbamate, mix of Trans diastereomers (Compound 43)

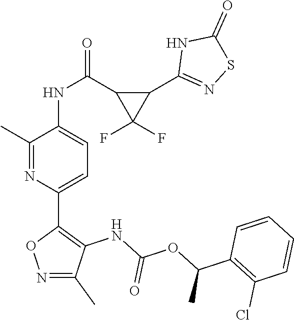 [SMILES: Cc1nc(-c2onc(C)c2NC(=O)O[C@H](C)c2ccccc2Cl)ccc1NC(=O)C1C(c2nsc(=O)[nH]2)C1(F)F]

### Step 1: (R)-1-(2-chlorophenyl)ethyl (5-(5-(2,2-difluoro-3-(5-oxo-4,5-dihydro-1,2,4-thiadiazol-3-yl)cyclopropane-1-carboxamido)-6-methylpyridin-2-yl)-3-methylisoxazol-4-yl)carbamate, mix of Trans diastereomers (Compound 43)

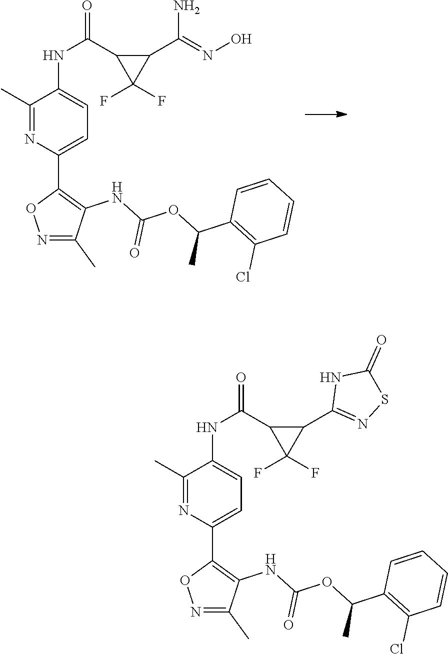 [SMILES: Cc1nc(-c2onc(C)c2NC(=O)O[C@H](C)c2ccccc2Cl)ccc1NC(=O)C1C(/C(N)=N/O)C1(F)F ; Cc1nc(-c2onc(C)c2NC(=O)O[C@H](C)c2ccccc2Cl)ccc1NC(=O)C1C(c2nsc(=O)[nH]2)C1(F)F]

A mixture of (R)-1-(2-chlorophenyl)ethyl (5-(5-(2,2-difluoro-3-((Z)—N′-hydroxycarbamimidoyl)cyclopropane-1-carboxamido)-6-methylpyridin-2-yl)-3-methylisoxazol-4-yl)carbamate, mix of Trans diastereomers (Intermediate Q1, 200.0 mg, 0.360 mmol), and bis(1-imidazolyl)methanethione (97.39 mg, 0.550 mmol) in THE (0.760 mL) was stirred at RT for 1h, then was diluted with water and extracted with EtOAc. The organic phase was washed with water, dried over Na₂SO₄, filtered and the filtrate concentrated under reduced pressure. The residue was dissolved in THE (0.30 mL) then boron trifluoride diethyl etherate (0.22 mL, 1.82 mmol) was added at 0° C., and the mixture was left at RT for 1h. The mixture was diluted with water and extracted with EtOAc. The organic layer was washed with 1M HCl, dried over Na₂SO₄, filtered and concentrated under reduced pressure. The crude was purified by reversed phase flash chromatography using a gradient of MeCN in water modified with 0.1% of HCOOH from 0% to 50% to afford the title compound (5.9 mg, 0.010 mmol, 3% yield) as a white solid.

LC-MS (ESI): m/z (M+1): 591.3 (Method 1)

1H NMR (400 MHz, DMSO-d6) δ ppm 12.82 (br s, 1H), 10.20 (br s, 1H), 9.27 (br s, 1H), 7.99-8.37 (m, 1H), 7.64 (d, J=8.6 Hz, 1H), 7.12-7.75 (m, 4H), 5.83-6.12 (m, 1H), 3.74 (br dd, J=14.0, 7.8 Hz, 1H), 3.21-3.28 (m, 1H), 2.47 (br s, 3H), 2.17 (s, 3H), 1.52 (br s, 3H)

### Example 19

### Process synthesis of (R)-1-(2-chlorophenyl)ethyl (5-(5-((1S,3S)-2,2-difluoro-3-(4-methyl-5-thioxo-4,5-dihydro-1H-1,2,4-triazol-3-yl)cyclopropane-1-carboxamido)-6-methylpyridin-2-yl)-3-methylisoxazol-4-yl)carbamate (Compound 44)

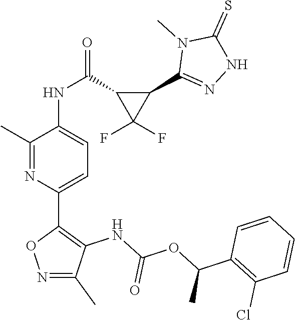 [SMILES: Cc1nc(-c2onc(C)c2NC(=O)O[C@H](C)c2ccccc2Cl)ccc1NC(=O)[C@@H]1[C@@H](c2n[nH]c(=S)n2C)C1(F)F]

### Step 1: (R)-1-(2-chlorophenyl)ethyl (5-(5-((1S,3S)-2,2-difluoro-3-(hydrazinecarbonyl) cyclopropane-1-carboxamido)-6-methylpyridin-2-yl)-3-methylisoxazol-4-yl)carbamate (Intermediate R1)

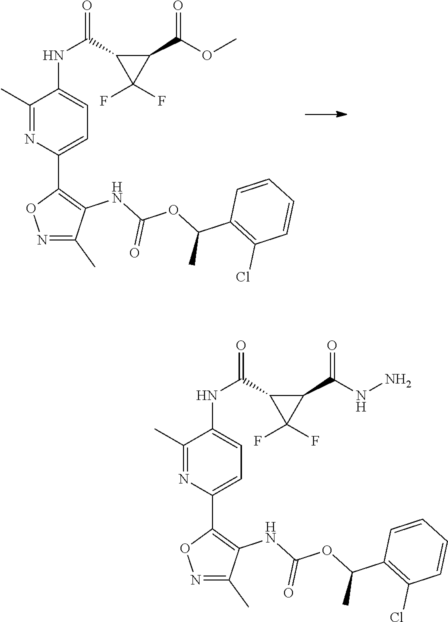 [SMILES: COC(=O)[C@@H]1[C@@H](C(=O)Nc2ccc(-c3onc(C)c3NC(=O)O[C@H](C)c3ccccc3Cl)nc2C)C1(F)F ; Cc1nc(-c2onc(C)c2NC(=O)O[C@H](C)c2ccccc2Cl)ccc1NC(=O)[C@@H]1[C@@H](C(=O)NN)C1(F)F]

To a solution of methyl (1S,3S)-3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylate (Intermediate A17, 300.0 mg, 0.550 mmol) in THE (6 mL), hydrazine hydrate (0.41 mL, 5.47 mmol) was added and the mixture was refluxed for 3 h. The solvent was evaporated to give the title compound (450 mg, crude) that was used in the next step without further purification.

LC-MS (ESI): m/z (M+1): 549.2 (Method 1)

### Step 2: (R)-1-(2-chlorophenyl)ethyl (5-(5-((1S,3S)-2,2-difluoro-3-(4-methyl-5-thioxo-4,5-dihydro-1H-1,2,4-triazol-3-yl)cyclopropane-1-carboxamido)-6-methylpyridin-2-yl)-3-methylisoxazol-4-yl)carbamate (Compound 44)

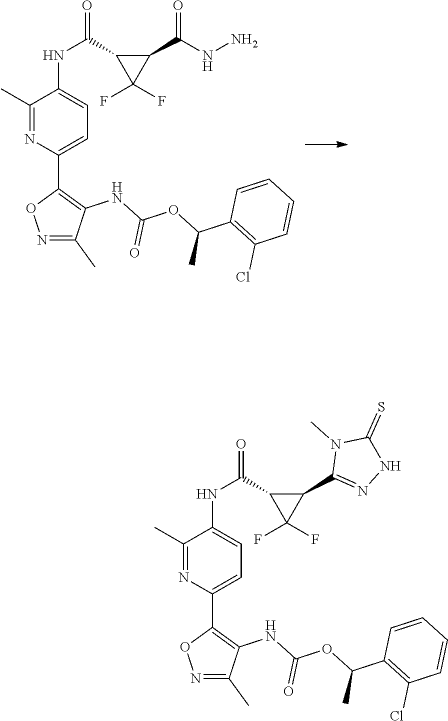 [SMILES: Cc1nc(-c2onc(C)c2NC(=O)O[C@H](C)c2ccccc2Cl)ccc1NC(=O)[C@@H]1[C@@H](C(=O)NN)C1(F)F ; Cc1nc(-c2onc(C)c2NC(=O)O[C@H](C)c2ccccc2Cl)ccc1NC(=O)[C@@H]1[C@@H](c2n[nH]c(=S)n2C)C1(F)F]

To a solution (R)-1-(2-chlorophenyl)ethyl (5-(5-((1S,3S)-2,2-difluoro-3-(hydrazinecarbonyl)cyclopropane-1-carboxamido)-6-methylpyridin-2-yl)-3-methylisoxazol-4-yl)carbamate (Intermediate R1, 150.0 mg, 0.270 mmol) in THE (4 mL), TEA (0.08 mL, 0.550 mmol) was added, followed by methylimino(sulfanylidene)methane (20 mg, 0.270 mmol). The mixture was stirred at RT overnight. The solvent was evaporated, NaOH 1 M was added (4 ml) and the mixture was stirred at 50° C. for 3 h. The mixture was acidified with 1M HCl, then water and EtOAc were added. The layers were separated and the aqueous phase extracted with EtOAc; collected organic phases were dried over Na₂SO₄, filtered and evaporated to dryness. The crude was purified by reversed phase flash chromatography using a gradient of MeCN (+0.1% HCOOH) in water (+0.1% HCOOH) from 0% to 50% to give the title compound (75.6 mg, 0.125 mmol, 45.8% yield) as an off-white solid.

LC-MS (ESI): m/z (M+1): 604.2 (Method 1)

1H NMR (400 MHz, DMSO-d6) δ ppm 13.53-14.07 (m, 1H), 10.28 (br s, 1H), 9.02-9.50 (m, 1H), 8.15 (d, J=8.3 Hz, 1H), 7.67 (d, J=8.3 Hz, 1H), 7.06-7.82 (m, 4H), 5.83-6.14 (m, 1H), 3.68-3.97 (m, 2H), 3.52 (s, 3H), 2.39-2.54 (m, 3H), 2.18 (s, 3H), 1.24-1.76 (m, 3H)

### Pharmacological Activity of the Compounds of the Invention

In vitro Assays

The effectiveness of compounds of the present invention as LPA1 antagonist can be determined at the human recombinant LPA1 expressed in CHO cells, using a FLIPR assay in 384 well format.

CHO-hLPA1 cell lines are cultured in a humidified incubator at 5% CO2 in DMEM/F-12 (1:1) MIXTURE with 2 mM Glutamax, supplemented with 10% of Foetal Bovine Serum, 1 mM Sodium Pyruvate, 11 mM Hepes and 1× Penicillin/Streptomycin. CHO hLPA1 cells are seeded into black walled clear-bottom 384-well plates (#781091, Greiner Bio-One GmbH) at a density of 7,500 cells per well in 50 μl culture media and grown overnight in a 37° C. humidified CO2-incubator. Serial dilutions (1:3 or 1:4, 11 points CRC) of compounds are performed in 100% DMSO at 200× the final concentration. The compounds are diluted 1:50 prior to the experiment with Assay Buffer (20 mM HEPES, 145 mM NaCl, 5 mM KCl, 5.5 mM glucose, 1 mM MgCl2 and 2 mM CaCl2), pH 7.4 containing 0.01% Pluronic F-127) to obtain a solution corresponding to 5-fold the final concentration in the assay (4×, 2% DMSO). The final concentration of DMSO in the assay will be 0.5% in each well. Medium is removed by aspiration and cells are then incubated with 30 μl of a loading solution containing 5 μM of the cytoplasmic Ca2+ indicator Cal-520 AM in Assay Buffer containing 2.5 mM probenecid for 30 min at 37° C. incubator (cell loading). The loaded cell plates are transferred into the FLIPR instrument and calcium responses are monitored during the on-line addition protocols. For testing of compounds, after the cell loading, 10 μl/well of 4× antagonists' solution was added onto the cells. After 30 min pre-incubation (at 37° C.), 10 μl/well of 5× concentrated LPA EC80 was added and Ca2+ mobilization responses was followed during the on-line addition protocol. Intracellular peak fluorescence values subtracted by baseline fluorescence are exported and analysed to determine IC₅₀ values, respectively. The calcium response is expressed as percentage of the maximal inhibition of the EC80 agonist response.

The raw data obtained in unstimulated controls (DMSO, no LPA) are set as “100% inhibition”, while the raw data obtained in negative controls, i.e. in the absence of compounds and stimulating with LPA EC80, are set as “0% inhibition”.

The raw data (peak height expressed as relative fluorescence units) are normalized and transformed into “percent of inhibition”. Curve fitting and pIC₅₀ (−Log IC₅₀) estimations are carried out using a four-parameter logistic model using XLfit Software.

The results for individual compounds are provided below in Table 4 and are expressed as range of activity.

TABLE 4

| Example No. | LPA1 IC₅₀ |
|---|---|
| 8, 22, 30, 36, 38, 39, 40 | + |
| 9, 16, 37, 44 | ++ |
| 1, 2, 4, 5, 6, 11, 12, 13, 14, 17, 18, 19, 20, 21, 23, 24, | +++ |
| 25, 27, 31, 32, 33, 34, 35, 41,42, 43 | |

wherein the compounds are classified in term of potency with respect to their inhibitory activity on LPA1 receptors according to the following classification criterion:
+: LPA1 IC₅₀ comprised between about 200 nM and 100 nM
++: LPA1 IC₅₀ comprised between 100 nM and 50 nM
+++: LPA1 IC₅₀ less than 50 nM

As it can be appreciated, all the compounds of Table 4 show an antagonist activity on LPA1 receptor. In fact, it can be recognized that the symbol+indicate a good and sufficient level of activity, which can be even increased up to +++, thus confirming the high activity receptor LPA1 of the compounds of the invention.

### Permeability

The permeability of the compounds of the present invention was evaluated performing the assay on Caco-2 cells monolayers (human colon carcinoma cell line) by measuring the transport of compound (absorption and secretion) in both directions: apical to basolateral direction (AtoB) and basolateral to apical (BtoA) with and without P-gp inhibitor (Elacridar).

The Caco-2 cells was provided by ATCC (American Type Culture Collection, product code HTB 37) and they have been validated in house for their use in permeability study.

Caco 2 cells were kept in culture using MEM containing 20% FBS, 1 mM Sodium Pyruvate, 2 mM Glutamine and Pen/Strep. Caco 2 cells were seeded onto microporous PET (Polyethylene Terephthalate) membranes in HTS 96 Multiwell Insert plates, at a density of 3.9×104 cells/cm2 (5,600 cells/well; 100 μL/well) using a different growth medium (Dulbecco's modified Eagle's medium (DMEM) with Glutamax, 10% FBS) and incubated for 21-28 days at 37° C. 5% CO2. Medium was changed twice per week.

Monolayer integrity of Caco-2 cells was evaluated before and after the experiment with TEER measurements using a Millicell ERS meter (Electrical Resistance System, from Millipore) and after the experiment using the paracellular permeability marker Lucifer yellow.

Moreover, reference controls for high and low permeability (i.e. metoprolol, atenolol) and for P-gp substrate (i.e. Digoxin) were also tested in parallel with test compounds.

Test compound transport was measured in two directions (apical to basolateral [AtoB] and basolateral to apical [BtoA]) in HBSS (Hank's Balanced Salt Solution) transport buffer at pH 7.4 (n=3). This assay was also run in presence of a P-gp inhibitor, GF120918, to investigate any potential interaction of the testing compound with P-gp.

Test compounds were investigated at a single concentration (i.e. 10 μM or other concentration) at one timepoint (i.e. 60 minutes or other timepoint).

The sample solution was prepared dissolving test compound in DMSO at the concentration of 10 mM and then diluted in the Assay Buffer (Hank's Balance Salt Solution) warmed at 37° C. before use, to give the 10 μM Compound working solution with and without 10 μM Elacridar. These working solutions were added to donor compartment (apical for AtoB direction and basolateral for BtoA direction) and Assay Buffer (Hank's Balance Salt Solution) to the receiver compartment (basolateral for AtoB direction and apical for BtoA direction). The plate was incubated at 37° C. for 60 min, all incubation were conducted in triplicates. At the end of incubation, samples from donor and receiver compartments were collected for HPLC-MS/MS analysis.

The permeability coefficients (Papp) in both directions: apical to basolateral (AtoB) and basolateral to apical (BtoA) with and without P-gp inhibitor (Elacridar) was calculated in nm/sec, using the following equation:

$$Papp = \frac{Cr \cdot Vr}{t \cdot A \cdot C 0} \cdot 10000000 [ \frac{nm}{sec} ]$$

where:
Cr=measured concentration in the receiver well at the time t (expressed as IS ratio)
Vr=volume of the receiver well (ml)
t=time (sec)
A=membrane surface area (cm²)
C0=initial donor concentration
Passive Papp is considered Papp AtoB with P-gp inhibitor Elacridar.

The results for individual compounds are provided below in Table 5.

TABLE 5

| | Passive Permeability |
|---|---|
| Example No. | nm/sec |
| 1, 2, 4, 5, 6, 8, 9, 11, 12, 13, 14, 16, 17, 18, | ≤5 nm/sec |
| 19, 20, 21, 23, 24, 25, 27, 31, 32, 33, 34, 35, | |
| 36, 37, 39, 40, 41, 42, 43, 44 | |

### Comparative Example A

### Single Diastereomer 1 of Trans-3-((4-(4-((((R)-1-(2-chlorophenyl)ethoxy) carbonyl)amino)-3-methylisoxazol-5-yl)phenyl)carbamoyl)-2,2-difluorocyclopropane-1-carboxylic acid

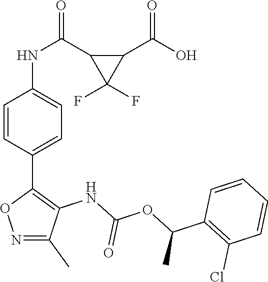 [SMILES: Cc1noc(-c2ccc(NC(=O)C3C(C(=O)O)C3(F)F)cc2)c1NC(=O)O[C@H](C)c1ccccc1Cl]

The activity of comparative Example A as has been tested in the in vitro assay for the determination of activity on LPA1 receptor as described above along with the permeability assays.

Differently from the compounds of formula (I) of the present invention, the comparative Example A shows a passive permeability about 18 and thus not suitable for administration by inhalation.

1: A compound of formula (I) 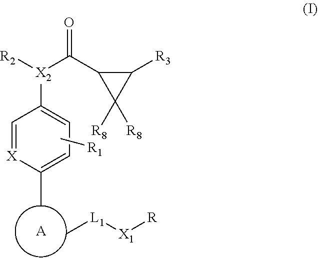
wherein X is —CH— or N,
X₁ is —NH— or —O—,
X₂ is —N— or —CH₂—N—,
L₁ is —(CH₂)ₙ
n is an integer between 0 and 3;
A is selected from the group consisting of 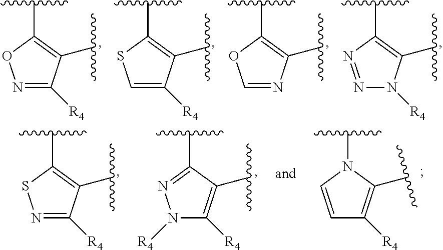
R₁ is H or (C₁-C₄)alkyl;
R₂ is H or (C₁-C₄)alkyl;
R₃ is —C(O)OR₂ or heteroaryl wherein said heteroaryl may be optionally substituted by oxo or thioxo,
R₄ is H or (C₁-C₄)alkyl,
R is selected from the group consisting of —C(O)O(C₁-C₅)alkyl-R₅, —C(O)NR₆R₇, and heteroaryl, wherein said heteroaryl may be optionally substituted by one or more groups selected from the group consisting of —OR₂, (C₁-C₄)alkyl, (C₁-C₄)haloalkyl, (C₃-C₆)cycloalkyl, aryl, and heteroaryl;
R₅ is H or selected from the group consisting of —(C₁-C₄)alkyl and aryl, wherein said aryl is optionally substituted by one or more groups selected from the group consisting of —(C₁-C₄)alkyl and halo;
R₆ and R₇ are at each occurrence independently H or selected from the group consisting of (C₁-C₄)alkyl, (C₃-C₆)cycloalkyl, (C₁-C₆)haloalkyl and halo, and
R₈ is H or halo,
with the proviso that when A is 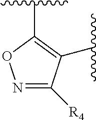
and X is CH, R₂ is (C₁-C₄)alkyl or R₃ is heteroaryl, or n is 1 or greater than 1.

2: The compound according to claim 1, wherein X is —N, represented by formula (Ia) 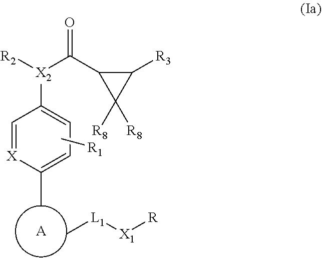
wherein
X₁ is —NH— or —O—,
X₂ is —N— or —CH₂—N—,
L₁ is —(CH₂)ₙ
n is an integer between 0 and 3;
A is selected from the group consisting of 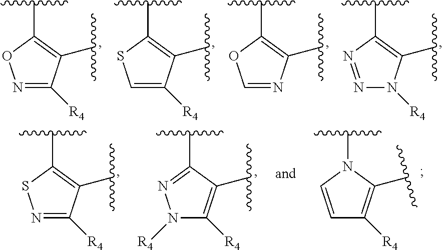
R₁ is H or (C₁-C₄)alkyl;
R₂ is H or (C₁-C₄)alkyl;
R₃ is —C(O)OR₂ or heteroaryl wherein said heteroaryl may be optionally substituted by oxo or thioxo,
R₄ is H or (C₁-C₄)alkyl,
R is selected from the group consisting of —C(O)O(C₁-C₅)alkyl-R₅, —C(O)NR₆R₇, and heteroaryl, wherein said heteroaryl may be optionally substituted by one or more groups selected from the group consisting of —OR₂, (C₁-C₄)alkyl, (C₁-C₄)haloalkyl, (C₃-C₆)cycloalkyl, aryl, and heteroaryl;
R₅ is H or selected from the group consisting of —(C₁-C₄)alkyl and aryl, wherein said aryl is optionally substituted by one or more groups selected from the group consisting of —(C₁-C₄)alkyl and halo;
R₆ and R₇ are at each occurrence independently H or selected from the group consisting of (C₁-C₄)alkyl, (C₃-C₆)cycloalkyl, (C₁-C₆)haloalkyl and halo, and
R₈ is H or halo.

3: The compound according to claim 1, wherein X is —N, A is 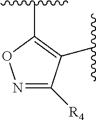
R₄ represented by the formula (Ib) 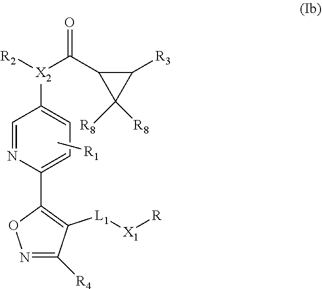
wherein
X₁ is —NH— or —O—,
X₂ is —N— or —CH₂—N—,
L₁ is —(CH₂)ₙ
n is an integer between 0 and 3;
R₁ is H or (C₁-C₄)alkyl;
R₂ is H or (C₁-C₄)alkyl;
R₃ is —C(O)OR₂ or heteroaryl wherein said heteroaryl may be optionally substituted by oxo or thioxo,
R₄ is H or (C₁-C₄)alkyl,
R is selected from the group consisting of —C(O)O(C₁-C₅)alkyl-R₅, —C(O)NR₆R₇, and heteroaryl, wherein said heteroaryl may be optionally substituted by one or more groups selected from the group consisting of —OR₂, (C₁-C₄)alkyl, (C₁-C₄)haloalkyl, (C₃-C₆)cycloalkyl, aryl, and heteroaryl;
R₅ is H or selected from the group consisting of —(C₁-C₄)alkyl and aryl, wherein said aryl is optionally substituted by one or more groups selected from the group consisting of —(C₁-C₄)alkyl and halo;
R₆ and R₇ are at each occurrence independently H or selected from the group consisting of (C₁-C₄)alkyl, (C₃-C₆)cycloalkyl, (C₁-C₆)haloalkyl and halo, and
R₈ is H or halo.

4: The compound according to claim 1, selected from the group consisting of:
(1S,3S)-3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylic acid,
(1R,3R)-3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylic acid,
3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid, mix of Trans diastereomers,
3-((6-(4-((((R)-1-(2-chlorophenyl)ethoxy)carbonyl)amino)-3-methylisoxazol-5-yl)-2-methylpyridin-3-yl)carbamoyl)-2,2-difluorocyclopropane-1-carboxylic acid, mix of Trans diastereomers,
Single Diastereomer 1 of Trans-3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid,
Single Diastereomer 2 of Trans-3-((6-(4-((((R)-1-(2-chlorophenyl)ethoxy)carbonyl)amino)-3-methylisoxazol-5-yl)pyridin-3-yl)carbamoyl)-2,2-difluorocyclopropane-1-carboxylic acid,
3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid, mix of Trans diastereomers,
Single Diastereomer 1 of Trans 3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid,
Single Diastereomer 2 of Trans 3-((4-(4-((((R)-1-(2-chlorophenyl)ethoxy)carbonyl)amino)-3-methylisoxazol-5-yl)phenyl)(methyl)carbamoyl)-2,2-difluorocyclopropane-1-carboxylic acid,
2,2-difluoro-3-((6-(4-((6-isopropoxypyrazin-2-yl)amino)-3-methylisoxazol-5-yl)-2-methylpyridin-3-yl)carbamoyl)cyclopropane-1-carboxylic acid, mix of Trans diastereomers,
Single Enantiomer 2 of Trans-2,2-difluoro-3-((6-(4-((6-isopropoxypyrazin-2-yl)amino)-3-methylisoxazol-5-yl)-2-methylpyridin-3-yl)carbamoyl)cyclopropane-1-carboxylic acid,
(1S,3S)-2,2-difluoro-3-((2-methyl-6-(3-methyl-4-((6-phenylpyrazin-2-yl)amino) isoxazol-5-yl)pyridin-3-yl)carbamoyl)cyclopropane-1-carboxylic acid
(R)-1-(2-chlorophenyl)ethyl (5-(4-(2,2-difluoro-3-(1H-tetrazol-5-yl)cyclopropane-1-carboxamido)phenyl)-3-methylisoxazol-4-yl)carbamate, mix of Trans diastereomers,
(R)-1-(2-chlorophenyl)ethyl (5-(5-(2,2-difluoro-3-(1H-tetrazol-5-yl)cyclopropane-1-carboxamido)-6-methylpyridin-2-yl)-3-methylisoxazol-4-yl)carbamate, mix of Trans diastereomers,
3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic acid, mix of Cis and Trans diastereomers,
3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic acid, mix of Cis diastereomers,
3-(((6-(4-((((R)-1-(2-chlorophenyl)ethoxy)carbonyl)amino)-3-methylisoxazol-5-yl)-2-methylpyridin-3-yl)methyl)carbamoyl)-2,2-difluorocyclopropane-1-carboxylic acid, mix of Trans diastereomers,
3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid, mix of Trans diastereomers,
Single Enantiomer 1 of Trans-3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid,
Single Enantiomer 2 of Trans-3-((6-(4-(((4-cyclohexylpyrimidin-2-yl)amino)methyl)-3-methylisoxazol-5-yl)-2-methylpyridin-3-yl)carbamoyl)-2,2-difluorocyclopropane-1-carboxylic acid,
(1S,3S)-2,2-difluoro-3-((2-methyl-6-(3-methyl-4-(((4-(thiophen-3-yl)pyrimidin-2-yl)amino)methyl)isoxazol-5-yl)pyridin-3-yl)carbamoyl)cyclopropane-1-carboxylic acid,
(1S,3S)-2,2-difluoro-3-((6-(4-(((4-(furan-2-yl)pyrimidin-2-yl)amino)methyl)-3-methylisoxazol-5-yl)-2-methylpyridin-3-yl)carbamoyl)cyclopropane-1-carboxylic acid,
(1S,3S)-2,2-difluoro-3-((2-methyl-6-(3-methyl-4-(((4-(pyrazin-2-yl)pyrimidin-2-yl)amino)methyl)isoxazol-5-yl)pyridin-3-yl)carbamoyl)cyclopropane-1-carboxylic acid,
(1S,3S)-2,2-difluoro-3-((2-methyl-6-(3-methyl-4-(((4-(thiophen-2-yl)pyrimidin-2-yl)amino)methyl)isoxazol-5-yl)pyridin-3-yl)carbamoyl)cyclopropane-1-carboxylic acid,
(1S,3S)-2,2-difluoro-3-((2-methyl-6-(3-methyl-4-(((4-(pyridin-3-yl)pyrimidin-2-yl)amino)methyl)isoxazol-5-yl)pyridin-3-yl)carbamoyl)cyclopropane-1-carboxylic acid,
3-((4-(3-((((R)-1-(2-chlorophenyl)ethoxy)carbonyl)amino)thiophen-2-yl)phenyl)carbamoyl)-2,2-difluorocyclopropane-1-carboxylic acid, mix of Trans diastereomers,
Single Diastereomer 2 of Trans-3-((4-(3-((((R)-1-(2-chlorophenyl)ethoxy)carbonyl)amino)thiophen-2-yl)phenyl)carbamoyl)-2,2-difluorocyclopropane-1-carboxylic acid,
3-((6-(3-((((R)-1-(2-chlorophenyl)ethoxy)carbonyl)amino)thiophen-2-yl)-2-methylpyridin-3-yl)carbamoyl)-2,2-difluorocyclopropane-1-carboxylic acid, mix of Trans diastereomers,
3-((6-(4-((((R)-1-(2-chlorophenyl)ethoxy)carbonyl)amino)-3-methylisothiazol-5-yl)-2-methylpyridin-3-yl)carbamoyl)-2,2-difluorocyclopropane-1-carboxylic acid, mix of Trans diastereomers,
Single Diastereomer 2 of Trans-3-((6-(3-((((R)-1-(2-chlorophenyl)ethoxy)carbonyl)amino)thiophen-2-yl)-2-methylpyridin-3-yl)carbamoyl)-2,2-difluorocyclopropane-1-carboxylic acid,
Single Diastereomer, 1 of Trans-3-((6-(4-((((R)-1-(2-chlorophenyl)ethoxy)carbonyl)amino)-3-methylisothiazol-5-yl)-2-methylpyridin-3-yl)carbamoyl)-2,2-difluorocyclopropane-1-carboxylic acid,
3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid, mix of Trans diastereomers,
Single Diastereomer 1 of Trans-3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid,
Single Diastereomer 2 of Trans-3-((6-(5-((((R)-1-(2-chlorophenyl)ethoxy)carbonyl)amino)-1-methyl-1H-1,2,3-triazol-4-yl)-2-methylpyridin-3-yl)carbamoyl)-2,2-difluorocyclopropane-1-carboxylic acid,
(1S,3S)-3-((4-(5-((((R)-1-(2-chlorophenyl)ethoxy)carbonyl)amino)-1-methyl-1H-1,2,3-triazol-4-yl)phenyl)carbamoyl)-2,2-difluorocyclopropane-1-carboxylic acid,
3-((4-(4-(((cyclopentyl(methyl)carbamoyl)oxy)methyl)-3-methylisoxazol-5-yl)phenyl)carbamoyl)-2,2-difluorocyclopropane-1-carboxylic acid, mix of Trans diastereomers,
3-((6-(4-(((cyclopentyl(methyl)carbamoyl)oxy)methyl)-3-methylisoxazol-5-yl)-2-methylpyridin-3-yl)carbamoyl)-2,2-difluorocyclopropane-1-carboxylic acid, mix of Trans diastereomers,
(1S,3S)-3-((4-(4-((((R)-1-(2-chlorophenyl)ethoxy)carbonyl)amino)-1-methyl-1H-pyrazol-3-yl)phenyl)carbamoyl)-2,2-difluorocyclopropane-1-carboxylic acid,
(1S,3S)-3-((4-(2-((((R)-1-(2-chlorophenyl)ethoxy)carbonyl)amino)-1H-pyrrol-1-yl)phenyl)carbamoyl)-2,2-difluorocyclopropane-1-carboxylic acid,
(1S,3S)-3-((4-(4-((((R)-1-(2-chlorophenyl)ethoxy)carbonyl)amino)oxazol-5-yl)phenyl)carbamoyl)-2,2-difluorocyclopropane-1-carboxylic acid,
(R)-1-(2-chlorophenyl)ethyl (5-(5-(2,2-difluoro-3-(5-thioxo-4,5-dihydro-1,2,4-oxadiazol-3-yl)cyclopropane-1-carboxamido)-6-methylpyridin-2-yl)-3-methylisoxazol-4-yl)carbamate, mix of Trans diastereomers,
(R)-1-(2-chlorophenyl)ethyl (5-(5-(2,2-difluoro-3-(5-oxo-4,5-dihydro-1,2,4-oxadiazol-3-yl)cyclopropane-1-carboxamido)-6-methylpyridin-2-yl)-3-methylisoxazol-4-yl)carbamate, mix of Trans diastereomers,
(R)-1-(2-chlorophenyl)ethyl (5-(5-(2,2-difluoro-3-(5-oxo-4,5-dihydro-1,2,4-thiadiazol-3-yl)cyclopropane-1-carboxamido)-6-methylpyridin-2-yl)-3-methylisoxazol-4-yl)carbamate, mix of Trans diastereomers, and
(R)-1-(2-chlorophenyl)ethyl (5-(5-((1S,3S)-2,2-difluoro-3-(4-methyl-5-thioxo-4,5-dihydro-1H-1,2,4-triazol-3-yl)cyclopropane-1-carboxamido)-6-methylpyridin-2-yl)-3-methylisoxazol-4-yl)carbamate.

5: A pharmaceutical composition comprising the compound according to claim 1, in admixture with one or more pharmaceutically acceptable carriers or excipients.

6: The pharmaceutical composition according to claim 5, which is formulated for administration by inhalation.

7. (canceled)

8: A method for treating a disease, disorder, or
condition associated with dysregulation of lysophosphatidic acid receptor 1 (LPA1) comprising administering the compound of claim 1 to a subject in need thereof.

9: A method of treating fibrosis and/or a disease, disorder, or condition that involves fibrosis, comprising administering the compound of claim 1 to a subject in need thereof.

10: The method according to claim 9, wherein the fibrosis is at least one selected from the group consisting of pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

11: The method according to claim 10, wherein the fibrosis comprises idiopathic pulmonary fibrosis (IPF).
